(19) United States
(12) Patent Application Publication
(10) Pub. No.: US 2022/0106391 A1
(43) Pub. Date: Apr. 7, 2022

(54) ANTI-NGF ANTIBODIES AND METHODS OF USE THEREOF

(71) Applicant: Zoetis Services LLC, Parsippany, NJ (US)

(72) Inventors: Lisa Marie Bergeron, Boulder, CO (US); Catherine J. Strietzel, Kalamazoo, MI (US); Gary F. Bammert, Kalamazoo, MI (US); Graeme Bainbridge, Kalamazoo, MI (US)

(21) Appl. No.: 17/496,597

(22) Filed: Oct. 7, 2021

Related U.S. Application Data
(60) Provisional application No. 63/088,729, filed on Oct. 7, 2020.

(51) Int. Cl. C07K 16/22; A61P 19/02

(52) U.S. Cl. CPC C07K 16/22; A61P 19/02; A61K 2039/505; C07K 2317/24; C07K 2317/565

(57) ABSTRACT

The present disclosure encompasses novel anti-NGF antibodies, antigen binding proteins and polynucleotides encoding the same. The disclosure further provides use of the novel antibodies, antigen binding proteins and/or nucleotide of the invention for the treatment and/or prevention of NGF related disorders, particularly in for the management of pain.

### CROSS REFERENCE TO RELATED APPLICATIONS

This application claims the benefit under 35 USC 119(e) to U.S. Provisional Application No. 63/088,729 filed on Oct. 7, 2020, which is hereby incorporated by reference in its entirety.

### FIELD OF THE INVENTION

The present invention relates to the field of immunology. More specifically, the present invention relates to anti-NGF antigen binding proteins that specifically bind to NGF that have been modified to become non-immunogenic in particular species. The invention further concerns use of such antigen binding proteins in the treatment and/or prevention of NGF related disorders, particularly pain.

### BACKGROUND OF THE INVENTION

Nerve growth factor (NGF) was the first neurotrophin to be identified, and its role in the development and survival of both peripheral and central neurons has been well characterized. NGF has been shown to be a critical survival and maintenance factor in the development of peripheral sympathetic and embryonic sensory neurons and of basal forebrain cholinergic neurons (Smeyne, et al., Nature 368:246-249 (1994) and Crowley, et al., Cell 76: 1001-101 I (1994)). NGF upregulates expression of neuropeptides in sensory neurons (Lindsay, et al, Nature 337:362-364 (1989)), and its activity is mediated through two different membrane-bound receptors, the TrkA tyrosine kinase receptor and the p75 common neurotrophin receptor (sometimes termed “high affinity” and “low affinity” NGF receptors, respectively) which is structurally related to other members of the tumor necrosis factor receptor family (Chao, et al., Science 232:518-521 (1986)).

In addition to its effects in the nervous system, NGF has been increasingly implicated in processes outside of the nervous system. For example, NGF has been shown to enhance vascular permeability (Otten, et al., Eur J Pharmacol. 106: 199-201 (1984)), enhance T- and B-cell immune responses (Otten, et al., Proc. Natl. Acad. Sci. USA 86:10059-10063 (1989)), induce lymphocyte differentiation and mast cell proliferation and cause the release of soluble biological signals from mast cells (Matsuda, et al., Proc. Natl. Acad. Sci. USA 85:6508-6512 (1988); Pearce, et al., J. Physiol. 372:379-393 (1986); Bischoff, et al., Blood 79:2662-2669 (1992); Horigome, et al., J. Bioi. Chem. 268:14881-14887 (1993)). NGF is produced by several cell types including mast cells (Leon, et al., Proc. Natl. Acad. Sci. USA 91:3739-3743 (1994)), B-lymphocytes (Torcia, et al., Cell 85:345-356 (1996), keratinocytes (Di Marco, et al., J. Biol. Chem. 268: 22838-22846)), smooth muscle cells (Ueyama, et al., J. Hypertens. 11: 1061-1065 (1993)), fibroblasts (Lindholm, et al., Eur. J. Neurosci. 2:795-801 (1990)), bronchial epithelial cells (Kassel, et al., Clin, Exp. Allergy 31:1432-40 (2001)), renal mesangial cells (Steiner, et al., Am. J. Physiol. 261: F792-798 (1991)) and skeletal muscle myotubes (Schwartz, et al., J Photochem. Photobiol. 866: 195-200 (2002)). NGF receptors have been found on a variety of cell types outside of the nervous system. For example, TrkA has been found on human monocytes, T- and B-lymphocytes and mast cells.

An association between increased NGF levels and a variety of inflammatory conditions has been observed in human patients as well as in several animal models. These include systemic lupus erythematosus (Bracci-Laudiero, et al., Neuroreport 4:563-565 (1993)), multiple sclerosis (BracciLaudiero, et al, Neurosci. Lett. 147:9-12 (1992)), psoriasis (Raychaudhuri, et al., Acta Derm. l'enereol. 78:84-86 (1998)), arthritis (Falcim, et al., Ann. Rheum. Dis. 55:745-748 (1996)), interstitial cystitis (Okragly, et al., J. Urology 161: 438-441 (1999)) and asthma (Braun, et al., Eur. J Immunol. 28:3240-3251 (1998)). A consistently elevated level of NGF in peripheral tissues is associated with hyperalgesia and inflammation and has been observed in a number of forms of arthritis. The synovium of patients affected by rheumatoid arthritis expresses high levels of NGF while in non-inflamed synovium NGF has been reported to be undetectable (Aloe, et al., Arch. Rheum. 35:351-355 (1992)). Similar results were seen in rats with experimentally induced rheumatoid arthritis (Aloe, et al., Clin. Exp. Rheumatol. 10:203-204 (1992)). Elevated levels of NGF have been reported in transgenic arthritic mice along with an increase in the number of mast cells (Aloe, et al., Int. J. Tissue Reactions-Exp. Clin. Aspects 15:139-143 (1993)).

Osteoarthritis (OA) is one of the most common chronic musculoskeletal diseases in dogs. The development of OA is mainly secondary to trauma, joint instability, and diseases such as hip dysplasia. Osteoarthritis is a disease condition of the entire joint, and both inflammatory and degenerative changes of all articular structures result in disability and clinical signs of lameness and pain. Pain is the most important clinical manifestation of canine OA and it is the result of a complex interplay between structural joint changes, biochemical and molecular alterations, as well as peripheral and central pain-processing mechanisms. Within this network, the activation and sensitization of peripheral nociceptors by inflammatory and hyperalgesic mediators (e.g. cytokines, prostaglandins and neuromediators) is one of the main peripheral mechanisms responsible for the joint pain.

Within the United States alone approximately 14.5 million dogs suffer from OA. Non-steroidal anti-inflammatory drugs (NSAIDs) are the most common drug category prescribed by veterinarians, but can be limited by their efficacy and tolerability. Market research indicates that approximately 9 million dogs are treated with NSAIDs within the US. Corticosteroids are used rarely and typically for a short period of time and as a last resort.

In felines, OA is a pathological change of a diarthrodial synovial articulation, characterized by the deterioration of articular cartilage, osteophyte formation, bone remodeling, soft tissue changes and a low-grade non-purulent inflammation. Even though radiographic features of feline OA have been well described, clinical signs of disease are generally poorly documented and can go undiagnosed. The difficulty in assessing lameness in cats results for their small size and natural agility which allows them to compensate. It is believed, however, that clinical signs of feline OA include weight loss, anorexia, depression, abnormal elimination habits, poor grooming, aggressive behavior and a gradual reduction in the ability to jump to overt lameness. Based on misdiagnosis feline OA remains generally untreated and is an unmet veterinary medicine need.

### SUMMARY OF THE INVENTION

The invention provides a novel anti-NGF antigen binding protein (antibody, antibody fragment, antagonist antibody, as defined and used interchangeably herein) that specifically binds Nerve Growth Factor (NGF) and inhibits the binding between NGF and TrkA thus blocking the biological activity of NGF, and polynucleotides encoding the same. The invention further provides methods of making and using of said antigen binding proteins and/or nucleotides in the treatment and/or prevention of NGF related disorders, particularly pain.

In one aspect the present invention provides an antigen binding protein that specifically binds Nerve Growth Factor (NGF) and inhibits the binding between NGF and TrkA thus blocking the biological activity of NGF, as defined herein, which comprises a heavy chain variable region (VH) comprising a Complimentary Determining Region 1 (CDR 1) comprising an amino acid sequence having at least about 90% sequence identity to SEQ ID NO.4 (amino acid sequence: GFTLTQYG), a Complimentary Determining Region 2 (CDR 2) comprising an amino acid sequence having at least about 90% sequence identity to SEQ ID NO.5 (amino acid sequence: VIWATGATD) and a Complimentary Determining Region 3 (CDR 3) comprising an amino acid sequence having at least about 90% sequence identity to SEQ ID NO. 6 (amino acid sequence: DGWWYATSWYFDV); and the antigen binding protein comprises a light chain variable region (VL) which comprises:
A. a Complimentary Determining Region 1 (CDR 1) comprising an amino acid sequence comprising at least about 90% sequence identity to the amino acid sequence comprising:
X1-Alanine[A]-Serine[S]-Glutamine[Q]-X2-Isoleucine[I]-X3-X4-X5-Leucine[L]-Asparagine[N](SEQ ID NO.177)
   wherein:
   X1 comprises Lysine (K) or Arginine (R),
   X2 comprises Serine (S) or Aspartic Acid (D),
   X3 comprises Asparagine (N) or Serine (S),
   X4 comprises Histidine (H) or Asparagine (N),
   X5 comprises Tyrosine [Y] or Asparagine [N]; and
B. a Complimentary Determining Region 2 (CDR 2) comprising an amino acid sequence having at least about 90% sequence identity to the amino acid sequence comprising:
   Tyrosine [Y]-X6-Serine [S]-X7-X8-Histidine [H]-Serine [S] (SEQ ID NO.178)
   wherein:
   X6 comprises Isoleucine [I] or Threonine [T],
   X7 comprises Arginine [R] or Serine [S],
   X8 comprises Leucine [L] or Phenylalanine [F]; and
C. a Complimentary Determining Region 3 (CDR 3) comprising an amino acid sequence having at least about 90% sequence identity to the amino acid sequence comprising:
   X9-X10-X11-X12-X13-X14-Proline [P]-X15-X16 (SEQ ID NO. 179)
   wherein:
   X9 comprises Glutamine [Q] or Histidine [H],
   X10 comprises Glutamine [Q] or Arginine [R],
   X11 comprises Glycine [G] or Alanine [A],
   X12 comprises Aspartic Acid [D], Serine [S], Threonine [T] or Asparagine [N],
   X13 comprises Histidine [H], Threonine [T] or Methionine [M],
   X14 comprises Phenylalanine [F], Lysine [L] or Serine [S],
   X15 comprises Arginine [R], Tyrosine [Y] or Glycine [G];
   X16 comprises Threonine [T] or Proline [P]; and
      any variants thereof having one or more conservative amino acid substitutions in at least one of CDR1, CDR2 or CDR3 within any of the variable light or variable heavy chain regions of said antigen binding protein.

In another aspect the present invention provides an antigen binding protein that specifically binds Nerve Growth Factor (NGF) and inhibits the binding between NGF and TrkA thus blocking the biological activity of NGF, as defined herein, which comprises a heavy chain variable region (VH) comprising a Complimentary Determining Region 1 (CDR 1) comprising an amino acid sequence having at least about 90% sequence identity to SEQ ID NO.4 (amino acid sequence: GFTLTQYG), a Complimentary Determining Region 2 (CDR 2) comprising an amino acid sequence having at least about 90% sequence identity to SEQ ID NO.5 (amino acid sequence: VIWATGATD) and a Complimentary Determining Region 3 (CDR 3) comprising an amino acid sequence having at least about 90% sequence identity to SEQ ID NO. 6 (amino acid sequence: DGWWYATSWYFDV); and the antigen binding protein comprises a light chain variable region (VL) which comprises an antigen binding protein that specifically binds Nerve Growth Factor (NGF) comprising a light chain variable region (VL) comprising
A. a Complimentary Determining Region 1 (CDR1) comprising an amino acid sequence having at least about 90% sequence identity to the amino acid sequence comprising: (X1)-Alanine[A]-Serine[S]-Glutamine[Q]-(X2)-Isoleucine [I]-(X3)-(X4)-(X5)-Leucine[L]-Asparagine[N] (SEQ ID NO.180)
   wherein:
   X1 comprises Lysine (K) or Arginine (R),
   X2 comprises Serine (S) or Aspartic Acid (D),
   X3 comprises Asparagine (N) or Serine (S),
   X4 comprises Histidine (H) or Asparagine (N),
   X5 comprises Tyrosine [Y] or Asparagine [N]; and
B. a Complimentary Determining Region 2 (CDR2) comprising an amino acid sequence having at least about 90% sequence identity to the amino acid sequence comprising:
   Threonine [T]-(X6)-(X7)-Leucine [L]-(X8)-(X9) (SEQ ID NO.181) wherein:
   X6 comprises Threonine [T], Histidine [H], Serine [S] or Alanine [A],
   X7 comprises Arginine [R] or Serine[S],
   X8 comprises Glutamine [Q] or Histidine [H],
   X9 comprises Alanine[A], Glutamine[Q], Glycine [G] or Valine [V]; and
C. a Complimentary Determining Region 3 (CDR3) comprising an amino acid sequence having at least about 90% sequence identity to the amino acid sequence comprising:
(X10)-(X11)-(X12)-(X13)-(X14)-(X15)-P-(X16)-(X17) (SEQ ID NO.182) wherein
   X10 comprises Q or H,
   X11 comprises Q or R,
   X12 comprises G or A,
   X13 comprises D, S, T or N,
   X14 comprises H, T or M,
   X15 comprises F, L or S,
   X16 comprises R, Y or G,
   X17 comprises T or P; and
any variants thereof having one or more conservative amino acid substitutions in at least one of CDR1, CDR2 or CDR3 within any of the variable light or variable heavy chain regions of said antigen binding protein.

In another aspect, the present invention provides an antigen binding protein that specifically binds Nerve Growth Factor (NGF) and inhibits the binding between NGF and TrkA thus blocking the biological activity of NGF, as defined herein, which comprises:
a heavy chain variable region (VH) comprising:
   a. a Complimentary Determining Region 1 (CDR 1) comprising an amino acid sequence having at least about 90% sequence identity to GFTLTQYG (SEQ ID NO.4):
   b. a Complimentary Determining Region 2 (CDR 2) comprising an amino acid sequence having at least about 90% sequence identity to VIWATGATD (SEQ ID NO.5); and
   c. a Complimentary Determining Region 3 (CDR 3) comprising an amino acid sequence having at least about 90% sequence identity to DGWWYATSWYFDV (SEQ ID NO. 6); and
a light chain variable region (VL) comprising Complimentary Determining Regions having at least about 90% sequence identity to the amino acid sequences selected from the group consisting of:
   d. a light chain variable region comprising:
      i. a CDR1 comprising the amino acid sequence KASQDINHYLN (SEQ ID NO. 7);
      ii. a CDR2 comprising an amino acid sequence YTSRLHS (SEQ ID. NO. 8); and
      iii. a CDR 3 comprising an amino acid sequence QQGDHFPRT (SEQ ID NO.9);
   e. a light chain variable region comprising:
      i. a CDR1 comprising the amino acid sequence RASQSISNNLN (SEQ ID NO. 10);
      ii. a CDR2 comprising an amino acid sequence YISSFHS (SEQ ID. NO. 11); and
      iii. a CDR 3 comprising an amino acid sequence QQGDHFPYT (SEQ ID NO.12);
   f. a light chain variable region comprising:
      i. a CDR1 comprising the amino acid sequence KASQDINHYLN (SEQ ID NO. 13);
      ii. a CDR2 comprising an amino acid sequence YTSSLHS (SEQ ID. NO. 14); and
      iii. a CDR 3 comprising an amino acid sequence QQGDHFPRT (SEQ ID NO.15);
   g. a light chain variable region comprising:
      i. a CDR1 comprising the amino acid sequence KASQSINHYLN (SEQ ID NO. 16);
      ii. a CDR2 comprising an amino acid sequence YTSRLHS (SEQ ID. NO. 17); and
      iii. a CDR 3 comprising an amino acid sequence QQGSTLPRT (SEQ ID NO.18);
   h. a light chain variable region comprising:
      i. a CDR1 comprising the amino acid sequence RASQDISNYLN (SEQ ID NO. 19);
      ii. a CDR2 comprising an amino acid sequence YTSRLHS (SEQ ID. NO. 20); and
      iii. a CDR 3 comprising an amino acid sequence HRATTSPGP (SEQ ID NO.21);
   i. a light chain variable region comprising:
      i. a CDR1 comprising the amino acid sequence KASQDINHYLN (SEQ ID NO. 22);
      ii. a CDR2 comprising an amino acid sequence YTSRLHS (SEQ ID. NO. 23); and
      iii. a CDR 3 comprising an amino acid sequence QQGSTLPRT (SEQ ID NO.24);
   j. a light chain variable region comprising:
      i. a CDR1 comprising the amino acid sequence RASQDISNYLN (SEQ ID NO. 25);
      ii. a CDR2 comprising an amino acid sequence YTSRLHS (SEQ ID. NO. 26); and
      iii. a CDR 3 comprising an amino acid sequence QQGSTLPRT (SEQ ID NO.27);
   k. a light chain variable region comprising:
      i. a CDR1 comprising the amino acid sequence RASQDISNYLN (SEQ ID NO. 28);
      ii. a CDR2 comprising an amino acid sequence YTSSLHS (SEQ ID. NO. 29); and
      iii. a CDR 3 comprising an amino acid sequence QQGSTLPRT (SEQ ID NO.30);
   l. a light chain variable region comprising:
      i. a CDR1 comprising the amino acid sequence KASQSINHYLN (SEQ ID NO. 31);
      ii. a CDR2 comprising an amino acid sequence YISSFHS (SEQ ID. NO. 32); and
      iii. a CDR 3 comprising an amino acid sequence QQSHTLPYT (SEQ ID NO.33);
   m. a light chain variable region comprising:
      i. a CDR1 comprising the amino acid sequence KASQDINHYLN (SEQ ID NO. 34);
      ii. a CDR2 comprising an amino acid sequence YVTTLHA (SEQ ID. NO. 35); and
      iii. a CDR 3 comprising an amino acid sequence QQGDHFPRT (SEQ ID NO.36);
   n. a light chain variable region comprising:
      i. a CDR1 comprising the amino acid sequence RASQDISNYLN (SEQ ID NO. 37);
      ii. a CDR2 comprising an amino acid sequence KTNSLQT (SEQ ID. NO. 38); and
      iii. a CDR 3 comprising an amino acid sequence QQGSTLPRT (SEQ ID NO.39);
   o. a light chain variable region comprising:
      i. a CDR1 comprising the amino acid sequence RASQDISNYLN (SEQ ID NO. 40);
      ii. a CDR2 comprising an amino acid sequence YVTSLHA (SEQ ID. NO. 41); and
      iii. a CDR 3 comprising an amino acid sequence QQGSTLPRT (SEQ ID NO.42);
   p. a light chain variable region comprising:
      i. a CDR1 comprising the amino acid sequence RASQDISNYLN (SEQ ID NO. 43);
      ii. a CDR2 comprising an amino acid sequence YTSRLHS (SEQ ID. NO. 44); and
      iii. a CDR 3 comprising an amino acid sequence QQGNMFPYT (SEQ ID NO.45);
   q. a light chain variable region comprising:
      i. a CDR1 comprising the amino acid sequence KASQDINHYLN (SEQ ID NO. 46);
      ii. a CDR2 comprising an amino acid sequence YTSRLHS (SEQ ID. NO. 47); and
      iii. a CDR 3 comprising an amino acid sequence QQGNMFPYT (SEQ ID NO.48);
   r. a light chain variable region comprising
      i. a CDR1 comprising the amino acid sequence KASQDINHYLN (SEQ ID NO. 193);
      ii. a CDR2 comprising an amino acid sequence TTRLQA (SEQ ID. NO. 194); and
      iii. a CDR 3 comprising an amino acid sequence QQGDHFPRT (SEQ ID NO.195); and
any variants thereof having one or more conservative amino acid substitutions in at least one of CDR1, CDR2 or CDR3 within the variable light and/or variable heavy chain regions of said antigen binding protein.

In one or more embodiments the present invention provides an antigen binding protein or antibody fragment that specifically binds Nerve Growth Factor (NGF) and inhibits the binding between NGF and TrkA thus blocking the biological activity of NGF, comprising a variable heavy chain (VH) comprising a Complimentary Determining Region (CDR) 1 comprising an amino acid sequence having at least about 90% sequence identity to the amino acid sequence of GFTLTQYG (SEQ ID NO.4), a Complimentary Determining Region (CDR) 2 comprising an amino acid sequence having at least about 90% sequence identity to the amino acid sequence of VIWATGATD (SEQ ID NO.5) and a Complimentary Determining Region (CDR) 3 an amino acid sequence having at least about 90% sequence identity to the amino acid sequence of DGWWYATSWYFDV (SEQ ID NO. 6), and a variable light chain comprises a CDR1 comprising an amino acid sequence having at least about 90% sequence identity to the amino acid sequence of KASQDINHYLN (SEQ ID NO.7), a CDR2 comprising the amino acid sequence having at least about 90% sequence identity to the amino acid sequence of YTSRLHS (SEQ ID NO.8) and a CDR 3 comprising an amino acid sequence having at least about 90% sequence identity to the amino acid sequence of QQGDHFPRT (SEQ ID NO.9); and any variants thereof having one or more conservative amino acid substitutions in at least one of CDR1, CDR2 or CDR3 within the variable heavy and/or variable light chains of said antigen binding protein.

In one or more embodiments the present invention provides an antigen binding protein or antibody fragment that specifically binds Nerve Growth Factor (NGF) and inhibits the binding between NGF and TrkA thus blocking the biological activity of NGF, comprising a variable heavy chain (VH) comprising a Complimentary Determining Region (CDR) 1 comprising an amino acid sequence having at least about 90% sequence identity to the amino acid sequence comprising GFTLTQYG (SEQ ID NO.4), a Complimentary Determining Region (CDR) 2 comprising an amino acid sequence having at least about 90% sequence identity to the amino acid sequence comprising VIWATGATD (SEQ ID NO.5) and a Complimentary Determining Region (CDR) 3 comprising an amino acid sequence having at least about 90% sequence identity to the amino acid sequence comprising DGWWYATSWYFDV (SEQ ID NO. 6), and a variable light chain comprises a CDR1 comprising an amino acid sequence having at least about 90% sequence identity to the amino acid sequence comprising RASQSISNNLN (SEQ ID NO.10), a CDR2 comprising an amino acid sequence having at least about 90% sequence identity to the amino acid sequence comprising YISSFHS (SEQ ID NO.11) and a CDR 3 comprising an amino acid sequence having at least about 90% sequence identity to the amino acid sequence comprising QQGDHFPYT (SEQ ID NO.12) and any variants thereof having one or more conservative amino acid substitutions in at least one of CDR1, CDR2 or CDR3 within the variable heavy and/or variable light chain regions of said antigen binding protein.

In one or more embodiments the present invention provides an antigen binding protein or antibody fragment that specifically binds Nerve Growth Factor (NGF) and inhibits the binding between NGF and TrkA thus blocking the biological activity of NGF, comprising a variable heavy chain (VH) comprising a Complimentary Determining Region (CDR) 1 comprising an amino acid sequence having at least about 90% sequence identity to the amino acid sequence comprising GFTLTQYG (SEQ ID NO.4), a Complimentary Determining Region (CDR) 2 comprising an amino acid sequence having at least about 90% sequence identity to the amino acid sequence comprising VIWATGATD (SEQ ID NO.5) and a Complimentary Determining Region (CDR) 3 comprising an amino acid sequence having at least about 90% sequence identity to the amino acid sequence comprising DGWWYATSWYFDV (SEQ ID NO. 6), and a variable light chain comprises a CDR1 comprising an amino acid sequence having at least about 90% sequence identity to the amino acid sequence comprising KASQDINHYLN (SEQ ID NO.13), a CDR2 comprising an amino acid sequence having at least about 90% sequence identity to the amino acid sequence comprising YTSSLHS (SEQ ID NO.14) and a CDR 3 comprising an amino acid sequence having at least about 90% sequence identity to the amino acid sequence comprising QQGDHFPRT (SEQ ID NO.15) and any variants thereof having one or more conservative amino acid substitutions in at least one of CDR1, CDR2 or CDR3 within the variable heavy and/or variable light chain regions of said antigen binding protein.

In one or more embodiments the present invention provides an antigen binding protein or antibody fragment that specifically binds Nerve Growth Factor (NGF) and inhibits the binding between NGF and TrkA thus blocking the biological activity of NGF, comprising a variable heavy chain (VH) comprising a

Complimentary Determining Region (CDR) 1 comprising an amino acid sequence having at least about 90% sequence identity to the amino acid sequence comprising GFTLTQYG (SEQ ID NO.4), a Complimentary Determining Region (CDR) 2 comprising an amino acid sequence having at least about 90% sequence identity to the amino acid sequence comprising VIWATGATD (SEQ ID NO.5) and a Complimentary Determining Region (CDR) 3 comprising an amino acid sequence having at least about 90% sequence identity to the amino acid sequence comprising DGWWYATSWYFDV (SEQ ID NO. 6), and a variable light chain comprises a CDR1 comprising an amino acid sequence having at least about 90% sequence identity to the amino acid sequence comprising KASQSINHYLN (SEQ ID NO.16), a CDR2 comprising an amino acid sequence having at least about 90% sequence identity to the amino acid sequence comprising YTSRLHS (SEQ ID NO.17) and a CDR 3 comprising an amino acid sequence having at least about 90% sequence identity to the amino acid sequence comprising QQGSTLPRT (SEQ ID NO.18) and any variants thereof having one or more conservative amino acid substitutions in at least one of CDR1, CDR2 or CDR3 within the variable heavy and/or variable light chain regions of said antigen binding protein.

In one or more embodiments the present invention provides an antigen binding protein or antibody fragment that specifically binds Nerve Growth Factor (NGF) and inhibits the binding between NGF and TrkA thus blocking the biological activity of NGF, comprising a variable heavy chain (VH) comprising a Complimentary Determining Region (CDR) 1 comprising an amino acid sequence having at least about 90% sequence identity to the amino acid sequence comprising GFTLTQYG (SEQ ID NO.4), a Complimentary Determining Region (CDR) 2 comprising an amino acid sequence having at least about 90% sequence identity to the amino acid sequence comprising VIWATGATD (SEQ ID NO.5) and a Complimentary Determining Region (CDR) 3 comprising an amino acid sequence having at least about 90% sequence identity to the amino acid sequence comprising DGWWYATSWYFDV (SEQ ID NO. 6), and a variable light chain comprises a CDR1 comprising an amino acid sequence having at least about 90% sequence identity to the amino acid sequence comprising RASQDISNYLN (SEQ ID NO.19), a CDR2 comprising an amino acid sequence having at least about 90% sequence identity to the amino acid sequence comprising YTSRLHS (SEQ ID NO.20) and a CDR 3 comprising an amino acid sequence having at least about 90% sequence identity to the amino acid sequence comprising HRATTSPGP (SEQ ID NO.21) and any variants thereof having one or more conservative amino acid substitutions in at least one of CDR1, CDR2 or CDR3 within the variable heavy and/or variable light chain regions of said antigen binding protein.

In one or more embodiments the present invention provides an antigen binding protein or antibody fragment that specifically binds Nerve Growth Factor (NGF) and inhibits the binding between NGF and TrkA thus blocking the biological activity of NGF, comprising a variable heavy chain (VH) comprising a Complimentary Determining Region (CDR) 1 comprising an amino acid sequence having at least about 90% sequence identity to the amino acid sequence comprising GFTLTQYG (SEQ ID NO.4), a Complimentary Determining Region (CDR) 2 comprising an amino acid sequence having at least about 90% sequence identity to the amino acid sequence comprising VIWATGATD (SEQ ID NO.5) and a Complimentary Determining Region (CDR) 3 comprising an amino acid sequence having at least about 90% sequence identity to the amino acid sequence comprising DGWWYATSWYFDV (SEQ ID NO. 6), and a variable light chain comprises a CDR1 comprising an amino acid sequence having at least about 90% sequence identity to the amino acid sequence comprising KASQDINHYLN (SEQ ID NO.22), a CDR2 comprising an amino acid sequence having at least about 90% sequence identity to the amino acid sequence comprising YTSRLHS (SEQ ID NO.23) and a CDR3 comprising an amino acid sequence having at least about 90% sequence identity to the amino acid sequence comprising QQGSTLPRT (SEQ ID NO.24) and any variants thereof having one or more conservative amino acid substitutions in at least one of CDR1, CDR2 or CDR3 within the variable heavy and/or variable light chain regions of said antigen binding protein.

In one or more embodiments the present invention provides an antigen binding protein or antibody fragment that specifically binds Nerve Growth Factor (NGF) and inhibits the binding between NGF and TrkA thus blocking the biological activity of NGF, comprising a variable heavy chain (VH) comprising a Complimentary Determining Region (CDR) 1 comprising an amino acid sequence having at least about 90% sequence identity to the amino acid sequence comprising GFTLTQYG (SEQ ID NO.4), a Complimentary Determining Region (CDR) 2 comprising an amino acid sequence having at least about 90% sequence identity to the amino acid sequence comprising VIWATGATD (SEQ ID NO.5) and a Complimentary Determining Region (CDR) 3 comprising an amino acid sequence having at least about 90% sequence identity to the amino acid sequence comprising DGWWYATSWYFDV (SEQ ID NO. 6), and a variable light chain comprises a CDR1 comprising an amino acid sequence having at least about 90% sequence identity to the amino acid sequence comprising RASQDISNYLN (SEQ ID NO.25), a CDR2 comprising an amino acid sequence having at least about 90% sequence identity to the amino acid sequence comprising YTSRLHS (SEQ ID NO.26) and a CDR3 comprising an amino acid sequence having at least about 90% sequence identity to the amino acid sequence comprising QQGSTLPRT (SEQ ID NO.27) and any variants thereof having one or more conservative amino acid substitutions in at least one of CDR1, CDR2 or CDR3 within the variable heavy and/or variable light chain regions of said antigen binding protein.

In one or more embodiments the present invention provides an antigen binding protein or antibody fragment that specifically binds Nerve Growth Factor (NGF) and inhibits the binding between NGF and TrkA thus blocking the biological activity of NGF, comprising a variable heavy chain (VH) comprising a Complimentary Determining Region (CDR) 1 comprising an amino acid sequence having at least about 90% sequence identity to the amino acid sequence comprising GFTLTQYG (SEQ ID NO.4), a Complimentary Determining Region (CDR) 2 comprising an amino acid sequence having at least about 90% sequence identity to the amino acid sequence comprising VIWATGATD (SEQ ID NO.5) and a Complimentary Determining Region (CDR) 3 comprising an amino acid sequence having at least about 90% sequence identity to the amino acid sequence comprising DGWWYATSWYFDV (SEQ ID NO. 6), and a variable light chain comprises a CDR1 comprising an amino acid sequence having at least about 90% sequence identity to the amino acid sequence comprising RASQDISNYLN (SEQ ID NO.28), a CDR2 comprising an amino acid sequence having at least about 90% sequence identity to the amino acid sequence comprising YYTSSLHS (SEQ ID NO.29) and a CDR3 comprising an amino acid sequence having at least about 90% sequence identity to the amino acid sequence comprising QQGSTLPRT (SEQ ID NO.30) and any variants thereof having one or more conservative amino acid substitutions in at least one of CDR1, CDR2 or CDR3 within the variable heavy and/or variable light chain regions of said antigen binding protein.

In one or more embodiments the present invention provides an antigen binding protein or antibody fragment that specifically binds Nerve Growth Factor (NGF) and inhibits the binding between NGF and TrkA thus blocking the biological activity of NGF, comprising a variable heavy chain (VH) comprising a Complimentary Determining Region (CDR) 1 comprising an amino acid sequence having at least about 90% sequence identity to the amino acid sequence comprising GFTLTQYG (SEQ ID NO.4), a Complimentary Determining Region (CDR) 2 comprising an amino acid sequence having at least about 90% sequence identity to the amino acid sequence comprising VIWATGATD (SEQ ID NO.5) and a Complimentary Determining Region (CDR) 3 comprising an amino acid sequence having at least about 90% sequence identity to the amino acid sequence comprising DGWWYATSWYFDV (SEQ ID NO. 6), and a variable light chain comprises a CDR1 comprising an amino acid sequence having at least about 90% sequence identity to the amino acid sequence comprising RASQDISNYLN (SEQ ID NO.43), a CDR2 comprising an amino acid sequence having at least about 90% sequence identity to the amino acid sequence comprising YTSRLHS (SEQ ID NO.44) and a CDR 3 comprising an amino acid sequence having at least about 90% sequence identity to the amino acid sequence comprising QQGNMFPYT (SEQ ID NO.45) and any variants thereof having one or more conservative amino acid substitutions in at least one of CDR1, CDR2 or CDR3 within the variable heavy and/or variable light chain regions of said antigen binding protein.

In one or more embodiments the present invention provides an antigen binding protein or antibody fragment that specifically binds Nerve Growth Factor (NGF) and inhibits the binding between NGF and TrkA thus blocking the biological activity of NGF, comprising a variable heavy chain (VH) comprising a Complimentary Determining Region (CDR) 1 comprising an amino acid sequence having at least about 90% sequence identity to the amino acid sequence comprising GFTLTQYG (SEQ ID NO.4), a Complimentary Determining Region (CDR) 2 comprising an amino acid sequence having at least about 90% sequence identity to the amino acid sequence comprising VIWATGATD (SEQ ID NO.5) and a Complimentary Determining Region (CDR) 3 comprising an amino acid sequence having at least about 90% sequence identity to the amino acid sequence comprising DGWWYATSWYFDV (SEQ ID NO. 6), and a variable light chain comprises a CDR1 comprising an amino acid sequence having at least about 90% sequence identity to the amino acid sequence comprising KASQDINHYLN (SEQ ID NO.46), a CDR2 comprising an amino acid sequence having at least about 90% sequence identity to the amino acid sequence comprising YTSRLHS (SEQ ID NO.47) and a CDR3 comprising an amino acid sequence having at least about 90% sequence identity to the amino acid sequence comprising QQGNMFPYT (SEQ ID NO.48) and any variants thereof having one or more conservative amino acid substitutions in at least one of CDR1, CDR2 or CDR3 within the variable heavy and/or variable light chain regions of said antigen binding protein. In one or more embodiment the antigen binding protein of the invention further comprises a canine light chain constant region comprising an amino acid sequence having at least about 90% sequence identity to the amino acid sequence comprising SEQ ID NO.160 and a canine heavy chain constant region comprising an amino acid sequence having at least about 90% sequence identity to the amino acid sequence comprising SEQ ID NO.158. In one embodiment the antibody of the invention comprises a canine heavy chain constant region comprising effector function mutations comprising an amino acid sequence having at least about 90% sequence identity to the amino acid sequence comprising SEQ ID NO.184.

In one or more embodiments, the present invention provides an isolated and recombinant caninized antigen binding protein, “ZTS-182 m6”, wherein the variable heavy chain comprises amino acid sequence having at least about 90% sequence identity to the amino acid sequence comprising SEQ ID NO. 49 and wherein the variable light chain comprises amino acid sequences having at least about 90% sequence identity to the amino acid sequence comprising SEQ ID NO. 175. Additionally, the variable heavy chain comprises Complementarity Determining Regions 1-3 comprising the amino acid sequences having at least about 90% sequence identity to SEQ ID NO. 4 (“01B12H3AHC” VH CDR1), amino acid sequence having at least about 90% sequence identity to the amino acid sequence comprising SEQ ID NO. 5 (“01B12H3AHC” VH CDR2), amino acid sequence having at least about 90% sequence identity to the amino acid sequence comprising SEQ ID NO. 6 (“01 B12H3AHC” VH CDR3); and wherein the variable light chain Complementarity Determining Regions 1-3 comprising the amino acid sequences having at least about 90% sequence identity to SEQ ID NO. 167, amino acid sequence having at least about 90% sequence identity to the amino acid sequence comprising SEQ ID NO. 168, and amino acid sequence having at least about 90% sequence identity to the amino acid sequence comprising SEQ ID NO. 169; and any variants thereof having one or more conservative amino acid substitutions in at least one of CDR1, CDR2 or CDR3 within any of the variable light or variable heavy chains of said antigen binding protein or within the amino acid sequence of the entire VH or VL sequences of the antigen binding protein of the invention. In one or more embodiment the antigen binding protein of the invention further comprises a canine light chain constant region comprising an amino acid sequence having at least about 90% sequence identity to the amino acid sequence comprising SEQ ID NO.160 and a canine heavy chain constant region comprising an amino acid sequence having at least about 90% sequence identity to the amino acid sequence comprising SEQ ID NO.158. In one embodiment the antibody of the invention comprises a canine heavy chain constant region comprising effector function mutations comprising an amino acid sequence having at least about 90% sequence identity to the amino acid sequence comprising SEQ ID NO.184.

In one or more embodiments, the present invention provides that the antigen binding protein of the invention, as defined herein comprises a chimeric antibody, a murine antibody, a caninized antibody, a felinized antibody, an equinized antibody or a humanized antibody. In one embodiment, the antigen binding protein of the invention comprises a chimeric antibody. In one embodiment, the antigen binding protein of the invention comprises a caninized antibody. In one embodiment of the invention the antigen binding protein comprises a felinized antibody. In one embodiment, the antigen binding protein comprises an equinized antibody. In one embodiment, the antigen binding protein comprises a humanized antibody.

In one aspect, the present invention provides an antigen binding protein that specifically binds Nerve Growth Factor (NGF) and inhibits the binding between NGF and TrkA thus blocking the biological activity of NGF, as defined herein, which comprises:
1) a heavy chain variable region (VH) comprising an amino acid sequence having at least about 90% sequence identity to the amino acid sequence:

(SEQ ID NO. 49)
EVQLVESGGDLARPGGSLKLSCVVSGFTLTQYG
INWVRQAPGKGLQWVTVIWATGATDYNSALKSR
FTVSRDNAMNTVYLQMNSLRVEDTAVYYCARDG
WWYATSWYFDVWGQGTLVTVSSASTTAPSVFPL
APSCGSTSGSTVALACLVSGYFPEPVTVSWNSG
SLTSGVHTFPSVLQSS;

and
2) a light chain variable region (VL) comprising an amino acid sequence having at least about 90% sequence identity to the amino acid sequence selected from the group consisting of:

a.
(SEQ ID NO. 51)
DIVMTQTPLSLSVSPGEPASISCKASQDINHYLNWFRQKPDG
TVKLLIYYTSRLHSGVPSRFSGSGSGTDFTLRISRVEADDAGV
YYCQQGDHFPRTFGQGT;
b.
(SEQ ID NO. 53)
DIVMTQTPLSLSVSPGEPASISCRASQSISNNLNWFRQKPDG
TVKLLIYYISSFHSGVPSRFSGSGSGTDFTLRISRVEADDAG
VYYCQQGDHFPYTFGQGT;
c.
(SEQ ID NO. 55)
DIVMTQTPLSLSVSPGEPASISCKASQDINHYLNWFRQKPDG
TVKLLIYYTSSLHSGVPSRTSGSGSGTDFTLRISRVEADDAG
VYYCQOGDHFPRTFGQGT;
d.
(SEQ ID NO. 57)
DIVMTQTPLSLSVSPGEPASISCKASQSINHYLNWFRQKPDG
TVKLLIYYTSRLHSGVPSRFSGSGSGTDFTLRISRVEADDAG
VYYCQQGSTLPRTFGQGT;
e.
(SEQ ID NO. 59)
DIVMTQTPLSLSVSPGEPASISGRASQDISNYLNWFRQKPDG
TVKLLIYYTSRLHSGVPSRFSGSGSGTDFTLRISRVEADDAG
VYYCHRATTSPGPSARV;
f.
(SEQ ID NO. 61)
DIVMTQTPLSLSVSPGEPASISCKASQSINHYLNWFRQKPDG
TVKLLIYYTSRLHSGVPSRFSGSGSGTDFTLRISRVEADDAG
VYYCQQGSTLPRTFGQGT;
g.
(SEQ ID NO. 63)
DIVMTQTPLSLSVSPGEPASISCRASQDISNYLNWFRQKPDG
TVKLLIYYTSRLHSGVPSRFSGSGSGTDFTLRISRVEADDAG
VYYCQQGSTLPRTFGQGT;
h.
(SEQ ID NO. 65)
DIVMTQTPLSLSVSPGEPASISCRASQDISNYLNWFRQKPDG
TVKLLIYYTSSLHSGVPSRFSGSGSGTDFTLRISRVEADDAG
VYYCQQGSTLPRTFGQGT;
i.
(SEQ ID NO. 67)
DIVMTQTPLSLSVSPGEPASISCKASQSINHYLNWFRQKPDG
TVKLLIYYISSFHSGVPSRFSGSGSGTDFTLRISRVEADDAG
VYYCQQSHTLPYTFGQGT;
j.
(SEQ ID NO. 69)
DIVMTQTPLSLSVSPGEPASISCKASQDINHYLNWFRQKPDG
TVKLLIYYVTSLHAGVPSRFSGSGSGTDFTLRISRVEADDAG
VYYCQQGDHFPRTFGQGT;
k.
(SEQ ID NO. 71)
DIVMTQTPLSLSVSPGEPASISCRASQDISNYLNWFRQKPDG
TVKLLIYKTNSLQTGVPSRFSGSGSGTDFTLRISRVEADDAG
VYYCQQGSTLPRTFGQGT;
l.
(SEQ ID NO. 73)
DIVMTQTPLSLSVSPGEPASISCRASQDISNYLNWFRQKPDG
TVKLLIYYVTSLHAGVPSRFSGSGSGTDFTLRISRVEADDAG
VYYCQQGSTLPRTFGQGT;
m.
(SEQ ID NO. 75)
EIVMTQSPASLSLSQEEKVTITCRASQDISNYLNWYQQKPG
QAPKLLIYYTSRLHSGVPSRFSGSGSGTDFSFTISSLEPED
VAVYYCQQGNMFPYTFGGGT;
n.
(SEQ ID NO. 77)
EIVMTQSPASLSLSQEEKVTITCKASQDINHYLNWYQQKPG
QAPKLLIYYTSRLHSGVPSRFSGSGSGTDFSFTISSLEPED
VAVYYCQQGNMFPYTFGGGT;
o.
(SEQ ID NO. 175)
DIVMTQTPLSLSVSPGEPASISCKASQDINHYLNWFRQKPD
GTVKLLIYTTRLQASGVPSRFSGSGSGTDFTLRISRVEADD
AGVYYCQQGDHFPRTFGQGT;

and any variants thereof having one or more conservative amino acid substitutions within the variable light and/or variable heavy chain regions of said antigen binding protein.

In one or more embodiment the antigen binding protein of the invention further comprises a canine light chain constant region comprising an amino acid sequence having at least about 90% sequence identity to the amino acid sequence comprising SEQ ID NO.160 and a canine heavy chain constant region comprising an amino acid sequence having at least about 90% sequence identity to the amino acid sequence comprising SEQ ID NO.158. In one embodiment the antibody of the invention comprises a canine heavy chain constant region comprising effector function mutations comprising an amino acid sequence having at least about 90% sequence identity to the amino acid sequence comprising SEQ ID NO.184.

In one or more embodiments the present invention provides an antigen binding protein that specifically binds Nerve Growth Factor (NGF) and inhibits the binding between NGF and TrkA thus blocking the biological activity of NGF, as defined herein, comprising a variable heavy chain (VH) comprising an amino acid sequence having at least about 90% sequence identity to the amino acid sequence comprising SEQ ID NO. 49 (amino acid sequence: EVQLVESGGDL ARPGGSLKLSCVVSG FTLTQYG INWVRQAPG KG LQWVTVIWATGATDYNSALKSRFTVSRDNAMNTVYL QMNSLRVEDTAVYYCARDGWWYATSWYFDVWGQGTLVTVSSASTTAPSVFPLAPSCGSTSGSTVALA CLVSGYFPEPVTVSWNSGSLTSGVHTFPSVLQSS) and a variable light chain (VL) comprising an amino acid sequence having at least about 90% sequence identity to the amino acid sequence comprising SEQ ID NO. 51 (amino acid sequence : DIVMTQTPLSLSVSPGEPASISCKASQDINHYLNWFRQKPDGTVK LLIYYTSRLHSGVPSRFSGSGSGTDFTLRISRVEADDAGVYYCQQGDHFPRTFGQGT); and any variants thereof having one or more conservative amino acid substitutions within the variable heavy and/or variable light chain regions of said antigen binding protein. In one or more embodiment the antigen binding protein of the invention further comprises a canine light chain constant region comprising an amino acid sequence having at least about 90% sequence identity to the amino acid sequence comprising SEQ ID NO.160 and a canine heavy chain constant region comprising an amino acid sequence having at least about 90% sequence identity to the amino acid sequence comprising SEQ ID NO.158. In one embodiment the antibody of the invention comprises a canine heavy chain constant region comprising effector function mutations comprising an amino acid sequence having at least about 90% sequence identity to the amino acid sequence comprising SEQ ID NO.184.

In one or more embodiments the present invention provides an antigen binding protein that specifically binds Nerve Growth Factor (NGF) and inhibits the binding between NGF and TrkA thus blocking the biological activity of NGF as defined herein, comprising a variable heavy chain (VH) comprising an amino acid sequence having at least about 90% sequence identity to the amino acid sequence comprising SEQ ID NO. 49 (amino acid sequence: EVQLVESGGDLARPGGSLKLSCV VSGFTLTQYGINWVRQAPGKGLQWVTVIWATGATDYNSALKSRFTVSRDNAMNTVYLQMNSLRVEDTA VYYCARDGWWYATSWYFDVWGQGTLVTVSSASTTAPSVFPLAPSCGSTSGSTVALACLVSGYFPEPVT VSWNSGSLTSGVHTFPSVLQSS) and a variable light chain (VL) comprising an amino acid sequence having at least about 90% sequence identity to the amino acid sequence comprising SEQ ID NO. 53 (amino acid sequence: DIVMTQTPLSLSVSPG EPASISCRASQSISNNLNWFRQKPDGTVKLLIY YISSFHSGVPSRFSGSGSGTDFTLRISRVEADDAGVYYCQQGDHFPYTFGQGT); and any variants thereof having one or more conservative amino acid substitutions within the variable heavy and/or variable light chain regions of said antigen binding protein.

In one or more embodiments the present invention provides an antigen binding that specifically binds Nerve Growth Factor (NGF) and inhibits the binding between NGF and TrkA as defined herein, comprising a variable heavy chain (VH) comprising an amino acid sequence having at least about 90% sequence identity to the amino acid sequence comprising SEQ ID NO. 49 (amino acid sequence: EVQLVESGG DLARPGGSLKLSCVVSG FTLTQYG I NWVRQAPG KG LQWVTVIWATGATDYNSALKSRFTV SRDNAMNTVYLQMNSLRVEDTAVYYCARDGWWYATSWYFDVWGQGTLVTVSSASTTAPSVFPLAPSC GSTSGSTVALACLVSGYFPEPVTVSWNSGSLTSGVHTFPSVLQSS) and a variable light chain (VL) comprising an amino acid sequence having at least about 90% sequence identity to the amino acid sequence comprising SEQ ID NO. 55 (amino acid sequence: DIVMTQTPLSLSVSPG EPASISCKASQDINHYLNWFRQKPDGTVKLLIYYTSSLHSGVPSRFSGSGSGTDF TLRISRVEADDAGVYYCQQGDHFPRTFGQGT); and any variants thereof having one or more conservative amino acid substitutions within the variable heavy and/or variable light chain regions of said antigen binding protein.

In one or more embodiments the present invention provides an antigen binding protein that specifically binds Nerve Growth Factor (NGF) and inhibits the binding between NGF and TrkA thus blocking the biological activity of NGF, as defined herein, comprising a variable heavy chain (VH) comprising an amino acid sequence having at least about 90% sequence identity to the amino acid sequence comprising SEQ ID NO. 49 (amino acid sequence: EVQLVESGGDLARPGGSLKL SCVVSGFTLTQYGINWVRQAPGKGLQWVTVIWATGATDYNSALKSRFTVSRDNAMNTVYLQMNSLRVE DTAVYYCARDGWWYATSWYFDVWGQGTLVTVSSASTTAPSVFPLAPSCGSTSGSTVALACLVSGYFPE PVTVSWNSGSLTSGVHTFPSVLQSS) and a variable light chain (VL) comprising an amino acid sequence having at least about 90% sequence identity to the amino acid sequence comprising SEQ ID NO. 57 (amino acid sequence: DIVMTQTPLSLSVSPG EPASISCKASQSINHYLNWFRQKPD GTVKLLIYYTSRLHSGVPSRFSGSGSGTDFTLRISRVEADDAGVYYCQQGSTLPRTFGQGT); and any variants thereof having one or more conservative amino acid substitutions within the variable heavy and/or variable light chain regions of said antigen binding protein.

In one or more embodiments the present invention provides an antigen binding protein that specifically binds Nerve Growth Factor (NGF) and inhibits the binding between NGF and TrkA thus blocking the biological activity of NGF as defined herein, comprising a variable heavy chain (VH) comprising an amino acid sequence having at least about 90% sequence identity to the amino acid sequence comprising SEQ ID NO. 49 (amino acid sequence: EVQLVESGGDLARPGGSLKLSCVVSG FTLTQYG INWVRQAPG KG LQWVTVIWATGATDYNSALKSRFTVSRDNAMNTVYLQMNSLRVE DTAVYY CARDGWWYATSWYFDVWGQGTLVTVSSASTTAPSVFPLAPSCGSTSGSTVALACLVSGYFPEPVTVSW NSGSLTSGVHTFPSVLQSS) and a variable light chain (VL) comprising an amino acid sequence having at least about 90% sequence identity to the amino acid sequence comprising SEQ ID NO. 59 (amino acid sequence : DIVMTQTPLSLSVSPGEPASISCRASQDISNYLNWFRQKPDGTVKLLIYYTSRLHSGVPSRFS GSGSGTDFTLRISRVEADDAGVYYCHRATTSPGPSARV); and any variants thereof having one or more conservative amino acid substitutions within the variable heavy and/or variable light chain regions of said antigen binding protein.

In one or more embodiments the present invention provides an antigen binding protein that specifically binds Nerve Growth Factor (NGF) and inhibits the binding between NGF and TrkA thus blocking the biological activity of NGF as defined herein, comprising a variable heavy chain (VH) comprising an amino acid sequence having at least about 90% sequence identity to the amino acid sequence comprising SEQ ID NO. 49 (amino acid sequence: EVQLVESGGDLARPGGSLKL SCVVSGFTLTQYGINWVRQAPGKGLQWVTVIWATGATDYNSALKSRFTVSRDNAMNTVYLQMNSLRVE DTAVYYCARDGWWYATSWYFDVWGQGTLVTVSSASTTAPSVFPLAPSCGSTSGSTVALACLVSGYFPE PVTVSWNSGSLTSGVHTFPSVLQSS) and a variable light chain (VL) comprising an amino acid sequence having at least about 90% sequence identity to the amino acid sequence comprising SEQ ID NO. 61 (amino acid sequence DIVMTQTPLSLSVSPG EPASISCKASQSINHYLNWFRQKPDGTVKL LIYYTSRLHSGVPSRFSGSGSGTDFTLRISRVEADDAGVYYCQQGSTLPRTFGQGT); and any variants thereof having one or more conservative amino acid substitutions within the variable heavy and/or variable light chain regions of said antigen binding protein.

In one or more embodiments the present invention provides an antigen binding protein that specifically binds Nerve Growth Factor (NGF) and inhibits the binding between NGF and TrkA thus blocking the biological activity of NGF as defined herein, comprising a variable heavy chain (VH) comprising an amino acid sequence having at least about 90% sequence identity to the amino acid sequence comprising SEQ ID NO. 49 (amino acid sequence: EVQLVESGGDLARPGGSLKLSCVVSGF TLTQYGINWVRQAPGKGLQWVTVIWATGATDYNSALKSRFTVSRDNAMNTVYLQMNSLRVEDTAVYYC ARDGWWYATSWYFDVWGQGTLVTVSSASTTAPSVFPLAPSCGSTSGSTVALACLVSGYFPEPVTVSWN SGSLTSGVHTFPSVLQSS) and a variable light chain (VL) comprising an amino acid sequence having at least about 90% sequence identity to the amino acid sequence comprising SEQ ID NO. 63 (amino acid sequence : DIVMTQTPLSLSVSPGEPASISCRASQDISNYLNWFRQKPDGTVKLLIYYTSRLHSGVPSRFS GSGSGTDFTLRISRVEADDAGVYYCQQGSTLPRTFGQGT); and any variants thereof having one or more conservative amino acid substitutions within the variable heavy and/or variable light chain regions of said antigen binding protein.

In one or more embodiments the present invention provides an antigen binding protein that specifically binds Nerve Growth Factor (NGF) and inhibits the binding between NGF and TrkA thus blocking the biological activity of NGF, as defined herein, comprising a variable heavy chain (VH) comprising an amino acid sequence having at least about 90% sequence identity to the amino acid sequence comprising SEQ ID NO. 49 (amino acid sequence: EVQLVESGGDLARPG GSLKLSCVVSGFTLTQYGINWVRQAPGKGLQWVTVIWATGATDYNSALKSRFTVSRDNAMNTVYLQMN SLRVEDTAVYYCARDGWWYATSWYFDVWGQGTLVTVSSASTTAPSVFPLAPSCGSTSGSTVALACLVS GYFPEPVTVSWNSGSLTSGVHTFPSVLQSS) and a variable light chain (VL) comprising an amino acid sequence having at least about 90% sequence identity to the amino acid sequence comprising SEQ ID NO. 65 (amino acid sequence : DIVMTQTPLSLSVSPGEPASISCKASQDINHYLNWFRQKPDGTVKLLIY YTSRLHSGVPSRFSGSGSGTDFTLRISRVEADDAGVYYCQQGDHFPRTFGQGT); and any variants thereof having one or more conservative amino acid substitutions within the variable heavy and/or variable light chain regions of said antigen binding protein.

In one or more embodiments the present invention provides an antigen binding protein that specifically binds Nerve Growth Factor (NGF) and inhibits the binding between NGF and TrkA thus blocking the biological activity of NGF, as defined herein, comprising a variable heavy chain (VH) comprising an amino acid sequence having at least about 90% sequence identity to the amino acid sequence comprising SEQ ID NO. 49 (amino acid sequence: EVQLVESGGDLARPGGSLKLSCVVS GFTLTQYGINWVRQAPGKGLQWVTVIWATGATDYNSALKSRFTVSRDNAMNTVYLQMNSLRVEDTAVY YCARDGWWYATSWYFDVWGQGTLVTVSSASTTAPSVFPLAPSCGSTSGSTVALACLVSGYFPEPVTVS WNSGSLTSGVHTFPSVLQSS) and a variable light chain (VL) comprising an amino acid sequence having at least about 90% sequence identity to the amino acid sequence comprising SEQ ID NO. 67 (amino acid sequence DIVMTQTPLSLSVSPG EPASISCKASQSINHYLNWFRQKPDGTVKLLIYYISSF HSGVPSRFSGSGSGTDFTLRISRVEADDAGVYYCQQSHTLPYTFGQGT); and any variants thereof having one or more conservative amino acid substitutions within the variable heavy and/or variable light chain regions of said antigen binding protein.

In one or more embodiments the present invention provides an antigen binding protein that specifically binds Nerve Growth Factor (NGF) and inhibits the binding between NGF and TrkA thus blocking the biological activity of NGF as defined herein, comprising a variable heavy chain (VH) comprising an amino acid sequence having at least about 90% sequence identity to the amino acid sequence comprising SEQ ID NO. 49 (amino acid sequence: EVQLVESGGDLARPGGS LKLSCVVSGFTLTQYGINWVRQAPGKGLQWVTVIWATGATDYNSALKSRFTVSRDNAMNTVYLQMNSLR VEDTAVYYCARDGWWYATSWYFDVWGQGTLVTVSSASTTAPSVFPLAPSCGSTSGSTVALACLVSGYF PEPVTVSWNSGSLTSGVHTFPSVLQSS) and a variable light chain (VL) comprising an amino acid sequence having at least about 90% sequence identity to the amino acid sequence comprising SEQ ID NO. 69 (amino acid sequence: DIVMTQTPLSLSVSPGEPASISCKASQDINHYLNWFR QKPDGTVKLLIYYVTSLHAGVPSRFSGSGSGTDFTLRISRVEADDAGVYYCQQGDHFPRTFGQGT); and any variants thereof having one or more conservative amino acid substitutions within the variable heavy and/or variable light chain regions of said antigen binding protein.

In one or more embodiments the present invention provides an antigen binding protein that specifically binds Nerve Growth Factor (NGF) and inhibits the binding between NGF and TrkA thus blocking the biological activity of NGF as defined herein, comprising a variable heavy chain (VH) comprising an amino acid sequence having at least about 90% sequence identity to the amino acid sequence comprising SEQ ID NO. 49 (amino acid sequence: EVQLVESGGDLARPGGSLKLSC VVSGFTLTQYGINWVRQAPGKGLQWVTVIWATGATDYNSALKSRFTVSRDNAMNTVYLQMNSLRVEDT AVYYCARDGWWYATSWYFDVWGQGTLVTVSSASTTAPSVFPLAPSCGSTSGSTVALACLVSGYFPEPV TVSWNSGSLTSGVHTFPSVLQSS) and a variable light chain (VL) comprising an amino acid sequence having at least about 90% sequence identity to the amino acid sequence comprising SEQ ID NO. 69 (amino acid sequence DIVMTQTPLSLSVSPGEPASISCKASQDINHYLNWFRQKPDGTVKLLIY YTSRLHSGVPSRFSGSGSGTDFTLRISRVEADDAGVYYCQQGDHFPRTFGQGT); and any variants thereof having one or more conservative amino acid substitutions within the variable heavy and/or variable light chain regions of said antigen binding protein.

In one or more embodiments the present invention provides an antigen binding protein that specifically binds Nerve Growth Factor (NGF) and inhibits the binding between NGF and TrkA thus blocking the biological activity of NGF as defined herein, comprising a variable heavy chain (VH) comprising an amino acid sequence having at least about 90% sequence identity to the amino acid sequence comprising SEQ ID NO. 49 (amino acid sequence: EVQLVESGGDLARPGGSLKLSCVVS GFTLTQYGINWVRQAPGKGLQWVTVIWATGATDYNSALKSRFTVSRDNAMNTVYLQMNSLRVEDTAVY YCARDGWWYATSWYFDVWGQGTLVTVSSASTTAPSVFPLAPSCGSTSGSTVALACLVSGYFPEPVTVS WNSGSLTSGVHTFPSVLQSS) and a variable light chain (VL) comprising an amino acid sequence having at least about 90% sequence identity to the amino acid sequence comprising SEQ ID NO. 71 (amino acid sequence: DIVMTQTPLSLSVSPG EPASISCRASQDISNYLNWFRQKPDGTVKLLIYKTN SLQTGVPSRFSGSGSGTDFTLRISRVEADDAGVYYCQQGSTLPRTFGQGT); and any variants thereof having one or more conservative amino acid substitutions within the variable heavy and/or variable light chain regions of said antigen binding protein.

In one or more embodiments the present invention provides an antigen binding protein that specifically binds Nerve Growth Factor (NGF) and inhibits the binding between NGF and TrkA thus blocking the biological activity of NGF as defined herein, comprising a variable heavy chain (VH) comprising an amino acid sequence having at least about 90% sequence identity to the amino acid sequence comprising SEQ ID NO. 49 (amino acid sequence: EVQLVESGGDLARPGGSLKLSCVVS GFTLTQYGINWVRQAPGKGLQWVTVIWATGATDYNSALKSRFTVSRDNAMNTVYLQMNSLRVEDTAVY YCARDGWWYATSWYFDVWGQGTLVTVSSASTTAPSVFPLAPSCGSTSGSTVALACLVSGYFPEPVTVS WNSGSLTSGVHTFPSVLQSS) and a variable light chain (VL) comprising an amino acid sequence having at least about 90% sequence identity to the amino acid sequence comprising SEQ ID NO. 73 (amino acid sequence: DIVMTQTPLSLSVSPG EPASISCRASQDISNYLNWFRQKPDGTVKLLIYYV TSLHAGVPSRFSGSGSGTDFTLRISRVEADDAGVYYCQQGSTLPRTFGQGT); and any variants thereof having one or more conservative amino acid substitutions within the variable heavy and/or variable light chain regions of said antigen binding protein.

In one or more embodiments the present invention provides an antigen binding protein that specifically binds Nerve Growth Factor (NGF) and inhibits the binding between NGF and TrkA thus blocking the biological activity of NGF as defined herein, comprising a variable heavy chain (VH) comprising an amino acid sequence having at least about 90% sequence identity to the amino acid sequence comprising SEQ ID NO. 49 (amino acid sequence: EVQLVESGGDLARPGG SLKLSCVVSGFTLTQYGINWVRQAPGKGLQWVTVIWATGATDYNSALKSRFTVSRDNAMNTVYLQMNSL RVEDTAVYYCARDGWWYATSWYFDVWGQGTLVTVSSASTTAPSVFPLAPSCGSTSGSTVALACLVSGY FPEPVTVSWNSGSLTSGVHTFPSVLQSS) and a variable light chain (VL) comprising an amino acid sequence having at least about 90% sequence identity to the amino acid sequence comprising SEQ ID NO. 75 (amino acid sequence: EIVMTQSPASLSLSQEEKVTITCRASQDISNYLNWYQQKPGQAPKL LIYYTSRLHSGVPSRFSGSGSGTDFSFTISSLEPEDVAVYYCQQGNMFPYTFGGGT); and any variants thereof having one or more conservative amino acid substitutions within the variable heavy and/or variable light chain regions of said antigen binding protein.

In one or more embodiments the present invention provides an antigen binding protein that specifically binds Nerve Growth Factor (NGF) and inhibits the binding between NGF and TrkA thus blocking the biological activity of NGF as defined herein, comprising a variable heavy chain (VH) comprising an amino acid sequence having at least about 90% sequence identity to the amino acid sequence comprising SEQ ID NO. 49 (amino acid sequence: EVQLVESGGDLARPGGSLKL SCVVSGFTLTQYGINWVRQAPGKGLQWVTVIWATGATDYNSALKSRFTVSRDNAMNTVYLQMNSLRVE DTAVYYCARDGWWYATSWYFDVWGQGTLVTVSSASTTAPSVFPLAPSCGSTSGSTVALACLVSGYFPE PVTVSWNSGSLTSGVHTFPSVLQSS) and a variable light chain (VL) comprising an amino acid sequence having at least about 90% sequence identity to the amino acid sequence comprising SEQ ID NO. 77 (amino acid sequence: EIVMTQSPASLSLSQEEKVTITCKASQDINHYLNWYQQKPGQAP KLLIYYTSRLHSGVPSRFSGSGSGTDFSFTISSLEPEDVAVYYCQQGNMFPYTFGGGT); and any variants thereof having one or more conservative amino acid substitutions within the variable heavy and/or variable light chain regions of said antigen binding protein.

In one or more embodiments the present invention provides an antigen binding protein that specifically binds Nerve Growth Factor (NGF) and inhibits the binding between NGF and TrkA thus blocking the biological activity of NGF as defined herein, comprising a variable heavy chain (VH) comprising an amino acid sequence having at least about 90% sequence identity to the amino acid sequence comprising SEQ ID NO. 49 (amino acid sequence: EVQLVESGGDLARPGG SLKLSCVVSGFTLTQYGINWVRQAPGKGLQWVTVIWATGATDYNSALKSRFTVSRDNAMNTVYLQMNSL RVEDTAVYYCARDGWWYATSWYFDVWGQGTLVTVSSASTTAPSVFPLAPSCGSTSGSTVALACLVSGY FPEPVTVSWNSGSLTSGVHTFPSVLQSS) and a variable light chain (VL) comprising an amino acid sequence having at least about 90% sequence identity to the amino acid sequence comprising SEQ ID NO. 201 (amino acid sequence: DIVMTQTPLSLSVSPGEPASISCKASQDINHYLNWFRQKPDG TVKLLIYTTRLQASGVPSRFSGSGSGTDFTLRISRVEADDAGVYYCQQGDH FPRTFGQGT); and any variants thereof having one or more conservative amino acid substitutions within the variable heavy and/or variable light chain regions of said antigen binding protein.

In one or more embodiments, the present invention provides an antigen binding protein of the invention that comprises a caninized antibody.

In another aspect the present invention provides an antigen binding protein that specifically binds Nerve Growth Factor (NGF) and inhibits the binding between NGF and TrkA thus blocking the biological activity of NGF, as defined herein, comprising: a heavy chain variable region (VH) having at least 90% sequence identity to the amino acid sequences selected from SEQ ID NO. 85 or SEQ. ID NO. 92; and a light chain variable region (VL) having at least 90% sequence identity to the amino acid sequences selected from SEQ ID NO. 87, SEQ ID. NO.89, SEQ ID or SEQ ID NO. 94; and any variants thereof having one or more conservative amino acid substitutions in at least one of the heavy chain variable regions or one of the light chain variable regions of said antigen binding protein.

In one or more embodiments the present invention provides an antigen binding protein that specifically binds Nerve Growth Factor (NGF) and inhibits the binding between NGF and TrkA thus blocking the biological activity of NGF as defined herein, comprising a variable heavy chain (VH) comprising an amino acid sequence having at least about 90% sequence identity to the amino acid sequence comprising SEQ ID NO. 85 (amino acid sequence: DVQLVESGGDLVQPGGS LRLTCVASGFTLTQYGINWVRQAPGKGLQWVAVIWATGATDYNSALKSRFTISRDNAKNTLYLQMNSLKT EDTATYYCARDGWWYATSWYFDVWGQGALVTVSS) and a variable light chain (VL) comprising an amino acid sequence having at least about 90% sequence identity to the amino acid sequence comprising SEQ ID NO. 87 (amino acid sequence DIVMTQTPLSLSVTPGEPASISC KASQDINHYLNWYLQKPDGTVKLLIYYTSRLHSGVPSRFSGSGSGTDFTLRISRVEADDVGVYYCQQGD HFPRTFGPGT); and any variants thereof having one or more conservative amino acid substitutions within the variable heavy and/or variable light chain regions of said antigen binding protein.

In one or more embodiments the present invention provides an antigen binding protein as defined herein, comprising a variable heavy chain (VH) comprising an amino acid sequence having at least about 90% sequence identity to the amino acid sequence comprising SEQ ID NO. 85 (amino acid sequence: DVQLVESGGDLVQPGGSLRLTCVASGFTLTQYGINWVRQAPGKGLQWVAVIWATGATDYNSALKSRFTI SRDNAKNTLYLQMNSLKTEDTATYYCARDGWWYATSWYFDVWGQGALVTVSS) and a variable light chain (VL) comprising an amino acid sequence having at least about 90% sequence identity to the amino acid sequence comprising SEQ ID NO. 89 (amino acid sequence: DIVMTQTPLSLPVTPGEP ASISCKASQDINHYLNWYLQKPGQSPRLLIYYTSRLHSGVPSRFSGSGSGTDFTLRISSVEADDVGVYYC QQGDHFPRTFGQGT); and any variants thereof having one or more conservative amino acid substitutions within the variable heavy and/or variable light chain regions of said antigen binding protein.

In one or more embodiments the present invention provides an antigen binding protein, as defined herein, comprising a variable heavy chain (VH) comprising an amino acid sequence having at least about 90% sequence identity to the amino acid sequence comprising SEQ ID NO. 92 (amino acid sequence: DVQLVESGGDLVKPGGSLRLTCVASGFTLTQYGINWVRQAPGKGLQWVAVIWATGATDYN SALKSRFTMSRDNARNTLYLQMNSLKTEDTATYYCARDGWWYATSWYFDVWGQGTLVTVSS) and a variable light chain (VL) comprising an amino acid sequence having at least about 90% sequence identity to the amino acid sequence comprising SEQ ID NO. 89 (amino acid sequence: DIVMTQTPLSLPVTPGEPASISCKASQDINHYLNWYLQKPGQSPRLLIYYTSRLHSGVPSRFSGSGSGTDF TLRISSVEADDVGVYYCQQGDHFPRTFGQGT); and any variants thereof having one or more conservative amino acid substitutions within the variable heavy and/or variable light chain regions of said antigen binding protein.

In one or more embodiments the present invention provides an antigen binding protein, as defined herein, comprising a variable heavy chain (VH) comprising an amino acid sequence having at least about 90% sequence identity to the amino acid sequence comprising SEQ ID NO. 85 (amino acid sequence: DVQLVESGGDLVQPGGSLRLTCVASGFTLTQYGINWVRQAPGKGLQWVAVIWATGATDY NSALKSRFTISRDNAKNTLYLQMNSLKTEDTATYYCARDGWWYATSWYFDVWGQGALVTVSS) and a variable light chain (VL) comprising an amino acid sequence having at least about 90% sequence identity to the amino acid sequence comprising SEQ ID NO. 94 (amino acid sequence: EIQMTQSPSSLSASPGDRVTITCKASQDINHYLNWYQQKPGKVPKLLIYYTSRLHSGVPSRFSGSGSGTD FTLTISSLEPEDAATYYCQQGDHFPRTFGGGT); and any variants thereof having one or more conservative amino acid substitutions within the variable heavy and/or variable light chain regions of said antigen binding protein.

In one or more embodiments the present invention provides an antigen binding protein, as defined herein, comprising a variable heavy chain (VH) comprising an amino acid sequence having at least about 90% sequence identity to the amino acid sequence comprising SEQ ID NO. 92 (amino acid sequence: DVQLVESGGDLVKPGGSLRLTCVASGFTLTQYGINWVRQAPGKGLQWVAVIWATGATDYN SALKSRFTMSRDNARNTLYLQMNSLKTEDTATYYCARDGWWYATSWYFDVWGQGTLVTVSS) and a variable light chain (VL) comprising an amino acid sequence having at least about 90% sequence identity to the amino acid sequence comprising SEQ ID NO. 87 (amino acid sequence: DIVMTQTPL SLSVTPG EPASISCKASQDINHYLNWYLQKPDGTVKLLIYYTSRLHSGVPSRFSGSGSGTDFTLRISRVEA DDVGVYYCQQGDHFPRTFGPGT); and any variants thereof having one or more conservative amino acid substitutions within the variable heavy and/or variable light chain regions of said antigen binding protein.

In one or more embodiments, the present invention provides an antigen binding protein that comprises a felinized antibody.

In another aspect the present invention provides a nucleic acid sequence that encodes the antigen binding protein of the invention comprising at least about 90% sequence identity to the nucleic acids that encode the variable heavy chain (VH) of the amino acid sequence comprising SEQ.ID NO.49 which comprises the nucleic acid sequence SEQ ID NO.50 (nucleic acid sequence: AAGCTTCCACCATGAAGCACCTGTGGTTCTTTCTGCTGCTGGTGGCCGCTCCCAGATGGGTGCTGA GCGAGGTGCAGCTGGTGGAATCTGGCGGCGACCTGGCCAGACCTGGCGGCAGCCTGAAGCTGAG CTGCGTGGTGTCCGGCTTCACCCTGACCCAGTACGGCATCAACTGGGTCCGCCAGGCCCCTGGCA AGGGCCTGCAGTGGGTCACAGTGATCTGGGCCACCGGCGCCACCGACTACAACAGCGCCCTGAAG TCCCGGTTCACCGTGTCTCGGGACAACGCCATGAACACCGTGTACCTGCAGATGAACAGCCTGCGG GTGGAAGATACCGCCGTGTACTACTGCGCCAGAGACGGCTGGTGGTACGCCACCAGCTGGTACTTC GACGTGTGGGGCCAGGGCACACTGGTCACAGTCTCGAGC);

and a nucleic acid sequence having at least 90% sequence identity to the nucleic acids selected from the group consisting of:

SEQ ID NO. 52:
nucleic acid sequence (encoding the amino
acid sequence comprising SEQ ID NO. 51):
GATATTGTGATGACCCAGACCCCGCTGAGCCTGAG
CGTGAGCCCGGGCGAACCGGCGAGCATTAGCTGCA
AAGCGAGCCAGGATATTAACCATTATCTGAACTGG
TTTCGCCAGAAACCGGATGGCACCGTGAAACTGCT
GATTTATTATACCAGCCGCCTGCATAGCGGCGTGC
CGAGCCGCTTTAGCGGCAGCGGCAGCGGCACCGAT
TTTACCCTGCGCATTAGCCGCGTGGAAGCGGATGA
TGCGGGCGTGTATTATTGCCAGCAGGGCGATCATT
TTCCGCGCACCTTTGGCCAGGGCACCAAACTGGAA
ATTAAACGCAACGATGCGCAGCCGGCGGTGTATCT
GTTTCAGCCGAGCCCGGATCAGCTGCATACCGGCA
GCGCGAGCGTGGTGTGCCTGCTGAACAGCTTTTAT
CCGAAAGATATTAACGTGAAATGGAAAGTGGATGG
CGTGATTCAGGATACCGGCATTCAGGAAAGCGTGA
CCGAACAGGATAAAGATAGCACCTATAGCCTGAGC
AGCACCCTGACCATGAGCAGCACCGAATATCTGAG
CCATGAACTGTATAGCTGCGAAATTACCCATAAAA
GCCTGCCGAGCACCCTGATTAAAAGCTTTCAGCGC
AGCGAATGC;
SEQ ID NO. 54:
nucleic acid sequence (encoding the amino
acid sequence comprising SEQ ID NO. 53):
AAGCTTGCCACCATGGTGCTGCAGACACAGGTGTT
CATCTCTCTGCTGCTGTGGATTAGTGGAGCCTACG
GCGACATCGTGATGACCCAGACACCTCTGTCACTG
AGCGTGTCCCCAGGGGAACCCGCCTCTATCAGTTG
CCGGGCCAGCCAGAGCATCAGCAACAACCTGAACT
GGTTCAGACAGAAGCCAGATGGGACCGTCAAGCTA
CTGATCTACTACATCAGCTCGTTCCACAGCGGAGT
GCCCTCTCGCTTTTCAGGCAGCGGGTCCGGAACAG
ACTTTACTCTGCGGATCTCCAGAGTGGAAGCCGAC
GATGCTGGCGTGTACTATTGCCAGCAGGGCGACCA
CTTCCCCTACACCTTCGGCCAGGGTACC
SEQ ID NO. 56
nucleic acid sequence (encoding the amino
acid sequence comprising SEQ ID NO. 55):
AAGCTTCCACCATGAAGCACCTGTGGTTCTTTCTG
CTGCTGGTGGCCGCTCCCAGATGGGTGCTGAGCGA
GGTGCAGCTGGTGGAATCTGGCGGCGACCTGGCCA
GACCTGGCGGCAGCCTGAAGCTGAGCTGCGTGGTG
TCCGGCTTCACCCTGACCCAGTACGGCATCAACTG
GGTCCGCCAGGCCCCTGGCAAGGGCCTGCAGTGGG
TCACAGTGATCTGGGCCACCGGCGCCACCGACTAC
AACAGCGCCCTGAAGTCCCGGTTCACCGTGTCTCG
GGACAACGCCATGAACACCGTGTACCTGCAGATGA
ACAGCCTGCGGGTGGAAGATACCGCCGTGTACTAC
TGCGCCAGAGACGGCTGGTGGTACGCCACCAGCTG
GTACTTCGACGTGTGGGGCCAGGGCACACTGGTCA
CAGTCTCGAGC
SEQ ID NO. 58
nucleic acid sequence (encoding the amino
acid sequence comprising SEQ ID NO. 57):
AAGCTTGCCACCATGGTGCTGCAGACACAGGTGTT
CATCTCTCTGCTGCTGTGGATTAGTGGAGCCTACG
GCGACATCGTGATGACCCAGACACCTCTGTCACTG
AGCGTGTCCCCAGGGGAACCCGCCTCTATCAGTTG
CAAGGCCAGCCAGAGCATCAACCACTACCTGAACT
GGTTCAGACAGAAGCCAGATGGGACCGTCAAGCTA
CTGATCTACTACACATCAAGGCTGCATTCAGGAGT
GCCCTCTCGCTTTTCAGGCAGCGGGTCCGGAACAG
ACTTTACTCTGCGGATCTCCAGAGTGGAAGCCGAC
GATGCTGGCGTGTACTATTGCCAACAGGGGAGTAC
CCTGCCCAGGACCTTCGGCCAGGGTACC
SEQ ID NO. 60
nucleic acid sequence (encoding the amino
acid sequence comprising SEQ ID NO. 59):
AAGCTTGCCACCATGGTGCTGCAGACACAGGTGTT
CATCTCTCTGCTGCTGTGGATTAGTGGAGCCTACG
GCGACATCGTGATGACCCAGACACCTCTGTCACTG
AGCGTGTCCCCAGGGGAACCCGCCTCTATCAGTTG
CAGAGCTTCTCAAGATATTAGCAACTATCTGAATT
GGTTCAGACAGAAGCCAGATGGGACCGTCAAGCTA
CTGATCTACTACACATCAAGGCTGCATTCAGGAGT
GCCCTCTCGCTTTTCAGGCAGCGGGTCCGGAACAG
ACTTTACTCTGCGGATCTCCAGAGTGGAAGCCGAC
GATGCTGGCGTGTACTATTGCCACAGGGCGACCAC
TTCCCCCGGACCTTCGGCCAGGGTACC
SEQ ID NO. 62
nucleic acid sequence (encoding the amino
acid sequence comprising SEQ ID NO. 61):
AAGCTTGCCACCATGGTGCTGCAGACACAGGTGTT
CATCTCTCTGCTGCTGTGGATTAGTGGAGCCTACG
GCGACATCGTGATGACCCAGACACCTCTGTCACTG
AGCGTGTCCCCAGGGGAACCCGCCTCTATCAGTTG
CAAGGCCAGCCAGGACATCAACCACTACCTGAACT
GGTTCAGACAGAAGCCAGATGGGACCGTCAAGCTA
CTGATCTACTACACATCAAGGCTGCATTCAGGAGT
GCCCTCTCGCTTTTCAGGCAGCGGGTCCGGAACAG
ACTTTACTCTGCGGATCTCCAGAGTGGAAGCCGAC
GATGCTGGCGTGTACTATTGCCAACAGGGGAGTAC
CCTGCCCAGGACCTTCGGCCAGGGTACC
SEQ ID NO. 64
nucleic acid sequence (encoding the amino
acid sequence comprising SEQ ID NO 63
AAGCTTGCCACCATGGTGCTGCAGACACAGGTGTT
CATCTCTCTGCTGCTGTGGATTAGTGGAGCCTACG
GCGACATCGTGATGACCCAGACACCTCTGTCACTG
AGCGTGTCCCCAGGGGAACCCGCCTCTATCAGTTG
CAGAGCTTCTCAAGATATTAGCAACTATCTGAATT
GGTTCAGACAGAAGCCAGATGGGACCGTCAAGCTA
CTGATCTACTACACATCAAGGCTGCATTCAGGAGT
GCCCTCTCGCTTTTCAGGCAGCGGGTCCGGAACAG
ACTTTACTCTGCGGATCTCCAGAGTGGAAGCCGAC
GATGCTGGCGTGTACTATTGCCAACAGGGGAGTAC
CCTGCCCAGGACCTTCGGCCAGGGTACC
SEQ ID NO. 66
(nucleic acid sequence (encoding the amino
acid sequence comprising SEQ ID NO. 65):
GATATTGTGATGACCCAGACCCCGCTGAGCCTGAG
CGTGAGCCCGGGCGAACCGGCGAGCATTAGCTGCC
GCGCGAGCCAGGATATTAGCAACTATCTGAACTGG
TTTCGCCAGAAACCGGATGGCACCGTGAAACTGCT
GATTTATTATACCAGCAGCCTGCATAGCGGCGTGC
CGAGCCGCTTTAGCGGCAGCGGCAGCGGCACCGAT
TTTACCCTGCGCATTAGCCGCGTGGAAGCGGATGA
TGCGGGCGTGTATTATTGCCAGCAGGGCAGCACCC
TGCCGCGCACCTTTGGCCAGGGCACCAAACTGGAA
ATTAAACGCAACGATGCGCAGCCGGCGGTGTATCT
GTTTCAGCCGAGCCCGGATCAGCTGCATACCGGCA
GCGCGAGCGTGGTGTGCCTGCTGAACAGCTTTTAT
CCGAAAGATATTAACGTGAAATGGAAAGTGGATGG
CGTGATTCAGGATACCGGCATTCAGGAAAGCGTGA
CCGAACAGGATAAAGATAGCACCTATAGCCTGAGC
AGCACCCTGACCATGAGCAGCACCGAATATCTGAG
CCATGAACTGTATAGCTGCGAAATTACCCATAAAA
GCCTGCCGAGCACCCTGATTAAAAGCTTTCAGCGC
AGCGAATGC
SEQ ID NO. 68
nucleic acid sequence (encoding the amino
acid sequence comprising SEQ ID NO. 67):
AAGCTTGCCACCATGGTGCTGCAGACACAGGTGTT
CATCTCTCTGCTGCTGTGGATTAGTGGAGCCTACG
GCGACATCGTGATGACCCAGACACCTCTGTCACTG
AGCGTGTCCCCAGGGGAACCCGCCTCTATCAGTTG
CAAGGCCAGCCAGAGCATCAACCACTACCTGAACT
GGTTCAGACAGAAGCCAGATGGGACCGTCAAGCTA
CTGATCTACTACATCAGCTCGTTCCACAGCGGAGT
GCCCTCTCGCTTTTCAGGCAGCGGGTCCGGAACAG
ACTTTACTCTGCGGATCTCCAGAGTGGAAGCCGAC
GATGCTGGCGTGTACTATTGCCAGCAGAGCCACAC
CCTGCCCTACACCTTCGGCCAGGGTACC
SEQ ID NO. 70
nucleic acid sequence (encoding the amino
acid sequence comprising SEQ ID NO. 69):
GATATTGTGATGACCCAGACCCCGCTGAGCCTGAG
CGTGAGCCCGGGCGAACCGGCGAGCATTAGCTGCA
AAGCGAGCCAGGATATTAACCATTATCTGAACTGG
TTTCGCCAGAAACCGGATGGCACCGTGAAACTGCT
GATTTATTATGTGACCAGCCTGCATGCGGGCGTGC
CGAGCCGCTTTAGCGGCAGCGGCAGCGGCACCGAT
TTTACCCTGCGCATTAGCCGCGTGGAAGCGGATGA
TGCGGGCGTGTATTATTGCCAGCAGGGCGATCATT
TTCCGCGCACCTTTGGCCAGGGCACCAAACTGGAA
ATTAAACGCAACGATGCGCAGCCGGCGGTGTATCT
GTTTCAGCCGAGCCCGGATCAGCTGCATACCGGCA
GCGCGAGCGTGGTGTGCCTGCTGAACAGCTTTTAT
CCGAAAGATATTAACGTGAAATGGAAAGTGGATGG
CGTGATTCAGGATACCGGCATTCAGGAAAGCGTGA
CCGAACAGGATAAAGATAGCACCTATAGCCTGAGC
AGCACCCTGACCATGAGCAGCACCGAATATCTGAG
CCATGAACTGTATAGCTGCGAAATTACCCATAAAA
GCCTGCCGAGCACCCTGATTAAAAGCTTTCAGCGC
AGCGAATGC
SEQ ID NO. 72
nucleic acid sequence (encoding the amino
acid sequence comprising SEQ ID NO. 71):
GATATTGTGATGACCCAGACCCCGCTGAGCCTGAG
CGTGAGCCCGGGCGAACCGGCGAGCATTAGCTGCC
GCGCGAGCCAGGATATTAGCAACTATCTGAACTGG
TTTCGCCAGAAACCGGATGGCACCGTGAAACTGCT
GATTTATAAAACCAACAGCCTGCAGACCGGCGTGC
CGAGCCGCTTTAGCGGCAGCGGCAGCGGCACCGAT
TTTACCCTGCGCATTAGCCGCGTGGAAGCGGATGA
TGCGGGCGTGTATTATTGCCAGCAGGGCAGCACCC
TGCCGCGCACCTTTGGCCAGGGCACCAAACTGGAA
ATTAAACGCAACGATGCGCAGCCGGCGGTGTATCT
GTTTCAGCCGAGCCCGGATCAGCTGCATACCGGCA
GCGCGAGCGTGGTGTGCCTGCTGAACAGCTTTTAT
CCGAAAGATATTAACGTGAAATGGAAAGTGGATGG
CGTGATTCAGGATACCGGCATTCAGGAAAGCGTGA
CCGAACAGGATAAAGATAGCACCTATAGCCTGAGC
AGCACCCTGACCATGAGCAGCACCGAATATCTGAG
CCATGAACTGTATAGCTGCGAAATTACCCATAAAA
GCCTGCCGAGCACCCTGATTAAAAGCTTTCAGCGC
AGCGAATGC
SEQ ID NO. 74
nucleic acid sequence (encoding the amino
acid sequence comprising SEQ ID NO. 73):
GATATTGTGATGACCCAGACCCCGCTGAGCCTGAG
CGTGAGCCCGGGCGAACCGGCGAGCATTAGCTGCC
GCGCGAGCCAGGATATTAGCAACTATCTGAACTGG
TTTCGCCAGAAACCGGATGGCACCGTGAAACTGCT
GATTTATTATGTGACCAGCCTGCATGCGGGCGTGC
CGAGCCGCTTTAGCGGCAGCGGCAGCGGCACCGAT
TTTACCCTGCGCATTAGCCGCGTGGAAGCGGATGA
TGCGGGCGTGTATTATTGCCAGCAGGGCAGCACCC
TGCCGCGCACCTTTGGCCAGGGCACCAAACTGGAA
ATTAAACGCAACGATGCGCAGCCGGCGGTGTATCT
GTTTCAGCCGAGCCCGGATCAGCTGCATACCGGCA
GCGCGAGCGTGGTGTGCCTGCTGAACAGCTTTTAT
CCGAAAGATATTAACGTGAAATGGAAAGTGGATGG
CGTGATTCAGGATACCGGCATTCAGGAAAGCGTGA
CCGAACAGGATAAAGATAGCACCTATAGCCTGAGC
AGCACCCTGACCATGAGCAGCACCGAATATCTGAG
CCATGAACTGTATAGCTGCGAAATTACCCATAAAA
GCCTGCCGAGCACCCTGATTAAAAGCTTTCAGCGC
AGCGAATGC
SEQ ID NO. 76
nucleic acid sequence (encoding the amino
acid sequence comprising SEQ ID NO. 75):
AAGCTTGGCCACCATGAGTGTTCCTACCCAAGTGC
TGGGACTGCTGCTGCTGTGGCTGACAGATGCTCGG
TGCGAGATAGTCATGACCCAGTCACCGGCATCTCT
GAGCCTGAGCCAGGAAGAGAAGGTAACTATCACGT
GTCGGGCCTCTCAGGACATTAGCAACTACCTGAAT
TGGTATCAGCAGAAACCAGGACAGGCCCCCAAGTT
GTTGATATACTACACTTCCCGCCTGCACAGTGGGG
TCCCCTCCCGATTCAGCGGATCCGGGTCCGGCACG
GACTTCAGCTTTACTATCTCCAGTTTGGAGCCCGA
AGATGTTGCTGTGTATTACTGTCAGCAGGGTAATA
TGTTTCCGTATACATTCGGCGGAGGTACC
SEQ ID NO. 78
nucleic acid sequence (encoding the amino
acid sequence comprising SEQ ID NO. 77):
AGCTTGGCCACCATGAGTGTTCCTACCCAAGTGCT
GGGACTGCTGCTGCTGTGGCTGACAGATGCTCGGT
GCGAGATAGTCATGACCCAGTCACCGGCATCTCTG
AGCCTGAGCCAGGAAGAGAAGGTAACTATCACGTG
TAAGGCCAGCCAGGACATCAACCACTACCTGAACT
GGTATCAGCAGAAACCAGGACAGGCCCCCAAGTTG
TTGATATACTACACTTCCCGCCTGCACAGTGGGGT
CCCCTCCCGATTCAGCGGATCCGGGTCCGGCACGG
ACTTCAGCTTTACTATCTCCAGTTTGGAGCCCGAA
GATGTTGCTGTGTATTACTGTCAGCAGGGTAATAT
GTTTCCGTATACATTCGGCGGAGGTACC;
and
SEQ ID NO. 176
nucleic acid sequence (encoding the amino
acid sequence comprising SEQ ID NO. 183):
GATATTGTGATGACCCAGACCCCGCTGAGCCTGAG
CGTGAGCCCGGGCGAACCGGCGAGCATTAGCTGCA
AAGCGAGCCAGGATATTAACCATTATCTGAACTGG
TTTCGCCAGAAACCGGATGGCACCGTGAAACTGCT
GATTTATACCACCCGCCTGCAGGCGAGCGGCGTGC
CGAGCCGCTTTAGCGGCAGCGGCAGCGGCACCGAT
TTTACCCTGCGCATTAGCCGCGTGGAAGCGGATGA
TGCGGGCGTGTATTATTGCCAGCAGGGCGATCATT
TTCCGCGCACCTTTGGCCAGGGCACC;

and any variants thereof having one or more nucleic acid substitutions that encode conservative amino acid substitutions with the variable heavy and/or light chain regions of said antigen binding protein.

In one or more embodiments the present invention provides a nucleic acid sequence that encodes the antigen binding protein of the invention comprising at least about 90% sequence identity to the nucleic acid sequence that encodes the variable heavy chain (VH) which comprises SEQ ID NO. 50 and a nucleic acid sequence having at least 90% sequence identity the nucleic acid sequence that encodes the variable light chain (VL) comprising SEQ ID NO.52; and any variants thereof having any one or more nucleic acid substitutions that encode conservative amino acid substitutions with the variable heavy and/or light chain regions of said antigen binding protein.

In one or more embodiments the present invention provides a nucleic acid sequence that encodes the antigen binding protein of the invention comprising at least about 90% sequence identity to the nucleic acid sequence that encodes the variable heavy chain (VH) comprising SEQ ID NO.50, and a nucleic acid sequence having at least 90% sequence identity the nucleic acid sequence that encodes the variable light chain (VL) comprising SEQ ID NO.54; and any variants thereof having any one or more nucleic acid substitutions that encode conservative amino acid substitutions with the variable heavy and/or light chain regions of said antigen binding protein.

In one or more embodiments the present invention provides a nucleic acid sequence that encodes the antigen binding protein of the invention comprising at least about 90% sequence identity to the nucleic acid sequence that encodes the variable heavy chain (VH) comprising SEQ ID NO.50 and a nucleic acid sequence having at least 90% sequence identity the nucleic acid sequence that encodes the variable light chain (VL) which comprises the nucleic acid sequence comprising SEQ ID NO.56; and any variants thereof having any one or more nucleic acid substitutions that encode conservative amino acid substitutions with the variable heavy and/or light chain regions of said antigen binding protein.

In one or more embodiments the present invention provides a nucleic acid sequence that encodes the antigen binding protein of the invention comprising at least about 90% sequence identity to the nucleic acid sequence that encodes the variable heavy chain (VH) comprising SEQ ID NO. 50 and a nucleic acid sequence having at least 90% sequence identity the nucleic acid sequence that encodes the variable light chain (VL) which comprises the nucleic acid sequence comprising SEQ ID NO.58; and any variants thereof having any one or more nucleic acid substitutions that encode conservative amino acid substitutions with the variable heavy and/or light chain regions of said antigen binding protein.

In one or more embodiments the present invention provides a nucleic acid sequence that encodes the antigen binding protein of the invention comprising at least about 90% sequence identity to the nucleic acid sequence that encodes the variable heavy chain (VH) comprising SEQ ID NO. 50 and a nucleic acid sequence having at least 90% sequence identity the nucleic acid sequence that encodes the variable light chain (VL) which comprises the nucleic acid sequence comprising SEQ ID NO.60; and any variants thereof having any one or more nucleic acid substitutions that encode conservative amino acid substitutions with the variable heavy and/or light chain regions of said antigen binding protein.

In one or more embodiments the present invention provides a nucleic acid sequence that encodes the antigen binding protein of the invention comprising at least about 90% sequence identity to the nucleic acid sequence that encodes the variable heavy chain (VH) comprising SEQ ID NO. 50 and a nucleic acid sequence having at least 90% sequence identity the nucleic acid sequence that encodes the variable light chain (VL) which comprises the nucleic acid sequence comprising SEQ ID NO.62; and any variants thereof having any one or more nucleic acid substitutions that encode conservative amino acid substitutions with the variable heavy and/or light chain regions of said antigen binding protein.

In one or more embodiments the present invention provides a nucleic acid sequence that encodes the antigen binding protein of the invention comprising at least about 90% sequence identity to the nucleic acid sequence that encodes the variable heavy chain (VH) comprising SEQ ID NO. 50 and a nucleic acid sequence having at least 90% sequence identity the nucleic acid sequence that encodes the variable light chain (VL) which comprises the nucleic acid sequence comprising SEQ ID NO.64; and any variants thereof having any one or more nucleic acid substitutions that encode conservative amino acid substitutions with the variable heavy and/or light chain regions of said antigen binding protein.

In one or more embodiments the present invention provides a nucleic acid sequence that encodes the antigen binding protein of the invention comprising at least about 90% sequence identity to the nucleic acid sequence that encodes the variable heavy chain (VH) comprising SEQ ID NO. 50 and a nucleic acid sequence having at least 90% sequence identity the nucleic acid sequence that encodes the variable light chain (VL) which comprises the nucleic acid sequence comprising SEQ ID NO.66; and any variants thereof having any one or more nucleic acid substitutions that encode conservative amino acid substitutions with the variable heavy and/or light chain regions of said antigen binding protein.

In one or more embodiments the present invention provides a nucleic acid sequence that encodes the antigen binding protein of the invention comprising at least about 90% sequence identity to the nucleic acid sequence that encodes the variable heavy chain (VH) comprising SEQ ID NO. 50 and a nucleic acid sequence having at least 90% sequence identity the nucleic acid sequence that encodes the variable light chain (VL) which comprises the nucleic acid sequence comprising SEQ ID NO.176; and any variants thereof having any one or more nucleic acid substitutions that encode conservative amino acid substitutions with the variable heavy and/or light chain regions of said antigen binding protein.

In one or more embodiment, the nucleic acid sequence of the present invention encodes an antigen binding protein comprising a caninized antigen binding protein.

In one further aspect the present invention provides a nucleic acid sequence that encodes the antigen binding protein of the invention comprising the amino acid sequence comprising SEQ ID NO.85 and is at least about 90% sequence identity to the nucleic acid sequence that encodes the variable heavy chain (VH) which comprises SEQ ID NO. 86 (nucleic acid sequence:ATGGAGTGGTCTTGGGTCTTTCTGTTCTTTCTGAGTGTTACCACCGGCGTGCACTCAGA CGTGCAGCTGGTGGAATCTGGCGGCGACCTGGTGCAGCCTGGCGGCTCTCTGAGACTGACCTGCG TGGCCTCCGGCTTCACCCTGACCCAGTACGGCATCAACTGGGTGCGACAGGCCCCTGGCAAGGGC CTGCAGTGGGTGGCCGTGATCTGGGCCACCGGCGCCACCGACTACAACTCCGCCCTGAAGTCCCG GTTCACCATCAGCCGGGACAACGCCAAGAACACCCTGTACCTGCAGATGAACTCCCTGAAAACCGA GGACACCGCCACCTACTACTGCGCCAGGGACGGCTGGTGGTACGCCACCTCCTGGTACTTCGACGT GTGGGGCCAGGGCGCTCTGGTGACAGTCTCGAGC) and a nucleic acid sequence having at least 90% sequence identity the nucleic acid sequence that encodes the variable light chain (VL) comprising SEQ ID NO.88 the amino acid sequence comprising SEQ ID NO.87 (nucleic acid sequence: ATGAGTGTTCCTACCCAAGTGCTGGGACTGCTGCTGCTGTGGCTGACAGATGCTCGGTGCGACATC GTGATGACCCAGACCCCACTGTCCCTGTCCGTGACACCTGGCGAGCCTGCCTCCATCTCCTGCAAG GCCTCCCAGGACATCAACCACTACCTGAACTGGTATCTGCAGAAGCCCGACGGCACCGTGAAGCTG CTGATCTACTACACCTCCCGGCTGCACTCCGGCGTGCCCTCCAGATTCTCCGGCTCTGGCTCCGGC ACCGACTTCACCCTGCGGATCTCCCGGGTGGAAGCCGACGACGTGGGCGTGTACTACTGCCAGCA GGGCGACCACTTCCCCCGGACCTTTGGCCCTGGTACC; and any variants thereof having any one or more nucleic acid substitutions that encode conservative amino acid substitutions with the variable heavy and/or light chain regions of said antigen binding protein.

In one or more embodiments the present invention provides a nucleic acid sequence that encodes the antigen binding protein of the invention comprising at least about 90% sequence identity to the nucleic acid sequence that encodes the variable heavy chain (VH) which comprises SEQ ID NO. 86 and a nucleic acid sequence having at least 90% sequence identity the nucleic acid sequence that encodes the variable light chain (VL) comprising SEQ ID NO.90 (nucleic acid sequence: ATGAGTGTTCCTACCCAAGTGCTGGGACTGCTGCTGCTGTGGCTGACAGATGCTCGGTGCGACATC GTGATGACCCAGACCCCACTGTCCCTGCCCGTGACACCTGGCGAGCCTGCCTCCATCTCCTGCAAG GCCTCCCAGGACATCAACCACTACCTGAACTGGTATCTGCAGAAGCCCGGCCAGTCCCCTCGGCTG CTGATCTACTACACCTCCCGGCTGCACTCCGGCGTGCCCTCCAGATTCTCCGGCTCTGGCTCCGGC ACCGACTTCACCCTGCGGATCTCCAGCGTGGAAGCCGACGACGTGGGCGTGTACTACTGCCAGCA GGGCGACCACTTCCCCCGGACCTTTGGCCAGGGTACC); and any variants thereof having any one or more nucleic acid substitutions that encode conservative amino acid substitutions with the variable heavy and/or light chain regions of said antigen binding protein.

In one or more embodiments the present invention provides a nucleic acid sequence that encodes the antigen binding protein of the invention comprising at least about 90% sequence identity to the nucleic acid sequence that encodes the variable heavy chain (VH) which comprises SEQ ID NO. 93 (nucleic acid sequence: ATGGAGTG GTCTTG GGTCTTTCTGTTCTTTCTGAGTGTTACCACCGG CGTGCACTCAGAC GTGCAGCTGGTGGAATCTGGCGGCGACCTGGTGAAACCTGGCGGCTCTCTGAGACTGACCTGCGT GGCCTCCGGCTTCACCCTGACCCAGTACGGCATCAACTGGGTGCGACAGGCCCCTGGCAAGGGCC TGCAGTGGGTGGCCGTGATCTGGGCCACCGGCGCCACCGACTACAACTCCGCCCTGAAGTCCCGG TTCACCATGAGCCGGGACAACGCCCGGAACACCCTGTACCTGCAGATGAACTCCCTGAAAACCGAG GACACCGCCACCTACTACTGCGCCAGGGACGGCTGGTGGTACGCCACCTCCTGGTACTTCGACGTG TGGGGCCAGGGCACCCTGGTGACAGTCTCGAGC): and a nucleic acid sequence having at least 90% sequence identity the nucleic acid sequence that encodes the variable light chain (VL) comprising SEQ ID NO.90 (nucleic acid sequence: ATGAGTGTTCCTACCCAAGTGCTGGGACTGCTGCTGCTGTGGCTGACAGATGCTCGGTGCGACATC GTGATGACCCAGACACCACTGTCTCTGCCTGTAACTCCGGGAGAACCAGCCAGCATTAGTTGTAAG GCTAGCCAGGACATCAACCACTATCTGAACTGGTATCTGCAGAAACCTGGCCAATCACCGCGCCTG CTGATCTATTACACCTCTCGACTGCATTCTGGAGTCCCATCCAGGTTCTCAGGGTCCGGGTCCGGCA CTGACTTCACCTTGCGCATATCTTCAGTGGAAGCCGATGACGTCGGAGTTTACTATTGTCAACAGGG CGACCACTTTCCACGGACATTCGGACAGGGTACC); and any variants thereof having any one or more nucleic acid substitutions that encode conservative amino acid substitutions with the variable heavy and/or light chain regions of said antigen binding protein.

In one or more embodiments the present invention provides a nucleic acid sequence that encodes the antigen binding protein of the invention comprising at least about 90% sequence identity to the nucleic acid sequence that encodes the variable heavy chain (VH) which comprises SEQ ID NO. 86 and a nucleic acid sequence having at least 90% sequence identity the nucleic acid sequence that encodes the variable light chain (VL) comprising SEQ ID NO.95 (nucleic acid sequence: ATGAGTGTTCCTACCCAAGTGCTGGGACTGCTGCTGCTGTGGCTGACAGATGCTCGGTGCGAGATC CAGATGACCCAGTCCCCATCCTCCCTGTCCGCCTCTCCCGGCGACAGAGTGACAATCACATGCAAG GCCTCCCAGGACATCAACCACTACCTGAACTGGTATCAGCAGAAGCCCGGCAAAGTGCCTAAGCTG CTGATCTACTACACCTCCCGGCTGCACTCCGGCGTGCCCTCCAGATTCTCCGGCTCTGGCTCCGGC ACCGACTTCACCCTGACCATCTCCAGCCTGGAACCCGAGGACGCCGCCACCTACTACTGCCAGCAG GGCGACCACTTCCCCCGGACCTTTGGCGGAGGTACC); and any variants thereof having any one or more nucleic acid substitutions that encode conservative amino acid substitutions with the variable heavy and/or light chain regions of said antigen binding protein.

In one or more embodiments the present invention provides a nucleic acid sequence that encodes the antigen binding protein of the invention comprising at least about 90% sequence identity to the nucleic acid sequence that encodes the variable heavy chain (VH) which comprises SEQ ID NO. 93 and a nucleic acid sequence having at least 90% sequence identity the nucleic acid sequence that encodes the variable light chain (VL) comprising SEQ ID NO.88; and any variants thereof having any one or more nucleic acid substitutions that encode conservative amino acid substitutions with the variable heavy and/or light chain regions of said antigen binding protein.

In one or more embodiments, the present invention provides a nucleic acid sequence encoding the antigen binding protein of the invention that comprises a felinized antibody.

In one or more embodiments, the present invention provides an antigen binding protein comprising a heavy chain variable region (VH) which comprises a Complimentary Determining Region (CDR) 1 comprising an amino acid sequence comprising at least about 90% sequence identity to the amino acid sequence comprising GFSLTGYGVN (SEQ ID NO.145), a Complimentary Determining Region (CDR) 2 comprising an amino acid sequence comprising at least about 90% sequence identity to the amino acid sequence comprising MIWGDGSTDYNSALKS (SEQ ID NO.146), and a Complimentary Determining Region (CDR) 3 selected from the group consisting of: DGYYYGTTWYFDV (SEQ ID NO.147); GGYDYDVPFFDY (SEQ ID NO.151) and GGYDYDVSFFDY (SEQ ID NO.153); and a light chain variable region (VL) which comprises a Complimentary Determining Region (CDR) 1 comprising an amino acid sequence comprising at least about 90% sequence identity to the amino acid sequences selected from RASQDISNYLN (SEQ ID NO. 148) or RSSQSIVHINRHTYLG (SEQ ID NO. 154); a Complimentary Determining Region (CDR) 2 comprising an amino acid sequence comprising at least about 90% sequence identity to the amino acid sequences selected from YTSRLHS (SEQ ID NO. 149) or GVSNRFS (SEQ ID NO. 155); and a Complimentary Determining Region (CDR) 3 comprising an amino acid sequence comprising at least about 90% sequence identity to the amino acid sequences selected from the group consisting of: QQGSTLPRT (SEQ ID NO. 150); QQGNMFPYT (SEQ ID NO. 152); FQGTHVPFT (SEQ ID NO. 156); and QQGNTLPYT (SEQ ID NO. 157), and any variants thereof having one or more conservative amino acid substitutions in at least one of CDR1, CDR2 or CDR3 within any of the variable light or variable heavy chain regions of said antigen binding protein.

In one or more embodiments, the present invention provides an antigen binding protein comprising a heavy chain variable region (VH) which comprises a Complimentary Determining Region (CDR) 1 comprising an amino acid sequence comprising at least about 90% sequence identity to the amino acid sequence comprising GFSLTGYGVN (SEQ ID NO.145, a Complimentary Determining Region (CDR) 2 comprising an amino acid sequence comprising at least about 90% sequence identity to the amino acid sequence comprising MIWGDGSTDYNSALKS (SEQ ID NO.146), and a Complimentary Determining Region (CDR) 3 comprising an amino acid sequence comprising at least about 90% sequence identity to the amino acid sequence comprising DGYYYGTTWYFDV (SEQ ID NO.147: and a light chain variable region (VL) which comprises a Complimentary Determining Region (CDR) 1 comprising an amino acid sequence comprising at least about 90% sequence identity to the amino acid sequence RASQDISNYLN (SEQ ID NO. 148); a Complimentary Determining Region (CDR) 2 comprising an amino acid sequence comprising at least about 90% sequence identity to the amino acid sequences YTSRLHS (SEQ ID NO. 149); and a Complimentary Determining Region (CDR) 3 comprising an amino acid sequence comprising at least about 90% sequence identity to the amino acid sequence QQGSTLPRT (SEQ ID NO. 150) and any variants thereof having one or more conservative amino acid substitutions in at least one of CDR1, CDR2 or CDR3 within any of the variable light or variable heavy chain regions of said antigen binding protein.

In one or more embodiments, the present invention provides an antigen binding protein comprising a heavy chain variable region (VH) which comprises an amino acid sequence comprising at least 90% sequence identity to the amino acid sequence comprising: EVKLQESG PGLVAPSQSLSITCTVSGFSLTGYGVNWVRQPPGKGLEWLGMIWGDGSTDYNSALKSRLSI SKDNSKSQVFLKMNSLQTDDTARYYCARDGYYYGTTWYFDVWGAGTTVTVSS (SEQ ID NO. 96); and a light chain variable region (VL) which comprises an amino acid sequence comprising at least 90% sequence identity to the amino acid sequence comprising: DIVMTQSTSSLSASLGDRVTISC RASQDISNYLNWYQQKPDGTIKLLIYYTSRLHSGVPSRFSGSGSGTDYSLTISNLEQEDIATYFCQQGSTL PRTFGGGT (SEQ ID NO. 100); and any variants thereof having one or more conservative amino acid substitutions within the variable heavy and/or variable light chain regions of said antigen binding protein. In one or more embodiments the antigen binding protein of the present invention comprises a nucleic acid sequence comprising at least about 90% sequence identity to the nucleic acid sequence that encodes the variable heavy chain (VH) which comprises SEQ ID NO. 97: GAAGTGAAACTGCAGGAAAGCGGCCCGGGCCTGGTGGCGCCGAGCCAGAGCCTGAGCATTACCTG CACCGTGAGCGGCTTTAGCCTGACCGGCTATGGCGTGAACTGGGTGCGCCAGCCGCCGGGCAAAG GCCTGGAATGGCTGGGCATGATTTGGGGCGATGGCAGCACCGATTATAACAGCGCGCTGAAAAGCC GCCTGAGCATTAGCAAAGATAACAGCAAAAGCCAGGTGTTTCTGAAAATGAACAGCCTGCAGACCGA TGATACCGCGCGCTATTATTGCGCGCGCGATGGCTATTATTATGGCACCACCTGGTATTTTGATGTG TGGGGCGCGGGCACCACCGTGACCGTGAGC AGC and a nucleic acid sequence having at least 90% sequence identity the nucleic acid sequence that encodes the variable light chain (VL) comprising SEQ ID NO.101: GATATTGTGATGACCCAGAGCACCAGCAGCCTGAGCGCGAGCCTGGGCGATCGCGTGACCATTAGC TGCCGCGCGAGCCAGGATATTAGCAACTATCTGAACTGGTATCAGCAGAAACCGGATGGCACCATTA AACTGCTGATTTATTATACCAGCCGCCTGCATAGCGGCGTGCCGAGCCGCTTTAGCGGCAGCGGCA GCGGCACCGATTATAGCCTGACCATTAGCAACCTGGAACAGGAAGATATTGCGACCTATTTTTGCCA GCAGGGCAGCACCCTGCCGCGCACCTTTGGCGGCGGCACC and any variants thereof having any one or more nucleic acid substitutions that encode conservative amino acid substitutions with the variable heavy and/or light chain regions of said antigen binding protein.

In one or more embodiments, the present invention provides an antigen binding protein comprising a heavy chain variable region (VH) which comprises an amino acid sequence comprising at least 90% sequence identity to the amino acid sequence comprising QVQLKESGPGLVAPSQSLSITCTVSG FSLTGYGVNWVRQPPGKGLEWLGMIWG DGSTDYNSALKSRLSISKDNSKSQVFLKMNSLQTDDTARYY CARDGYYYGTTWYFDVWGAGTTVTV (SEQ ID NO. 98); and a light chain variable region (VL) which comprises an amino acid sequence comprising at least 90% sequence identity to the amino acid sequence comprising :DIQMTQTTSSLSASLG DRVTISCRASQDISNYLNWYQQKP DGTIKLLIYYTSRLHSGVPSRFS GSGSGTDYSLTISNLEQEDIATYFCQQGSTLPRTFGGG (SEQ ID NO. 102); and any variants thereof having one or more conservative amino acid substitutions within the variable heavy and/or variable light chain regions of said antigen binding protein.

In one or more embodiments the antigen binding protein of the present invention comprises a nucleic acid sequence comprising at least about 90% sequence identity to the nucleic acid sequence that encodes the variable heavy chain (VH) which comprises SEQ ID NO. 99: CAGGTGCAGCTGAAAGAAAGCGGCCCGGGCCTGGTGGCGCCGAGCCAGAGCCTGAGCATTACCTG CACCGTGAGCGGCTTTAGCCTGACCGGCTATGGCGTGAACTGGGTGCGCCAGCCGCCGGGCAAAG GCCTGGAATGGCTGGGCATGATTTGGGGCGATGGCAGCACCGATTATAACAGCGCGCTGAAAAGCC GCCTGAGCATTAGCAAAGATAACAGCAAAAGCCAGGTGTTTCTGAAAATGAACAGCCTGCAGACCGA TGATACCGCGCGCTATTATTGCGCGCGCGATGGCTATTATTATGGCACCACCTGGTATTTTGATGTG TGGGGCGCGGGCACCACCGTGACCGTG and a nucleic acid sequence having at least 90% sequence identity the nucleic acid sequence that encodes the variable light chain (VL) comprising SEQ ID NO.103; and any variants thereof having any one or more nucleic acid substitutions that encode conservative amino acid substitutions with the variable heavy and/or light chain regions of said antigen binding protein

In one or more embodiments, the present invention provides an antigen binding protein comprising a heavy chain variable region (VH) which comprises an amino acid sequence comprising at least 90% sequence identity to the amino acid sequence comprising EVKLEESGPGLVAPSQSLSITCTVSGFSLTG YGVNWVRQPPGKGLEWLGMIWGDGSTDYNSALKSRLNISKDNSKSQVFLKMDSLQTDDTARYYCARG GYDYDVPFFDYWGQGTTLTVSS (SEQ ID NO. 104); and a light chain variable region (VL) which comprises an amino acid sequence comprising at least 90% sequence identity to the amino acid sequence comprising D IVMTQTTSSLSASLG DRVTISCRASQDISNYLNWYQQKPDGTVKLLIYYTSRLHSGVPS RFSGSGSGTDYSLTISNLEQEDIATYFCQQGNMFPYTLGGGT (SEQ ID NO. 108); and any variants thereof having one or more conservative amino acid substitutions within the variable heavy and/or variable light chain regions of said antigen binding protein.

In one or more embodiments the antigen binding protein of the present invention comprises a nucleic acid sequence comprising at least about 90% sequence identity to the nucleic acid sequence that encodes the variable heavy chain (VH) which comprises SEQ ID NO. 105 and a nucleic acid sequence having at least 90% sequence identity the nucleic acid sequence that encodes the variable light chain (VL) comprising SEQ ID NO.109; and any variants thereof having any one or more nucleic acid substitutions that encode conservative amino acid substitutions with the variable heavy and/or light chain regions of said antigen binding protein.

In one or more embodiments, the present invention provides an antigen binding protein comprising a heavy chain variable region (VH) which comprises an amino acid sequence comprising at least 90% sequence identity to the amino acid sequence comprising QVQLKESGPGLVAPSQSLSITCTVSGFSLTG YGVNWVRQPPGKGLEWLGMIWGDGSTDYNSALKSRLNISKDNSKSQVFLKMDSLQTDDTARYYCARG GYDYDVPFFDYWGQGTTLTV (SEQ ID NO. 106); and a light chain variable region (VL) which comprises an amino acid sequence comprising at least 90% sequence identity to the amino acid sequence comprising DIQMTQTTSSLSASLGDRVTISCRASQDISNYLNWYQQKPDGTVKLLIYYTSRLHSGVPSRFSGSGSGTD YSLTISNLEQEDIATYFCQQGNMFPYTLGGG (SEQ ID NO. 110); and any variants thereof having one or more conservative amino acid substitutions within the variable heavy and/or variable light chain regions of said antigen binding protein.

In one or more embodiments the antigen binding protein of the present invention comprises a nucleic acid sequence comprising at least about 90% sequence identity to the nucleic acid sequence that encodes the variable heavy chain (VH) which comprises SEQ ID NO. 107 and a nucleic acid sequence having at least 90% sequence identity the nucleic acid sequence that encodes the variable light chain (VL) comprising SEQ ID NO.111; and any variants thereof having any one or more nucleic acid substitutions that encode conservative amino acid substitutions with the variable heavy and/or light chain regions of said antigen binding protein.

In one or more embodiments, the present invention provides an antigen binding protein comprising a heavy chain variable region (VH) which comprises an amino acid sequence comprising at least 90% sequence identity to the amino acid sequence comprising EVQLEQSGPGLVAPSQSLSITCTVSGFSLTGY GVNWVRQPPGKGLEWLGMIWGDGSTDYNSALKSRLSISKDNSKSQVFLKMNSLQTDDTARYYCARDGY YYGTTWYFDVWGAGTTVTVSS (SEQ ID NO. 112); and a light chain variable region (VL) which comprises an amino acid sequence comprising at least 90% sequence identity to the amino acid sequence comprising DIVLTQSTSSLSASLG DRVTISCRASQDISNYLNWYQQKPDGTIKLLIYYTSRLHSGVPSRFSGSGSGTDY SLTISNLEQEDIATYFCQQGSTLPRTFGGGT (SEQ ID NO. 114); and any variants thereof having one or more conservative amino acid substitutions within the variable heavy and/or variable light chain regions of said antigen binding protein.

In one or more embodiments the antigen binding protein of the present invention comprises a nucleic acid sequence comprising at least about 90% sequence identity to the nucleic acid sequence that encodes the variable heavy chain (VH) which comprises SEQ ID NO. 113 and a nucleic acid sequence having at least 90% sequence identity the nucleic acid sequence that encodes the variable light chain (VL) comprising SEQ ID NO.115; and any variants thereof having any one or more nucleic acid substitutions that encode conservative amino acid substitutions with the variable heavy and/or light chain regions of said antigen binding protein.

In one or more embodiments, the present invention provides an antigen binding protein comprising a heavy chain variable region (VH) which comprises an amino acid sequence comprising at least 90% sequence identity to the amino acid sequence comprising EVQLQESGAELVKPGASVKLSCKASGY TFTNYWMHWVKQRPGQGLEWIGHIDPSDGETHYNQKFKDKATLTVDKSSSTAYMQLTGLTSEDSAVYY CARFLPDYWGQGTSVTVSS (SEQ ID NO. 116); and a light chain variable region (VL) which comprises an amino acid sequence comprising at least 90% sequence identity to the amino acid sequence comprising DIVLTQTPAIMSASPGEKVTMTCRASSSVSSIYLHWYQQKPGSSPKLWIYSTSNLASGVPARFSGSGSGT SYSLTVSSVEAEDAATYYCQLYDNSPLTFGAGT (SEQ ID NO. 120); and any variants thereof having one or more conservative amino acid substitutions within the variable heavy and/or variable light chain regions of said antigen binding protein.

In one or more embodiments the antigen binding protein of the present invention comprises a nucleic acid sequence comprising at least about 90% sequence identity to the nucleic acid sequence that encodes the variable heavy chain (VH) which comprises SEQ ID NO. 117 and a nucleic acid sequence having at least 90% sequence identity the nucleic acid sequence that encodes the variable light chain (VL) comprising SEQ ID NO.121; and any variants thereof having any one or more nucleic acid substitutions that encode conservative amino acid substitutions with the variable heavy and/or light chain regions of said antigen binding protein.

In one or more embodiments, the present invention provides an antigen binding protein comprising a heavy chain variable region (VH) which comprises an amino acid sequence comprising at least 90% sequence identity to the amino acid sequence comprising QVQLQQPGAELVKPGASVKLSCKASGY TFTNYWMHWVKQRPGQGLEWIGHIDPSDGETHYNQKFKDKATLTVDKSSSTAYMQLTGLTSEDSAVYY CARFLPDYWGQGTSVTV (SEQ ID NO. 118); and a light chain variable region (VL) which comprises an amino acid sequence comprising at least 90% sequence identity to the amino acid sequence comprising DIVLTQSPAIMSASPGEKVTMTCRASSSVSSIYLHWYQQKPGSSPKLWIYSTSNLASGVPARFSGSGSGT SYSLTVSSVEAEDAATYYCQLYDNSPLTFGAG (SEQ ID NO. 122); and any variants thereof having one or more conservative amino acid substitutions within the variable heavy and/or variable light chain regions of said antigen binding protein.

In one or more embodiments the antigen binding protein of the present invention comprises a nucleic acid sequence comprising at least about 90% sequence identity to the nucleic acid sequence that encodes the variable heavy chain (VH) which comprises SEQ ID NO. 119 and a nucleic acid sequence having at least 90% sequence identity the nucleic acid sequence that encodes the variable light chain (VL) comprising SEQ ID NO.123; and any variants thereof having any one or more nucleic acid substitutions that encode conservative amino acid substitutions with the variable heavy and/or light chain regions of said antigen binding protein.

In one or more embodiments, the present invention provides an antigen binding protein comprising a heavy chain variable region (VH) which comprises an amino acid sequence comprising at least 90% sequence identity to the amino acid sequence comprising EVQLEESGPGLVAPSQSLSITCTVSGFSLT GYGVNWVRQPPGKGLEWLGMIWGDGSTDYNSALKSRLSISKDNSKSQVFLKMNSLQTDDTARYYCARD GYYYGTTWYFDVWGAGTTVTVSS (SEQ ID NO. 124); and a light chain variable region (VL) which comprises an amino acid sequence comprising at least 90% sequence identity to the amino acid sequence comprising: DIVITQTPLSLPVSLGDQASISCRSSQSIVHINRHTYLGWYLQKPGQSLKLLIYGVSNRFS GVPDRFSGSGSGTDFTLKISRVEAEDMGVYYCFQGTHVPFTFGSGT (SEQ ID NO. 126); and any variants thereof having one or more conservative amino acid substitutions within the variable heavy and/or variable light chain regions of said antigen binding protein.

In one or more embodiments the antigen binding protein of the present invention comprises a nucleic acid sequence comprising at least about 90% sequence identity to the nucleic acid sequence that encodes the variable heavy chain (VH) which comprises SEQ ID NO. 125 and a nucleic acid sequence having at least 90% sequence identity the nucleic acid sequence that encodes the variable light chain (VL) comprising SEQ ID NO.127; and any variants thereof having any one or more nucleic acid substitutions that encode conservative amino acid substitutions with the variable heavy and/or light chain regions of said antigen binding protein.

In one or more embodiments, the present invention provides an antigen binding protein comprising a heavy chain variable region (VH) which comprises an amino acid sequence comprising at least 90% sequence identity to the amino acid sequence comprising EVKLEESGPGLVAPSQSLSITCTVSGFSLTGYGVNWVRQPPGKGLEWLGMIWGDGSTDYNSALKSRLSI SKDNSKSQVFLKMNSLQTDDTARYYCARGGYDYDVSFFDYWGQGTTLTVSS (SEQ ID NO. 130); and a light chain variable region (VL) which comprises an amino acid sequence comprising at least 90% sequence identity to the amino acid sequence comprising DIVLTQTTSSLSASLGDRVTISCRASQDISNYLNWYQQKP DGTVKLLIYYTSRFHSGVPSRFSGSGSGTDYSLTISNLEHEDIATYFCQQGNTLPYTFGGGT (SEQ ID NO. 134); and any variants thereof having one or more conservative amino acid substitutions within the variable heavy and/or variable light chain regions of said antigen binding protein.

In one or more embodiments the antigen binding protein of the present invention comprises a nucleic acid sequence comprising at least about 90% sequence identity to the nucleic acid sequence that encodes the variable heavy chain (VH) which comprises SEQ ID NO. 131 and a nucleic acid sequence having at least 90% sequence identity the nucleic acid sequence that encodes the variable light chain (VL) comprising SEQ ID NO.135; and any variants thereof having any one or more nucleic acid substitutions that encode conservative amino acid substitutions with the variable heavy and/or light chain regions of said antigen binding protein.

In one or more embodiments, the present invention provides an antigen binding protein comprising a heavy chain variable region (VH) which comprises an amino acid sequence comprising at least 90% sequence identity to the amino acid sequence comprising QVQLKESGPGLVAPSQSLSITCT VSGFSLTGYGVNWVRQPPGKGLEWLGMIWGDGSTDYNSALKSRLSISKDNSKSQVFLKMNSLQTDDTA RYYCARGGYDYDVSFFDYWGQGTTLTV (SEQ ID NO. 132); and a light chain variable region (VL) which comprises an amino acid sequence comprising at least 90% sequence identity to the amino acid sequence comprising: DIQMTQTTSSLSASLGDRVTISCRASQDISNYLNWYQQKPDGTVKLLIYYTSRFHSGVPSRFSGSGSGTD YSLTISNLEHEDIATYFCQQGNTLPYTFGGG (SEQ ID NO. 136); and any variants thereof having one or more conservative amino acid substitutions within the variable heavy and/or variable light chain regions of said antigen binding protein.

In one or more embodiments the antigen binding protein of the present invention comprises a nucleic acid sequence comprising at least about 90% sequence identity to the nucleic acid sequence that encodes the variable heavy chain (VH) which comprises SEQ ID NO. 133 and a nucleic acid sequence having at least 90% sequence identity the nucleic acid sequence that encodes the variable light chain (VL) comprising SEQ ID NO.137; and any variants thereof having any one or more nucleic acid substitutions that encode conservative amino acid substitutions with the variable heavy and/or light chain regions of said antigen binding protein.

In one or more embodiments, the present invention provides an antigen binding protein comprising a heavy chain variable region (VH) which comprises an amino acid sequence comprising at least 90% sequence identity to the amino acid sequence comprising QVKLEESGPGLVAPSQSLSIT CTVSG FSLTGYGVNWVRQPPGKGLEWLGMIWGDGSTDYNSALKSRLSISKDNSKSQVFLKMNSLQTDD TARYYCARDGYYYGTTWYFDVWGAGTTVTVSS (SEQ ID NO. 138); and a light chain variable region (VL) which comprises an amino acid sequence comprising at least 90% sequence identity to the amino acid sequence comprising DIVLTQTTSSLSASLGDRVTISCRASQDISNYLNWYQQKPDGTIK LLIYYTSRLHSGVPSRFSGSGSGTDYSLTISNLEQEDIATYFCQQGSTLPRTFGGGT (SEQ ID NO. 140); and any variants thereof having one or more conservative amino acid substitutions within the variable heavy and/or variable light chain regions of said antigen binding protein.

In one or more embodiments the antigen binding protein of the present invention comprises a nucleic acid sequence comprising at least about 90% sequence identity to the nucleic acid sequence that encodes the variable heavy chain (VH) which comprises SEQ ID NO. 139 and a nucleic acid sequence having at least 90% sequence identity the nucleic acid sequence that encodes the variable light chain (VL) comprising SEQ ID NO.141; and any variants thereof having any one or more nucleic acid substitutions that encode conservative amino acid substitutions with the variable heavy and/or light chain regions of said antigen binding protein.

In one or more embodiments, the present invention provides an antigen binding protein comprising a heavy chain variable region (VH) which comprises an amino acid sequence comprising at least 90% sequence identity to the amino acid sequence comprising EVQLQQSGPGLVAPSQSLSIT CTVSGFSLTGYGVNWVRQPPGKGLEWLGMIWGDGSTDYNSALKSRLSISKDNSKSQVFLKMNSLQTDD TARYYCARDGYYYGTTWYFDVWGAGTTVTVSS (SEQ ID NO. 142); and a light chain variable region (VL) which comprises an amino acid sequence comprising at least 90% sequence identity to the amino acid sequence comprising DIQMTQTTSSLSASLGDRVTISCRASQDISNYLNWYQQKPDGTIKLLIYYT SRLHSGVPSRFSGSGSGTDYSLTISNLEQEDIATYFCQQGSTLPRTFGGGT (SEQ ID NO. 144); and any variants thereof having one or more conservative amino acid substitutions within the variable heavy and/or variable light chain regions of said antigen binding protein.

In one or more embodiments, the antigen binding protein of the invention further comprises a canine light chain constant region comprising an amino acid sequence that comprises at least about 95% sequence identity to SEQ ID NO. 160. In one embodiment, the antigen binding protein further comprises a canine light chain constant region comprising SEQ ID NO. 160.

In one or more embodiments, the antigen binding protein of the invention further comprises a canine heavy chain constant region comprising an amino acid sequence that comprises at least about 95% sequence identity to SEQ ID NO.158. In one embodiment the heavy chain constant region comprises SEQ ID NO. 158. In one or more embodiment the heavy chain constant region comprises a mutation that reduces or eliminates effector function of the antigen binding protein comprising SEQ ID NO. 184.

In one or more embodiments, the antigen binding protein of the invention further comprises a feline light chain constant region comprising an amino acid sequence that comprises at least about 95% sequence identity to SEQ ID NO. 165. In one embodiment, the antigen binding protein further comprises a feline light chain constant region comprising SEQ ID NO. 165.

In one or more embodiments, the antigen binding protein of the invention further comprises a feline heavy chain constant region comprising an amino acid sequence that comprises at least about 95% sequence identity to SEQ ID NO.162. In one embodiment the heavy chain constant region comprises SEQ ID NO. 162. In one or more embodiment the heavy chain constant region comprises a mutation that reduces or eliminates effector function of the antigen binding protein.

In one or more embodiments, the antigen binding protein of the invention provides that said binding protein does not cause an immunological reaction within the species in which it is being administered.

In one or more embodiments, the present invention provides an antigen binding protein that comprises a chimeric antibody. In one or more embodiments, the antigen binding protein of the present invention is speciated. In one or more embodiments, the antigen binding protein of the present invention is a caninized antigen binding protein. In one or more embodiments, the antigen binding protein of the invention is a felinized antigen binding protein. In one or more embodiments, the antigen binding protein of the invention is an equinized antigen binding protein. In one or more embodiments, the antigen binding protein of the invention is a humanized antigen binding protein.

In one or more embodiments, the antigen binding protein of the present invention specifically binds to Nerve Growth Factor (NGF). In one embodiment, the NGF is canine NGF. In one embodiment, the NGF is feline NGF. In one embodiment, the NGF is human NGF. In one embodiment, the NGF is a rodent NGF.

In one or more embodiments, the antigen binding protein of the present invention specifically binds to NGF and prevents NGF from binding to TrkA thus inhibiting signaling through TrkA, which has been shown to reduce the signaling through sensory neurons and thus reducing levels of pain. In one embodiment, the NGF is canine NGF. In one embodiment, the NGF is feline NGF. In one embodiment, the NGF is human NGF. In one embodiment, the NGF is rodent NGF. In one or more embodiments, the antigen binding protein of the invention has no significant adverse effect on the immune system. In some embodiments, there is no significant adverse effect of the immune system of a canine. In some embodiments, there is no significant adverse effect of the immune system in a feline. In some embodiments, there is no significant adverse effect of the immune system of a human

In one or more embodiments, the antigen binding protein of the present invention provides that said antigen binding protein inhibits the biological function of NGF. In one embodiment, the inhibition is functional inhibition of canine NGF. In one embodiment, the inhibition of NGF is functional inhibition of feline NGF. In one embodiment, the inhibition is functional inhibition of human NGF.

In one or more embodiments, the isolated and recombinant antigen binding protein that specifically binds to NGF reduces or eliminates an NGF related disorder. In some embodiments, the NGF related disorder is selected from the group consisting of: cardiovascular diseases, atherosclerosis, obesity, diabetes, metabolic syndrome, pain and inflammation. In some embodiments, the NGF related disorder is pain. In some embodiments, the NGF related disorder is inflammation. In some embodiments the type of pain is selected from the group consisting of: chronic pain; inflammatory pain, post-operative incision pain, neuropathic pain, fracture pain, osteoporotic fracture pain, post-herpetic neuralgia, cancer pain, pain resulting from burns, pain associated with wounds, pain associated with trauma, neuropathic pain, pain associated with musculoskeletal disorders such as rheumatoid arthritis, osteoarthritis, ankylosing spondylitis, seronegative (non-rheumatoid) arthropathies, non-articular rheumatism and periarticular disorders and peripheral neuropathy. In some embodiments, the type of pain is chronic pain. In some embodiments, the type of pain is osteoarthritis pain. In some embodiments, the type of pain is inflammatory pain. In some embodiments, the type of pain is post-operative pain. In some embodiments, the type of pain is cancer pain.

In one or more embodiments the antigen binding protein of the present invention is selected from the group consisting of: a monoclonal antibody, a chimeric antibody, a single chain antibody, a tetrameric antibody, a tetravalent antibody, a multispecific antibody, a domain-specific antibody, a domain-deleted antibody, a fusion protein, an ScFc fusion protein, an Fab fragment, an Fab′ fragment, an F(ab′)₂ fragment, an Fv fragment, an ScFv fragment, an Fd fragment, a single domain antibody, a dAb fragment, a small modular immunopharmaceutical (SMIP) a nanobody, and IgNAR molecule. In some embodiments said antibody is a monoclonal antibody. In some embodiments said antibody is chimeric.

In one or more embodiments, the present invention provides a pharmaceutical composition comprising a therapeutically effective amount of any one or more of the isolated and recombinant antigen binding proteins. In one or more embodiments, the pharmaceutical composition of the invention has no significant adverse effect on the immune system of a canine. In one embodiment, the composition of the invention has no significant adverse effect on the immune system of a feline. In one embodiment, the composition of the invention has no significant adverse effect on the immune system of a human. In one embodiment, the pharmaceutical composition is a veterinary composition.

In one or more embodiments, the present invention provides a host cell that produces any one or more of the antigen binding proteins of the present invention.

In one or more embodiments, the invention provides a vector comprising the any one or more of the nucleic acids of the present invention.

In one or more embodiments, the invention provides a host cell comprising the any one or more of the nucleic acids of the present invention.

In one or more embodiments, the invention provides a host cell comprising the vector that comprises any one or more of the nucleic acids of the present invention.

In one or more embodiments, the present invention provides a method of treating a subject for an NGF related disorder comprising administering a therapeutically effective amount of the pharmaceutical composition of the invention. In some embodiments, the subject of the invention comprises canines, felines, or humans. In some embodiments, the subject comprises canines. In some embodiment, the subject comprises felines. In some embodiments, the subject comprises humans. In some embodiments, the NGF related disorder is selected from the group consisting of: cardiovascular diseases, atherosclerosis, obesity, diabetes, metabolic syndrome, pain and inflammation. In some embodiments of the present invention the type of pain is selected from the group consisting of: chronic pain; inflammatory pain, post-operative incision pain, neuropathic pain, fracture pain, osteoporotic fracture pain, post-herpetic neuralgia, cancer pain, pain resulting from burns, pain associated with wounds, pain associated with trauma, neuropathic pain, pain associated with musculoskeletal disorders such as rheumatoid arthritis, osteoarthritis, ankylosing spondylitis, seronegative (non-rheumatoid) arthropathies, non-articular rheumatism and periarticular disorders and peripheral neuropathy. In some embodiments, the type of pain is osteoarthritis pain. In some embodiments, the type of pain is inflammatory pain. In some embodiments, the type of pain is chronic pain.

In some embodiments, the type of pain is post-operative pain. In some embodiments, the type of pain is cancer pain.

In one embodiment, the invention provides a method of producing an antigen binding protein comprising culturing any of the host cells of the present invention as described herein, under conditions that result in production of the caninized antigen binding protein, and isolating the caninized antibody antigen binding protein from the host cell or culture medium of the host cell.

In one or more embodiments, the present invention provides a method of inhibiting NGF activity by administering the pharmaceutical composition of the present invention.

In one or more embodiments, the present invention provides a method of detecting or quantitating NGF levels in a biological sample, the method comprising:
(a) incubating a clinical or biological sample containing NGF in the presence of any one of the caninized antibody, antigen binding protein or fragments of the present invention; and
(b) detecting the antigen binding protein or fragments which are bound to NGF in the sample.

In some embodiments, the antigen binding protein or fragments is detectably labeled. In some embodiments, the antigen binding protein or fragments is unlabeled is used in combination with a second antigen binding protein or fragments which is detectably labeled. In one embodiment, the invention comprises a kit comprising the antigen binding protein of the invention.

### BRIEF DESCRIPTION OF THE FIGURES

FIG. 1: is a schematic representation of the general structure of a mouse immunoglobulin G (IgG) molecule highlighting the antigen binding site.

FIG. 2: is a schematic representation of the general structure of a mouse/canine chimeric IgG.

FIG. 3: is an illustration showing speciation or “caninization” of a mouse IgG, mouse CDRs grafted onto canine frameworks.

FIG. 4 is an illustration of a “heterochimeric” monoclonal antibody paring the chimeric light chain with a fully caninized heavy chain.

FIG. 5 is a representation of the amino acid comparisons between rat, mouse, human, feline and canine NGF.

FIG. 6 is a graphical representation of the reactivity of the serial dilutions of hybridomas made after the immunization of mouse 3-5.

FIG. 7 is a graphical representation of the OD measured for each of the supernatants tested from the hybridomas generated after immunization.

FIG. 8 is a graphical representation of the selected anti-NGF hybridoma subclones.

FIG. 9 is a graphical representation of the isolated monoclonal antibodies in a canine NGF ELISA assay.

FIG. 10 is a graphical representation of the dog serum of ZTS-182 concentration-time graph for 8 different dogs at a dose of 1.4 mg/kg.

FIG. 11 is a graphical representation of the dog serum of ZTS-182 concentration-time graph for 8 different dogs at a dose of 4.0 mg/kg.

FIG. 12 is a graphical representation of the dog serum of ZTS-182 concentration-time graph for 8 different dogs at a dose of 12.0 mg/kg.

FIG. 13 is a graphical representation of the dog serum of ZTS-182 concentration-time graph for 8 different dogs at a dose of 20.0 mg/kg.

FIG. 14A is a graphical representation of the percent inhibition of neurite length after treatment of rat PC12 cells with various anti-NGF mAbs.

FIG. 14B is a graphical representation of neurite length after treatment of rat PC12 cells with various anti-NGF mAbs.

FIG. 15 is a schematic representation of the rat MIA assay.

FIG. 16 is a graphical representation of different concentrations of ZTS-182 administered and the percent weight bearing in the MIA model.

FIG. 17 is a graphical representation of post-synovitis inductions vs. lameness scores after ZTS-182 administrations over time.

FIG. 18A is a graphical representation of cat serum of ZTS-082 concentration-time graph for 8 different cats at a dose of 3.0 mg/kg.

FIG. 18B is a graphical representation of cat serum of ZTS-082 concentration-time graph for 8 different cats at a dose of 3.0 mg/kg.

### BRIEF DESCRIPTION OF THE SEQUENCES

SEQ ID NO: 1 is the amino acid sequence for human NGF.
SEQ ID NO: 2 is the amino acid sequence for canine NGF.
SEQ ID NO: 3 is the amino acid sequence for feline NGF.
SEQ ID NO: 4 is the amino acid sequence that describes the variable heavy chain CDR 1 referred to herein as 01 B12H3AHC VH CDR1.
SEQ ID NO: 5 is the amino acid sequence that describes the variable heavy chain CDR 2 referred to herein as 01 B12H3AHC VH CDR 2.
SEQ ID NO: 6 is the amino acid sequence that describes the variable heavy chain CDR3 referred to herein as 01 B12H3AHC VH CDR3.
SEQ ID NO: 7 is the amino acid sequence that describes the variable light chain CDR1 referred to herein as 01 B12H3AHC VL CDR1.
SEQ ID NO: 8 is the amino acid sequence that describes the variable light chain CDR2 referred to herein as 01 B12H3AHC VL CDR2.
SEQ ID NO: 9 is the amino acid sequence that describes the variable light chain CDR3 referred to herein as 01 B12H3AHC VL CDR3.
SEQ ID NO: 10 is the amino acid sequence that describes the variable light chain CDR1 referred to herein as QC23L2AL3 VL CDR1.
SEQ ID NO: 11 is the amino acid sequence that describes the variable light chain CDR2 referred to herein as QC23L2AL3 VL CDR2.
SEQ ID NO: 12 is the amino acid sequence that describes the variable light chain CDR3 referred to herein as QC23L2AL3 VL CDR3.
SEQ ID NO: 13 is the amino acid sequence that describes the variable light chain CDR1 referred to herein as QC23L5A VL CDR1.
SEQ ID NO: 14 is the amino acid sequence that describes the variable light chain CDR2 referred to herein as QC23L5A VL CDR2.
SEQ ID NO: 15 is the amino acid sequence that describes the variable light chain CDR3 referred to herein as QC23L5A VL CDR3.
SEQ ID NO: 16 is the amino acid sequence that describes the variable light chain CDR1 referred to herein as QC2301 B12L2AL1 VL CDR1.
SEQ ID NO: 17 is the amino acid sequence that describes the variable light chain CDR2 referred to herein as QC2301 B12L2AL1 VL CDR 2.
SEQ ID NO: 18 is the amino acid sequence that describes the variable light chain CDR3 referred to herein as QC2301 B12L2AL1 VL CDR3.
SEQ ID NO: 19 is the amino acid sequence that describes the variable light chain CDR1 referred to herein as QC2301 B12L1 AL3 VL CDR1.
SEQ ID NO: 20 is the amino acid sequence that describes the variable light chain CDR2 referred to herein as QC2301 B12L1 AL3 VL CDR2.
SEQ ID NO: 21 is the amino acid sequence that describes the variable light chain CDR3 referred to herein as QC2301 B12L1 AL3 VL CDR3.
SEQ ID NO: 22 is the amino acid sequence that describes the variable light chain CDR1 referred to herein as QC2301 B12L1 AL1 VL CDR1.
SEQ ID NO: 23 is the amino acid sequence that describes the variable light chain CDR2 referred to herein as QC2301 B12L1 AL1 VL CDR2.
SEQ ID NO: 24 is the amino acid sequence that describes the variable light chain CDR3 referred to herein as QC2301B12L1AL1 VL CDR3.
SEQ ID NO: 25 is the amino acid sequence that describes the variable light chain CDR1 referred to herein as QC2301 B12VK VL CDR1.
SEQ ID NO: 26 is the amino acid sequence that describes the variable light chain CDR2 referred to herein as QC2301 B12VK VL CDR2.
SEQ ID NO: 27 is the amino acid sequence that describes the variable light chain CDR3 referred to herein as QC2301 B12VK VL CDR3.
SEQ ID NO: 28 is the amino acid sequence that describes the variable light chain CDR1 referred to herein as QC2301 B12L5AL2 VL CDR1.
SEQ ID NO: 29 is the amino acid sequence that describes the variable light chain CDR2 referred to herein as QC2301 B12L5AL2 VL CDR2.
SEQ ID NO: 30 is the amino acid sequence that describes the variable light chain CDR3 referred to herein as QC2301 B12L5AL2 VL CDR3.
SEQ ID NO: 31 is the amino acid sequence that describes the variable light chain CDR1 referred to herein as QC23L2AL1 VL CDR1.
SEQ ID NO: 32 is the amino acid sequence that describes the variable light chain CDR2 referred to herein as QC23L2AL1 VL CDR2.
SEQ ID NO: 33 is the amino acid sequence that describes the variable light chain CDR3 referred to herein as QC23L2AL1 VL CDR3.
SEQ ID NO: 34 is the amino acid sequence that describes the variable light chain CDR1 referred to herein as QC23L6A VL CDR1.
SEQ ID NO: 35 is the amino acid sequence that describes the variable light chain CDR2 referred to herein as QC23L6A VL CDR2.
SEQ ID NO: 36 is the amino acid sequence that describes the variable light chain CDR3 referred to herein as QC23L6A VL CDR3.
SEQ ID NO: 37 is the amino acid sequence that describes the variable light chain CDR1 referred to herein as QC23L1A02D9L2 VL CDR1.
SEQ ID NO: 38 is the amino acid sequence that describes the variable light chain CDR2 referred to herein as QC23L1A02D9L2 VL CDR2.
SEQ ID NO: 39 is the amino acid sequence that describes the variable light chain CDR3 referred to herein as QC23L1A02D9L3 VL CDR3.
SEQ ID NO: 40 is the amino acid sequence that describes the variable light chain CDR1 referred to herein as QC23L6A VL CDR1.
SEQ ID NO: 41 is the amino acid sequence that describes the variable light chain CDR2 referred to herein as QC23L6A VL CDR2.
SEQ ID NO: 42 is the amino acid sequence that describes the variable light chain CDR3 referred to herein as QC23L6A VL CDR3.
SEQ ID NO: 43 is the amino acid sequence that describes the variable light chain CDR1 referred to herein as 02B4VL1 VL CDR1.
SEQ ID NO: 44 is the amino acid sequence that describes the variable light chain CDR2 referred to herein as 02B4VL1 VL CDR2.
SEQ ID NO: 45 is the amino acid sequence that describes the variable light chain CDR3 referred to herein as 02B4VL1 VL CDR3.
SEQ ID NO: 46 is the amino acid sequence that describes the variable light chain CDR1 referred to herein as 02B4L1AL1 VL CDR1.
SEQ ID NO: 47 is the amino acid sequence that describes the variable light chain CDR2 referred to herein as 02B4L1AL1 VL CDR2.
SEQ ID NO: 48 is the amino acid sequence that describes the variable light chain CDR3 referred to herein as 02B4L1AL1 VL CDR3.
SEQ ID NO. 49 is the amino acid sequence that describes the variable heavy chain referred to herein as 01B12H3AHC VH.
SEQ ID NO. 50 is the nucleotide sequence encoding the variable heavy chain referred to herein as 01B12H3AHC VH.
SEQ ID NO. 51 is the amino acid sequence that describes the variable light chain referred to herein as QC2301B12L1AL1L3 VL.
SEQ ID NO. 52 is the nucleotide sequence encoding the variable light chain referred to herein as QC2301B12L1AL1L3 VL.
SEQ ID NO. 53 is the amino acid sequence that describes the variable light chain referred to herein as QC23L2AL3 VL.
SEQ ID NO. 54 is the nucleotide sequence encoding the variable light chain referred to herein as QC23L2AL3 VL.
SEQ ID NO. 55 is the amino acid sequence that describes the variable light chain referred to herein as QC23L5A VL.
SEQ ID NO. 56 is the nucleotide sequence encoding the variable light chain referred to herein as QC23L5A VL.
SEQ ID NO. 57 is the amino acid sequence that describes the variable light chain referred to herein as QC2301B12L2AL1 VL.
SEQ ID NO. 58 is the nucleotide sequence encoding the variable light chain referred to herein as QC2301B12L2AL1 VL.
SEQ ID NO. 59 is the amino acid sequence that describes the variable light chain referred to herein as QC2301B12L1AL3 VL.
SEQ ID NO. 60 is the nucleotide sequence encoding the variable light chain referred to herein as QC2301B12L1AL3 VL.
SEQ ID NO. 61 is the amino acid sequence that describes the variable light chain referred to herein as QC2301B12L1AL1 VL.
SEQ ID NO. 62 is the nucleotide sequence encoding the variable light chain referred to herein as QC2301B12L1AL1 VL.
SEQ ID NO. 63 is the amino acid sequence that describes the variable light chain referred to herein as QC2301B12VK VL.
SEQ ID NO. 64 is the nucleotide sequence encoding the variable light chain referred to herein as QC2301B12VK VL.
SEQ ID NO. 65 is the amino acid sequence that describes the variable light chain referred to herein as QC2301B12L5AL2 VL.
SEQ ID NO. 66 is the nucleotide sequence encoding the variable light chain referred to herein as QC2301B12L5AL2 VL.
SEQ ID NO. 67 is the amino acid sequence that describes the variable light chain referred to herein as QC23L2AL1 VL.
SEQ ID NO. 68 is the nucleotide sequence encoding the variable light chain referred to herein as QC23L2AL1 VL.
SEQ ID NO. 69 is the amino acid sequence that describes the variable light chain referred to herein as QC23L6A VL.
SEQ ID NO. 70 is the nucleotide sequence encoding the variable light chain referred to herein as QC23L6A VL.
SEQ ID NO. 71 is the amino acid sequence that describes the variable light chain referred to herein as QC23L1A02D9L2 VL.
SEQ ID NO. 72 is the nucleotide sequence encoding the variable light chain referred to herein as QC23L1A02D9L2 VL.
SEQ ID NO. 73 is the amino acid sequence that describes the variable light chain referred to herein as QC2301B12L6AL2 VL.
SEQ ID NO. 74 is the nucleotide sequence encoding the variable light chain referred to herein as QC2301B12L6AL2 VL.
SEQ ID NO. 75 is the amino acid sequence that describes the variable light chain referred to herein as 02B4VL1 VL.
SEQ ID NO. 76 is the nucleotide sequence encoding the variable light chain referred to herein as 02B4VL1 VL.
SEQ ID NO. 77 is the amino acid sequence that describes the variable light chain referred to herein as 02B4L1AL1 VL.
SEQ ID NO. 78 is the nucleotide sequence encoding the variable light chain referred to herein as 02B4L1AL1 VL.
SEQ ID NO. 79 is the amino acid sequence that describes the variable heavy chain CDR 1 referred to herein as ZTS-082VH CDR1.
SEQ ID NO. 80 is the amino acid sequence that describes the variable heavy chain CDR 2 referred to herein as ZTS-082VH CDR2.
SEQ ID NO. 81 is the amino acid sequence that describes the variable heavy chain CDR 3 referred to herein as ZTS-082- VH CDR3.
SEQ ID NO. 82 is the amino acid sequence that describes the variable light chain CDR 1 referred to herein as ZTS-082 VL CDR1.
SEQ ID NO. 83 is the amino acid sequence that describes the variable light chain CDR 2 referred to herein as ZTS-082VL CDR2.
SEQ ID NO. 84 is the amino acid sequence that describes the variable light chain CDR 3 referred to herein as ZTS-082 VL CDR3.
SEQ ID NO. 85 is the amino acid sequence that describes the variable heavy chain referred to herein as H1-23 VH.
SEQ ID NO. 86 is the nucleotide sequence that describes the variable light chain referred to herein as H1-23 VH.
SEQ ID NO. 87 is the amino acid sequence that describes the variable light chain referred to herein as KPL VL.
SEQ ID NO. 88 is the nucleotide sequence that encodes the variable light chain referred to herein as KPL VL.
SEQ ID NO. 89 is the amino acid sequence that describes the variable light chain referred to herein as L3-K36 VL.
SEQ ID NO. 90 is the first nucleotide sequence that encodes the variable light chain referred to herein as L3-K36 VL.
SEQ ID NO. 91 is the second nucleotide sequence that encodes the variable light chain referred to herein as L3-K36 VL.
SEQ ID NO. 92 is the amino acid sequence that describes the variable heavy chain referred to herein as H733 VH.
SEQ ID NO. 93 is the nucleotide sequence that encodes the variable heavy chain referred to herein as H733 VH.
SEQ ID NO. 94 is the amino acid sequence that describes the variable light chain referred to herein as K643 VL.
SEQ ID NO. 95 is the nucleotide sequence that encodes the variable light chain referred to herein as K643 VL.
SEQ ID NO. 96 is the amino acid sequence that describes the variable heavy chain referred to herein as MU-01 B12-02B08-VH.
SEQ ID NO. 97 is the nucleotide sequence that describes the variable heavy chain referred to herein as MU-01 B12-02B08-VH.
SEQ ID NO. 98 is the amino acid that describes the variable heavy chain referred to herein as Chim-01B12-VH.
SEQ ID NO. 99 is the nucleotide sequence that describes the variable heavy chain referred to herein as Chim-01 B12-VH.
SEQ ID NO. 100 is the amino acid sequence that describes the variable light chain referred to herein as Mu-01B12-02B08-VL.
SEQ ID NO. 101 is the nucleotide sequence that describes the variable light chain referred to herein as Mu-01B12-02B08-VL.
SEQ ID NO. 102 is the amino acid sequence that describes the variable light chain referred to herein as Chim-01 B12-VL.
SEQ ID NO. 103 is the nucleotide sequence that describes the variable light chain referred to herein as Chim-01 B12-VL.
SEQ ID NO. 104 is the amino acid sequence that describes the variable light chain referred to herein as Mu-02B04-02A08-VH.
SEQ ID NO. 105 is the nucleotide sequence that describes the variable light chain referred to herein as Mu-02B04-02A08-VH.
SEQ ID NO. 106 is the amino acid sequence that describes the variable heavy chain referred to herein as Chim-02B04-VH.
SEQ ID NO.107 is the nucleotide sequence that describes the variable heavy chain referred to herein as Chim-02B04-VH.
SEQ ID NO.108 is the amino acid sequence that describes the variable light chain referred to herein as Mu-02B04-02A08-VL.
SEQ ID NO.109 is the nucleotide sequence that describes the variable light chain referred to herein as Mu-02B04-02A08-VL.
SEQ ID NO.110 is the amino acid sequence that describes the variable light chain referred to herein as Chim-02B04-VL.
SEQ ID NO.111 is the nucleotide sequence that describes the variable light chain referred to herein as Chim-02B04-VL.
SEQ ID NO. 112 is the amino acid sequence that describes the variable heavy chain referred to herein as Mu-15H02-02E01-VH.
SEQ ID NO.113 is the nucleotide sequence that describes the variable heavy chain referred to herein as Mu-15H02-02E01-VH.
SEQ ID NO. 114 is the amino acid sequence that describes the variable light chain referred to herein as Mu-15H02-02E01 VL.
SEQ ID NO.115 is the nucleotide sequence that describes the variable light chain referred to herein as Mu-15H02-02E01 VL.
SEQ ID NO.116 is the amino acid sequence that describes the variable heavy chain referred to herein as Mu-16G01-02F03-02D06-VH.
SEQ ID NO.117 is the nucleotide sequence that describes the variable heavy chain referred to herein as Mu-16G01-02F03-02D06-VH.
SEQ ID NO.118 is the amino acid sequence that describes the variable heavy chain referred to herein as Chim-16G01-VH.
SEQ ID NO.119 is the nucleotide sequence that describes the variable heavy chain referred to herein as Chim-16G01-VH.
SEQ ID NO.120 is the amino acid sequence that describes the variable light chain referred to herein as Mu-16G01-02F03-02D06-VL.
SEQ ID NO.121 is the nucleotide sequence that describes the variable light chain referred to herein as Mu-16G01-02F03-02D06-VL.
SEQ ID NO.122 is the amino acid sequence that describes the variable light chain referred to herein as Chim-16G01-VL.
SEQ ID NO.123 is the nucleotide sequence that describes the variable light chain referred to herein as Chim-16G01-VL.
SEQ ID NO.124 is the amino acid sequence that describes the variable heavy chain referred to herein as Mu-20D11-02E10-VH.
SEQ ID NO.125 is the nucleotide sequence that describes the variable heavy chain referred to herein as Mu-20D11-02E10-VH.
SEQ ID NO.126 is the amino acid sequence that describes the variable light chain referred to herein as Mu-20D11-02E10-VL.
SEQ ID NO.127 is the nucleotide sequence that describes the variable light chain referred to herein as Mu-20D11-02E10-VL.
SEQ ID NO.128 is the amino acid sequence that describes the variable light chain referred to herein as Chim-20D11-VL.
SEQ ID NO.129 is the nucleotide sequence that describes the variable light chain referred to herein as Chim-20D11-VL.
SEQ ID NO.130 is the amino acid sequence that describes the variable heavy chain referred to herein as Mu-26C08-02F06-VH.
SEQ ID NO.131 is the nucleotide sequence that describes the variable heavy chain referred to herein as Mu-26C08-02F06-VH.
SEQ ID NO.132 is the amino acid sequence that describes the variable heavy chain referred to herein as Chim-26C08-VH.
SEQ ID NO.133 is the nucleotide sequence that describes the variable heavy chain referred to herein as Chim-26C08-VH.
SEQ ID NO.134 is the amino acid sequence that describes the variable light chain referred to herein as Mu-26C08-02F06-VL.
SEQ ID NO.135 is the nucleotide sequence that describes the variable light chain referred to herein as Mu-26C08-02F06-VL.
SEQ ID NO.136 is the amino acid sequence that describes the variable light chain referred to herein as Chim-26C08-VL.
SEQ ID NO.137 is the nucleotide sequence that describes the variable light chain referred to herein as Chim-26C08-VL.
SEQ ID NO.138 is the amino acid sequence that describes the variable heavy chain referred to herein as Mu-30E01-02A11-VH.
SEQ ID NO.139 is the nucleotide sequence that describes the variable heavy chain referred to herein as Mu-30E01-02A11-VH.
SEQ ID NO.140 is the amino acid sequence that describes the variable light chain referred to herein as Mu-30E01-02A11-VL.
SEQ ID NO.141 is the nucleotide sequence that describes the variable light chain referred to herein as Mu-30E01-02A11-VL.
SEQ ID NO.142 is the amino acid sequence that describes the variable heavy chain referred to herein as Mu-31 F05-02B03-VH.
SEQ ID NO.143 is the nucleotide sequence that describes the variable heavy chain referred to herein as Mu-31 F05-02B03-VH.
SEQ ID NO.144 is the amino acid sequence that describes the variable light chain referred to herein as Mu-31 F05-02B03-VL.
SEQ ID NO: 145 is the amino acid sequence that describes the variable heavy chain CDR1 referred to herein as MU 01 B12 VH CDR1.
SEQ ID NO:146 is the amino acid sequence that describes the variable heavy chain CDR2 referred to herein as MU 01B12 VH CDR2.
SEQ ID NO:147 is the amino acid sequence that describes the variable heavy chain CDR3 referred to herein as MU 01B12 VH CDR3.
SEQ ID NO:148 is the amino acid sequence that describes the variable light chain CDR1 referred to herein as MU 01B12 VL CDR1.
SEQ ID NO:149 is the amino acid sequence that describes the variable light chain CDR2 referred to herein as MU 01B12 VL CDR2.
SEQ ID NO:150 is the amino acid sequence that describes the variable light chain CDR3 referred to herein as MU 01B12 VL CDR3.
SEQ ID NO:151 is the amino acid sequence that describes the variable heavy chain CDR3 referred to herein as MU 02B04 VH CDR3.
SEQ ID NO: 152 is the amino acid sequence that describes the variable light chain CDR3 referred to herein as MU 02B04 VL CDR3.
SEQ ID NO:153 is the amino acid sequence that describes the variable heavy chain CDR3 referred to herein as MU 26C08 VH CDR3.
SEQ ID NO:154 is the amino acid sequence that describes the variable light chain CDR1 referred to herein as Mu 20D11 VL CDR1.
SEQ ID NO:155 is the amino acid sequence that describes the variable light chain CDR2 referred to herein as MU 20D11 VL CDR2.
SEQ ID NO:156 is the amino acid sequence that describes the variable light chain CDR3 referred to herein as MU 20D11 VL CDR3.
SEQ ID NO:157 is the amino acid sequence that describes the variable light chain CDR3 referred to herein as MU 26C08 VL CDR3.
SEQ ID NO: 158 is the amino acid sequence that describes the canine heavy chain constant region IgGB.
SEQ ID NO: 159 is the nucleotide sequence that encodes the canine heavy chain constant region IgGB.
SEQ ID NO: 160 is the amino acid sequence that describes the canine light chain constant region.
SEQ ID NO: 161 is the nucleotide sequence that encodes the canine light chain constant region.
SEQ ID NO: 162 is the amino acid sequence that describes the feline heavy chain constant region.
SEQ ID NO: 163 is the nucleotide sequence that encodes the feline heavy chain constant region.
SEQ ID NO: 164 is the amino acid sequence that describes the feline light chain constant region.
SEQ ID NO: 165 is the amino acid sequence that describes the feline light chain constant region.
SEQ ID NO: 166is the nucleotide sequence that describes the feline light chain constant region.
SEQ ID NO: 167 is the amino acid sequence that describes the variable light chain CDR1 referred to herein as Can-182-m6 VL CDR1.
SEQ ID NO: 168 is the amino acid sequence that describes the variable light chain CDR2 referred to herein as Can-182-m6 VL CDR2.
SEQ ID NO: 169 is the amino acid sequence that describes the variable light chain CDR3 referred to herein as Can-182-m6 VL CDR3.
SEQ ID NO: 170 is the amino acid sequence that describes the variable light chain CDR2 referred to herein as Can-182-m43 VL CDR2.
SEQ ID NO: 171 is the amino acid sequence that describes the variable light chain CDR2 referred to herein as Can-182-m70 VL CDR2.
SEQ ID NO: 172 is the amino acid sequence that describes the variable light chain CDR2 referred to herein as Can-182-m72 VL CDR2.
SEQ ID NO: 173is the amino acid sequence that describes the variable light chain CDR2 referred to herein as Can-182-m75 VL CDR2.
SEQ ID NO: 174 is the amino acid sequence that describes the variable light chain CDR2 referred to herein as Can-182-m114 VL CDR2.
SEQ ID NO: 175 is the amino acid sequence that describes the variable light chain referred to herein as Can-182m6 VL.
SEQ ID NO: 176 is the nucleic acid sequence that encodes the variable light chain referred to herein as Can-182 m6 VL.
SEQ ID NO: 177 is the amino acid sequence formula that describes variants of SEQ ID NO.4.
SEQ ID NO: 178 is the amino acid sequence formula that describes variants of SEQ ID NO.5.
SEQ ID NO:179 is the amino acid sequence formula that describes variants of SEQ ID NO.6.
SEQ ID NO: 180 is the amino acid sequence formula that describes variants of SEQ ID NO.4.
SEQ ID NO: 181 is the amino acid sequence formula that describes variants of SEQ ID NO.5.
SEQ ID NO:182 is the amino acid sequence formula that describes variants of SEQ ID NO.6.
SEQ ID NO. 183 is the amino acid sequence that describes the variable light chain referred to herein as 182m6VL.
SEQ ID NO: 184 is the amino acid sequence that describes the canine IgGB Fc region comprising effector function mutations.
SEQ ID NO:185 is the nucleic acid sequence that encodes the canine IgGB Fc region comprising effector function mutations.

### DETAILED DESCRIPTION OF THE INVENTION

The invention disclosed herein provides anti-NGF antigen binding proteins that bind NGF with high affinity. The invention further provides antigen binding proteins and polypeptides that also bind to NGF that are variants of said antigen binding proteins as well as methods of making and using these antigen binding proteins. In some embodiments, the invention also provides polynucleotides encoding said antigen binding proteins and/or polypeptide. The invention disclosed herein also provides methods for preventing and/or treating pain by administration of a therapeutically effective amount of the anti-NGF antigen binding proteins of the invention.

### General Techniques

It should be understood that this invention is not limited to the particular methodology, protocols, and reagents, etc., described herein and as such may vary. The terminology used herein is for the purpose of describing particular embodiments only, and is not intended to limit the scope of the present invention, which is defined solely by the claims.

Unless otherwise defined, scientific and technical terms used in connection with the antigen binding proteins described herein shall have the meanings that are commonly understood by those of ordinary skill in the art. Further, unless otherwise required by context, singular terms shall include pluralities and plural terms shall include the singular. Generally, nomenclatures utilized in connection with, and techniques of, cell and tissue culture, molecular biology, and protein and oligo- or polynucleotide chemistry and hybridization described herein are those well-known and commonly used in the art and are not limited to a single description. It is well known in the art that different techniques may be substituted for what is described.

All patents and other publications identified are expressly incorporated herein by reference for the purpose of describing and disclosing, for example, the methodologies described in such publications that might be used in connection with the present invention. These publications are provided solely for their disclosure prior to the filing date of the present application

Standard techniques are used for recombinant DNA, oligonucleotide synthesis, and tissue culture and transfection (ex. electroporation, lipofection). Enzymatic reactions and purification techniques are performed according to manufacturer's specifications or as commonly accomplished in the art or as described herein. The foregoing techniques and procedures are generally performed according to conventional methods well known in the art and as described, but not limited to the various general and more specific references that are cited and discussed throughout the present specification, See ex. Sambrook et al. MOLECULAR CLONING: LAB. MANUAL (3^{rd} ed., Cold Spring Harbor Lab. Press, Cold Spring Harbor, N.Y., 2001) and Ausubel et al. Current Protocols in Molecular Biology (New York: Greene Publishing Association J Wiley Interscience), Oligonucleotide Synthesis (M. J. Gait, ed.,1984); Methods in Molecular Biology, Humana Press; Cell Biology: A Laboratory Notebook (J. E. Cellis, ed., 1998) Academic Press; Animal Cell Culture (R. 1. Freshney, ed. 1987); Introduction to Cell and Tissue Culture (1. P. Mather and P. E. Roberts, 1998) Plenum Press; Cell and Tissue Culture: Laboratory Procedures (A. Doyle, J. B. Griffiths, and D. G. Newell, eds., 1993-1998) J. Wiley and Sons; Methods in Enzymology (Academic Press, Inc.); Handbook of Experimental Immunology (D. M. Weir and C. C. Blackwell, eds.); Gene Transfer Vectors for Mammalian Cells (J. M. Miller and M. P. Calos, eds., 1987); Current Protocols in Molecular Biology (F. M. Ausubel et al., eds., 1987); PCR: The Polymerase Chain Reaction, (Mullis et al., eds., 1994); Current Protocols in Immunology (E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: a practical approach (D. Catty., ed., IRL Press, 1988-1989); Monoclonal antibodies: a practical approach (P. Shepherd and C. Dean, eds., Oxford University Press, 2000); Using antibodies: a laboratory manual (E. Harlow and D. Lane (Cold Spring Harbor Laboratory Press, 1999); The Antibodies (M. Zanetti and J. D. Capra, eds., Harwood Academic Publishers, 1995); and Cancer: Principles and Practice of Oncology (Y. T. DeVita et al., eds., J. B. Lippincott Company, 1993).

Other than in the operating examples, or where otherwise indicated, all numbers expressing quantities of ingredients or reaction conditions used herein should be understood as modified in all instances by the term “about.”

### Definitions

Before describing the present invention in detail, several terms used in the context of the present invention will be defined. In addition to these terms, others are defined elsewhere in the specification as necessary. Unless otherwise expressly defined herein, terms of art used in this specification will have their art-recognized meanings.

As used in the specification and claims, the singular form “a”, “an” and “the” includes plural references unless the context clearly dictates otherwise. For example, reference to “an antibody” includes a plurality of such antibodies.

As used herein, the term “comprising” is intended to mean that the compositions and methods include the recited elements, but not excluding others.

As used herein, the term “nerve growth factor” and “NGF” refers to nerve growth factor and variants thereof that retain at least part of the biological activity of NGF.

“NGF receptor” refers to a polypeptide that is bound by or activated by NGF. NGF receptors include the TrkA receptor and to a lesser extent the p75 receptor of canines.

“Biological activity” of NGF generally refers to the ability to bind NGF receptors and/or activate NGF receptor signaling pathways. Without limitation, a biological activity includes anyone or more of the following: the ability to bind an NGF receptor (such as TrkA and/or p75); the ability to promote TrkA receptor dimerization and/or autophosphorylation; the ability to activate an NGF receptor signaling pathway; the ability to promote cell differentiation, proliferation, survival, growth and other changes in cell physiology, including (in the case of neurons, including peripheral and central neuron) change in neuronal morphology, synaptogenesis, synaptic function, neurotransmitter and/or neuropeptide release and regeneration following damage; the ability to promote survival of mouse E13.5 trigeminal neurons; and the ability to mediate pain, including post-surgical pain.

As used herein, an “anti-NGF antigen binding protein” (interchangeably termed “anti-NGF antibody” and “anti-NGF antagonist antibody”) refers to an antigen binding protein which is able to bind to NGF and inhibit NGF biological activity and/or downstream pathway(s) mediated by NGF signaling. An anti-NGF antigen binding protein encompass binding proteins and antibodies that block, antagonize, suppress or reduce (including significantly) NGF biological activity, including downstream pathways mediated by NGF signaling and/or inhibit NGF from binding to its receptor TrkA, such as receptor binding and/or elicitation of a cellular response to NGF. For purpose of the present invention, it will be explicitly understood that the term “anti-NGF antigen binding protein” or “anti-NGF-antagonist antibody” encompass all the previously identified terms, titles, and functional states and characteristics whereby the NGF itself, an NGF biological activity (including but not limited to its ability to ability to mediate any aspect of osteoarthritis pain, inflammatory pain, post-surgical pain, cancer pain and the like, all described herein), or the consequences of the biological activity, are substantially nullified, decreased, or neutralized in any meaningful degree. In some embodiments, an anti-NGF antagonist antibody binds NGF and prevent NGF dimerization and/or binding to an NGF receptor (such as TrkA and/or p75). In other embodiments, an anti-NGF antigen binding protein binds to NGF and prevents TrkA receptor dimerization and/or TrkA autophosphorylation. Examples of anti-NGF antagonist antibodies are provided herein.

As used herein, the term “antigen binding protein”, “antibody” “antigen binding protein” and the like, which may be used interchangeably, refers to a polypeptide, or fragment thereof, comprising an antigen binding site. In one embodiment of the present invention the antigen binding protein of the invention further provides an intact immunoglobulin capable of specific binding to a target, such as a carbohydrate, polynucleotide, lipid, polypeptide, etc., through at least one antigen recognition site located in the variable region of the immunoglobulin molecule. An intact antibody has two light and two heavy chains. Thus, a single isolated intact antibody may be a polyclonal antibody, a monoclonal antibody, a synthetic antibody, a recombinant antibody, a chimeric antibody, a heterochimeric antibody. The term “antigen binding protein” “antibody’ and the like preferably refers to monoclonal antibodies and fragments thereof, and immunologic binding equivalents thereof that can bind to the NGF protein and fragments thereof. The term antibody and antigen binding protein are used to refer to a homogeneous molecular, or a mixture such as a serum product made up of a plurality of different molecular entities. As used herein, the term encompasses not only intact polyclonal or monoclonal antibodies, but also fragments thereof. For the purposes of the present invention, “antibody” and “antigen binding protein” also includes antibody fragments, unless otherwise stated. Exemplary antibody fragments include Fab, Fab′, F(ab′)2, Fv, scFv, Fd, dAb, diabodies, their antigen-recognizing fragments, small modular immunopharmaceuticals (SMIPs) nanobodies, IgNAR molecules and the equivalents that are recognized by one of skill in the art to be an antigen binding protein or antibody fragment and any of above mentioned fragments and their chemically or genetically manipulated counterparts, as well as other antibody fragments and mutants thereof, fusion proteins comprising an antibody portion, and any other modified configuration of the immunoglobulin molecule that comprises an antigen recognition site. Antibodies and antigen binding proteins can be made, for example, via traditional hybridoma techniques (Kohler et al., Nature 256:495-499 (1975)), recombinant DNA methods (U.S. Pat. No. 4,816,567), or phage display techniques using antibody libraries (Clackson et al., Nature 352:624-628 (1991); Marks et al., J. Mol. Biol. 222:581-597 (1991)). For various other antibody production techniques, see Antibodies: A Laboratory Manual, eds. Harlow et al., Cold Spring Harbor Laboratory, 1988 as well as other techniques that are well known to those skilled in the art.

A “monoclonal antibody” as defined herein is an antibody produced by a single clone of cells (specifically, a single clone of hybridoma cells) and therefore a single pure homogeneous type of antibody. All monoclonal antibodies produced from the same clone are identical and have the same antigen specificity. Monoclonal antibodies are a homogeneous antibody population wherein the monoclonal antibody is comprised of amino acids (naturally occurring and non-naturally occurring) that are involved in the selective binding of an antigen. A population of monoclonal antibodies is highly specific, being directed against a single antigenic site. The term “monoclonal antibody” encompasses not only intact monoclonal antibodies and full-length monoclonal antibodies, but also fragments thereof (Fab, Fab′, F(ab′)₂, Fv, scFv, Fd, dAb, diabodies, their antigen-recognizing fragments, small modular immunopharmaceuticals (SMIPs) nanobodies, IgNAR molecules and the like), mutants thereof, fusion proteins comprising an antibody portion, and any other modified configuration of the immunoglobulin molecule that comprises an antigen recognition site of the required specificity and the ability to bind to an antigen. It is not intended to be limited in regards to the source of the antibody or the manner in which it is made (ex. by hybridoma, phage selection, recombinant expression, transgenic animals, etc.).

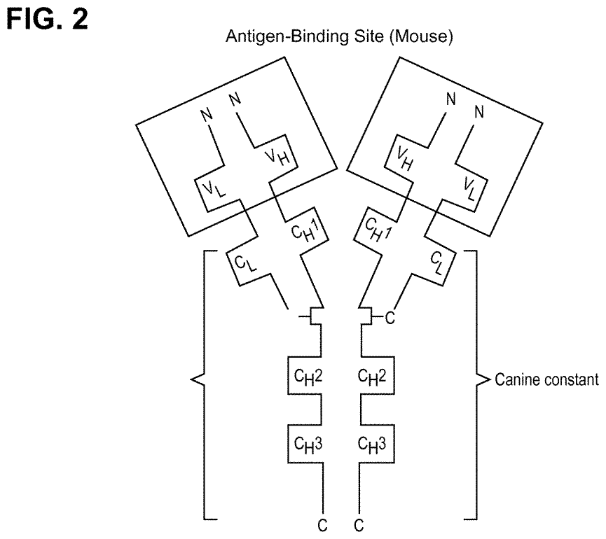

The monoclonal antibodies herein specifically include “chimeric” antibodies (immunoglobulins) in which a portion of the heavy and/or light chain is identical with or homologous to corresponding sequences in antibodies derived from a particular species, while the remainder of the chain(s) is identical with or homologous to corresponding sequences in antibodies derived from another species, as well as fragments of such antibodies, so long as they exhibit the desired biological activity. Typically, chimeric antibodies are antibodies whose light and heavy chain genes have been constructed, typically by genetic engineering, from antibody variable and constant region genes belonging to different species. For example, the variable segments of the genes from a mouse monoclonal antibody may be joined to canine constant segments. FIG. 2 is a schematic representation of the general structure of one embodiment of a mouse: canine IgG. In this embodiment, the antigen binding site is derived from mouse while the Fc portion is canine.

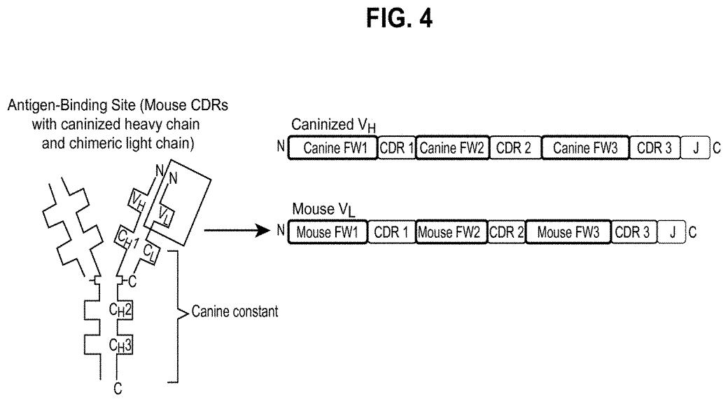

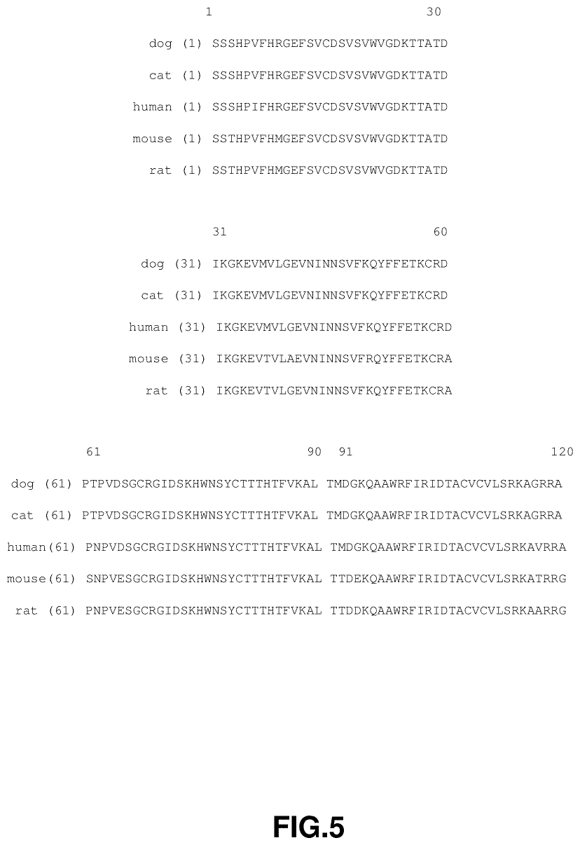

The term “heterochimeric” as defined herein, refers to an antibody in which one of the antibody chains (heavy or light) is caninized while the other is chimeric. FIG. 4 depicts one embodiment of a heterochimeric molecule. In this embodiment, a caninized variable heavy chain (where all of the CDRs are mouse and all FRs are canine) is paired with a chimeric variable light chain (where all of the CDRs are mouse and all FRs are mouse. In this embodiment, both the variable heavy and variable light chains are fused to a canine constant region.

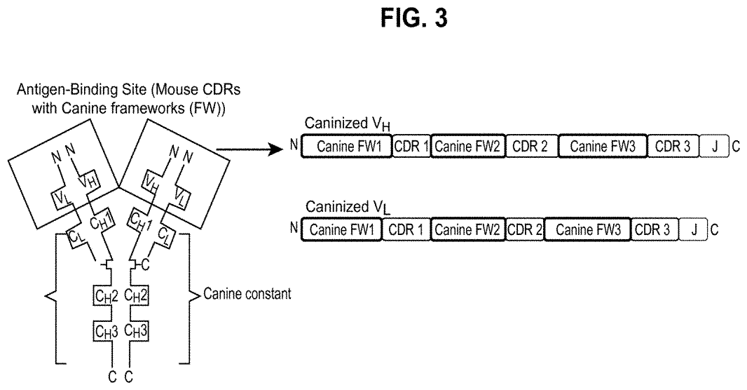

For the sake of simplicity, the following describes “caninized” antibodies, however the same can be applied to felinized, equinized, humanized or any other “speciated” antigen binding protein. As an example, “Caninization” is defined as a method for transferring non-canine antigen-binding information from a donor antibody to a less immunogenic canine antibody acceptor to generate treatments useful as therapeutics in dogs. Caninized antibodies are canine immunoglobulins (recipient antibody) in which hypervariable region residues of the recipient are replaced by hypervariable region residues from a non-canine species (donor antibody) such as such as mouse, rat, rabbit, cat, dogs, goat, chicken, bovine, horse, llama, camel, dromedaries, sharks, non-human primates, human, humanized, recombinant sequence, or an engineered sequence having the desired properties, specificity, affinity, and capacity. In some instances, framework region (FR) residues of the canine immunoglobulin are replaced by corresponding non-canine residues. Furthermore, caninized antibodies may include residues that are not found in the recipient antibody or in the donor antibody. These modifications are made to further refine antibody performance. The modifications to the hypervariable regions and/or the framework regions, as described herein, are determined for each separately engineered speciated (caninized) antibody based on experimentation known to those in the art and cannot be predicted prior to said experimentation. In general, the caninized antibody will include substantially all of at least one, and typically two, variable domains, in which all or substantially all of the hypervariable regions correspond to those of a non-canine immunoglobulin and all or substantially all of the FRs are those of a canine immunoglobulin sequence. The caninized antibody optionally also will comprise a complete, or at least a portion of an immunoglobulin constant region (Fc), typically that of a canine immunoglobulin. FIG. 3 is an illustration of one embodiment showing speciation or caninization of a mouse IgG. In this embodiment, mouse CDRs are grafted onto canine framework sequences. In some cases, mouse frameworks or residues therein that are outside of the hypervariable region are maintained. All descriptions of caninization of an antigen binding protein and that of a caninized antigen binding protein can be applicable, in concept, to any speciated antibody, whether it is caninization, felinization, equinization, humanization etc.

The phrase “recombinant canine antibody”, “recombinant feline antibody”, “recombinant human antibody” and the like all include speciated antibodies that are prepared, expressed, created or isolated by recombinant means, such as antibodies expressed using a recombinant expression vector transfected into a host cell, antibodies isolated from a recombinant, combinatorial canine (or feline, human etc.) antibody library, antibodies isolated from an animal (ex. a mouse) that is transgenic for canine immunoglobulin genes (see ex. Taylor, L. D., et al. (1992) Nucl. Acids Res. 20:6287-6295) or antibodies prepared, expressed, created or isolated by any other means that involves splicing of canine (or feline, human etc.) immunoglobulin gene sequences to other DNA sequences.

The term “canine antibody”, “feline antibody”, “human antibody” and the like, as used herein, refers to an antibody (antigen binding protein) that is generated against a target and is prepared by hybridoma methods well known to one skilled in the art and described herein.

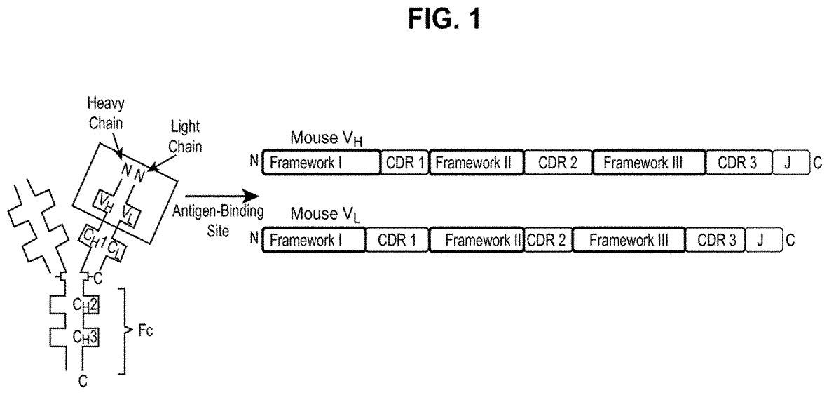

“Native antibodies” and “native immunoglobulins” are usually heterotetrameric glycoproteins of about 150,000 Daltons, composed of two identical light (I) chains and two identical heavy (H) chains. Each light chain is linked to a heavy chain by one covalent disulfide bond, while the number of disulfide linkages varies among the heavy chains of different immunoglobulin isotypes. Each heavy and light chain also has regularly spaced intrachain disulfide bridges. Each heavy chain has at one end a variable domain (VH) followed by a number of constant domains. Each light chain has a variable domain at one end (VL) and a constant domain at its other end; the constant domain of the light chain is aligned with the first constant domain of the heavy chain, and the light-chain variable domain is aligned with the variable domain of the heavy chain. Particular amino acid residues are believed to form an interface between the light- and heavy-chain variable domains. FIG. 1 is an example of the general structure of a native mouse immunoglobulin G (IgG) highlighting the antigen binding site.

The “parent” antibody herein is one that is encoded by an amino acid sequence used for the preparation of the variant. Preferably, the parent antibody has a canine framework region and, if present, has canine antibody constant region(s). For example, the parent antibody may be a caninized or canine antibody.

Depending on the amino acid sequence of the constant domain of the heavy chains of antibodies, immunoglobulins can be assigned to different classes. Presently there are five major classes of immunoglobulins: IgA, IgD, IgE, IgG, and IgM, and several of these may be further divided into subclasses (isotypes), ex. IgG₁, IgG₂, IgG₃, IgG₄, IgA, and IgA₂ (as defined by mouse and human designation). The heavy-chain constant domains that correspond to the different classes of immunoglobulins are called alpha, delta, epsilon, gamma, and mu, respectively. The subunit structures and three-dimensional configurations of different classes of immunoglobulins are well known in multiple species. The prevalence of individual isotypes and functional activities associated with these constant domains are species-specific and must be experimentally defined.

The “light chains” of antibodies (immunoglobulins) from any vertebrate species can be assigned to one of two clearly distinct types, called kappa (K) and lambda (A), based on the amino acid sequences of their constant domains.

A “variable region” of an antibody refers to the variable region of the antibody light chain or the variable region of the antibody heavy chain, either alone or in combination. The variable regions of the heavy and light chain each consist of four framework regions (FR) connected by three complementarity determining regions (CDRs) also known as hypervariable regions. The CDRs in each chain are held together in close proximity by the FRs and, with the CDRs from the other chain, contribute to the formation of the antigen-binding site of antibodies. There are at least two techniques for determining CDRs: (I) an approach based on cross-species sequence variability (i.e., Kabat et al. Sequences of Proteins of Immunological Interest, (5th ed., 1991, National Institutes of Health, Bethesda Md.)); and (2) an approach based on crystallographic studies of antigen-antibody complexes (Chothia et al. (1989) Nature 342:877; Al-Iazikani et al (1997) J. Molec. Bioi. 273:927-948)). As used herein, a CDR may refer to CDRs defined by either approach or by a combination of both approaches.

The term “hypervariable region” when used herein refers to the amino acid residues of an antibody which are responsible for antigen binding. The hypervariable region comprises amino acid residues from a “complementarity determining region” or “CDR” (Kabat, et al. (1991), above) and/or those residues from a “hypervariable loop” (Chothia and Lesk J. Mol. Biol. 196:901-917 (1987). “Framework” or “FR” residues are those variable domain residues other than the hypervariable region residues as herein defined.

As used herein, the term “antigen binding region” refers to that portion of an antibody molecule which contains the amino acid residues that interact with an antigen and confer on the antibody its specificity and affinity for the antigen. The antibody binding region includes the “framework” amino acid residues necessary to maintain the proper conformation of the antigen-binding residues.

A “functional Fc region” possesses at least one effector function of a native sequence Fc region. Exemplary “effector functions” include C1q binding; complement dependent cytotoxicity (CDC); Fc receptor binding; neonatal receptor binding; antibody-dependent cell-mediated cytotoxicity (ADCC); phagocytosis; down-regulation of cell surface receptors (e.g. B cell receptor; BCR), etc. Such effector functions generally require the Fc region to be combined with a binding domain (e.g. an antibody variable domain) and can be assessed using various assays known in the art for evaluating such antibody effector functions.

A “native sequence Fc region” comprises an amino acid sequence identical to the amino acid sequence of an Fc region found in nature. A “variant Fc region” or a “mutated” or “mutant” Fc region comprises an amino acid sequence which differs from that of a native sequence Fc region by virtue of at least one amino acid modification, and may or may not retain at least one effector function of the native sequence Fc region. Preferably, the variant Fc region has at least one amino acid substitution compared to a native sequence Fc region or to the Fc region of a parent polypeptide, e.g. from about one to about ten amino acid substitutions, and preferably from about one to about five amino acid substitutions in a native sequence Fc region or in the Fc region of the parent polypeptide. The variant Fc region herein will preferably possess at least about 80% sequence identity with a native sequence Fc region and/or with an Fc region of a parent polypeptide, and most preferably at least about 90% sequence identity therewith, more preferably at least about 95% sequence identity therewith. A variant or mutated Fc region may also essentially eliminate the function of the Fc region of the antibody. For example Fc region mutations may eliminate effector function of the antibody. In one embodiment of the invention the antibody of the invention comprises a mutated Fc region. In one embodiment of the invention the antibody of the invention comprises a mutated Fc region that no longer has effector function.

As used herein, “Fc receptor” and “FcR” describe a receptor that binds to the Fc region of an antibody. The preferred FcR is a native sequence FcR. Moreover, a preferred FcR is one which binds an IgG antibody (a gamma receptor) and includes receptors of the FcyRI, FcyRII, and FcyRIII subclasses, including allelic variants and alternatively spliced forms of these receptors. FcyRII receptors include FcyRIIA (an “activating receptor”) and FcyRIIB (an “inhibiting receptor”), which have similar amino acid sequences that differ primarily in the cytoplasmic domains thereof. FcRs are reviewed in Ravetch and Kinet, 1991, Ann. Rev. Immunol., 9:457-92; Capel et al., 1994, Immunomethods, 4:25-34; and de Haas et al., 1995, J. Lab. Clin. Med., 126:330-41. “FcR” also includes the neonatal receptor, FcRn, which is responsible for the transfer of maternal IgGs to the fetus (Guyer et al., 1976, J. Immunol., 117:587; and Kim et al., 1994, J. Immunol., 24:249).

As used herein “antibody-dependent cell-mediated cytotoxicity” and “ADCC” refer to a cell-mediated reaction in which nonspecific cytotoxic cells that express Fc receptors (FcRs) (e.g. natural killer (NK) cells, neutrophils, and macrophages) recognize bound antibody on a target cell and subsequently cause lysis of the target cell. ADCC activity of a molecule of interest can be assessed using an in vitro ADCC assay, such as that described in U.S. Pat. No. 5,500,362 or 5,821,337. Useful effector cells for such assays include peripheral blood mononuclear cells (PBMC) and NK cells. Additionally, ADCC activity of the molecule of interest may be assessed in vivo, for example, in an animal model such as that disclosed in Clynes et al., 1998, PNAS (USA), 95:652-656.

“Complement dependent cytotoxicity” and “CDC” refer to the lysing of a target in the presence of complement. The complement activation pathway is initiated by the binding of the first component of the complement system (C1q) to a molecule (e.g. an antibody) complexed with a cognate antigen. To assess complement activation, a CDC assay, e.g. as described in Gazzano-Santoro et al., J. Immunol. Methods, 202: 163 (1996), may be performed.

Papain digestion of antibodies produces two identical antigen-binding fragments, called “Fab” fragments, each with a single antigen-binding site, and a residual “Fc” fragment, whose name reflects its ability to crystallize readily. Pepsin treatment yields an F(ab′)₂ fragment that has two antigen-combining sites and is still capable of cross-linking antigen.

The Fab fragment also contains the constant domain of the light chain and the first constant domain (CH1) of the heavy chain. Fab′ fragments differ from Fab fragments by the addition of a few residues at the carboxyl terminus of the heavy chain CH1 domain including one or more cysteine(s) from the antibody hinge region. Fab′-SH is the designation herein for Fab′ in which the cysteine residue(s) of the constant domains bear a free thiol group. F(ab′)₂ antibody fragments originally were produced as pairs of Fab′ fragments which have hinge cysteines between them. Other chemical couplings of antibody fragments are also known.

“Fv’ is the minimum antibody fragment that contains a complete antigen-recognition and binding site. This region consists of a dimer of one heavy chain and one light chain variable domain in tight, non-covalent association. It is in this configuration that the three hypervariable regions of each variable domain interact to define an antigen-binding site on the surface of the V_{H}-V_{L} dimer. Collectively, the six hypervariable regions confer antigen-binding specificity to the antibody. However, even a single variable domain (or half of an Fv comprising only three hypervariable regions specific for an antigen) has the ability to recognize and bind antigen, although at a lower affinity than the entire binding site.

An “antigen”, as used herein, refers to the antigenic determinant recognized by the CDRs of the antigen binding protein or antibody as described herein. In other words, epitope refers to that portion of any molecule capable of being recognized by, and bound by, an antibody. Unless indicated otherwise, the term “epitope” as used herein, refers to the region of NGF to which an anti-NGF antigen binding protein/antibody/agent binds.

The term “antigen binding domain,” “active fragments of an antibody” or the like refers to the part of an antibody or antigen binding protein that comprises the area specifically binding to or complementary to a part or all of an antigen. Where an antigen is large, an antibody may only bind to a particular part of the antigen. The “epitope,” “active fragments of an epitope,” or “antigenic determinant” or the like is a portion of an antigen molecule that is responsible for specific interactions with the antigen binding domain of an antibody. An antigen binding domain may be provided by one or more antibody variable domains (for example a so-called Fd antibody fragment consisting of a VH domain). An antigen binding domain may comprise an antibody light chain variable domain (VL) and an antibody heavy chain variable domain (VH) (U.S. Pat. No. 5,565,332).

The terms “binding portion” of an antibody (or “antibody portion”) or antigen-binding polypeptide or the like includes one or more complete domains, for example, a pair of complete domains, as well as fragments of an antibody that retain the ability to specifically bind to an antigen, for example, NGF. It has been shown that the binding function of an antibody can be performed by fragments of a full-length antibody. Binding fragments are produced by recombinant DNA techniques, or by enzymatic or chemical cleavage of intact immunoglobulins. Binding fragments include Fab, Fab′, F(ab′)₂, Fd, dAb, Fv, single chains, single-chain antibodies, for example, scFv, and single domain antibodies (Muyldermans et al., 2001, 26:230-5), and an isolated complementarity determining region (CDR). Fab fragment is a monovalent fragment consisting of the VL, VH, CL and CH1 domains. F(ab′)₂ fragment is a bivalent fragment comprising two Fab fragments linked by a disulfide bridge at the hinge region. Fd fragment consists of the VH and CH1 domains, and Fv fragment consists of the VL and VH domains of a single arm of an antibody. A dAb fragment consists of a VH domain (Ward et al., (1989) Nature 341:544-546). While the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv) (Bird et al., 1988, Science 242:423-426). Such single chain antibodies are also intended to be encompassed within the term “binding portion” of an antibody. Other forms of single chain antibodies, such as diabodies are also encompassed. Diabodies are bivalent, bispecific antibodies in which VH and VL domains are expressed on a single polypeptide chain, but using a linker that is too short to allow for pairing between the two domains on the same chain, thereby forcing the domains to pair with complementary domains of another chain and creating two antigen binding sites (see for example, Holliger, et al., 1993, Proc. Natl. Acad. Sci. USA 90:6444-6448). An antibody or binding portion thereof also may be part of a larger immunoadhesion molecules formed by covalent or non-covalent association of the antibody or antibody portion with one or more other proteins or peptides. Examples of such immunoadhesion molecules include use of the streptavidin core region to make a tetrameric scFv molecule (Kipriyanov, S. M., et al. (1995) Human Antibodies and Hybridomas 6:93-101) and use of a cysteine residue, a marker peptide and a C-terminal polyhistidine tag to make bivalent and biotinylated scFv molecules (Kipriyanov, S. M., et al. (1994) Mol. Immunol. 31:1047-1058). Binding fragments such as Fab and F(ab′)₂ fragments, can be prepared from whole antibodies using conventional techniques, such as papain or pepsin digestion, respectively, of whole antibodies. Moreover, antibodies, antibody portions and immunoadhesion molecules can be obtained using standard recombinant DNA techniques, as described herein and as known in the art. Other than “bispecific” or “bifunctional” antibodies, an antibody is understood to have each of its binding sites identical. A “bispecific” or “bifunctional antibody” is an artificial hybrid antibody having two different heavy/light chain pairs and two different binding sites. A bispecific antibody can also include two antigen binding regions with an intervening constant region. Bispecific antibodies can be produced by a variety of methods including fusion of hybridomas or linking of Fab′ fragments. See, for example, Songsivilai et al., Clin. Exp. Immunol. 79:315-321, 1990.; Kostelny et al., 1992, J. Immunol. 148, 1547-1553.

The term “backmutation” refers to a process in which some or all of the somatically mutated amino acids of a canine antibody are replaced with the corresponding germline residues from a homologous germline antibody sequence. The heavy and light chain sequences of the canine antibody of the invention are aligned separately with the germline sequences to identify the sequences with the highest homology. Differences in the canine antibody of the invention are returned to the germline sequence by mutating defined nucleotide positions encoding such different amino acid. The role of each amino acid thus identified as candidate for backmutation should be investigated for a direct or indirect role in antigen binding and any amino acid found after mutation to affect any desirable characteristic of the canine antibody should not be included in the final canine antibody; as an example, activity enhancing amino acids identified by the selective mutagenesis approach will not be subject to backmutation. To minimize the number of amino acids subject to backmutation those amino acid positions found to be different from the closest germline sequence but identical to the corresponding amino acid in a second germline sequence can remain, provided that the second germline sequence is identical and co-linear to the sequence of the canine antibody of the invention. Back mutation of selected target framework residues to the corresponding donor residues might be required to restore and or improved affinity.

As used herein, “immunospecific” binding of antibodies refers to the antigen specific binding interaction that occurs between the antigen-combining site of an antibody and the specific antigen recognized by that antibody (i.e., the antibody reacts with the protein in an ELISA or other immunoassay, and does not react detectably with unrelated proteins). An epitope that “specifically binds”, or “preferentially binds” (used interchangeably herein) to an antibody or a polypeptide is a term well understood in the art, and methods to determine such specific or preferential binding are also well known in the art. A molecule is said to exhibit “specific binding” or “preferential binding” if it reacts or associates more frequently, more rapidly, with greater duration and/or with greater affinity with a particular cell or substance than it does with alternative cells or substances. An antibody “specifically binds” or “preferentially binds” to a target if it binds with greater affinity, avidity, more readily, and/or with greater duration than it binds to other substances. For example, an antibody that specifically or preferentially binds to an NGF epitope is an antibody that binds this epitope with greater affinity, avidity, more readily, and/or with greater duration than it binds to other NGF epitopes or non-NGF epitopes. It is also understood by reading this definition that, for example, an antibody (or moiety or epitope) that specifically or preferentially binds to a first target mayor may not specifically or preferentially bind to a second target. As such, “specific binding” or “preferential binding” does not necessarily require (although it can include) exclusive binding. Generally, but not necessarily, reference to binding means preferential binding.

The term “specifically” in the context of antibody binding, refers to high avidity and/or high affinity binding of an antibody to a specific antigen, i.e., a polypeptide, or epitope. Antibody specifically binding an antigen is stronger than binding of the same antibody to other antigens. Antibodies which bind specifically to a polypeptide may be capable of binding other polypeptides at a weak, yet detectable level (for example, 10% or less of the binding shown to the polypeptide of interest). Such weak binding, or background binding, is readily discernible from the specific antibody binding to a subject polypeptide, e.g. by use of appropriate controls. In general, specific antibodies bind to an antigen with a binding affinity with a K_{d} of 10^{.7} M or less, 10⁻⁸ M or less 10⁻⁹ M or less, 10⁻¹⁰ M or less, 10^{.11} M or less, 10^{.12} M or less, or 10^{.13} M or less etc.

As used herein, the term “affinity” refers to the strength of the binding of a single antigen-combining site with an antigenic determinant. Affinity depends on the closeness of stereochemical fit between antibody or antigen binding protein combining sites and antigen determinants, on the size of the area of contact between them, on the distribution of charged and hydrophobic groups, etc. Antibody affinity can be measured by equilibrium analysis or by the Surface Plasmon Resonance “SPR” method (for example BIACORE™) The SPR method relies on the phenomenon of surface plasmon resonance (SPR), which occurs when surface plasmon waves are excited at a metal/liquid interface. Light is directed at, and reflected from, the side of the surface not in contact with sample, and SPR causes a reduction in the reflected light intensity at a specific combination of angle and wavelength. Bimolecular binding events cause changes in the refractive index at the surface layer, which are detected as changes in the SPR signal.

The term “Kd’, as used herein, is intended to refer to the dissociation constant of an antibody-antigen interaction. The dissociation constant, Kd, and the association constant, Ka, are quantitative measures of affinity. At equilibrium, free antigen (Ag) and free antibody (Ab) are in equilibrium with antigen-antibody complex (Ag-Ab), and the rate constants, ka and kd, quantitate the rates of the individual reactions. At equilibrium, ka [Ab][Ag]=kd [Ag-Ab]. The dissociation constant, Kd, is given by: Kd=kd/ka=[Ag][Ab]/[Ag-Ab]. Kd has units of concentration, most typically M, mM, μM, nM, pM, etc. When comparing antibody affinities expressed as Kd, having greater affinity for NGF is indicated by a lower value. The association constant, Ka, is given by: Ka=ka/kd=[Ag-Ab]/[Ag][Ab]. Ka has units of inverse concentration, most typically M⁻¹, mM⁻¹, μ.M⁻¹, nM⁻¹, pM⁻¹, etc. As used herein, the term “avidity” refers to the strength of the antigen-antibody bond after formation of reversible complexes. Anti-NGF antibodies may be characterized in terms of the Kd for their binding to a NGF protein, as binding “with a dissociation constant (Kd) in the range of from about (lower Kd value) to about (upper Kd value).”

The terms “polypeptide”, “oligopeptide”, “peptide” and “protein” are used interchangeably herein to refer to polymers of amino acids of any length. The polymer may be linear or branched, it may comprise modified amino acids, and it may be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also, included within the definition are, for example, polypeptides containing one or more analogs of an amino acid (including, for example, un-natural amino acids, etc.), as well as other modifications known in the art. It is understood that, because the polypeptides of this invention are based upon an antibody, the polypeptides can occur as single chains or associated chains.

The term ‘conservative amino acid substitution” indicates any amino acid substitution for a given amino acid residue, where the substitute residue is so chemically similar to that of the given residue that no substantial decrease in polypeptide function (for example, enzymatic activity) results. Conservative amino acid substitutions are commonly known in the art and examples thereof are described, ex., in U.S. Pat. Nos. 6,790,639, 6,774,107, 6,194,167, or 5,350,576. In a preferred embodiment, a conservative amino acid substitution will be anyone that occurs within one of the following six groups:
Small aliphatic, substantially non-polar residues: Ala, Gly, Pro, Ser, and Thr;
Large aliphatic, non-polar residues: lie, Leu, and Val; Met;
Polar, negatively charged residues and their amides: Asp and Glu;
Amides of polar, negatively charged residues: Asn and Gin; His;
Polar, positively charged residues: Arg and Lys; His; and
Large aromatic residues: Trp and Tyr; Phe.

In a preferred embodiment, a conservative amino acid substitution will be any one of the following, which are listed as Native Residue (Conservative Substitutions) pairs: Ala (Ser); Arg (Lys); Asn (Gin; His); Asp (Glu); Gin (Asn); Glu (Asp); Gly (Pro); His (Asn; Gin); Ile (Leu; Val); Leu (Ile; Val); Lys (Arg; Gin; Glu); Met (Leu; Ile); Phe (Met; Leu; Tyr); Ser (Thr); Thr (Ser); Trp (Tyr); Tyr (Trp; Phe); and Val (Ile; Leu).

The terms “nucleic acid”, “polynucleotide”, “nucleic acid molecule” and the like may be used interchangeably herein and refer to a series of nucleotide bases (also called “nucleotides”) in DNA and RNA. The nucleic acid may contain deoxyribonucleotides, ribonucleotides, and/or their analogs. The term “nucleic acid” includes, for example, single-stranded and double-stranded molecules. A nucleic acid can be, for example, a gene or gene fragment, exons, introns, a DNA molecule (ex. cDNA), an RNA molecule (ex. mRNA), recombinant nucleic acids, plasmids, and other vectors, primers and probes. Both 5′ to 3′ (sense) and 3′ to 5′ (antisense) polynucleotides are included. The nucleotides can be deoxyribonucleotides, ribonucleotides, modified nucleotides or bases, and/or their analogs, or any substrate that can be incorporated into a polymer by DNA or RNA polymerase. A poly-nucleotide may comprise modified nucleotides, such as methylated nucleotides and their analogs. If present, modification to the nucleotide structure may be imparted before or after assembly of the polymer. The sequence of nucleotides may be interrupted by non-nucleotide components. A polynucleotide may be further modified after polymerization, such as by conjugation with a labeling component. Other types of modifications include, for example, “caps”, substitution of one or more of the naturally occurring nucleotides with an analog, internucleotide modifications such as, for example, those with uncharged linkages (for example, methyl phosphonates, phosphotriesters, phosphoamidates, cabamates, etc.) and with charged linkages (ex. phosphorothioates, phosphorodithioates, etc.), those containing pendant moieties, such as, for example, proteins (ex. nucleases, toxins, antibodies, signal peptides, poly-L-lysine, etc.), those with intercalators (ex. acridine, psoralen, etc.), those containing chelators (ex., metals, radioactive metals, boron, oxidative metals, etc.), those containing alkylators, those with modified linkages (ex. alpha anomeric nucleic acids, etc.), as well as unmodified forms of the polynucleotide(s). Further, any of the hydroxyl groups ordinarily present in the sugars may be replaced, for example, by phosphonate groups, phosphate groups, protected by standard protecting groups, or activated to prepare additional linkages to additional nucleotides, or may be conjugated to solid supports. The 5′ and 3′ terminal OH can be phosphorylated or substituted with amines or organic capping groups moieties of from 1 to 20 carbon atoms. Other hydroxyls may also be derivatized to standard protecting groups. Polynucleotides can also contain analogous forms of ribose or deoxyribose sugars that are generally known in the art, including, for example, 2′-0-methyl-, 2′-0-allyl, 2′-fluoro- or 2′-azido-ribose, carbocyclic sugar analogs, anomeric sugars, epimeric sugars such as arabinose, xyloses or lyxoses, pyranose sugars, furanose sugars, sedoheptuloses, acyclic analogs and abasic nucleoside analogs such as methyl riboside. One or more phosphodiester linkages may be replaced by alternative linking groups. These alternative linking groups include, but are not limited to, embodiments wherein phosphate is replaced by P(O)S(“thioate”), P(S)S (“dithioate”), “(O)NR2 (”amidate“), P(O)R, P(O)OR′, CO or CH2 (“formacetal”), in which each R or R′ is independently H or substituted or unsubstituted alkyl (1-20 C) optionally containing an ether (-0-) linkage, aryl, alkenyl, cycloalkyl, cycloalkenyl or araldyl. Not all linkages in a polynucleotide need be identical. The preceding description applies to all polynucleotides referred to herein, including RNA and DNA.

As used herein, “vector” means a construct, which is capable of delivering, and preferably expressing, one or more gene(s) or sequence(s) of interest in a host cell. Examples of vectors include, but are not limited to, viral vectors, naked DNA or RNA expression vectors, plasmid, cosmid or phage vectors, DNA or RNA expression vectors associated with cationic condensing agents, DNA or RNA expression vectors encapsulated in liposomes, and certain eukaryotic cells, such as producer cells. Vectors, as described herein, have expression control sequences meaning that a nucleic acid sequence that directs transcription of a nucleic acid. An expression control sequence can be a promoter, such as a constitutive or an inducible promoter, or an enhancer. The expression control sequence is ‘operably linked’ to the nucleic acid sequence to be transcribed. A nucleic acid is “operably linked” when it is placed into a functional relationship with another nucleic acid sequence. For example, DNA for a pre-sequence or secretory leader is operably linked to DNA for a polypeptide if it is expressed as a pre-protein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in reading phase. However, enhancers do not have to be contiguous. Linking is accomplished by ligation at convenient restriction sites. If such sites do not exist, the synthetic oligonucleotide adaptors or linkers are used in accordance with conventional practice.

Just as a polypeptide may contain conservative amino acid substitution(s), a polynucleotide thereof may contain conservative codon substitution(s). A codon substitution is considered conservative if, when expressed, it produces a conservative amino acid substitution, as described above. Degenerate codon substitution, which results in no amino acid substitution, is also useful in polynucleotides according to the present invention. Thus, for example, a polynucleotide encoding a selected polypeptide useful in an embodiment of the present invention may be mutated by degenerate codon substitution in order to approximate the codon usage frequency exhibited by an expression host cell to be transformed therewith, or to otherwise improve the expression thereof.

A “variant” anti-NGF antigen binding protein refers herein to a molecule which differs in amino acid sequence from a “parent” anti-NGF antibody amino acid sequence by virtue of addition, deletion, and/or substitution of one or more amino acid residue(s) in the parent antibody sequence and retains at least one desired activity of the parent anti-NGF-antibody. The variant anti-NGF may comprise conservative amino acid substitutions in the hypervariable region of the antibody, as described herein. Desired activities can include the ability to bind the antigen specifically, the ability to reduce, inhibit or neutralize NGF activity in an animal. In one embodiment, the variant comprises one or more amino acid substitution(s) in one or more hypervariable and/or framework region(s) of the parent antibody. For example, the variant may comprise at least one, e.g. from about one to about ten, and preferably from about two to about five, substitutions in one or more hypervariable and/or framework regions of the parent antibody. Ordinarily, the variant will have an amino acid sequence having at least 50% amino acid sequence identity with the parent antibody heavy or light chain variable domain sequences, more preferably at least about between 60%, 65%, 70%, 75%, 80% 85% 90% 95%, 96%, 97%, 98% or 99% sequence identity. Identity or homology with respect to this sequence is defined herein as the percentage of amino acid residues in the candidate sequence that are identical with the parent antibody residues, after aligning the sequences and introducing gaps, if necessary, to achieve the maximum percent sequence identity. None of N-terminal, C-terminal, or internal extensions, deletions, or insertions into the antibody sequence shall be construed as affecting sequence identity or homology. The variant retains the ability to bind NGF and preferably has desired activities which are equal to or superior to those of the parent antibody. For example, the variant may have a stronger binding affinity, enhanced ability to reduce, inhibit or neutralize NGF activity in an animal, and/or enhanced ability to inhibit NGF binding to Trk A and p75.

Trk A, considered the high affinity NGF receptor is a member of the neurotrophic tyrosine kinase receptor (NTKR) family. This kinase is a membrane-bound receptor that, upon neurotrophin binding, phosphorylates itself (autophosphorylation) and members of the MAPK pathway. The presence of this kinase leads to cell differentiation and may play a role in specifying sensory neuron subtypes. The p75 receptor is considered the low affinity NGF receptor.

A ‘Variant” nucleic acid, refers herein to a molecule which differs in sequence from a “parent” nucleic acid. Polynucleotide sequence divergence may result from mutational changes such as deletions, substitutions, or additions of one or more nucleotides. Each of these changes may occur alone or in combination, one or more times in a given sequence.

The term “isolated” means that the material (for example, antigen binding protein as described herein or nucleic acid) is separated and/or recovered from a component of its natural environment. Contaminant components of its natural environment are materials that would interfere with diagnostic or therapeutic uses for the material, and may include enzymes, hormones, and other proteinaceous or non-proteinaceous solutes. With respect to nucleic acid, an isolated nucleic acid may include one that is separated from the 5′ to 3′ sequences with which it is normally associated in the chromosome. In preferred embodiments, the material will be purified to greater than 95% by weight of the material, and most preferably more than 99% by weight. Isolated material includes the material in situ within recombinant cells since at least one component of the material's natural environment will not be present. Ordinarily, however, isolated material will be prepared by at least one purification step.

As used herein, the terms “cell”, “cell line”, and “cell culture” may be used interchangeably. All of these terms also include their progeny, which are any and all subsequent generations. It is understood that all progeny may not be identical due to deliberate or inadvertent mutations. In the context of expressing a heterologous nucleic acid sequence, “host cell” refers to a prokaryotic or eukaryotic cell (for example, bacterial cells, yeast cells, mammalian cells, and insect cells) whether located in vitro or in vivo. For example, host cells may be located in a transgenic animal. Host cell can be used as a recipient for vectors and may include any transformable organism that is capable of replicating a vector and/or expressing a heterologous nucleic acid encoded by a vector.

The word “label” when used herein refers to a detectable compound or composition that is conjugated directly or indirectly to the antibody or nucleic acid. The label may itself be detectable by itself (for example, radioisotope labels or fluorescent labels) or, in the case of an enzymatic label, may catalyze chemical alteration of a substrate compound or composition that is detectable.

A “subject” or “patient” refers to a mammal in need of treatment that can be affected by molecules of the invention. Mammals that can be treated in accordance with the invention include vertebrates, with mammals such as canine, feline and human being particularly preferred examples.

A “composition” is intended to mean a combination of active agent, whether chemical composition, biological composition or biotherapeutic (particularly antigen binding proteins as described herein) and another compound or composition which can be inert (for example, a label), or active, such as an adjuvant.

As defined herein, “pharmaceutically acceptable carriers” suitable for use in the invention are well known to those of skill in the art. Such carriers include, without limitation, water, saline, buffered saline, phosphate buffer, alcohol/aqueous solutions, emulsions or suspensions. Other conventionally employed diluents, adjuvants and excipients, may be added in accordance with conventional techniques. Such carriers can include ethanol, polyols, and suitable mixtures thereof, vegetable oils, and injectable organic esters. Buffers and pH adjusting agents may also be employed. Buffers include, without limitation, salts prepared from an organic acid or base. Representative buffers include, without limitation, organic acid salts, such as salts of citric acid, ex. citrates, ascorbic acid, gluconic acid, histidine-Hel, carbonic acid, tartaric acid, succinic acid, acetic acid, or phthalic acid, Tris, trimethanmine hydrochloride, or phosphate buffers. Parenteral carriers can include sodium chloride solution, Ringer's dextrose, dextrose, trehalose, sucrose, and sodium chloride, lactated Ringer's or fixed oils. Intravenous carriers can include fluid and nutrient replenishers, electrolyte replenishers, such as those based on Ringer's dextrose and the like. Preservatives and other additives such as, for example, antimicrobials, antioxidants, chelating agents (ex. EDTA), inert gases and the like may also be provided in the pharmaceutical carriers. The present invention is not limited by the selection of the carrier. The preparation of these pharmaceutically acceptable compositions, from the above-described components, having appropriate pH isotonicity, stability and other conventional characteristics is within the skill of the art. See, for example, texts such as Remington: The Science and Practice of Pharmacy, 20th ed, Lippincott Williams & Wilkins, publ., 2000; and The Handbook of Pharmaceutical Excipients, 4.sup.th edit., eds. R. C. Rowe et al, APhA Publications, 2003.

A “therapeutically effective amount” (or “effective amount”) refers to an amount of an active ingredient, for example, an agent according to the invention, sufficient to effect beneficial or desired results when administered to a subject or patient. An effective amount can be administered in one or more administrations, applications or dosages. A therapeutically effective amount of a composition according to the invention may be readily determined by one of ordinary skill in the art. In the context of this invention, a “therapeutically effective amount” is one that produces an objectively measured change in one or more parameters associated NGF related condition sufficient to effect beneficial or desired results including clinical results such as alleviation or reduction in pain sensation. An effective amount can be administered in one or more administrations. For purposes of this invention, an effective amount of drug, compound, or pharmaceutical composition is an amount sufficient to treat, ameliorate, reduce the intensity of and/or prevent pain, including post-surgical pain, rheumatoid arthritis pain, and/or osteoarthritis pain. In some embodiments, the “effective amount” may reduce pain at rest (resting pain) or mechanically- induced pain (including pain following movement), or both, and it may be administered before, during or after a painful stimulus. As is understood in the clinical context, an effective amount of a drug, compound, or pharmaceutical composition may or may not be achieved in conjunction with another drug, compound, or pharmaceutical composition. Thus, an “effective amount” may be considered in the context of administering one or more therapeutic agents, and a single agent may be considered to be given in an effective amount if, in conjunction with one or more other agents, a desirable result may be or is achieved. Of course, the therapeutically effective amount will vary depending upon the particular subject and condition being treated, the weight and age of the subject, the severity of the condition, the particular compound chosen, the dosing regimen to be followed, timing of administration, the manner of administration and the like, all of which can readily be determined by one of ordinary skill in the art.

As used herein, the term “therapeutic” encompasses the full spectrum of treatments for a disease, condition or disorder. A “therapeutic” agent of the invention may act in a manner that is prophylactic or preventive, including those that incorporate procedures designed to target animals that can be identified as being at risk (pharmacogenetics); or in a manner that is ameliorative or curative in nature; or may act to slow the rate or extent of the progression of at least one symptom of a disease or disorder being treated.

In a further aspect, the invention features veterinary compositions in which antibodies of the present invention are provided for therapeutic or prophylactic uses. The invention features a method for treating a dog subject having a particular antigen, for example, one associated with a disease or condition. The method includes administering a therapeutically effective amount of a recombinant antibody specific for the particular antigen, with the recombinant antibody described herein.

The amount of antibody useful to produce a therapeutic effect can be determined by standard techniques well known to those of ordinary skill in the art. The antibodies will generally be provided by standard technique within a pharmaceutically acceptable buffer, and may be administered by any desired route. The route of administration of the antibody or antigen-binding moiety of the invention may be oral, parenteral, by inhalation or topical. In a preferred embodiment, the route of administration is parenteral. The term parenteral as used herein includes intravenous, intramuscular, subcutaneous, rectal, vaginal or intraperitoneal administration.

“Pain” as used herein refers to pain of any etiology, including acute and chronic pain, and any pain with an inflammatory component. Examples of pain include including inflammatory pain, post-operative incision pain, neuropathic pain, fracture pain, osteoporotic fracture pain, post-herpetic neuralgia, cancer pain, pain resulting from burns, pain associated with burn or wound, pain associated with trauma (including traumatic head injury), neuropathic pain, pain associated with musculoskeletal disorders such as rheumatoid arthritis, osteoarthritis, ankylosing spondylitis, seronegative (non-rheumatoid) arthropathies, non-articular rheumatism and periarticular disorders, and pain associated with cancer (including “break-through pain” and pain associated with terminal cancer), peripheral neuropathy and post-herpetic neuralgia.

As used herein, “treatment” is an approach for obtaining beneficial or desired clinical results. For purposes of this invention, beneficial or desired clinical results include, but are not limited to, one or more of the following: improvement or alleviation of any aspect of pain, including acute, chronic, inflammatory, neuropathic, post-surgical pain, rheumatoid arthritis pain, or osteoarthritis pain. For purposes of this invention, beneficial or desired clinical results include, but are not limited to, one or more of the following: including lessening severity, alleviation of one or more symptoms associated with pain including any aspect of pain (such as shortening duration of pain, reduction of pain sensitivity or sensation).

NGF Related Disorder, as described herein, refers to a disorder including cardiovascular diseases, atherosclerosis, obesity, type 2 diabetes, metabolic syndrome, pain and inflammation. In some embodiments of the present invention an NGF related disorder refers to pain, in particular chronic pain, inflammatory pain, post-operative incision pain, neuropathic pain, fracture pain, osteoporotic fracture pain, post-herpetic neuralgia, cancer pain, pain resulting from burns, pain associated with burn or wound, pain associated with trauma (including traumatic head injury), neuropathic pain, pain associated with musculoskeletal disorders such as rheumatoid arthritis, osteoarthritis, ankylosing spondylitis, seronegative (non-rheumatoid) arthropathies, non-articular rheumatism and periarticular disorders, and pain associated with cancer (including “break-through pain” and pain associated with terminal cancer), peripheral neuropathy and post-herpetic neuralgia.

“Reducing incidence” of pain means any of reducing severity (which can include reducing need for and/or amount of (ex. exposure to) other drugs and/or therapies generally used for this conditions, including, for example, opiates), duration, and/or frequency (including, for example, delaying or increasing time to post-surgical pain in an individual). As is understood by those skilled in the art, individuals may vary in terms of their response to treatment, and, as such, for example, a “method of reducing incidence of rheumatoid arthritis pain or osteoarthritis pain in an individual” reflects administering the anti-NGF antagonist antibody based on a reasonable expectation that such administration may likely cause such a reduction in incidence in that particular individual.

“Ameliorating” a pain or one or more symptoms of a pain (such as rheumatoid arthritis pain or osteoarthritis pain) means a lessening or improvement of one or more symptoms of a pain as compared to not administering an anti-NGF antagonist antibody. “Ameliorating” also includes shortening or reduction in duration of a symptom.

“Palliating” a pain or one or more symptoms of a pain (such as rheumatoid arthritis pain or osteoarthritis pain) means lessening the extent of one or more undesirable clinical manifestations of post-surgical pain in an individual or population of individuals treated with an anti-NGF antagonist antibody in accordance with the invention.

As used therein, “delaying” the development of pain means to defer, hinder, slow, retard, stabilize, and/or postpone progression of pain, such as post-surgical pain, rheumatoid arthritis pain, or osteoarthritis pain. This delay can be of varying lengths of time, depending on the history of the disease and/or individuals being treated. As is evident to one skilled in the art, a sufficient or significant delay can, in effect, encompass prevention, in that the individual does not develop pain. A method that “delays” development of the symptom is a method that reduces probability of developing the symptom in a given time frame and/or reduces extent of the symptoms in a given time frame, when compared to not using the method. Such comparisons are typically based on clinical studies, using a statistically significant number of subjects.

“Post-surgical pain” (interchangeably termed “post-incisional” or “post-traumatic pain”) refers to pain arising or resulting from an external trauma such as a cut, puncture, incision, tear, or wound into tissue of an individual (including that that arises from all surgical procedures, whether invasive or non-invasive). As used herein, post-surgical pain does not include pain that occurs (arises or originates) without an external physical trauma. In some embodiments, post-surgical pain is internal or external (including peripheral) pain, and the wound, cut, trauma, tear or incision may occur accidentally (as with a traumatic wound) or deliberately (as with a surgical incision). As used herein, “pain” includes nociception and the sensation of pain, and pain can be assessed objectively and subjectively, using pain scores and other methods well-known in the art. Post-surgical pain, as used herein, includes allodynia (i.e., increased response to a normally non-noxious stimulus) and hyperalgesia (i.e., increased response to a normally noxious or unpleasant stimulus), which can in turn, be thermal or mechanical (tactile) in nature. In some embodiments, the pain is characterized by thermal sensitivity, mechanical sensitivity and/or resting pain. In some embodiments, the post-surgical pain comprises mechanically-induced pain or resting pain. In other embodiments, the post-surgical pain comprises resting pain. The pain can be primary or secondary pain, as is well-known in the art.

Before the present methods are described, it is to be understood that this invention is not limited to particular methods, and experimental conditions described, as such methods and conditions may vary. It is also to be understood that the terminology used herein is for the purpose of describing particular embodiments only, and is not intended to be limiting, since the scope of the present invention will be limited only by the appended claims.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are now described. All publications mentioned herein are incorporated herein by reference in their entirety.

The invention disclosed herein concerns antigen binding proteins (used interchangeably with the terms “antibodies”, “antagonist antibodies” “antibody fragments” and the like, as described herein), that specifically bind to Nerve Growth Factor (NGF) and in particular antibodies, whether it be caninized, felinized, humanized etc. antibodies produced by hybridoma or phage display technology or fully “caninized” (speciated) monoclonal antibodies that specifically bind to NGF and thus prevent NGF from binding to canine TrkA and to a lesser extent canine p75 receptors, thus serving as an antagonist in that the signaling pathway is prevented from being activated by NGF.

NGF was the first neurotrophin to be identified, and its role in the development and survival of both peripheral and central neurons has been well characterized. NGF has been shown to be a critical survival and maintenance factor in the development of peripheral sympathetic and embryonic sensory neurons and of basal forebrain cholinergic neurons (Smeyne et al. (1994) Nature 368:246-249; Crowley et al. (1994)

Cell 76:1001-1011). NGF upregulates expression of neuropeptides in sensory neurons (Lindsay et al. (1989) Nature 337:362-364) and its activity is mediated through two different membrane-bound receptors, the TrkA receptor and what is considered the low affinity p75 common neurotrophin receptor. NGF has been shown to be elevated in NGF related disorders in which an elevated amount of NGF is present in injured or diseased tissues. An NGF related disorder, can be defined as an increase in pain due to the elevation of NGF in an injured, diseased or damaged tissue. Pain, as used herein, is defined as described herein, refers to a disorder including chronic pain, inflammatory pain, post-operative incision pain, neuropathic pain, fracture pain, osteoporotic fracture pain, post-herpetic neuralgia, cancer pain, pain resulting from burns, pain associated with burn or wound, pain associated with trauma (including traumatic head injury), neuropathic pain, pain associated with musculoskeletal disorders such as chronic pain, rheumatoid arthritis, osteoarthritis, ankylosing spondylitis, seronegative (non-rheumatoid) arthropathies, non-articular rheumatism and periarticular disorders, and pain associated with cancer (including “break-through pain” and pain associated with terminal cancer), peripheral neuropathy and post-herpetic neuralgia.

In an embodiment of the present invention, an NGF disorder is defined as osteoarthritis in a subject (humans, canines and felines.). Osteoarthritis (OA) is a slowly-progressive degenerative joint disease characterized by a loss of joint cartilage and the subsequent exposure of subchondral bone in canines. This eventually results in a self-perpetuating insidious disorder characterized by joint pain. New bone formation occurs in response to the chronic inflammation, and local tissue damage in an attempt to limit both movement and pain. Macroscopically, there is loss of joint cartilage, a narrowing of the joint space, sclerosis of subchondral bone, and the production of joint osteophytes (Veterinary Focus: Vol 17 No 3; 2007)

In different species, such as canines and felines, the onset of primary OA depends on breed. For canines, the onset mean age is 3.5 years in Rottweilers and 9.5 years in Poodles for examples, with a wide range of onset for other breeds as well as mixed breeds. The developmental orthopedic diseases and associated osteoarthritis are the most common articular diseases in dogs, they account for some 70% of medical visits for articular disease and related problems within the appendicular skeleton. Twenty two percent of cases were dogs aged one year or under. The incidence of OA is increased by trauma as well as obesity, aging and genetic abnormalities. In particular, age can be a factor in OA incidence wherein >50% of arthritis cases are observed in dogs aged between 8-13 years. The musculoskeletal diseases are very common in geriatric patients, and nearly 20% of elderly dogs show orthopedic disorders. In Labrador Retrievers aged >8 years, OA in several joints (elbow, shoulder, hip, knee) is typical. Additionally, the size of the canine plays a role in OA onset as well. 45% of dogs with arthritis are large breed dogs. Among these, >50% are giant breed dogs, while only 28% are medium breed dogs and 27% are small breed dogs. The need for pharmaceutical intervention for the alleviation of OA pain in canines is very high.

As stated herein, elevated levels of NGF are indicative of a NGF related disorder, particularly in OA. Elevated levels of NGF have been reported in transgenic arthritic mice along with an increase in the number of mast cells (Aloe, et al., Int. J. Tissue Reactions-Exp. Clin. Aspects 15:139-143 (1993)). PCT Publication No. WO 02/096458 discloses use of anti NGF antibodies of certain properties in treating various NGF related disorders such as inflammatory condition (for example, rheumatoid arthritis). It has been reported that a purified anti-NGF antibody injected into arthritic transgenic mice carrying the human tumor necrosis factor gene caused reduction in the number of mast cells, as well as a decrease in histamine and substance P levels within the synovium of arthritis mice (Aloe et al., Rheumatol. Int. 14: 249-252 (1995)). It has been shown that exogenous administration of an NGF antibody reduced the enhanced level of TNFα, occurring in arthritic mice (Marmi et al., Rheumatol. Int. 18: 97-102 (1998)). Rodent anti-NGF antagonist antibodies have been reported. See, ex. Hongo et al., Hybridoma (2000) 19(3): 215-227; Ruberti et al. (1993) Cell. Molec. Neurobiol. 13(5): 559-568. However, when rodent antibodies are used therapeutically in non-murine mammals, an anti-murine antibody response develops in significant numbers of treated individuals. Thus, there is a serious need for anti-NGF antagonist antigen binding proteins, including anti-NGF antagonist antibodies of the present invention for canine use particularly for use in treating OA.

While the properties of antibodies make them very attractive therapeutic agents, there are a number of limitations. The vast majority of monoclonal antibodies (mAbs) are of rodent origin, as previously noted. When such antibodies are administered in a different species, patients can mount their own antibody response to such xenogenic antibodies. Such response may result in the eventual neutralization and elimination of the antibody. As described above mice are used extensively in the production of monoclonal antibodies. One problem in the use of using antibodies produced by a particular species, generally initially in the mouse, is that a non-murine subjects being treated with said antibodies react to the mouse antibodies as if they were a foreign substance thus creating a new set of antibodies to the mouse antibodies. Mouse antibodies are “seen” by the non-murine, for example canine, immune system as foreign, and the subject then mounts an immune response against the molecule. Those skilled in the field will recognize the need to be able to treat a subject with an antigen specific antibody, but have that antibody species specific. Part of the reaction generated from cross species antibody administration, for example a mouse monoclonal antibody being administered to a canine, can range from a mild form, like a rash, to a more extreme and life-threatening response, such as renal failure. This immune response can also decrease the effectiveness of the treatment, or create a future reaction if the subject is given a subsequent treatment containing mouse antibodies. Accordingly, we set forth to overcome this disadvantage by “caninization” of an antibody. In particular, this process focuses on the framework regions of the immunoglobulin variable domain, but could also include the compliment determinant regions (CDR's) of the variable domain. The enabling steps and reduction to practice for this process are described in this disclosure.

The process of modifying a monoclonal antibody (antigen binding protein, antagonist antibody etc. as described herein and terms used interchangeably) from an animal to render it less immunogenic for therapeutic administration to species has been aggressively pursued and has been described in a number of publications (e.g. Antibody Engineering: A practical Guide. Carl A. K. Borrebaeck ed. W. H. Freeman and Company, 1992). However, this process has not been applied for the development of therapeutic or diagnostics for non-humans, particularly canines, until recently. In fact, very little has been published about canine variable domains at all. Wasserman and Capra, Biochem. 6, 3160 (1977), determined the amino acid sequence of the variable regions of both a canine IgM and a canine IgA heavy chain. Wasserman and Capra, Immunochem. 15, 303 (1978), determined the amino acid sequence of the K light chain from a canine IgA. McCumber and Capra, Mol. Immunol. 16, 565 (1979), disclose the complete amino-acid sequence of a canine mu chain. Tang et al., Vet. Immunology Immunopathology 80, 259 (2001), discloses a single canine IgG-A y chain cDNA and four canine IgG-A y chain protein sequences. It describes PCR amplification of a canine spleen cDNA library with a degenerate oligonucleotide primer designed from the conserved regions of human, mouse, pig, and bovine IgGs. The paucity of information available on canine antibodies has prevented their development as therapeutics for the treatment canine disease.

These noted limitations have prompted the development of engineering technologies known as “speciation” and is well known to those in the art in terms of “humanization” of therapeutic antibodies. Humanized antibodies can be generated as chimeric antibodies or fragments thereof which contain minimal sequence derived from non-human immunoglobulin. For the most part, humanized antibodies are human antibodies (i.e. “recipient antibody” or “target species antibody”) in which residues from a complementarity determining region (CDR) of the recipient are replaced by residues from a CDR of a non-human species (i.e. “donor antibody” or “originating species antibody”) such as mouse, having the desired. properties such as specificity, affinity, and potency. In some instances, framework region (FR) residues of the human immunoglobulin are replaced by corresponding non-human residues. This humanization strategy is referred to as “CDR grafting” as reported for the making of humanized antibodies (Winter, U.S. Pat. No. 5,225,539). Back mutation of selected target framework residues to the corresponding donor residues might be required to restore and or improved affinity. Structure-based methods may also be employed for humanization and affinity maturation, for example as described for humanization in U.S. patent application Ser. No. 10/153,159 and related applications. Comparison of the essential framework residues required in humanization of several antibodies, as well as computer modeling based on antibody crystal structures revealed a set of framework residues termed as “Vernier zone residues” (Foote, J. Mol. Biol. 224:487-499 (1992)). In addition, several residues in the VH-VL interface zone have been suggested to be important in maintaining affinity for the antigen (Santos, Prog Nucleic Acid Res Mol Biol. 60: 169-94 (1998); Kettleborough, et al., Protein Engin., 4:773-783 (1991)). Similar strategies for “caninization” of antibodies for use in dogs are described in U.S. Pat. No. 7,261,890.

Alternatively, humanized antibodies may contain the CDRs from a non-human sequence grafted into pools (e.g. libraries) of individual human framework regions. This newly engineered antibody is able to bind to the same antigen as the original antibody. The antibody constant region is derived from a human antibody. The methodology for performing this aspect is generally described as framework shuffling (Dall'Acqua, Methods, 36:43-60 (2005)). Furthermore, the humanized antibody may contain sequences from two or more framework regions derived from at least two human antibody germline sequences with high homology to the donor species. Antibodies designed using this method are described as hybrid antibodies (Rother et al., U.S. Pat. No. 7,393,648) and may be applicable to speciation outside of humanization, for example for caninization.

The approaches described above utilize essentially entire framework regions from one or more antibody variable heavy chains or variable light chains of the target species which are engineered to receive CDRs from the donor species. This approach is also utilized when felinizing an antibody to make it less antigenic when administered to felines, in the same fashion as caninization. In some cases, back mutation of selected residues in the variable region is used to enhance presentation of the CDRs. Designing antibodies that minimize immunogenic reaction in a subject to non-native sequences in the antibody, while at the same time preserving antigen binding regions of the antibody sufficiently to maintain efficacy, has proven challenging.

Another challenge for developing therapeutic antibodies targeting proteins is that epitopes on the homologous protein in a different species are frequently different, and the potential for cross-reactivity with other proteins is also different. As a consequence, antibodies have to be made, tested and developed for the specific target in the particular species to be treated.

Antibodies target an antigen through its binding of a specific epitope on an antigen by the interaction with the variable region of the antibody molecule. Furthermore, antibodies have the ability to mediate, inhibit (as in the case of the antagonistic anti-NGF antigen binding protein of the present invention) and/or initiate a variety of biological activities. There are a wide range of functions for therapeutic antibodies, for example, antibodies can modulate receptor-ligand interactions as agonists or antagonists. Antibody binding can initiate intracellular signaling to stimulate cell growth, cytokine production, or apoptosis. Antibodies can deliver agents bound to the Fe region to specific sites. Antibodies also elicit antibody-mediated cytotoxicity (ADCC), complement-mediated cytotoxicity (CDC), and phagocytosis. There are also antibodies that have been altered where the ADCC, CDC, Cl q binding and phagocytosis functions have been eliminated. In one embodiment of the present invention the antibody of the present invention comprises alterations in the Fc region of the antibody that alters effector function of said antibody.

### Caninization and Felinization

As used herein, “caninized antibody” means an antibody having an amino acid sequence corresponding to that of an antibody produced by a canine and/or has been made using any of the techniques known in the art or disclosed herein. The same process is undertaken for the felinization process and should be applied to the description herewith. For the sake of simplicity caninization will primarily be used as the example, however these examples are not limited only to canine. The same concepts and designs apply to the speciation of other antigen binding proteins, for example feline, and human and the like). This definition of a caninized antibody includes antibodies comprising at least one canine heavy chain polypeptide or at least one canine light chain polypeptide. “Speciation”, per se, of antibodies, and in particular the humanization of antibodies is a field of study well known to one skilled in the art. It has been unknown until recently whether the speciation of antibodies beyond humanization would yield a therapeutic antibody that could be efficacious in any other species. The present invention exemplifies the caninization and felinization of an anti-NGF antigen binding protein for therapeutic use in dogs and cats respectively.

Chimeric antibodies comprise sequences from at least two different species. As one example, recombinant cloning techniques may be used to include variable regions, which contain the antigen-binding sites, from a non-recipient antibody (i.e., an antibody prepared in a donor species immunized with the antigen) and constant regions derived from a recipient immunoglobulin.

Speciated (caninized, felinized and the like) antibodies are a type of chimeric antibody wherein variable region residues responsible for antigen binding (i.e., residues of a complementarity determining region, abbreviated complementarity determining region, or any other residues that participate in antigen binding) are derived from a non-canine (or non-feline) species, while the remaining variable region residues (i.e., residues of the framework regions) and constant regions are derived, at least in part, from canine (or feline) antibody sequences. A subset of framework region residues and constant region residues of a speciated antibody may be derived from non-canine (or feline) sources. Variable regions of a speciated antibody are also described as speciated (i.e., a speciated light or heavy chain variable region). The non-speciated species is typically that used for immunization with antigen, such as mouse, rat, rabbit, non-human primate, or other non-canine or non-feline mammalian species.

Complementarity determining regions (CDRs) are residues of antibody variable regions that participate in antigen binding. Several numbering systems for identifying CDRs are in common use. The Kabat definition is based on sequence variability, and the Clothia definition is based on the location of the structural loop regions. The AbM definition is a compromise between the Kabat and Clothia approaches. A speciated antibody of the invention may be constructed to comprise one or more CDRs. Still further, CDRs may be used separately or in combination in synthetic molecules such as SMIPs and small antibody mimetics.

Framework residues are those residues of antibody variable regions other than hypervariable or CDR residues. Framework residues may be derived from a naturally occurring canine (for example, but applicable in concept with other species such as feline and human. For the sake of simplicity canine will be used as the representative species but the examples are not limited to canine as such) antibody, such as a canine framework that is substantially similar to a framework region of the antibody of the invention. Artificial framework sequences that represent a consensus among individual sequences may also be used. When selecting a framework region for caninization, sequences that are widely represented in canines may be preferred over less populous sequences. Additional mutations of the canine framework acceptor sequences may be made to restore murine residues believed to be involved in antigen contacts and/or residues involved in the structural integrity of the antigen-binding site, or to improve antibody expression.

Grafting of CDRs is performed by replacing one or more CDRs of an acceptor antibody (ex., a caninized antibody or other antibody comprising desired framework residues) with CDRs of a donor antibody (ex. a non-canine antibody). Acceptor antibodies may be selected based on similarity of framework residues between a candidate acceptor antibody and a donor antibody. For example, canine framework regions are identified as having substantial sequence homology to each framework region of the relevant non-canine antibody, and CDRs of the non-canine antibody are grafted onto the composite of the different canine framework regions.

Analysis of the three-dimensional structures of antibody-antigen complexes, combined with analysis of the available amino acid sequence data may be used to model sequence variability based on structural dissimilarity of amino acid residues that occur at each position within the CDR. CDRs of the present invention can also be utilized in small antibody mimetics, which comprise two CDR regions and a framework region (Qui et al. Nature Biotechnology Vol 25; 921-929; August 2007).

Acceptor frameworks for grafting of CDRs or abbreviated CDRs may be further modified to introduce desired residues. For example, acceptor frameworks may comprise a heavy chain variable region of a canine consensus sequence, optionally with non-canine donor residues at one or more of positions. Following grafting, additional changes may be made in the donor and/or acceptor sequences to optimize antibody binding and functionality. See ex. International Publication No. WO 91/09967.

The present invention further provides cells and cell lines expressing antibodies of the invention. Representative host cells include bacterial, yeast, mammalian and human cells, such as CHO cells, HEK-293 cells, HeLa cells, CV-1 cells, and COS cells. Methods for generating a stable cell line following transformation of a heterologous construct into a host cell are known in the art. Representative non-mammalian host cells include insect cells (Potter et al. (1993) Int. Rev. Immunol. 10(2-3):103-112). Antibodies may also be produced in transgenic animals (Houdebine (2002) Curr. Opin. Biotechnol. 13(6):625-629) and transgenic plants (Schillberg et al. (2003) Cell Mol. Life Sci. 60(3):433-45).

As discussed above, monoclonal, chimeric and speciated antibodies, which have been modified by, ex. deleting, adding, or substituting other portions of the antibody, ex. the constant region, are also within the scope of the invention. For example, an antibody can be modified as follows: (i) by deleting the constant region; (ii) by replacing the constant region with another constant region, ex., a constant region meant to increase half-life, stability or affinity of the antibody, or a constant region from another species or antibody class; or (iii) by modifying one or more amino acids in the constant region to alter, for example, the number of glycosylation sites, effector cell function, Fc receptor (FcR) binding, complement fixation, among others. In one embodiment of the present invention the antibody of the invention comprises an altered Fc region that alters effector function of the antibody.

Methods for altering an antibody constant region are known in the art. Antibodies with altered function, e.g. altered affinity for an effector ligand, such as FcR on a cell, or the C1 component of complement can be produced by replacing at least one amino acid residue in the constant portion of the antibody with a different residue (see ex., EP 388,151 A1, U.S. Pat. No. 5,624,821 and U.S. Pat. No. 5,648,260, the contents of all of which are hereby incorporated by reference).

For example, it is possible to alter the affinity of an Fc region of an antibody for an FcR (ex. Fc gamma R1), or for C1q binding by replacing the specified residue(s) with a residue(s) having an appropriate functionality on its side chain, or by introducing a charged functional group, such as glutamate or aspartate, or perhaps an aromatic non-polar residue such as phenylalanine, tyrosine, tryptophan or alanine (see ex., U.S. Pat. No. 5,624,821). The antibody or binding fragment thereof may be conjugated with a cytotoxin, a therapeutic agent, or a radioactive metal ion. In one embodiment, the protein that is conjugated is an antibody or fragment thereof. A cytotoxin or cytotoxic agent includes any agent that is detrimental to cells. Non-limiting examples include, calicheamicin, taxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, puromycin, and analogs, or homologs thereof. Therapeutic agents include, but are not limited to, antimetabolites (ex., methotrexate, 6-mercaptopurine, 6-thioguanine, cytarabine, and 5-fluorouracil decarbazine), alkylating agents (ex., mechlorethamine, thioepa chlorambucil, melphalan, carmustine (BSNU) and lomustine (CCNU), cyclothosphamide, busulfan, dibromomannitol, streptozotocin, mitomycin C, and cis-dichlorodiamine platinum (II) (DDP), cisplatin), anthracyclines (ex., daunorubicin and doxorubicin), antibiotics (ex., dactinomycin, bleomycin, mithramycin, and anthramycin), and anti-mitotic agents (ex., vincristine and vinblastine). Techniques for conjugating such moieties to proteins are well known in the art.

### Compositions, Derived Compositions, and Methods of Making the Compositions

This invention encompasses compositions, including pharmaceutical compositions, comprising antigen binding proteins (“antibodies”, “antibody fragments”, “antagonist antibodies” and the like as used interchangeably herein), polypeptides and polynucleotides comprising sequences encoding antigen binding proteins or polypeptides of the invention.

As used herein, compositions comprise one or more antibodies, antigen binding proteins or polypeptides (which may or may not be an antibody) that bind to NGF, and/or one or more polynucleotides comprising sequences encoding one or more antibodies or polypeptides that bind to NGF. These compositions may further comprise suitable excipients, such as pharmaceutically/veterinary acceptable excipients including buffers, which are well known in the art. The invention also encompasses isolated antibody, polypeptide and polynucleotide embodiments. The invention also encompasses substantially pure antibody, polypeptide and polynucleotide embodiments.

In one or more embodiment, the present invention provides for novel antigen binding proteins that specifically bind to NGF. In one or more embodiments, the antigen binding protein is defined as an antibody or antibody fragment. In one or more embodiments, the antigen binding protein is caninized, felinized, equinized or humanized. In one or more embodiments, the antigen binding protein of the present invention binds to canine, feline or human NGF. In one embodiment, the antigen binding protein is a monoclonal antibody. In one embodiment, a monoclonal antibody of the invention binds to NGF and prevents its binding to, and activation of, its receptors Trk A and to a lesser extent p75, thus preventing the signaling cascade as described herein.

In another aspect the present invention provides an antigen binding protein that specifically binds Nerve Growth Factor (NGF) and inhibits the binding between NGF and TrkA thus blocking the biological activity of NGF, as defined herein, which comprises a heavy chain variable region (VH) comprising a Complimentary Determining Region 1 (CDR 1) comprising an amino acid sequence having at least about 90% sequence identity to SEQ ID NO.4 (amino acid sequence: GFTLTQYG), a Complimentary Determining Region 2 (CDR 2) comprising an amino acid sequence having at least about 90% sequence identity to SEQ ID NO.5 (amino acid sequence: VIWATGATD) and a Complimentary Determining Region 3 (CDR 3) comprising an amino acid sequence having at least about 90% sequence identity to SEQ ID NO. 6 (amino acid sequence: DGWWYATSWYFDV); and the antigen binding protein comprises a light chain variable region (VL) which comprises an antigen binding protein that specifically binds Nerve Growth Factor (NGF) comprising a light chain variable region (VL) comprising:

a Complimentary Determining Region 1 (CDR1) comprising an amino acid sequence having at least about 90% sequence identity to the amino acid sequence comprising:
(X1)-Alanine[A]-Serine[S]-Glutamine[Q]-(X2)-Isoleucine [I]-(X3)-(X4)-(X5)-Leucine[L]-Asparagine[N]
   wherein:
   X1 comprises Lysine (K) or Arginine (R),
   X2 comprises Serine (S) or Aspartic Acid (D),
   X3 comprises Asparagine (N) or Serine (S),
   X4 comprises Histidine (H) or Asparagine (N),
   X5 comprises Tyrosine [Y] or Asparagine [N]; and
   a Complimentary Determining Region 2 (CDR2) comprising an amino acid sequence having at least about 90% sequence identity to the amino acid sequence comprising:
   Threonine [T]-(X6)-(X7)-Leucine [L]-(X8)-(X9) wherein:
   X6 comprises Threonine [T], Histidine [H], Serine [S] or Alanine [A],
   X7 comprises Arginine [R] or Serine[S],
   X8 comprises Glutamine [Q] or Histidine [H],
   X9 comprises Alanine[A], Glutamine[Q], Glycine [G] or Valine [V]; and
   a Complimentary Determining Region 3 (CDR3) comprising an amino acid sequence having at least about 90% sequence identity to the amino acid sequence comprising:
   (X10)-(X11)-(X12)-(X13)-(X14)-(X15)-P-(X16)-(X17) wherein
   X10 comprises Q or H,
   X11 comprises Q or R,
   X12 comprises G or A,
   X13 comprises D, S, T or N,
   X14 comprises H, T or M,
   X15 comprises F, L or S,
   X16 comprises R, Y or G,
   X17 comprises T or P; and
   any variants thereof having one or more conservative amino acid substitutions in at least one of CDR1, CDR2 or CDR3 within any of the variable light or variable heavy chain regions of said antigen binding protein.

In one or more embodiments, the present invention provides an isolated and recombinant antigen binding protein, “H3AQC2301B12L1AL1L3” (or “ZTS-182”), wherein the variable heavy chain comprises amino acid sequence having at least about 90% sequence identity to the amino acid sequence comprising SEQ ID NO. 49 and wherein the variable light chain comprises amino acid sequences having at least about 90% sequence identity to the amino acid sequence comprising SEQ ID NO. 51. Additionally, the variable heavy chain comprises Complementarity Determining Regions 1-3 comprising the amino acid sequences having at least about 90% sequence identity to SEQ ID NO. 4 (“01B12H3AHC” VH CDR1), amino acid sequence having at least about 90% sequence identity to the amino acid sequence comprising SEQ ID NO. 5 (“01B12H3AHC” VH CDR2), amino acid sequence having at least about 90% sequence identity to the amino acid sequence comprising SEQ ID NO. 6 (“01B12H3AHC” VH CDR3); and wherein the variable light chain Complementarity Determining Regions 1-3 comprising the amino acid sequences having at least about 90% sequence identity to SEQ ID NO. 7 (“QC2301B12L1AL1L3” VL CDR1), amino acid sequence having at least about 90% sequence identity to the amino acid sequence comprising SEQ ID NO. 8 (“QC2301B12L1AL1L3” VL CDR2), and amino acid sequence having at least about 90% sequence identity to the amino acid sequence comprising SEQ ID NO. 9 (“QC2301B12L1AL1L3” VL CDR3); and any variants thereof having one or more conservative amino acid substitutions in at least one of CDR1, CDR2 or CDR3 within any of the variable light or variable heavy chains of said antigen binding protein or within the amino acid sequence of the entire VH or VL sequences of the antigen binding protein of the invention.

In one or more embodiments, the present invention provides an isolated and recombinant antigen binding protein, “H3AQC2301B12L1AL1L3” or “ZTS-182”, wherein the variable heavy chain is encoded by a nucleic acid sequence having at least about 90% sequence identity to the nucleotide sequence comprising SEQ ID NO. 50 and the variable light chain is encoded by the nucleic acid sequence having at least about 90% sequence identity to the nucleotide sequence comprising SEQ ID NO. 52; and any variants thereof having nucleic acid sequences encoding proteins comprising one or more conservative amino acid substitutions within any of the variable light or variable heavy chains of said antigen binding protein or wherein the degeneracy of the genetic code is taken into account.

In one or more embodiments, the present invention provides an isolated and recombinant antigen binding protein comprising the variable heavy chain that is encoded by a nucleic acid sequence having at least about 90% sequence identity to the nucleotide sequence comprising SEQ ID NO. 50 and the variable light chain is encoded by the nucleic acid sequence having at least about 90% sequence identity to the nucleotide sequence comprising SEQ ID NO. 52; and any variants thereof having nucleic acid sequences encoding proteins comprising one or more conservative amino acid substitutions within any of the variable light or variable heavy chains of said antigen binding protein or wherein the degeneracy of the genetic code is taken into account; further comprising a canine light chain constant region that is encoded by a nucleic acid having at least about 90% sequence identity to the nucleotide sequence comprising SEQ ID NO.161 and further comprising a canine heavy chain constant region encoded by a nucleic acid having at least about 90% sequence identity to the nucleotide sequence comprising SEQ ID NO.159. In one or more embodiments the present invention provides a nucleic acid sequence encoding an antigen binding protein comprising a canine heavy chain constant region comprising effector mutations comprising SEQ ID NO.185. In one or more embodiments, the present invention provides an isolated and recombinant antigen binding protein comprising the variable heavy chain that is encoded by a nucleic acid sequence comprising SEQ ID NO. 50 and a variable light chain is encoded by the nucleic acid sequence SEQ ID NO. 52; and any variants thereof having nucleic acid sequences encoding proteins comprising one or more conservative amino acid substitutions within any of the variable light or variable heavy chains of said antigen binding protein or wherein the degeneracy of the genetic code is taken into account; further comprising a canine light chain constant region that is encoded by a nucleic acid comprising SEQ ID NO.161 and further comprising a canine heavy chain constant region encoded by a nucleic acid comprising SEQ ID NO.159. In one or more embodiments the present invention provides a nucleic acid sequence encoding an antigen binding protein comprising a canine heavy chain constant region comprising effector mutations comprising SEQ ID NO.185.

In another aspect the present invention provides an antigen binding protein that specifically binds Nerve Growth Factor (NGF) and inhibits the binding between NGF and TrkA thus blocking the biological activity of NGF, as defined herein, which comprises a heavy chain variable region (VH) comprising a Complimentary Determining Region 1 (CDR 1) comprising an amino acid sequence having at least about 90% sequence identity to SEQ ID NO.4 (amino acid sequence: GFTLTQYG), a Complimentary Determining Region 2 (CDR 2) comprising an amino acid sequence having at least about 90% sequence identity to SEQ ID NO.5 (amino acid sequence: VIWATGATD) and a Complimentary Determining Region 3 (CDR 3) comprising an amino acid sequence having at least about 90% sequence identity to SEQ ID NO. 6 (amino acid sequence: DGWWYATSWYFDV); and the antigen binding protein comprises a light chain variable region (VL) which comprises an antigen binding protein that specifically binds Nerve Growth Factor (NGF) comprising a light chain variable region (VL) comprising:
a Complimentary Determining Region 1 (CDR1) comprising an amino acid sequence having at least about 90% sequence identity to the amino acid sequence comprising:
   (X1)-A-S-Q-(X2)-I-(X3)-(X4)-(X5)-L-N wherein:
   X1 comprises K or R,
   X2 comprises S or D,
   X3 comprises N or S,
   X4 comprises H or N,
   X5 comprises Y or N; and
a Complimentary Determining Region 2 (CDR2) comprising an amino acid sequence having at least about 90% sequence identity to the amino acid sequence comprising:
   T-(X6)-(X7)-L-(X8)-(X9) wherein:
   X6 comprises T, H, S or A,
   X7 comprises R or S,
   X8 comprises Q or H,
   X9 comprises A, Q, G or V; and
a Complimentary Determining Region 3 (CDR3) comprising an amino acid sequence having at least about 90% sequence identity to the amino acid sequence comprising:
   (X10)-(X11)-(X12)-(X13)-(X14)-(X15)-P-(X16)-(X17) wherein
   X10 comprises Q or H,
   X11 comprises Q or R,
   X12 comprises G or A,
   X13 comprises D, S, T or N,
   X14 comprises H, T or M,
   X15 comprises F, L or S,
   X16 comprises R, Y or G,
   X17 comprises T or P; and
any variants thereof having one or more conservative amino acid substitutions in at least one of CDR1, CDR2 or CDR3 within any of the variable light or variable heavy chain regions of said antigen binding protein.

In one or more embodiments, the present invention provides an isolated and recombinant caninized antigen binding protein, “ZTS-182 m6”, wherein the variable heavy chain comprises amino acid sequence having at least about 90% sequence identity to the amino acid sequence comprising SEQ ID NO. 49 and wherein the variable light chain comprises amino acid sequences having at least about 90% sequence identity to the amino acid sequence comprising SEQ ID NO. 175. Additionally, the variable heavy chain comprises Complementarity Determining Regions 1-3 comprising the amino acid sequences having at least about 90% sequence identity to SEQ ID NO. 4 (“01B12H3AHC” VH CDR1), amino acid sequence having at least about 90% sequence identity to the amino acid sequence comprising SEQ ID NO. 5 (“01B12H3AHC” VH CDR2), amino acid sequence having at least about 90% sequence identity to the amino acid sequence comprising SEQ ID NO. 6 (“01 B12H3AHC” VH CDR3); and wherein the variable light chain Complementarity Determining Regions 1-3 comprising the amino acid sequences having at least about 90% sequence identity to SEQ ID NO. 167, amino acid sequence having at least about 90% sequence identity to the amino acid sequence comprising SEQ ID NO. 168 , and amino acid sequence having at least about 90% sequence identity to the amino acid sequence comprising SEQ ID NO. 169; and any variants thereof having one or more conservative amino acid substitutions in at least one of CDR1, CDR2 or CDR3 within any of the variable light or variable heavy chains of said antigen binding protein or within the amino acid sequence of the entire VH or VL sequences of the antigen binding protein of the invention. In one or more embodiment the antigen binding protein of the invention further comprises a canine light chain constant region comprising an amino acid sequence having at least about 90% sequence identity to the amino acid sequence comprising SEQ ID NO.160 and a canine heavy chain constant region comprising an amino acid sequence having at least about 90% sequence identity to the amino acid sequence comprising SEQ ID NO.158. In one embodiment the antibody of the invention comprises a canine heavy chain constant region comprising effector function mutations comprising an amino acid sequence having at least about 90% sequence identity to the amino acid sequence comprising SEQ ID NO.184.

In one or more embodiments, the present invention provides an isolated and recombinant caninized antigen binding protein, “ZTS-182m6”, wherein the variable heavy chain is encoded by a nucleic acid sequence having at least about 90% sequence identity to the nucleotide sequence comprising SEQ ID NO. 50 and the variable light chain is encoded by the nucleic acid sequence having at least about 90% sequence identity to the nucleotide sequence comprising SEQ ID NO. 176; and any variants thereof having nucleic acid sequences encoding proteins comprising one or more conservative amino acid substitutions within any of the variable light or variable heavy chains of said antigen binding protein or wherein the degeneracy of the genetic code is taken into account.

In one or more embodiments, the present invention provides an isolated and recombinant felinized antigen binding protein, ZTS-082-1B, wherein the variable heavy chain comprises amino acid sequence having at least about 90% sequence identity to the amino acid sequence comprising SEQ ID NO. 85 (“H1-23” VH),and wherein the variable light chain comprises amino acid sequence having at least about 90% sequence identity to the amino acid sequence comprising SEQ ID NO. 87 (“KPL”) and any variants thereof having one or more conservative amino acid substitutions in at least one of the variable light or variable heavy chains of said antigen binding protein.

In one or more embodiments, the present invention provides an isolated and recombinant felinized antigen binding protein, ZTS-082-1C, wherein the variable heavy chain comprises amino acid sequence having at least about 90% sequence identity to the amino acid sequence comprising SEQ ID NO. 85 (“H1-23” VH) and a variable light chain comprising amino acid sequence having at least about 90% sequence identity to the amino acid sequence comprising SEQ ID NO. 89 (“L3-K36” VL); and any variants thereof having one or more conservative amino acid substitutions in within any of the variable light or variable heavy chains of said antigen binding protein.

In one or more embodiments, the present invention provides an isolated and recombinant caninized antigen binding protein, ZTS-082-361 wherein the variable heavy chain comprises an amino acid sequence having at least about 90% sequence identity to the amino acid sequence comprising SEQ ID NO. 92 (“H733”) and the variable light chain comprising the amino acid sequence having at least about 90% sequence identity to the amino acid sequence comprising SEQ ID NO. 89 (“L3-K36”); and any variants thereof having amino acid sequences comprising one or more conservative amino acid substitutions within any of the variable light or variable heavy chains of said antigen binding protein.

In one or more embodiments, the present invention provides an isolated and recombinant felinized antigen binding protein, ZTS-082-2D, wherein the variable heavy chain comprises amino acid sequence having at least about 90% sequence identity to the amino acid sequence comprising SEQ ID NO. 85 (“H1-23” VH) and a variable light chain comprising amino acid sequence having at least about 90% sequence identity to the amino acid sequence comprising SEQ ID NO. 94 (“K643” VL); and any variants thereof having one or more conservative amino acid substitutions in within any of the variable light or variable heavy chains of said antigen binding protein.

In one or more embodiments, the present invention provides an isolated and recombinant felinized antigen binding protein, ZTS-082-2E, wherein the variable heavy chain comprises amino acid sequence having at least about 90% sequence identity to the amino acid sequence comprising SEQ ID NO. 92 (“H733” VH) and a variable light chain comprising amino acid sequence having at least about 90% sequence identity to the amino acid sequence comprising SEQ ID NO. 87 (“KPL” VL); and any variants thereof having one or more conservative amino acid substitutions in within any of the variable light or variable heavy chains of said antigen binding protein.

The present invention provides for recombinant antigen binding proteins, in some cases monoclonal antibodies, and antibody fragments as described herein and their uses in clinical administrations and scientific procedures, including diagnostic procedures. With the advent of methods of molecular biology and recombinant technology, it is possible to produce antibody and antibody-like molecules by recombinant means and thereby generate gene sequences that code for specific amino acid sequences found in the polypeptide structure of the antibodies. Such antibodies can be produced by either cloning the gene sequences encoding the polypeptide chains of said antibodies or by direct synthesis of said polypeptide chains, with assembly of the synthesized chains to form active tetrameric (H2L2) structures with affinity for specific epitopes and antigenic determinants. This has permitted the ready production of antibodies having sequences characteristic of neutralizing antibodies from different species and sources.

Regardless of the source of the antibodies, how they are recombinantly constructed, or how they are synthesized, in vitro or in vivo, using transgenic animals, large cell cultures of laboratory or commercial size, using transgenic plants, or by direct chemical synthesis employing no living organisms at any stage of the process, all antibodies have a similar overall 3-dimensional structure. This structure is often given as H2L2 and refers to the fact that antibodies commonly comprise two light (L) amino acid chains and 2 heavy (H) amino acid chains. Both chains have regions capable of interacting with a structurally complementary antigenic target. The regions interacting with the target are referred to as “variable” or ‘V” regions and are characterized by differences in amino acid sequence from antibodies of different antigenic specificity. The variable regions of either H or L chains contain the amino acid sequences capable of specifically binding to antigenic targets.

As used herein, the term “antigen binding region” refers to that portion of an antibody molecule which contains the amino acid residues that interact with an antigen and confer on the antibody its specificity and affinity for the antigen. The antibody binding region includes the “framework” amino acid residues necessary to maintain the proper conformation of the antigen-binding residues. Within the variable regions of the H or L chains that provide for the antigen binding regions are smaller sequences dubbed “hypervariable” because of their extreme variability between antibodies of differing specificity. Such hypervariable regions are also referred to as “complementarity determining regions” or “CDR” regions. These CDR regions account for the basic specificity of the antibody for a particular antigenic determinant structure.

The CDRs represent non-contiguous stretches of amino acids within the variable regions but, regardless of species, the positional locations of these critical amino acid sequences within the variable heavy and light chain regions have been found to have similar locations within the amino acid sequences of the variable chains. The variable heavy and light chains of all antibodies each have three CDR regions, each non-contiguous with the others. In all mammalian species, antibody peptides contain constant (i.e., highly conserved) and variable regions, and, within the latter, there are the CDRs and the so-called “framework regions” made up of amino acid sequences within the variable region of the heavy or light chain but outside the CDRs.

The present invention further provides a vector including at least one of the nucleic acids described above. Because the genetic code is degenerate, more than one codon can be used to encode a particular amino acid. Using the genetic code, one or more different nucleotide sequences can be identified, each of which would be capable of encoding the amino acid. The probability that a particular oligonucleotide will, in fact, constitute the actual encoding sequence can be estimated by considering abnormal base pairing relationships and the frequency with which a particular codon is actually used (to encode a particular amino acid) in eukaryotic or prokaryotic cells expressing an anti-NGF antibody or portion. Such “codon usage rules” are disclosed by Lathe, et al., 183 J. Molec. Biol. 1-12 (1985). Using the “codon usage rules” of Lathe, a single nucleotide sequence, or a set of nucleotide sequences that contains a theoretical “most probable” nucleotide sequence capable of encoding anti-NGF sequences can be identified. It is also intended that the antibody coding regions for use in the present invention could also be provided by altering existing antibody genes using standard molecular biological techniques that result in variants (agonists) of the antibodies and peptides described herein. Such variants include, but are not limited to deletions, additions and substitutions in the amino acid sequence of the anti-NGF antibodies or peptides.

For example, one class of substitutions is conservative amino acid substitutions. Such substitutions are those that substitute a given amino acid in an anti-NGF antibody peptide by another amino acid of like characteristics. Typically seen as conservative substitutions are the replacements, one for another, among the aliphatic amino acids Ala, Val, Leu, and lie; interchange of the hydroxyl residues Ser and Thr, exchange of the acidic residues Asp and Glu, substitution between the amide residues Asn and Gin, exchange of the basic residues Lys and Arg, replacements among the aromatic residues Phe, Tyr, and the like. Guidance concerning which amino acid changes are likely to be phenotypically silent is found in Bowie et al., 247 Science 1306-10 (1990).

Variant anti-NGF antigen binding proteins or antibody fragments may be fully functional or may lack function in one or more activities. Fully functional variants typically contain only conservative variations or variations in non-critical residues or in non-critical regions. Functional variants can also contain substitution of similar amino acids that result in no change or an insignificant change in function. Alternatively, such substitutions may positively or negatively affect function to some degree. Non-functional variants typically contain one or more non-conservative amino acid substitutions, deletions, insertions, inversions, or truncation or a substitution, insertion, inversion, or deletion in a critical residue or critical region.

Amino acids that are essential for function can be identified by methods known in the art, such as site-directed mutagenesis or alanine-scanning mutagenesis. Cunningham et al., 244 Science 1081-85 (1989). The latter procedure introduces single alanine mutations at every residue in the molecule. The resulting mutant molecules are then tested for biological activity such as epitope binding or in vitro ADCC activity. Sites that are critical for ligand-receptor binding can also be determined by structural analysis such as crystallography, nuclear magnetic resonance, or photoaffinity labeling. Smith et al., 224 J. Mol. Biol. 899-904 (1992); de Vos et al., 255 Science 306-12 (1992).

Moreover, polypeptides often contain amino acids other than the twenty “naturally occurring” amino acids. Further, many amino acids, including the terminal amino acids, may be modified by natural processes, such as processing and other post-translational modifications, or by chemical modification techniques well known in the art. Known modifications include, but are not limited to, acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent crosslinks, formation of cystine, formation of pyroglutamate, formylation, gamma carboxylation, glycosylation, GPI anchor formation, hydroxylation, iodination, methylation, myristoylation, oxidation, proteolytic processing, phosphorylation, prenylation, racemization, selenoylation, sulfation, transfer-RNA mediated addition of amino acids to proteins such as arginylation, and ubiquitination. Such modifications are well known to those of skill in the art and have been described in great detail in the scientific literature. Several particularly common modifications, glycosylation, lipid attachment, sulfation, gamma-carboxylation of glutamic acid residues, hydroxylation and ADP ribosylation, for instance, are described in most basic texts, such as Proteins-Structure and Molecular Properties (2nd ed., T. E. Creighton, W. H. Freeman & Co., NY, 1993). Many detailed reviews are available on this subject, such as by Wold, Posttranslational Covalent Modification of proteins, 1-12 (Johnson, ed., Academic Press, NY, 1983); Seifter et al. 182 Meth. Enzymol. 626-46 (1990); and Rattan et al. 663 Ann. NY Acad. Sci. 48-62 (1992).

Accordingly, the antibodies and peptides of the present invention also encompass derivatives or analogs in which a substituted amino acid residue is not one encoded by the genetic code. Similarly, the additions and substitutions in the amino acid sequence as well as variations, and modifications just described may be equally applicable to the amino acid sequence of the NGF antigen and/or epitope or peptides thereof, and are thus encompassed by the present invention. As mentioned above, the genes encoding a monoclonal antibody, according to the present invention, is specifically effective in the recognition of NGF.

### Antibody Derivatives

Included within the scope of this invention are antibody derivatives. A “derivative” of an antibody contains additional chemical moieties not normally a part of the protein. Covalent modifications of the protein are included within the scope of this invention. Such modifications may be introduced into the molecule by reacting targeted amino acid residues of the antibody with an organic derivatizing agent that is capable of reacting with selected side chains or terminal residues. For example, derivatization with bifunctional agents, well-known in the art, is useful for cross-linking the antibody or fragment to a water-insoluble support matrix or to other macromolecular carriers.

Derivatives also include radioactively labeled monoclonal antibodies that are labeled. For example, with radioactive iodine (251, 1311), carbon (4C), sulfur (35S), indium, tritium (H³) or the like; conjugates of monoclonal antibodies with biotin or avidin, with enzymes, such as horseradish peroxidase, alkaline phosphatase, beta-D-galactosidase, glucose oxidase, glucoamylase, carboxylic acid anhydrase, acetylcholine esterase, lysozyme, malate dehydrogenase or glucose 6-phosphate dehydrogenase; and also conjugates of monoclonal antibodies with bioluminescent agents (such as luciferase), chemoluminescent agents (such as acridine esters) or fluorescent agents (such as phycobiliproteins).

Another derivative bifunctional antibody of the present invention is a bispecific antibody, generated by combining parts of two separate antibodies that recognize two different antigenic groups. This may be achieved by crosslinking or recombinant techniques. Additionally, moieties may be added to the antibody or a portion thereof to increase half-life in vivo (ex., by lengthening the time to clearance from the blood stream. Such techniques include, for example, adding PEG moieties (also termed pegilation), and are well-known in the art. See U.S. Patent. Appl. Pub. No. 20030031671.

### Recombinant Expression of Antibodies

In some embodiments, the nucleic acids encoding a subject monoclonal antibody are introduced directly into a host cell, and the cell is incubated under conditions sufficient to induce expression of the encoded antibody. After the subject nucleic acids have been introduced into a cell, the cell is typically incubated, normally at 37° C., sometimes under selection, for a period of about 1-24 hours in order to allow for the expression of the antibody. In one embodiment, the antibody is secreted into the supernatant of the media in which the cell is growing. Traditionally, monoclonal antibodies have been produced as native molecules in murine hybridoma lines. In addition to that technology, the present invention provides for recombinant DNA expression of monoclonal antibodies. This allows the production of caninized antibodies, as well as a spectrum of antibody derivatives and fusion proteins in a host species of choice.

A nucleic acid sequence encoding at least one anti-NGF antibody, portion or polypeptide of the present invention may be recombined with vector DNA in accordance with conventional techniques, including blunt-ended or staggered-ended termini for ligation, restriction enzyme digestion to provide appropriate termini, filling in of cohesive ends as appropriate, alkaline phosphatase treatment to avoid undesirable joining, and ligation with appropriate ligases. Techniques for such manipulations are disclosed, ex. by Maniatis et al., MOLECULAR CLONING, LAB. MANUAL, (Cold Spring Harbor Lab. Press, NY, 1982 and 1989), and Ausubel et al. 1993 supra, may be used to construct nucleic acid sequences which encode a monoclonal antibody molecule or antigen binding region thereof.

A nucleic acid molecule, such as DNA, is said to be “capable of expressing” a polypeptide if it contains nucleotide sequences which contain transcriptional and translational regulatory information and such sequences are “operably linked” to nucleotide sequences which encode the polypeptide. An operable linkage is a linkage in which the regulatory DNA sequences and the DNA sequence sought to be expressed are connected in such a way as to permit gene expression as anti-NGF peptides or antibody portions in recoverable amounts. The precise nature of the regulatory regions needed for gene expression may vary from organism to organism, as is well known in the analogous art. See, ex. Sambrook et al., 2001 supra; Ausubel et al., 1993 supra.

The present invention accordingly encompasses the expression of an anti-NGF antibody or peptide, in either prokaryotic or eukaryotic cells. Suitable hosts include bacterial or eukaryotic hosts including bacteria, yeast, insects, fungi, bird and mammalian cells either in vivo, or in situ, or host cells of mammalian, insect, bird or yeast origin. The mammalian cell or tissue may be of human, primate, hamster, rabbit, rodent, cow, pig, sheep, horse, goat, dog or cat origin, but any other mammalian cell may be used.

In one embodiment, the nucleotide sequence of the invention will be incorporated into a plasmid or viral vector capable of autonomous replication in the recipient host. Any of a wide variety of vectors may be employed for this purpose. See, ex., Ausubel et al., 1993 supra. Factors of importance in selecting a particular plasmid or viral vector include: the ease with which recipient cells that contain the vector may be recognized and selected from those recipient cells which do not contain the vector; the number of copies of the vector which are desired in a particular host; and whether it is desirable to be able to “shuttle” the vector between host cells of different species.

Example prokaryotic vectors known in the art include plasmids such as those capable of replication in E. coli (such as but not limited to, for example, pBR322, ColE1, pSC101, pACYC 184, and the like). Such plasmids are, for example, disclosed by Maniatis et al., 1989 supra; Ausubel et al, 1993 supra. Bacillus plasmids include pC194, pC221, pT127, etc. Such plasmids are disclosed by Gryczan, in THE MOLEC. BIO. OF THE BACILLI 307-329 (Academic Press, NY, 1982). Suitable Streptomyces plasmids include pIJ101 (Kendall et al., 169 J. Bacteriol. 4177-83 (1987), and Streptomyces bacteriophages such as phLC31 (Chater et al., in SIXTH INT'L SYMPOSIUM ON ACTINOMYCETALES BIO. 45-54 (Akademiai Kaido, Budapest, Hungary 1986). Pseudomonas plasmids are reviewed in John et al., 8 Rev. Infect. Dis. 693-704 (1986); Izaki, 33 Jpn. J. Bacteriol. 729-42 (1978); and Ausubel et al., 1993 supra.

Alternatively, gene expression elements useful for the expression of cDNA encoding anti-NGF antibodies or peptides include, but are not limited to (a) viral transcription promoters and their enhancer elements, such as for example but not limited to the SV40 early promoter (Okayama et al., 3 Mol. Cell. Biol. 280 (1983), Rous sarcoma virus LTR (Gorman et al., 79 Proc. Natl. Acad. Sci., USA 6777 (1982), and Moloney murine leukemia virus LTR (Grosschedl et al., 41 Cell 885 (1985); (b) splice regions and polyadenylation sites such as those derived from the SV40 late region (Okayarea et al., 1983), and (c) polyadenylation sites such as in SV40 (Okayama et al., 1983).

Immunoglobulin cDNA genes can be expressed as described by Weidle et al., 51 Gene 21 (1987), using as expression elements the SV40 early promoter and its enhancer, the mouse immunoglobulin H chain promoter enhancers, SV40 late region mRNA splicing, rabbit S-globin intervening sequence, immunoglobulin and rabbit S-globin polyadenylation sites, and SV40 polyadenylation elements. For immunoglobulin genes comprised of part cDNA, part genomic DNA (Whittle et al., 1 Protein Engin. 499 (1987», the transcriptional promoter can be human cytomegalovirus, the promoter enhancers can be cytomegalovirus and mouse/human immunoglobulin, and mRNA splicing and polyadenylation regions can be the native chromosomal immunoglobulin sequences.

In one embodiment, for expression of cDNA genes in rodent cells, the transcriptional promoter is a viral LTR sequence, the transcriptional promoter enhancers are either or both the mouse immunoglobulin heavy chain enhancer and the viral LTR enhancer, the splice region contains an intron of greater than 31 bp, and the polyadenylation and transcription termination regions are derived from the native chromosomal sequence corresponding to the immunoglobulin chain being synthesized. In other embodiments, cDNA sequences encoding other proteins are combined with the above-recited expression elements to achieve expression of the proteins in mammalian cells.

Each fused gene can be assembled in, or inserted into, an expression vector. Recipient cells capable of expressing the chimeric immunoglobulin chain gene product are then transfected singly with an anti-NGF peptide or chimeric H or chimeric L chain-encoding gene, or are co-transfected with a chimeric H and a chimeric L chain gene. The transfected recipient cells are cultured under conditions that permit expression of the incorporated genes and the expressed immunoglobulin chains or intact antibodies or fragments are recovered from the culture.

In one embodiment, the fused genes encoding the anti-NGF peptide or chimeric H and L chains, or portions thereof are assembled in separate expression vectors that are then used to cotransfect a recipient cell. Alternatively, the fused genes encoding the chimeric H and L chains can be assembled on the same expression vector. For transfection of the expression vectors and production of the chimeric antibody, the recipient cell line may be a myeloma cell. Myeloma cells can synthesize, assemble and secrete immunoglobulins encoded by transfected immunoglobulin genes and possess the mechanism for glycosylation of the immunoglobulin. Myeloma cells can be grown in culture or in the peritoneal cavity of a mouse, where secreted immunoglobulin can be obtained from ascites fluid. Other suitable recipient cells include lymphoid cells such as B lymphocytes of human or nonhuman origin, hybridoma cells of human or non-human origin, or interspecies heterohybridoma cells.

The expression vector carrying a chimeric, caninized antibody construct or anti-NGF polypeptide of the present invention can be introduced into an appropriate host cell by any of a variety of suitable means, including such biochemical means as transformation, transfection, conjugation, protoplast fusion, calcium phosphate-precipitation, and application with polycations such as diethylaminoethyl (DEAE) dextran, and such mechanical means as electroporation, direct microinjection, and microprojectile bombardment. Johnston et at, 240 Science 1538 (1988).

Yeast can provide substantial advantages over bacteria for the production of immunoglobulin H and L chains. Yeasts carry out post-translational peptide modifications including glycosylation. A number of recombinant DNA strategies now exist which utilize strong promoter sequences and high copy number plasmids which can be used for production of the desired proteins in yeast. Yeast recognizes leader sequences of cloned mammalian gene products and secretes peptides bearing leader sequences (i.e., pre-peptides). Hitzman et al., 11th Int'l Conference on Yeast, Genetics & Molec. Biol. (Montpelier, France, 1982).

Yeast gene expression systems can be routinely evaluated for the levels of production, secretion and the stability of anti-NGF peptides, antibody and assembled murine and chimeric, heterochimeric, caninized, antibodies, fragments and regions thereof. Any of a series of yeast gene expression systems incorporating promoter and termination elements from the actively expressed genes coding for glycolytic enzymes produced in large quantities when yeasts are grown in media rich in glucose can be utilized. Known glycolytic genes can also provide very efficient transcription control signals. For example, the promoter and terminator signals of the phosphoglycerate kinase (PGK) gene can be utilized. A number of approaches can be taken for evaluating optimal expression plasmids for the expression of cloned immunoglobulin cDNAs in yeast. See Vol. II DNA Cloning, 45-66, (Glover, ed.,) IRL Press, Oxford, UK 1985).

Bacterial strains can also be utilized as hosts for the production of antibody molecules or peptides described by this invention. Plasmid vectors containing replicon and control sequences which are derived from species compatible with a host cell are used in connection with these bacterial hosts. The vector carries a replication site, as well as specific genes which are capable of providing phenotypic selection in transformed cells. A number of approaches can be taken for evaluating the expression plasmids for the production of murine, chimeric, heterochimeric, caninized antibodies, fragments and regions or antibody chains encoded by the cloned immunoglobulin cDNAs in bacteria (see Glover, 1985 supra; Ausubel, 1993 supra; Sambrook, 2001 supra; Colligan et al., eds. Current Protocols in Immunology, John Wiley & Sons, NY, NY (1994-2001); Colligan et al., eds. Current Protocols in Protein Science, John Wiley & Sons, NY, NY (1997-2001).

Host mammalian cells may be grown in vitro or in vivo. Mammalian cells provide posttranslational modifications to immunoglobulin protein molecules including leader peptide removal, folding and assembly of Hand L chains, glycosylation of the antibody molecules, and secretion of functional antibody protein. Mammalian cells which can be useful as hosts for the production of antibody proteins, in addition to the cells of lymphoid origin described above, include cells of fibroblast origin, such as Vero (ATCC CRL 81) or CHO-K1 (ATCC CRL 61) cells. Many vector systems are available for the expression of cloned anti-NGF peptides Hand L chain genes in mammalian cells (see Glover, 1985 supra). Different approaches can be followed to obtain complete H2L2 antibodies. It is possible to co-express Hand L chains in the same cells to achieve intracellular association and linkage of Hand L chains into complete tetrameric H2L2 antibodies and/or anti-NGF peptides. The co-expression can occur by using either the same or different plasmids in the same host. Genes for both Hand L chains and/or anti-NGF peptides can be placed into the same plasmid, which is then transfected into cells, thereby selecting directly for cells that express both chains. Alternatively, cells can be transfected first with a plasmid encoding one chain, for example the L chain, followed by transfection of the resulting cell line with an H chain plasmid containing a second selectable marker. cell lines producing anti-NGF peptides and/or H2L2 molecules via either route could be transfected with plasmids encoding additional copies of peptides, H, L, or H plus L chains in conjunction with additional selectable markers to generate cell lines with enhanced properties, such as higher production of assembled H2L2 antibody molecules or enhanced stability of the transfected cell lines.

For long-term, high-yield production of recombinant antibodies, stable expression may be used. For example, cell lines, which stably express the antibody molecule may be engineered. Rather than using expression vectors which contain viral origins of replication, host cells can be transformed with immunoglobulin expression cassettes and a selectable marker. Following the introduction of the foreign DNA, engineered cells may be allowed to grow for 1-2 days in enriched media, and then are switched to a selective media. The selectable marker in the recombinant plasmid confers resistance to the selection and allows cells to stably integrate the plasmid into a chromosome and grow to form foci which in turn can be cloned and expanded into cell lines. Such engineered cell lines may be particularly useful in screening and evaluation of compounds/components that interact directly or indirectly with the antibody molecule.

Once an antibody of the invention has been produced, it may be purified by any method known in the art for purification of an immunoglobulin molecule, for example, by chromatography (ex. ion exchange, affinity, particularly affinity for the specific antigen after Protein A, and sizing column chromatography), centrifugation, differential solubility, or by any other standard technique for the purification of proteins. In many embodiments, antibodies are secreted from the cell into culture medium and harvested from the culture medium.

### Pharmaceutical and Veterinary Applications

The anti-NGF antigen binding protein or antibody fragments as described herein of the present invention can be used for example in the treatment of NGF related disorders in dogs and cats. More specifically, the invention further provides for a pharmaceutical composition comprising a pharmaceutically acceptable carrier or diluent and, as active ingredient, an antibody or antibody fragment according to the invention. The antibody can be a chimeric, heterochimeric, caninized, felinized, equinized, humanized or speciated to accommodate a different species. Intact immunoglobulins or their binding fragments, such as Fab, are also envisioned. The antibody and pharmaceutical compositions thereof of this invention are useful for parenteral administration, ex., subcutaneously, intramuscularly or intravenously.

Anti-NGF antibodies and/or peptides of the present invention can be administered either as individual therapeutic agents or in combination with other therapeutic agents. They can be administered alone, but are generally administered with a pharmaceutical carrier selected on the basis of the chosen route of administration and standard pharmaceutical practice. Administration of the antibodies disclosed herein may be carried out by any suitable means, including parenteral injection (such as intraperitoneal, subcutaneous, or intramuscular injection), orally, or by topical administration of the antibodies (typically carried in a pharmaceutical formulation) to an airway surface. Topical administration to an airway surface can be carried out by intranasal administration (ex., by use of dropper, swab, or inhaler). Topical administration of the antibodies to an airway surface can also be carried out by inhalation administration, such as by creating respirable particles of a pharmaceutical formulation (including both solid and liquid particles) containing the antibodies as an aerosol suspension, and then causing the subject to inhale the respirable particles. Methods and apparatus for administering respirable particles of pharmaceutical formulations are well known, and any conventional technique can be employed.

In some desired embodiments, the antibodies are administered by parenteral injection. For parenteral administration, anti-NGF antibodies or peptides can be formulated as a solution, suspension, emulsion or lyophilized powder in association with a pharmaceutically acceptable parenteral vehicle. For example the vehicle may be a solution of the antibody or a cocktail thereof dissolved in an acceptable carrier, such as an aqueous carrier such vehicles are water, saline, Ringer's solution, dextrose solution, trehalose or sucrose solution, or 5% serum albumin, 0.4% saline, 0.3% glycine and the like. Liposomes and nonaqueous vehicles such as fixed oils can also be used. These solutions are sterile and generally free of particulate matter. These compositions may be sterilized by conventional, well known sterilization techniques. The compositions may contain pharmaceutically acceptable auxiliary substances as required to approximate physiological conditions such as pH adjusting and buffering agents, toxicity adjustment agents and the like, for example sodium acetate, sodium chloride, potassium chloride, calcium chloride, sodium lactate, etc. The concentration of antibody in these formulations can vary widely, for example from less than about 0.5%, usually at or at least about 1% to as much as 15% or 20% by weight and will be selected primarily based on fluid volumes, viscosities, etc., in accordance with the particular mode of administration selected. The vehicle or lyophilized powder can contain additives that maintain isotonicity (ex., sodium chloride, mannitol) and chemical stability (ex., buffers and preservatives). The formulation is sterilized by commonly used techniques. Actual methods for preparing parenterally administrable compositions will be known or apparent to those skilled in the art and are described in more detail in, for example, REMINGTON'S PHARMA. SCI. (15th ed., Mack Pub. Co., Easton, Pa., 1980).

The antibodies of this invention can be lyophilized for storage and reconstituted in a suitable carrier prior to use. This technique has been shown to be effective with conventional immune globulins. Any suitable lyophilization and reconstitution techniques can be employed. It will be appreciated by those skilled in the art that lyophilization and reconstitution can lead to varying degrees of antibody activity loss and that use levels may have to be adjusted to compensate. The compositions containing the present antibodies or a cocktail thereof can be administered for prevention of recurrence and/or therapeutic treatments for existing disease. Suitable pharmaceutical carriers are described in the most recent edition of REMINGTON'S PHARMACEUTICAL SCIENCES, a standard reference text in this field of art. In therapeutic application, compositions are administered to a subject already suffering from a disease, in an amount sufficient to cure or at least partially arrest or alleviate the disease and its complications. An amount adequate to accomplish this is defined as a “therapeutically effective dose” or a “therapeutically effective amount”. Amounts effective for this use will depend upon the severity of the disease and the general state of the subject's own immune system. In view of the minimization of extraneous substances and the lower probability of “foreign substance” rejections which are achieved by the present canine-like and antibodies of this invention, it may be possible to administer substantial excesses of these antibodies.

The dosage administered will, of course, vary depending upon known factors such as the pharmacodynamic characteristics of the particular agent, and its mode and route of administration; age, health, and weight of the recipient; nature and extent of symptoms kind of concurrent treatment, frequency of treatment, and the effect desired.

As a non-limiting example, treatment of NGF-related pathologies in dogs and cats can be provided as a biweekly or monthly dosage of anti-NGF antibodies of the present invention in the dosage range as needed. Example antibodies for canine therapeutic use are high affinity (these may also be high avidity) antibodies, and fragments, regions and derivatives thereof having potent in vivo anti-NGF activity, according to the present invention. Single or multiple administrations of the compositions can be carried out with dose levels and pattern being selected by the treating veterinarian. In any event, the pharmaceutical formulations should provide a quantity of the antibody(ies) of this invention sufficient to effectively treat the subject.

### Diagnostic Applications

The present invention also provides the above anti-NGF antibodies and peptides for use in diagnostic methods for detecting NGF in species, particularly canines and felines, known to be or suspected of having an NGF related disorder. In an embodiment of the invention the NGF related disorder is pain. In another embodiment, the NGF related disorder is osteoarthritis. Anti-NGF antibodies and/or peptides of the present invention are useful for immunoassays which detect or quantitate NGF, or anti-NGF antibodies, in a sample. An immunoassay for NGF typically comprises incubating a clinical or biological sample in the presence of a detectably labeled high affinity (or high avidity) anti-NGF antibody or polypeptide of the present invention capable of selectively binding to NGF, and detecting the labeled peptide or antibody which is bound in a sample. Various clinical assay procedures are well known in the art. See, ex. IMMUNOASSAYS FOR THE 80'S (Voller et al., eds., Univ. Park, 1981). Such samples include tissue biopsy, blood, serum, and fecal samples, or liquids collected from animal subjects and subjected to ELISA analysis as described below. Thus, an anti-NGF antibody or polypeptide can be fixed to nitrocellulose, or another solid support which is capable of immobilizing cells, cell particles or soluble proteins. The support can then be washed with suitable buffers followed by treatment with the detectably labeled NGF specific peptide, antibody or antigen binding protein. The solid phase support can then be washed with the buffer a second time to remove unbound peptide or antibody. The amount of bound label on the solid support can then be detected by known method steps.

“Solid phase support” or “carrier” refers to any support capable of binding peptide, antigen, or antibody. Well-known supports or carriers, include glass, polystyrene, polypropylene, polyethylene, polyvinylidenefluoride (PVDF), dextran, nylon, amylases, natural and modified celluloses, polyacrylamides, agaroses, and magnetite. The nature of the carrier can be either soluble to some extent or insoluble for the purposes of the present invention. The support material can have virtually any possible structural configuration so long as the coupled molecule is capable of binding to NGF or an anti-NGF antibody. Thus, the support configuration can be spherical, as in a bead, or cylindrical, as in the inside surface of a test tube, or the external surface of a rod. Alternatively, the surface can be flat, such as a sheet, culture dish, test strip, etc. For example, supports may include polystyrene beads. Those skilled in the art will know many other suitable carriers for binding antibody, peptide or antigen, or can ascertain the same by routine experimentation. Well known method steps can determine binding activity of a given lot of anti-NGF peptide and/or antibody or antigen binding protein. Those skilled in the art can determine operative and optimal assay conditions by routine experimentation.

Detectably labeling an NGF-specific peptide and/or antibody can be accomplished by linking to an enzyme for use in an enzyme immunoassay (EIA), or enzyme-linked immunosorbent assay (ELISA). The linked enzyme reacts with the exposed substrate to generate a chemical moiety which can be detected, for example, by spectrophotometric, fluorometric or by visual means. Enzymes which can be used to detectably label the NGF-specific antibodies of the present invention include, but are not limited to, malate dehydrogenase, staphylococcal nuclease, delta5-steroid isomerase, yeast alcohol dehydrogenase, alpha-glycerophosphate dehydrogenase, triose phosphate isomerase, horseradish peroxidase, alkaline phosphatase, asparaginase, glucose oxidase, beta-galactosidase, ribonuclease, urease, catalase, glucose-6-phosphate dehydrogenase, glucoamylase and acetylcholinesterase. By radioactively labeling the NGF-specific antibodies, it is possible to detect NGF through the use of a radioimmunoassay (RIA). See Work et al., LAB. TECHNIQUES & BIOCHEM. IN MOLEC. BIO (No. Holland Pub. Co., NY, 1978). The radioactive isotope can be detected by such means as the use of a gamma counter or a scintillation counter or by autoradiography. Isotopes which are particularly useful for the purpose of the present invention include: ³H, ¹²⁵I, ¹³¹I, ³⁵S, and ¹⁴C.

It is also possible to label the NGF-specific antibodies with a fluorescent compound. When the fluorescent labeled antibody is exposed to light of the proper wave length, its presence can then be detected due to fluorescence. Among the most commonly used fluorescent labeling compounds are fluorescein isothiocyanate, rhodamine, phycoerythrin, phycocyanin, allophycocyan in, o-phthaldehyde and fluorescamine. The NGF-specific antibodies or antigen binding proteins can also be delectably labeled using fluorescence-emitting metals such a ¹²⁵Eu, or others of the lanthanide series. These metals can be attached to the NGF specific antibody using such metal chelating groups as diethylenetriaminepentaacetic acid (DTPA) or ethylenediamine-tetraacetic acid (EDTA).

The NGF-specific antibodies also can be detectably labeled by coupling to a chemiluminescent compound. The presence of the chemiluminescently labeled antibody is then determined by detecting the presence of luminescence that arises during the course of a chemical reaction. Examples of useful chemiluminescent labeling compounds are luminol, isoluminol, theromatic acridinium ester, imidazole, acridinium salt and oxalate ester.

Likewise, a bioluminescent compound can be used to label the NGF-specific antibody, portion, fragment, polypeptide, or derivative of the present invention. Bioluminescence is a type of chemiluminescence found in biological systems in which a catalytic protein increases the efficiency of the chemiluminescent reaction. The presence of a bioluminescent protein is determined by detecting the presence of luminescence. Important bioluminescent compounds for purposes of labeling are luciferin, luciferase and aequorin.

Detection of the NGF-specific antibody, portion, fragment, polypeptide, or derivative can be accomplished by a scintillation counter, for example, if the detectable label is a radioactive gamma emitter, or by a fluorometer, for example, if the label is a fluorescent material. In the case of an enzyme label, the detection can be accomplished by colorometric methods which employ a substrate for the enzyme. Detection can also be accomplished by visual comparison of the extent of enzymatic reaction of a substrate in comparison with similarly prepared standards.

For the purposes of the present invention, the NGF which is detected by the above assays can be present in a biological sample. Any sample containing NGF may be used. For example, the sample is a biological fluid such as, for example, blood, serum, lymph, urine, feces, inflammatory exudate, cerebrospinal fluid, amniotic fluid, a tissue extract or homogenate, and the like. The invention is not limited to assays using only these samples, however, it being possible for one of ordinary skill in the art, in light of the present specification, to determine suitable conditions which allow the use of other samples.

In situ detection can be accomplished by removing a histological specimen from an animal subject, and providing the combination of labeled antibodies of the present invention to such a specimen. The antibody (or portion thereof) may be provided by applying or by overlaying the labeled antibody (or portion) to a biological sample. Through the use of such a procedure, it is possible to determine not only the presence of NGF but also the distribution of NGF in the examined tissue. Using the present invention, those of ordinary skill will readily perceive that any of a wide variety of histological methods (such as staining procedures) can be modified in order to achieve such in situ detection.

The antibody, fragment or derivative of the present invention can be adapted for utilization in an immunometric assay, also known as a “two-site” or “sandwich” assay. In a typical immunometric assay, a quantity of unlabeled antibody (or fragment of antibody) is bound to a solid support that is insoluble in the fluid being tested and a quantity of detectably labeled soluble antibody is added to permit detection and/or quantification of the ternary complex formed between solid phase antibody, antigen, and labeled antibody.

The antibodies may be used to quantitatively or qualitatively detect the NGF in a sample or to detect presence of cells that express the NGF. This can be accomplished by immunofluorescence techniques employing a fluorescently labeled antibody (see below) coupled with fluorescence microscopy, flow cytometric, or fluorometric detection. For diagnostic purposes, the antibodies may either be labeled or unlabeled. Unlabeled antibodies can be used in combination with other labeled antibodies (second antibodies) that are reactive with the antibody, such as antibodies specific for canine immunoglobulin constant regions. Alternatively, the antibodies can be directly labeled. A wide variety of labels may be employed, such as radionuclides, fluors, enzymes, enzyme substrates, enzyme cofactors, enzyme inhibitors, ligands (particularly haptens), etc. Numerous types of immunoassays, such as those discussed previously are available and are well known to those skilled in the art. Importantly, the antibodies of the present invention may be helpful in diagnosing an NGF related disorder in canines. More specifically, the antibody of the present invention may identify the overexpression of NGF in companion animals. Thus, the antibody of the present invention may provide an important immunohistochemistry tool. The antibodies of the present invention may be used on antibody arrays, highly suitable for measuring gene expression profiles.

### Kits

Also included within the scope of the present invention are kits for practicing the subject methods. The kits at least include one or more of the antibodies of the present invention, a nucleic acid encoding the same, or a cell containing the same. An antibody of the present invention may be provided, usually in a lyophilized form, in a container. The antibodies, which may be conjugated to a label or toxin, or unconjugated, are typically included in the kits with buffers, such as Tris, phosphate, carbonate, etc., stabilizers, biocides, inert proteins, e.g., serum albumin, or the like. Generally, these materials will be present in less than 5% wt. based on the amount of active antibody, and usually present in total amount of at least about 0.001% wt. based again on the antibody concentration. Frequently, it will be desirable to include an inert extender or excipient to dilute the active ingredients, where the excipient may be present in from about,1% to 99% wt. of the total composition. Where a second antibody capable of binding to the primary antibody is employed in an assay, this will usually be present in a separate vial. The second antibody is typically conjugated to a label and formulated in an analogous manner with the antibody formulations described above. The kit will generally also include a set of instructions for use.

The invention will now be described further by the non-limiting examples below.

### EXAMPLES

The present invention is further illustrated and supported by the following examples. However, these examples should in no way be considered to further limit the scope of the invention. To the contrary, one having ordinary skill in the art would readily understand that there are other embodiments, modifications, and equivalents of the present invention without departing from the spirit of the present invention and/or the scope of the appended claims.

### Example 1

### Synthesis and Purification of Canine NGF (cNGF)

PCR primers were designed with appropriate restriction sites to amplify canine pre-pro-ß-NGF (SEQ ID NO:2). The ß-NGF gene was cloned into plasmid pCTV927 (Chromos targeting plasmid) via EcoRV/KpnI sites. The pCTV927/ß-NGF plasmid was co-transfected, along with the plasmid encoding the Chromos system integrase pSIO343, using Lipofectamine 2000 transfection reagent into CHOK1SV cells. Individual stable clones were analyzed for expression and a high expressing clone was chosen for expansion and expression for subsequent purification. Canine ⊕-NGF (cNGF) produced from these transfections was purified using ion exchange chromatography. Initial cleanup was performed in flow-through batch mode over Q Sepharose FF (GE Healthcare #17-0510-01). The clarified supernatant was diluted 1:1 with water and pH adjusted to 8.5 with 1 M Tris. The diluted sample was mixed with Q Sepharose FF, at a ratio of 150:1, for >1.5 hours. The resin then was allowed to settle and the unbound portion collected. cNGF was further purified by cation exchange chromatography; it was diluted again 1:1 with water and loaded onto SP-Sepharose FF (GE Healthcare #17-0729-01) pre-equilibrated with 20 mM Tris, pH 8.5. After loading, the column was washed and then eluted via a linear gradient from 0 to 210 mM NaCl (each in 20 mM Tris, pH 8.5) over 20 column volumes. Fractions were analyzed by SDS-PAGE, pooled, dialyzed (3.5K mw) against PBS at 4° C. The dialysate was collected, sterile filtered, and concentration measured via absorbance at 280 nm (1 mg/mL=1.48 A₂₈₀).

### Example 2

### Identification of Mouse Monoclonal Antibodies Recognizing Nerve Growth Factor (NGF)

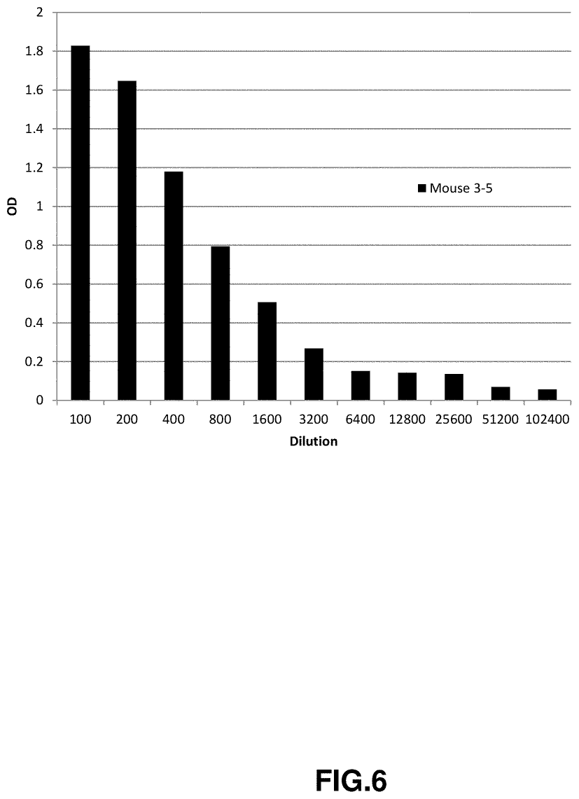

Mouse monoclonal antibodies were identified using standard immunizations of female CF-1 mice with recombinant cNGF produced in CHO cells according to procedures well known to those skilled in the art. Titers from immunized mice were determined using an enzyme linked immunosorbent assay (ELISA). cNGF (100 ng/well) was immobilized to polystyrene microplates and used as a capture antigen. Serum from immunized mice was diluted in phosphate buffered saline with 0.05% tween-20 (PBST) and added to the microtiter plate. Plates were washed and the presence of bound mouse anti-cNGF antibodies was detected with a Horse Radish Peroxidase (HRP)-conjugated goat anti-mouse secondary antibody (Kirkegard & Perry Laboratories, Inc. (KPL, Inc.), Gaithersburg, Md.). Following addition of a chromogenic substrate (ABTS 2-Component Microwell Peroxidase Substrate, KPL, Inc., Gaithersburg, Md.) and a ten minute incubation at room temperature (RT) the absorbance of each well was determined at an optical density (OD) of 450 nm and 490 nm. The antibody response of a single mouse (“3-5”) immunized with cNGF is shown in FIG. 6. A pool of donor splenocytes from this mouse was used for fusion.

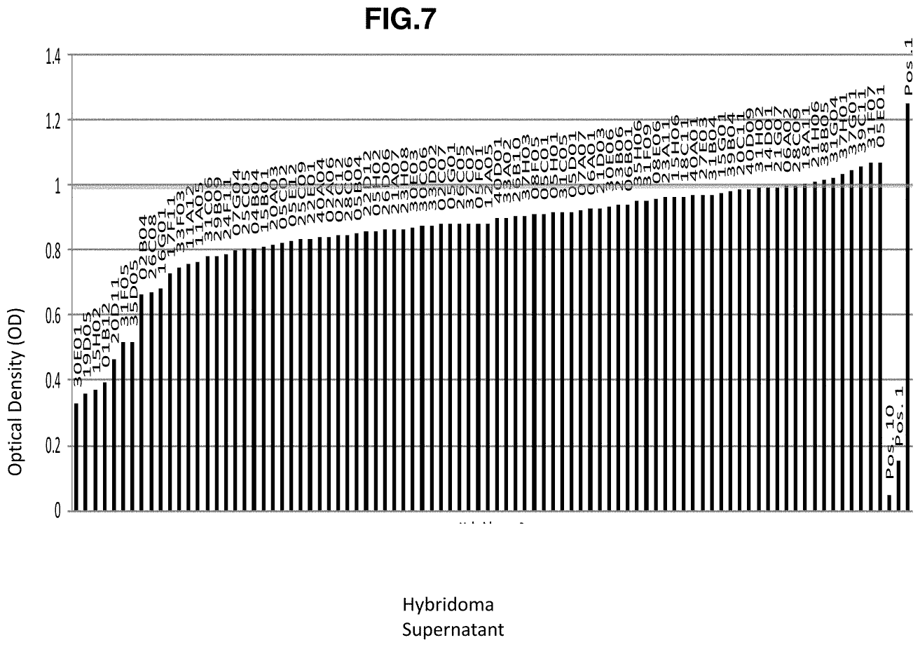

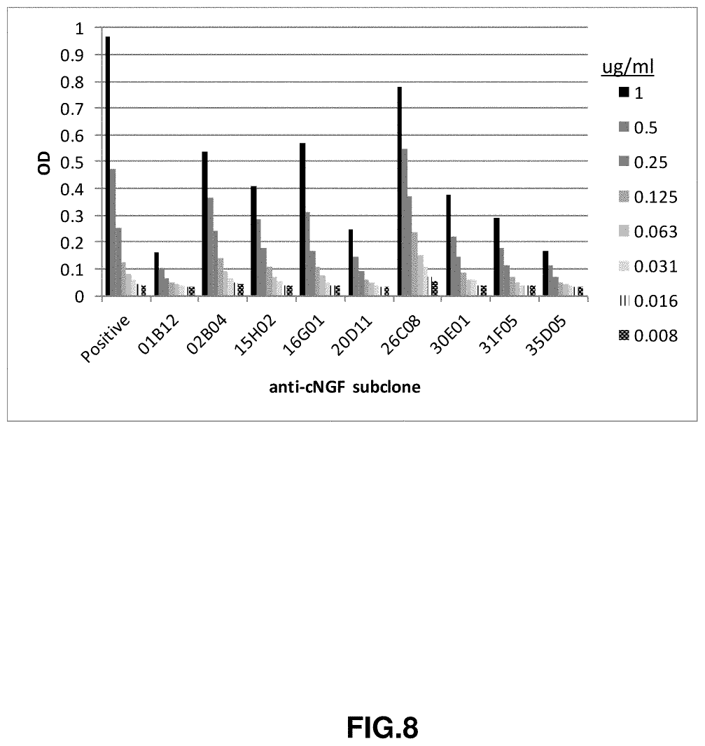

Following fusion and screening for anti-cNGF binding via direct ELISA, 87 wells were chosen to determine if they inhibit cNGF binding to a soluble form of the canine TrkA receptor using a competitive ELISA. 100 μl of cTrkA-Fc (1 μg/ml) was plated overnight in carb/bicarb buffer on an ELISA plate. Assay plates were then blocked with 200 μl of 1% BSA in PBS and incubate at 4 C. Hybridoma supernatants were tested neat, and at a 1:10 and a 1:50 dilution in PBS. 75 ul of supernatant dilutions were added to 75 ul (0.2 μg/ml) biotinylated NGF to achieve a final concentration of 0.1 ug/ml and incubated at room temp for 1 hr in a polypropylene plate. Following the 1 hr incubation the plate was moved to 4 C and incubated for an additional 15 minutes. The blocked assay plate was then washed with cold PBST and 100 ul of each supernatant:NGF mixture was added to each well of the TrkA assay plate. This assay plate was incubated at 4 C for 1 hr, washed with cold PBST then streptavidin HRP was added for a final incubation at RT for 1 hr. Following addition of ABTS substrate the plate was developed to an OD=1.0 for the PBS control wells. Supernatants containing antibodies capable of binding to cNGF and inhibiting its ability to bind to the cTRKa receptor were identified by comparing the OD signal to that of the positive control showing maximal cNGF:ctrkA binding. Data from this competitive ELISA are described in (FIG. 7). Select hits from these assays were purified, confirmed by ELISA, and subcloned by limiting dilution to produce pure anti-cNGF antibodies (FIG. 8).

### Example 3

### Potency of Anti-cNGF Antibodies Derived from Hybridomas

Potency was assessed by determining the percent inhibition by each antibody of cNGF induced phosphorylation of extracellular signal-regulated kinase 1 and 2 (pERK 1/2) signaling in a Chinese hamster ovary (CHO) cell line expressing the human tyrosine kinase A receptor (TrkA). Antibodies were diluted in HBSS and pre-incubated with 60 ng/ml cNGF in HBSS/0.1% BSA for 1 hour at room temperature. Following 1 hour co-incubation, 50 uL of mAb/cNGF mixture were added to human TrkA cells previously serum starved in 50 uL HBSS and allowed to incubate at 37 C for 10 minutes. Supernatants were then removed, cells lysed, and pERK signal assessed via Surefire AlphaScreen kit (Perkin Elmer). The final concentration of canine NGF was 15 ng/ml (EC80). Maximal response in the assay is defined as measured ERK 1/2 phosphorylation in the presence of cNGF only (no mAb). Minimal response is defined as the basal levels of ERK 1/2 phosphorylation (no stimulation). Calculated ICsovalues for anti-NGF antibodies and percentage of maximal inhibitory response are described in Table 1 below.

TABLE 1

| | | | Top | |
|---|---|---|---|---|
| | | Average | Conc. | |
| | | Max | Tested | IC50 |
| Subclone | Isotype | % Inhib. | (ug/ml) | (nM) |
| 01B12-02B08 | IgG1-kappa | 87.1 | 4.4 | 4.7 |
| 02B04-02A08 | IgG1-kappa | 82.2 | 21.3 | 4.0 |
| 15H02-02E01 | IgG1-kappa | 88.2 | 32.9 | 18.8 |
| 16G01-02F03 | IgG2a-kappa | 27.7 | 47.8 | n/a |
| 20D11-02E10 | IgG1-kappa | 78.1 | 5.5 | 5.5 |
| 26C08-02F06 | IgG1-kappa | 57.4 | 6.6 | 7.1 |
| 30E01-01H04 | IgG1-kappa | 4.0 | 7.7 | n/a |
| 30E01-02A11 | IgG1-kappa | 72.4 | 8.8 | 9.8 |
| 31F05-02B03 | IgG1-kappa | 86.2 | 4.2 | 4.1 |
| 35D05-02F02 | IgG1-kappa | 64.5 | 20.5 | 5.1 |
| Positive Control | IgG1-kappa | 98.7 | 2.0 | 0.68 |

### Example 4

### DNA Sequences Encoding Mouse Anti-cNGF Antibodies

Ribonucleic acid (RNA) was isolated from hybridoma cells using the RNEASY-mini kit (Qiagen, Inc., Germantown, Md.) as described by the manufacturer. One million frozen cells from each hybridoma were harvested by centrifugation and RNA was purified from cell lysates using the RNEASY spin column according to method described in the protocol. RNA was eluted from each column and used immediately for quantitation and cDNA preparation. The RNA was analyzed for yield and purity by measuring its absorbance at 260 nm and 280 nm using a GeneQuant pro spectrophotometer (GE Healthcare, Uppsala, Sweden). Following isolation, the remaining RNA was stored at −80° C. for further use.

Oligonucletide primers designed for amplification of the mouse immunoglobulin (Ig) variable domains were used according to the manufacturer's instructions (EMD Chemicals, Inc., Gibbstown, N.J.). cDNA was prepared from total hybridoma RNA by reverse transcription (RT) using the thermoscript RT kit (Invitrogen Corp., Carlsbad, Calif.) according to the manufacturer's instructions. 200-400 ng of RNA from each hybridoma was added to an individual reaction tube containing a 3′ Ig constant region primer. The 3′ constant Ig primer is positioned proximal to the variable Ig region and will transcribe first strand cDNA representing the variable region of the mouse antibody. For each hybridoma RNA, an individual RT reaction was performed using a 3′ constant heavy chain and 3′ constant kappa light chain primer.

cDNA from each hybridoma were used as a template in a polymerase chain reaction (PCR) to amplify the variable IgG heavy and kappa light chain cDNA for the purpose of sequence determination. Multiple reactions were performed for each PCR using a degenerate 5′ primer or primer pools designed to anneal to the signal sequence-coding regions of the mouse Ig variable domain. Separate PCR reactions were performed with a degenerate primer or primer pools for amplification of murine variable heavy and variable light chain regions. PCR was performed with 1 ul of the cDNA reaction using the Expand High

Fidelity DNA polymerase kit (Roche Diagnostics Corp., Indianapolis, Ind.) according to the manufacturer's protocol. Thermocycling parameters for the PCR were as follows; 94° C. for 2 min., 35 cycles (94° C. 15 sec., 55° C. 30 sec., 72 ° C. 1 min.), 72 ° C. 7 min. Fragments amplified from the PCR were separated by gel electrophoresis on a 1% agarose gel and purified using Qiagen gel extraction kit (Qiagen, Inc., Germantown, Md.). Forward primers for the heavy and light chain variable region incorporate EcoRI or SalI (New England Biolabs (NEB), Inc., Ipswich, Mass.) sites and reverse heavy and light chain variable, HindIII (NEB Inc., Ipswich, Mass.) to facilitate cloning into the pUC19 plasmid. Purified PCR fragments and pUC19 plasmid were digested with the above restriction endonucleases at 37° C. for 1-2 hrs. Following digestion, PCR fragments were purified using a Qiaquick PCR cleanup kit (Qiagen, Inc., Germantown, Md.). Digested plasmid was separated by gel electrophoresis on a 1% agarose gel and purified using Qiagen gel extraction kit. Purified PCR fragments representing variable IgG heavy and kappa light chain DNA were ligated into pUC19 plasmid using T4 DNA ligase and ligation buffer (NEB, Inc., Ipswich, Mass.) at 4° C. overnight. 3 ul of each ligation reaction was used to transform E. coli TOP10 cells (Invitrogen Corp., Carlsbad, Calif.).

Plasmids were isolated from positive clones representing the variable regions of each hybridoma using a Qiagen mini prep kit (Qiagen 27106) according to the manufacturer's protocol. M13 forward and reverse primers were used to amplify DNA sequence for each cloned insert using the BigDye sequencing reaction (Applied Biosystems by Life Technologies Corp., Carlsbad, Calif.) according to manufacturer's protocol. Sequencing reactions were purified using a 96 well purification kit (Zymo Research, Irvine, Calif.) according to the manufacturer's protocol. Samples were loaded onto an ABI-3730 capillary sequencer and resulting sequence traces were analyzed using Sequencher (GeneCodes v. 4.2) for presence of complete open reading frames.

Sequences identified from the positive hybridomas are listed as follows (CDRs are underlined):

TABLE 2

| SEQ | | |
|---|---|---|
| ID | SEQUENCE | |
| NO. | DESCRIPTION | AMINO ACID SEQUENCE |
| 96 | MU 01B12- | EVKLQESGPGLVAPSQSLSI |
| | 02B08 VH | TCTVSGFSLTGYGVNWVRQP |
| | | PGKGLEWLGMIWGDGSTDYN |
| | | SALKSRLSISKDNSKSQVFL |
| | | KMNSLQTDDTARYYCARDGY |
| | | YYGTTWYFDVWGAGTTVTVS |
| | | S |
| 100 | MU 01B12- | DIVMTQSTSSLSASLGDRVT |
| | 02B08 VL | ISCRASQDISNYLNWYQQKP |
| | | DGTIKLLIYYTSRLHSGVPS |
| | | RFSGSGSGTDYSLTISNLEQ |
| | | EDIATYFCQQGSTLPRTFGG |
| | | GT |
| 104 | MU 02B04 VH | EVKLEESGPGLVAPSQSLSI |
| | | TCTVSGFSLTGYGVNWVRQP |
| | | PGKGLEWLGMIWGDGSTDYN |
| | | SALKSRLNISKDNSKSQVFL |
| | | KMDSLQTDDTARYYCARGGY |
| | | DYDVPFFDYWGQGTTLTVSS |
| 108 | MU 02B04 VL | DIVMTQTTSSLSASLGDRVT |
| | | ISCRASQDISNYLNWYQQKP |
| | | DGTVKLLIYYTSRLHSGVPS |
| | | RFSGSGSGTDYSLTISNLEQ |
| | | EDIATYFCQQGNMFPYTLGG |
| | | GT |
| 112 | MU 15H02 VH | EVQLEQSGPGLVAPSQSLSI |
| | | TCTVSGFSLTGYGVNWVRQP |
| | | PGKGLEWLGMIWGDGSTDYN |
| | | SALKSRLSISKDNSKSQVFL |
| | | KMNSLQTDDTARYYCARDGY |
| | | YYGTTWYFDVWGAGTTVTVS |
| | | S |
| 114 | MU 15H02 VL | DIVLTQSTSSLSASLGDRVT |
| | | ISCRASQDISNYLNWYQQKP |
| | | DGTIKLLIYYTSRLHSGVPS |
| | | RFSGSGSGTDYSLTISNLEQ |
| | | EDIATYFCQQGSTLPRTFGG |
| | | GT |
| 116 | MU 16G01 VH | EVQLQESGAELVKPGASVKL |
| | | SCKASGYTFTNYWMHWVKQR |
| | | PGQGLEWIGHIDPSDGETHY |
| | | NQKFKDKATLTVDKSSSTAY |
| | | MQLTGLTSEDSAVYYCARFL |
| | | PDYWGQGTSVTVSS |
| 120 | MU 16GO1 VL | DIVLTQTPAIMSASPGEKVT |
| | | MTCRASSSVSSIYLHWYQQK |
| | | PGSSPKLWIYSTSNLASGVP |
| | | ARFSGSGSGTSYSLTVSSVE |
| | | AEDAATYYCQLYDNSPLTFG |
| | | AGT |
| 124 | MU20D11 VH | EVQLEESGPGLVAPSQSLSI |
| | | TCTVSGFSLTGYGVNWVRQP |
| | | PGKGLEWLGMIWGDGSTDYN |
| | | SALKSRLSISKDNSKSQVFL |
| | | KMNSLQTDDTARYYCARDGY |
| | | YYGTTWYFDVWGAGTTVTVS |
| | | S |
| 126 | MU 20D11 VL | DIVITQTPLSLPVSLGDQAS |
| | | ISCRSSQSIVHINRHTYLGW |
| | | YLQKPGQSLKLLIYGVSNRF |
| | | SGVPDRFSGSGSGTDFTLKI |
| | | SRVEAEDMGVYYCFQGTHVP |
| | | FTFGSGT |
| 130 | MU 26C08 VH | EVKLEESGPGLVAPSQSLSI |
| | | TCTVSGFSLTGYGVNWVRQP |
| | | PGKGLEWLGMIWGDGSTDYN |
| | | SALKSRLSISKDNSKSQVFL |
| | | KMNSLQTDDTARYYCARGGY |
| | | DYDVSFFDYWGQGTTLTVSS |
| 134 | MU 26CO8 VL | DIVLTQTTSSLSASLGDRVT |
| | | ISCRASQDISNYLNWYQQKP |
| | | DGTVKLLIYYTSRFHSGVPS |
| | | RFSGSGSGTDYSLTISNLEH |
| | | EDIATYFCQQGNTLPYTFGG |
| | | GT |
| 138 | MU 30E01 VH | QVKLEESGRGLVAPSQSLSI |
| | | TCTVSGFSLTGYGVNWVRQP |
| | | PGKGLEWLGMIWGDGSTDYN |
| | | SALKSRLSISKDNSKSQVFL |
| | | KMNSLQTDDTARYYCARDGY |
| | | YYGTTWYFDVWGAGTTVTVS |
| | | S |
| 140 | MU 30E01 VL | DIVLTQTTSSLSASLGDRVT |
| | | ISCRASQDISNYLNWYQQKP |
| | | DGTIKLLIYYTSRLHSGVPS |
| | | RFSGSGSGTDYSLTISNLEQ |
| | | EDIATYFCQQGSTLPRTFGG |
| | | GT |
| 142 | MU 31F05 VH | EVQIQQSGPGIVAPSQSISI |
| | | TCTVSGFSITGYGVNWVRQP |
| | | PGKGIEWIGMIWGDGSTDYN |
| | | SALKSRLSISKDNSKSQVFL |
| | | KMNSLQTDDTARYYCARDGY |
| | | YYGTTWYFDVWGAGTTVTVS |
| | | S |
| 144 | MU 31F05 VL | DIQMTQTTSSLSASLGDRVT |
| | | ISCRASQDISNYLNWYQQKP |
| | | DGTIKLLIYYTSRLHSGVPS |
| | | RFSGSGSGTDYSLTISNLEQ |
| | | EDIATYFCQQGSTLPRTFGG |
| | | GT |

### Example 5

### Construction of Chimeric Antibodies

Antibodies are composed of a homodimer pairing of two heterodimeric proteins. Each protein chain (one heavy and one light) of the heterodimer consists of a variable domain and a constant domain. Each variable domain contains three complementary determining regions (CDRs) which contribute to antigen binding. CDRs are separated in the variable domain by framework regions which provide a scaffold for proper spatial presentation of the binding sites on the antibody. Together, the CDR and framework regions contribute to the antibodies ability to bind its cognate antigen.

A chimeric antibody consists of the variable sequence (both CDR and framework) from the mouse antibody (as determined from the above sequence analysis) grafted onto the respective heavy and light constant regions of a canine IgG molecule. As the variable domain is responsible for antigen binding, grafting of the fully mouse variable domain onto canine constant region is expected to have little or no impact on the antibody's ability to bind the cNGF immunogen.

To simultaneously confirm that the correct sequence of the heavy and light chain variable regions were identified and to produce recombinant, homogenous material, expression vectors to produce the chimeric antibodies in mammalian expression systems were generated. Synthetic DNA constructs were designed to encode the mouse heavy and light chain variable region of antibody sequence derived from hybridomas 01B12, 16G01, 02B04, 20D11, and 26C08 (see sequence listing and sequence description above). Unique flanking restriction endonuclease sites, Kozak consensus sequence and, secretion leaders sequence were incorporated into each synthetic gene construct to facilitate expression and secretion of the recombinant antibody from a mammalian cell line. The gene containing each variable domain was cloned into a mammalian expression plasmid containing either the canine IgG heavy (IgG 65 e- SEQ ID NO: 186) or light chain (SEQ ID NO: 190) constant regions based on sequence from GenBank accession numbers AF354265 and XP_532962 respectively.

The plasmids encoding each heavy and light chain, under the control of the CMV promoter, were co-transfected into HEK 293 cells using standard lipofectamine methods. Following six days of expression, chimeric mAbs were purified from 30 ml of transiently transfected HEK293F cell supernatants using MabSelect SuRe protein A resin (GE Healthcare, Uppsala, Sweden) according to standard methods for protein purification. Eluted fractions were pooled, concentrated to ˜500 ul using a 10,000 nominal MW cutoff Nanosep Omega centrifugal device (Pall Corp., Port Washington, N.Y.), dialyzed overnight at 4° C. in 1× PBS, pH7.2 and stored at 4° C. for further use.

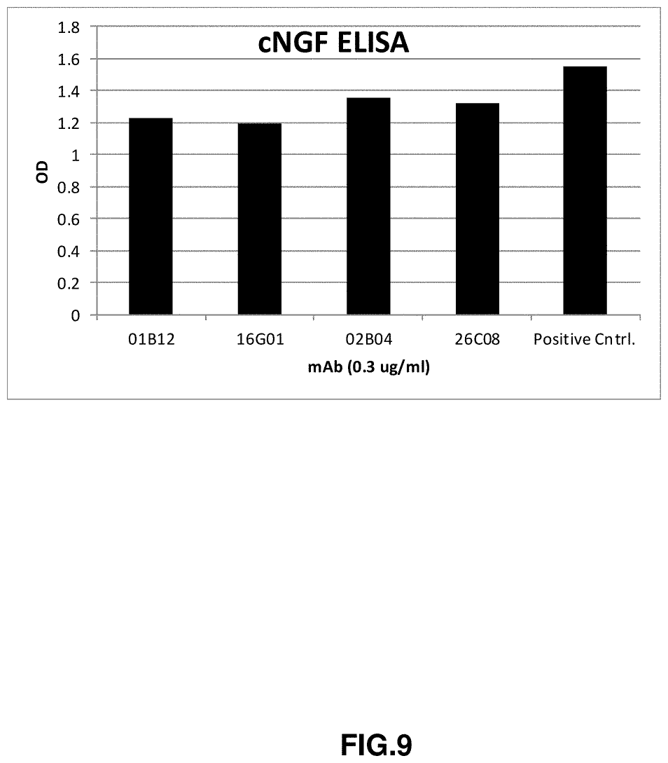

Chimeric mAbs showing expression from HEK 293 cells were further analyzed for affinity with which they bind NGF from multiple species (canine, human, and rat). Kinetic binding parameters were evaluated using surface plasmon resonance (SPR)-Biacore T200 (GE Healthcare) (FIG. 9). 2.3 μg/ml NGF was immobilized by amine coupling (carboxyl group activation by EDC-NGF mixture and deactivate excess reactive groups by Ethanolamine) for a final surface density of approximately 350 resonance units on CM5 sensor. The 3-fold serial dilutions of each mAb (20-0.25 nM, due to low mAb concentrations) were measured for 200 seconds followed by an extended dissociation period of 300 seconds with a flow rate of 30 μl/min. Regeneration was performed with Glycine pH1.5 and NaOH. Data were analyzed with Biacore T200 Evaluation software using 1:1 binding model following double referencing: the reference flow cell was subtracted from the flow cell containing immobilized NGF and then subtraction of a buffer only injection. Affinities <E-12 are below the lower limit of quantitation of detection for the instrument.

### Example 6

### Antibody Speciation Strategy

The generation of anti-drug antibodies (ADAs) can been associated with loss of efficacy for any biotherapeutic protein including monoclonal antibodies. Comprehensive evaluation of the literature has shown that speciation of monoclonal antibodies can reduce the propensity for mAbs to be immunogenic, although examples of immunogenic fully human mAbs and non-immunogenic chimeric mAbs can be found. To help mitigate risks associated with ADA formation for the mouse anti NGF monoclonal antibodies provided herein, caninization and felinization strategies was employed. The speciation strategy is based on identifying the most appropriate canine antibody framework sequence for CDR grafting. Following extensive analysis of all available canine and feline sequences, respectively, for both the heavy and light chain, sequences of the IgG variable regions were selected based on their homology to the mouse mAbs. The CDRs from the mouse progenitor mAbs were used to replace native canine CDRs. The objective was to retain high affinity and cell-based activity using fully canine frameworks to minimize the potential of immunogenicity in vivo. Constant regions were chosen based on biophysical and functional properties. Canine “IgG-B” which is functionally analogous to human IgG1 (Bergeron et al Vet Immunology and Immunopathology Vol 157, Issues 1-2, Jan. 15, 2014) As with the chimeric mAbs, SEQ ID NO.184 and SEQ ID NO. 160 canine constant regions were used.

### Example 7

### Caninization and Optimization of Anti-cNGF mAbs 011312 and 02B04

Two mAbs showing the highest potency as a chimeric form were chosen for caninization, 01B12 and 02B04. Synthetic nucleotide constructs representing the caninized variable heavy and light chains of mAbs 01B12 and 02B04 were made. Following subcloning of each variable chain into plasmids containing the respective canine heavy or kappa constant region, plasmids were co-transfected for antibody expression in HEK 293 cells. Binding to cNGF was initially characterized by ELISA and western blot. Those antibodies shown to retain binding following caninization were further analyzed for affinity using Biacore and functional activity in the TrkA cell based assay). Caninized forms of 01B12 are shown below in Table 3 in which murine CDRs were grafted into the identified canine framework regions and all combinations of heavy and light chains were expressed in transient HEK cells as described above. In the case of 01B12 mutations to the CDRs were necessary for caninization and were required to maintain the potency and expression observed with the chimeric forms. Caninized mAbs with high yields and which had high affinity to cNGF were identified to progress. Further CDR mutations to both caninized 02B04 and 01B12 were made via rational design and mutagenesis in order to optimize each caninized antibody. Table 3 below shows the constructs and the resulting mAb's potency.

TABLE 3

| | | | | IC50 |
|---|---|---|---|---|
| Alias | Heavy Chain | Light Chain | KD [M] | [nM] |
| 01B12 Chimera | Chim-01B12-VH | Chim-01B12-VL | 1.18E−09 | 1.02 |
| | (SEQ ID NO. 98) | (SEQ ID NO. 103) | | |
| 02B04 Chimera | Chim-02B04-VH | Chim-02B04-VL | 1.36E−09 | 0.97 |
| | (SEQ ID NO. 106) | (SEQ ID NO. 110) | | |
| H3AQC2301B12L1AL1L3* | 01B12H3AHC | QC2301B12L1AL1L3 | 6.1E−11 | 1.26 |
| | (SEQ ID NO. 49) | (SEQ ID NO. 51) | | |
| H3AQC23L5A | 01B12H3AHC | QC23L5A | 8.9E−11 | 2.32 |
| | (SEQ ID NO. 49) | (SEQ ID NO. 55) | | |
| H3A02B4VL1 | 01B12H3AHC | 02B4VL1 | 1.60E−10 | 1.4 |
| | (SEQ ID NO. 49) | (SEQ ID NO. 75) | | |
| H3AQC2301B12L2AL1 | 01B12H3AHC | QC2301B12L2AL1 | 4.35E−10 | 0.99 |
| | (SEQ ID NO. 49) | (SEQ ID NO. 57) | | |
| H3AQC2301B12L1AL3 | 01B12H3AHC | QC2301B12L1AL3 | 9.94E−10 | 1.44 |
| | (SEQ ID NO. 49) | (SEQ ID NO. 59) | | |
| H3AQC2301B12L1AL1 | 01B12H3AHC | QC2301B12L1AL1 | 1.07E−9 | 0.65 |
| | (SEQ ID NO. 49) | (SEQ ID NO. 61) | | |
| H3AQC2301B12LC | 01B12H3AHC | QC2301B12VK | 1.13E−9 | 0.8 |
| | (SEQ ID NO. 49) | (SEQ ID NO. 63) | | |
| H3A02B4L1AL1 | 01B12H3AHC | 02B4L1AL1 | 2E−9 | 1.56 |
| | (SEQ ID NO. 49) | (SEQ ID NO. 77) | | |
| H3AQC2301B12L5AL2 | 01B12H3AHC | QC2301B12L5AL2 | 1.25E−8 | 9.7 |
| | (SEQ ID NO. 49) | (SEQ ID NO. 65) | | |
| H3AQC23L1A02D9L2 | 01B12H3AHC | QC23L1A02D9L2 | no | no |
| | (SEQ ID NO. 49) | (SEQ ID NO. 71) | binding | activity |
| H3AQC23L6A | 01B12H3AHC | QC23L6A | weak | 16.89 |
| | (SEQ ID NO. 49) | (SEQ ID NO. 69) | | |
| H3AQC2301B12L6AL2 | 01B12H3AHC | QC2301B12L6AL2 | weak | 10 |
| | (SEQ ID NO. 49) | (SEQ ID NO. 73) | | |
| H3AQC23L2AL1 | 01B12H3AHC | QC23L2AL1 | | 4.4 |
| | (SEQ ID NO. 49) | (SEQ ID NO. 67) | | |
| H3AQC23L2AL3 | 01B12H3AHC | QC23L2AL3 | | 2.57 |
| | (SEQ ID NO. 49) | (SEQ ID NO. 53) | | |

Based on the characteristics listed above H3AQC2301B12L1AL1L3 was renamed ZTS-00103182 will be referred to herein as ZTS-182, This antigen binding protein was further characterized, as described below.

### Example 8

### Fc Region of '182

The Fc region for ZTS-182 is a modified version of canine IgGB (Vet Immunol Immunopathol. 2014 Jan. 15; 157(1-2):31-41) SEQ ID NO. 158 and was chosen for its half-life, biophysical properties, and lack of effector functions. As reported in Bergeron et al, canine IgGB has good affinity to canine FcRn and biophysical properties suitable for downstream processing. Differential Scanning calorimetry (DSC) done on the canine Fc alone indicates thermal stabilities of the constant regions are approximately 70° C. and 83° C. These melting temperatures are similar or higher than those reported for marketed humanized mAbs.

Three point mutations were made to the CH2 domain of canine IgGB to ablate ADCC and CDC activity. The mutated Fc is referred to herein as IgGB(e-) and comprise SEQ ID NO.184. Although NGF is a soluble target, effector functions were eliminated from the anti-NGF antibody to protect against any potential non-specific target or effector-function associated adverse effects. These mutations did not appear to influence immunogenicity of this mAb. Mutations to the Fc to eliminate effector functions did not affect FcRn or Protein A binding. Decreased binding to canine FcyRI and FcyRIII were observed as well as a reduction in ADCC activity. C1q protein is the first protein in the complement cascade and is required for cells to undergo Complement Dependent Cytotoxicity (CDC). IgGB(e-) does not bind to C1q protein.

### Example 9

### Pharmacokinetic/Pharmacodynamics Analysis of Caninized ZTS-182

Pharmacokinetics were evaluated in 4-8 normal beagle dogs using a dose regimen consisting of two subcutaneous (SC) doses and one intravenous (IV) dose administered at 28 day intervals. This design has the advantage of providing absolute bioavailability data and graphically making it possible to identify spot unwanted immunogenicity and determine whether it is transient or persistent even without performing an ADA assay.

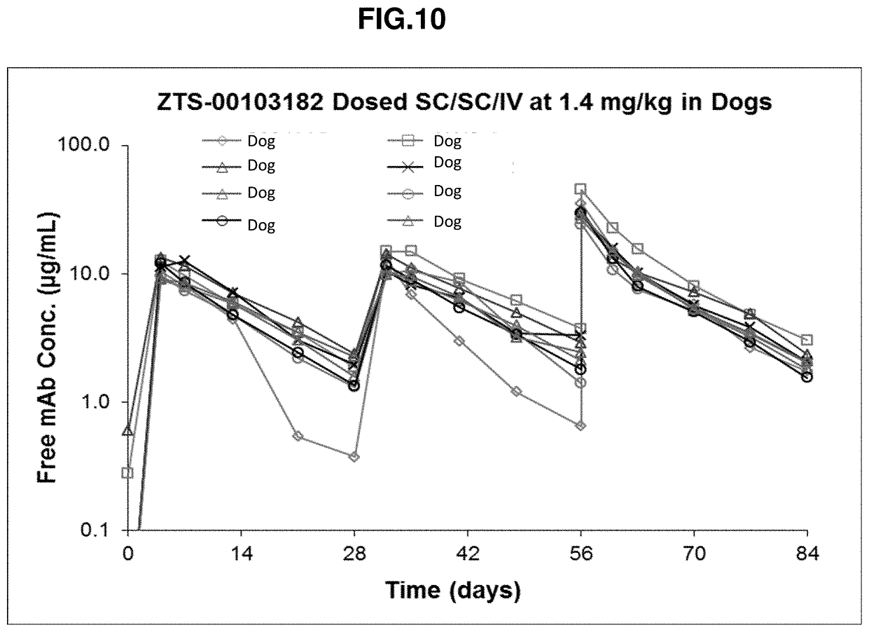

ZTS-182 was administered to four male and four female dogs at 1.4 mg/kg (FIG. 10). The subcutaneous bioavailability, which was calculated after correction for the overlap of the concentration-time profiles, averaged about 81%±13%. The clearance averaged 6.2±1.0 mL/d/kg and the terminal half-life was 9.3±0.9 days.

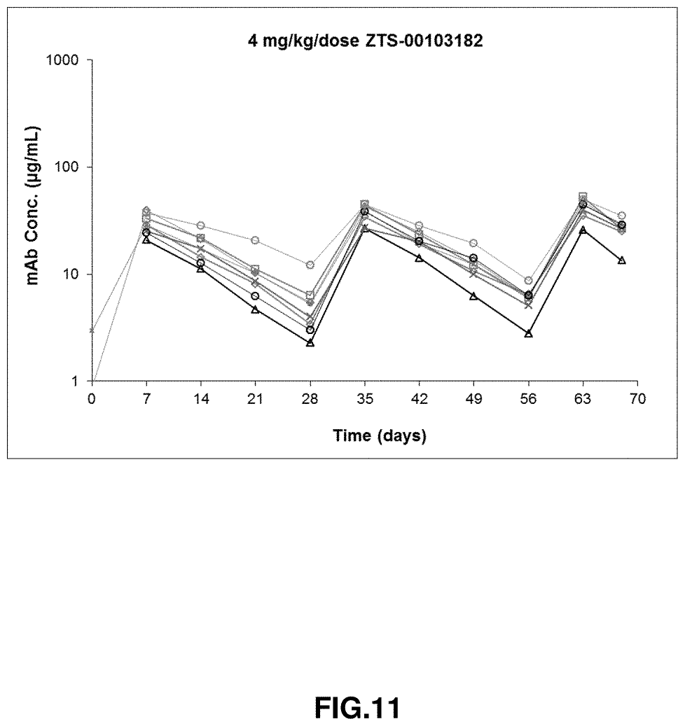

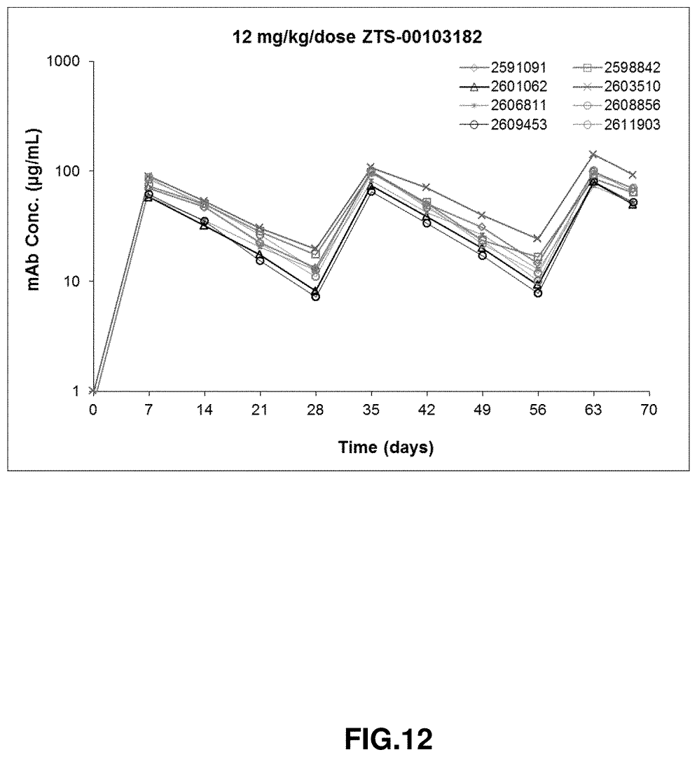

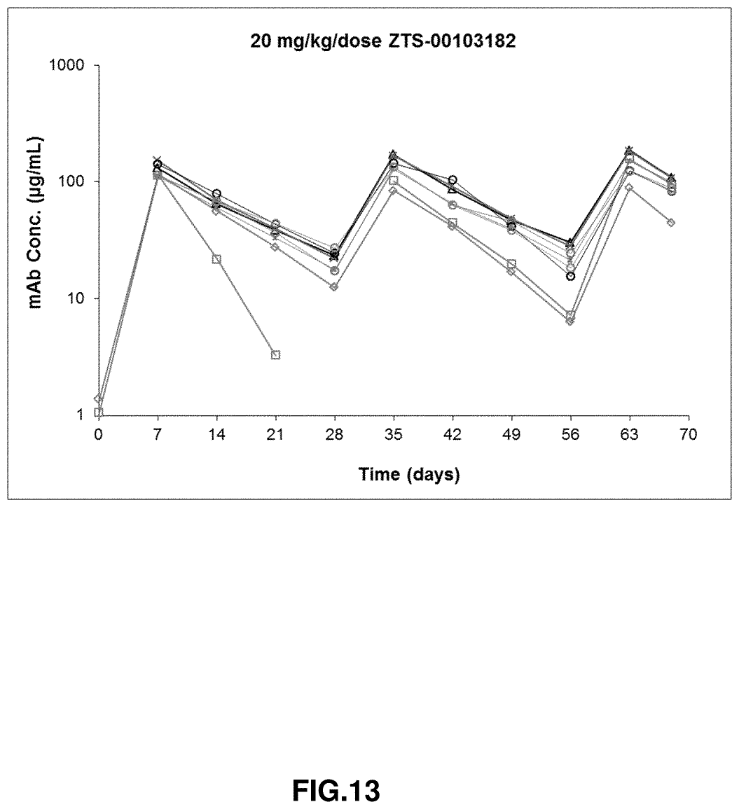

In a related study, once weekly serum samples were collected following three SC doses of ZTS-182 administered at 28 day intervals to groups of four male and four female beagle dogs at 0, 4, 12, and 20 mg/kg per dose. The pharmacokinetics at these higher doses were consistent with those in the PK study described about and repeated dosing resulted in only modest accumulation. The half-life was approximately 8 days at the two lower doses and 7 days at the highest dose (FIGS. 11-13)

Exposures to ZTS-00103182 increased in a dose-proportional manner over a wide dose range, based on the dose-normalized Cmax and AUC data tabulated below in Table 4 from several studies. No evidence of target-mediated disposition has been apparent.

TABLE 4

| Study≠ | Study #1 | | | Study #2 | Study #3 | | |
|---|---|---|---|---|---|---|---|
| Dose | (n = 4/gp) | | | (n = 8) | (n = 8/gp) | | |
| (mg/kg) | 0.2 | 1 | 2 | 1.4 | 4 | 12 | 20 |
| Cₘₐₓ ± SD | 1.6 ± 0.8 | 6.3 ± 1.5 | 9.3 ± 0.9 | 11.2 ± 1.9 | 30 ± 6 | 73 ± 13 | 126 ± 16 |
| (μg/mL) | | | | | | | |
| Dose- | 8.0 ± 3.8 | 6.3 ± 1.5 | 4.7 ± 0.5 | 8.1 ± 1.4 | 7.4 ± 1.6 | 6.1 ± 1.1 | 6.3 ± 0.8 |
| normalized | | | | | | | |
| Cₘₐₓ ± SD | | | | | | | |
| (μg/mL per | | | | | | | |
| mg/kg) | | | | | | | |
| AUC_{0-∞} ± | 19.1 ± 1.7 | 110 ± 19 | 188 ± 11 | 175 ± 29 | 494 ± 185 | 1180 ± 244 | 1926 ± 250 |
| SD (μg/mL) | | | | | | | |
| Dose- | 95 ± 9 | 110 ± 19 | 94 ± 6 | 127 ± 21 | 124 ± 46 | 98 ± 20 | 97 ± 13 |
| normalized | | | | | | | |
| AUC_{0-∞} ± | | | | | | | |
| SD (μg/mL | | | | | | | |
| per mg/kg) | | | | | | | |

### Example 10

### Directed Mutations of VL CDR2

In an effort to affect the binding and functional properties of ZTS-182 a series of rationally designed directed mutations within CDR 2 of the variable light chain were undertake. Positions 2,3, 5 and 6 were mutated as listed in Table 5 below. The VL listed below were paired with SEQ ID NO.49.

TABLE 5

| | Pos. | Pos | Pos. | Pos | Pos. | Pos. | SEQ ID |
|---|---|---|---|---|---|---|---|
| | 1 | 2 | 3 | 4. | 5 | 6 | NO. |
| wt 182 | T | S | R | L | H | S | 8 |
| m6 | T | T | R | L | Q | A | 194 |
| m43 | T | H | S | L | H | N | 196 |
| m70 | T | S | S | L | H | G | 197 |
| m72 | T | S | S | L | H | Q | 198 |
| m75 | T | S | S | L | H | V | 199 |
| m114 | T | A | S | L | H | Q | 200 |

Both binding and functional assays, as previously described herein, were performed using each of the antibodies listed in Table 5, the data for which is represented in Tables 6 (cNGF binding) and 7 (TF-1 proliferation inhibition). The sequences for the entire VL are identical to SEQ ID NO.51 with only the CDR2 amino acids, listed in Table 6, differ.

TABLE 6

| | can NGF | | |
|---|---|---|---|
| Sample Name | ka (M−1 s−1) | kd (s−1) | KD (M) |
| 182m6 | 3560 | 1.54E−08 | 4.34E−12 |
| 182m43 | 769 | 3.32E−06 | 4.32E−09 |
| 182m70 | 1930 | 9.46E−06 | 4.9E−09 |
| 182m72 | 3760 | 2.79E−07 | 7.41E−11 |
| 182m75 | 444 | 8.99E−07 | 2.03E−09 |
| 182m114 | 1790 | 1.27E−06 | 7.07E−10 |

TABLE 7

| | | | Top | | | |
|---|---|---|---|---|---|---|
| | | | Conc. | NGF | | |
| anti-NGF | IC50 | IC50 | Tested | Conc. | | |
| antibody | μg/ml | nM | μg/ml | ng/ml | comments | KD |
| 182m6** | 0.0131 | 0.087 | 0.100 | 2.0 | full dose- | 4.34E−12 |
| | | | | | response | |
| 182m43 | n/a | n/a | 0.100 | 2.0 | partial | 4.32E−09 |
| | | | | | response | |
| 182m70 | n/a | n/a | 0.100 | 2.0 | partial | 4.9E−09 |
| | | | | | response | |
| 182m72 | n/a | n/a | 0.100 | 2.0 | partial | 7.41E−11 |
| | | | | | response | |
| 182m75 | n/a | n/a | 0.100 | 2.0 | partial | 2.03E−09 |
| | | | | | response | |
| 182m114 | n/a | n/a | 0.100 | 2.0 | partial | 7.07E−10 |
| | | | | | response | |
| ZTS-182 | 0.006931 | 0.046 | 0.100 | 2.0 | full dose- | 2.7E−12 |
| | | | | | response | |

**182m6 VL (SEQ ID NO. 201)/182 VH (SEQ ID NO. 49)

### Example 11

### Neurite Outgrowth Inhibition Assays

Neurite outgrowth is a key process in the development of functional neuronal circuits and the regeneration of the nervous system. The rat pheochromocytoma-12 (PC12) cell line is derived from adrenal pheochromocytoma cells (malignant counterpart of chromaffin cells) and represents a well-established model system for investigation of neuronal differentiation and function. Treatment with soluble factors such as nerve growth factor (NGF) stimulates PC12 cells to differentiate into neuron-like cells. Treatment of PC12 cells with NGF induces activation of extracellular signal-regulated kinases 1 and 2 (ERK1/2), which are part of the mitogen-activated protein kinase (MAPK) family, via activation of the TrkA receptor. Activation of ERK1/2 leads to neurite elongation and development of neuron-like phenotypic characteristics in PC12 cells. Inhibition of NGF binding to the TrkA receptor should result in an inhibition of neurite outgrowth in this assay.

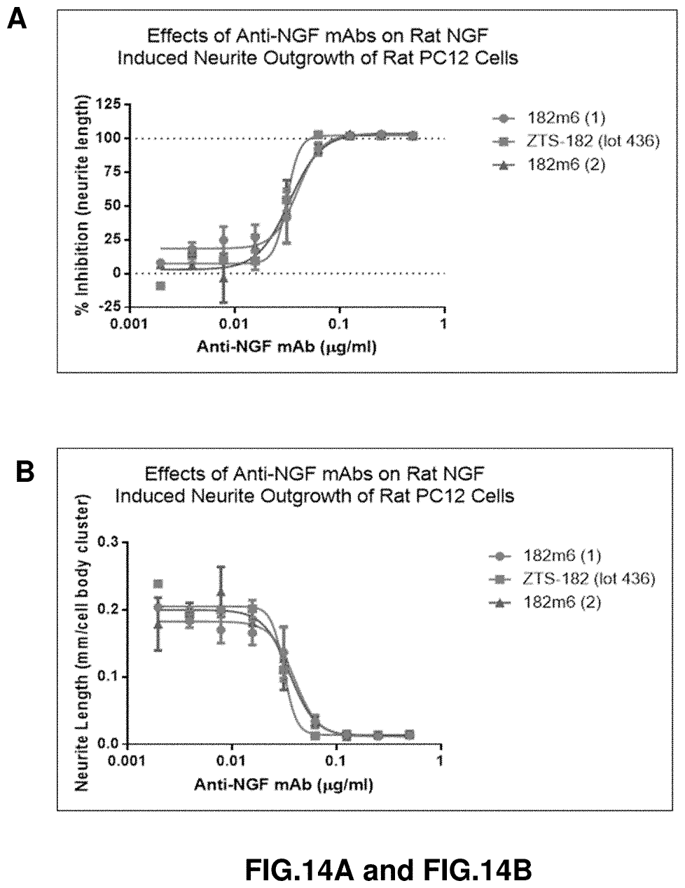

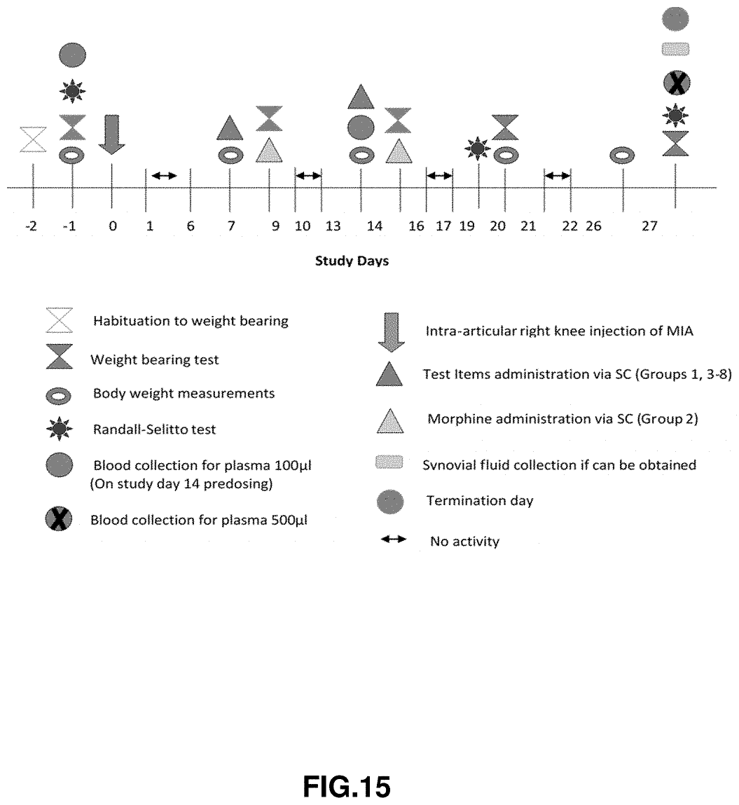

PC12 cells were maintained in a growth medium [Dulbecco's Modified Eagle's Medium (DMEM)] supplemented with 5% fetal bovine serum (Thermo Fisher Scientific, Waltham, Mass., USA), 5% horse serum (Thermo Fisher Scientific) at 37° C. For the cell growth assay or the neurite outgrowth assay, PC12 cells were seeded in growth medium at 1×10⁴ cells per well in 24-well tissue culture plates for the cell growth assay, or in collagen type IV-coated, 24-well culture plates for the neurite outgrowth assay, and allowed to grow for 24 h. The cells were then cultured in the growth medium continuously for the cell growth assay or placed in the differentiating medium (DMEM supplemented with 1% horse serum and penicillin/streptomycin) for the neurite outgrowth assay. PC12 cells were stimulated with 10 ng/ml of rat NGF and then various concentrations of either ZTS-182 or ZTS-182m6. The top concentration of antibody tested per well was 0.5 mg/ml, as shown in Table 8 below. PC12 cells were examined and measured. Inhibition of neurite outgrowth in a dose dependent fashion was observed, as shown in FIGS. 14A and 14B which shows the percent inhibition of both the ZTS-182 and the ZTS-182m6 antibodies as a function of neurite length.

TABLE 8

| | | | Top Conc. | |
|---|---|---|---|---|
| | IC50 | IC50 | Tested | Rat NGF |
| anti-NGF antibody | μg/ml | nM | μg/ml | ng/ml |
| ZTS-182 | 0.031 | 0.21 | 0.5 | 10.0 |
| 182m6 (Repeat 1) | 0.039 | 0.26 | 0.5 | 10.0 |
| 182m6 (Repeat 2) | 0.035 | 0.24 | 0.5 | 10.0 |

### Example 12

### Evaluation of Speciated Antigen Binding Protein in Rat MIA Model

Osteoarthritis (OA) is a degenerative joint disease characterized by joint pain and a progressive loss of articular cartilage. Intra-articular injection of MIA (sodium monoiodo acetate) induces loss of articular cartilage with progression of subchondral bone lesions that mimic those of OA. This model offers a rapid and minimally invasive method to reproduce OA-like lesions in a rodent species.

The analgesic effect of speciated (example caninized, felinized and the like) anti-NGF antibodies at one dose of MIA in the rat MIA model of osteoarthritis was demonstrated by dosing caninized monoclonal antibody ZTS-182 twice during the study on study day 7 and study day 14. Pain was assessed using weight bearing test for sustained pain and joint compression (Randall Selitto) test for mechanical hyperalgesia. See FIG. 14 for a schematic of the rat MIA procedure.

### Test Groups and Dose Levels.

The table below lists the experimental groups comprising the study

TABLE 9

| Group | Group | | | Dose | | Dosing | Testing |
|---|---|---|---|---|---|---|---|
| No. | Size | Treatment | Volume | (mg/kg) | Route | Regimen | Regimen |
| 1 | n = 10 | Vehicle | 1.6 | 0 | SC | Once on study days | |
| | | | ml/kg | | | 7 and 14 | |
| 2 | N = 10 | Positive | 5 | 10 | SC | Once on study days | Weight |
| | | Control | ml/kg | | | | bearing test |
| | | | | | | | on study |
| | | | | | | | days |
| | | (Morphine) | | | | 9 and 16 1 hour pre- | −1, 9, 16, 21 |
| | | | | | | weight bearing | and 28. |
| | | | | | | testing | |
| 3 | N = 10 | ZTS-182 | 0.5 | 8 | SC | Once on study days | |
| | | | ml/kg | | | | |
| 4 | N = 10 | mAb2 | 2.0 | 8 | SC | 7 and 14 | Randall- |
| | | | ml/kg | | | | Sellitto test |
| | | | | | | | on study |
| | | | | | | | days −1, 20 |
| | | | | | | | and 28. |
| 5 | N = 10 | mAb3 | 4.0 | 8 | SC | | |
| | | | ml/kg | | | | |

### Description of Study:

TABLE 10

| Study Day | Task |
|---|---|
| Day −2 | 1. Habituation to weight bearing apparatus. |
| Day −1 | 1. Body weight measurements (baseline). |
| | 2. Weight bearing (WB) test (baseline). |
| | 3. Randall-Selitto test (baseline). |
| | 4. Blood collection for plasma (baseline). 5 |
| 0 | 1. MIA intra-articular injection to right knee. |
| 1-6 | N/A |
| 7 | 1. Body weight measurements. |
| | 2. Test Items' administration (Groups 1, 3-8). |
| 9 | 1. Morphine administration (Group 2). |
| | 2. Wait 1 hour post dosing. |
| | 3. Weight bearing (WB) test. |
| 10-13 | N/A |
| 14 | 1. Body weight measurements. |
| | 2. Blood collection pre-dosing. |
| | 3. Test Items' administration (Groups 1, 3-8). |
| 16 | 1. Morphine administration (Group 2). |
| | 2. Wait 1 hour post dosing. |
| | 3. Weight bearing (WB) test. |
| 17-19 | N/A |
| 20 | 1. Randall-Selitto test. |
| 21 | 1. Body weight measurements. |
| | 2. Weight bearing test. |
| 22-26 | N/A |
| 27 | 1. Body weight measurements. |
| 28 | 1. Weight bearing test. |
| | 2. Randall-Selitto test. |
| | 3. Blood collection for plasma. |
| | 4. Synovial fluid (if can be obtained). |

Results were calculated and represented as the percentage of weight that the animal leaned on the injected right leg or intact left leg from the total amount of leaned weight on the two hind legs. The difference between the two values of intact left leg minus injected right leg was calculated. Weight bearing test measures the animal ability to carry its weight on the hind legs. In normal condition the animal carries its weight equally on both hind legs (50% on the right leg and 50% on the left leg). Therefore, the difference between the percentage of weight carried on each leg will be close to 0%. As the animal experienced pain, this situation changes. The animal tend carries more weight on the non-painful leg and less weight on the painful leg. As a result, the difference between the percentage of weight carried on both legs increased.

### Mechanical Hyperalgesia (Randall-Selitto Test):

Mechanical thresholds, expressed in grams, was measured in rats with the Randall-Selitto. The test was performed by applying a pressure to the hind paw. By pressing a pedal that activated a motor, the force increased at a constant rate on the linear scale. When pain is displayed withdrawal of the paw or vocalization is noted, the pedal was immediately released and the nociceptive threshold read on a scale.

### Body Weights:

Animals' body weight was measured at the beginning of the study (day −1) and once a week throughout the study; on days −1, 7, 14, 21 and 27.

### Statistics/Data Evaluation:

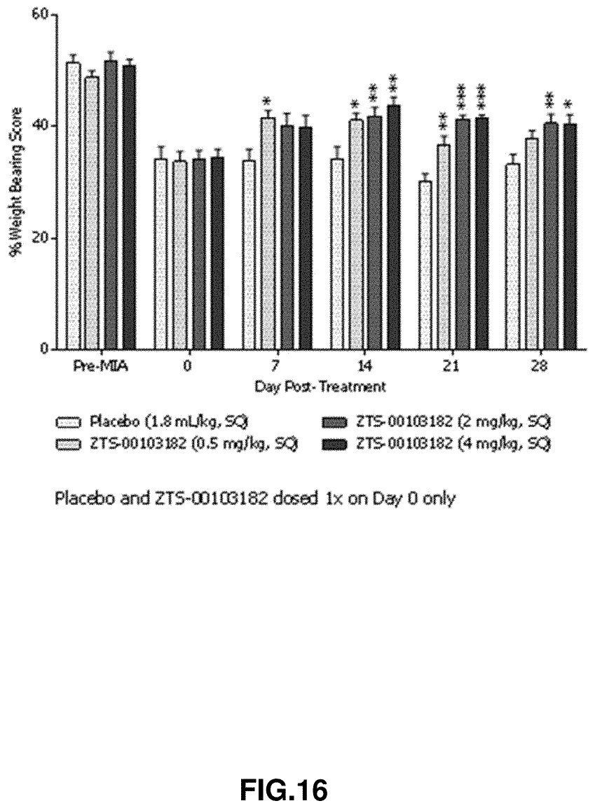

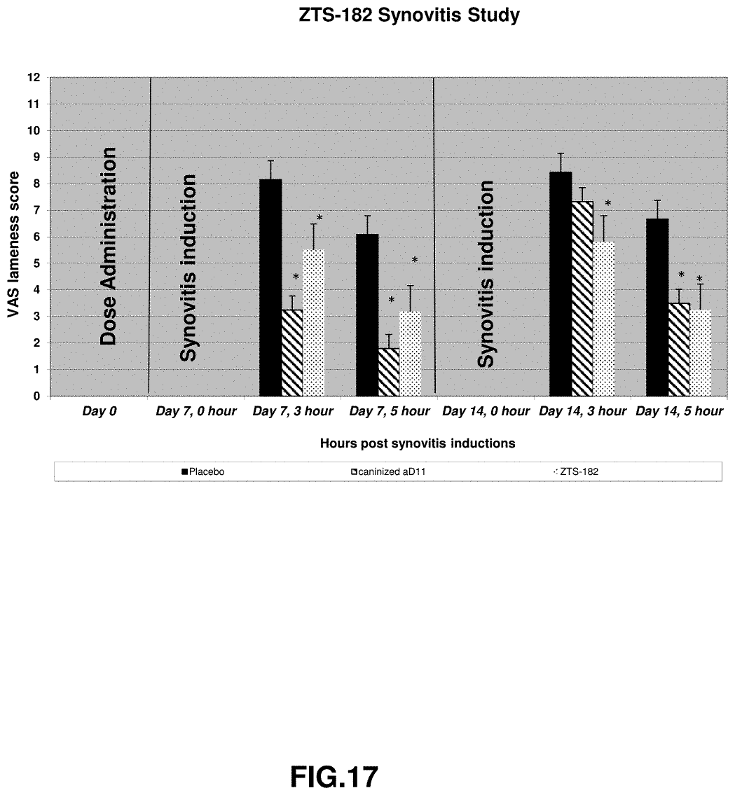

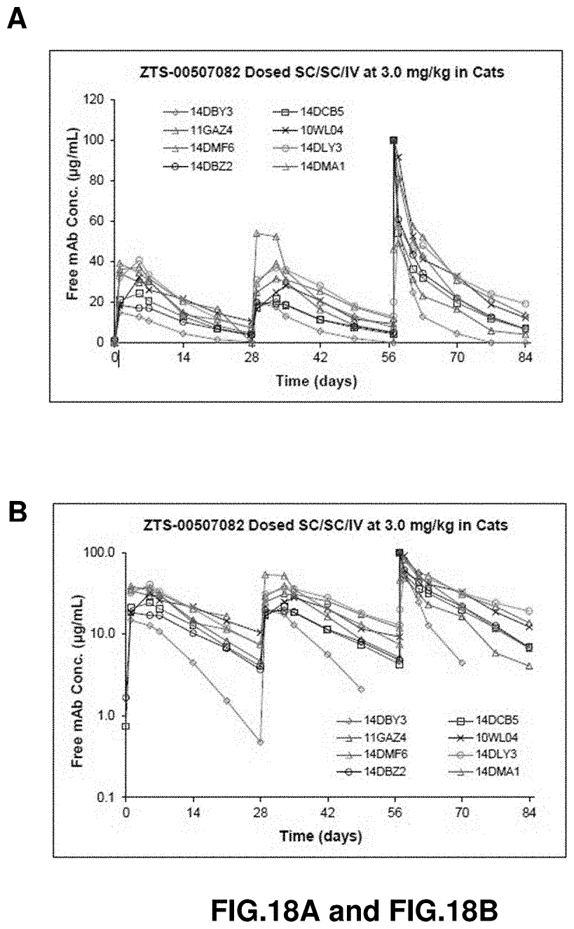

Evaluation was primarily based on the relative recorded changes in body weight bearing expressed as percentage (%) in all treated groups vs. those of the Vehicle Control. Where appropriate, analysis of the data by one-way ANOVA followed by Tukey test is applied to determine significance of treatment effects. FIG. 16 demonstrates the dose dependent positive effect of ZTS-182 on the ability of rats withstand pain by measuring weight bearing after administration of 0.5, 2.0 and 4.0 mg/kg of ZTS-182 to rats in the above noted MIA model. These results clearly demonstrates efficacy of ZTS-182 in alleviating pain.

### Example 13

### Effects on Lameness After Administration of Caninized Anti-NGF Antigen Binding Protein

Inflammatory processes in soft tissue are well recognized as one significant component of osteoarthritis. In the synovitis pain model, transient inflammation of the synovial membrane in a single stifle is induced via intra-articular injection of bacterial lipopolysaccharide (LPS). Quantifiable lameness occurs within 2 h of synovitis induction, peaks at 3-4 h, is waning by 6 h and is fully resolved after 24 h. This model has routinely been used to investigate targets for pain control.

A 5 mg/kg dose of ZTS-182 by intravenous injection administered once to intact male beagles reduced lameness, as compared to placebo and a positive control, in a canine LPS synovitis model. Efficacy was measured three and five hours post LPS synovitis induction. Synovitis induction was also conducted on Day 14 in the opposite stifle with efficacy measured three and five hours post synovitis induction.

FIG. 16 represents least squares means (with standard error) for lameness VAS for treatment groups at three, and five hours post synovitis induction on Days 7 and 14. Differences between 5 mg/kg caninized aD11 and Placebo were statistically significant on Day 7 three (p<0.001), 171 hours post dose administration and five hours (p<0.0001), as well as on Day 14 five hours (p=0.0005) post synovitis induction. Lameness assessments comparing 5 mg/kg ZTS-00103182 and Placebo on Day 7, three hours (p=0.0297) and five hours (p=0.0180) post synovitis induction were statistically significant, as were those recorded on Day 14, three hours (p=0.03130) and five hours (p=0.0057) post synovitis induction.

### Example 14

### Felinization Strategy

To help mitigate risks associated with Anti-drug Antibodies (ADA) formation in cats, the ZTS-182 canine monoclonal antibody CDR sequences were used to graft into a feline germline antibody sequence, as described herein. This felinization strategy is based on identifying the most appropriate feline germline antibody sequence for CDR grafting. Following extensive analysis of all available feline germline sequences for both the heavy and light chains, germline candidates were selected on their homology to canine ZTS-182, and the CDRs from canine ZTS-182 were used to replace native feline CDRs. The objective was to retain high affinity and cell-based activity using fully feline frameworks to minimize the potential of immunogenicity in vivo. Felinized mAbs were optimized for mammalian expression, expressed and characterized for their ability to bind NGF via SPR. These results are described below in Example 14. Only mAbs that retained both reliable expression levels and the ability to bind NGF following felinization were advanced for further characterization. Those mAbs that did not express transiently or lost the ability to bind NGF were not progressed.

### Example 15

### Felinization of Canine ZTS-182 Antibody

Recombinant constructs representing the felinized variable heavy and light chains of mAb canine ZTS-182 were made. Following subcloning of each variable chain into plasmids containing the respective feline heavy or kappa constant region, plasmids were co-transfected for antibody expression in HEK 293 cells. The feline heavy chain constant region of the present invention are not limited to any particular subtype, however in some embodiments the feline heavy chain constant region is described as fel IgG1a SEQ ID NO.162]. The feline kappa light chain constant regions are not limited to any particular sequences, however in some embodiments of the present invention the feline kappa constant region is described as SEQ ID NO. 165.

Affinities of selected felinized anti-NGF antibody to feline NGF were measured using SPR (Surface Plasmon Resonance), Biacore 3000. Kinetics of association and dissociation of a felinized mAb with/from feline NGF were measured at different concentrations. KD equilibrium binding constants are reported in Table 11.

The selected felinized mAbs were also tested in an NGF functional assay. Following pre-incubation of mAbs with NGF, a cell line expressing human TrkA (the receptor for NGF) is introduced. Activation of the receptor results in a cascade of intracellular signaling (pERK-1/2) which can be measured as an indicator of dose response for mAb inhibition of NGF binding to TrkA. Table 11 shows that the felinized mAbs all had IC₅₀s in the 1 nM range. These cell-based data are consistent with the Biacore data and thus indicate highly potent mAbs to NGF, thus circumventing the need for affinity maturation.

TABLE 11

| | Variable | | Variable | | | |
|---|---|---|---|---|---|---|
| | Heavy | | Light | | | |
| Alias | Chain | SEQ ID NO. | Chain | SEQ ID NO. | IC50 (nM) | KD (M) |
| mAb 1B (ZTS-082) | H1-23 | 85 | KPL | 87 | 1.28 | 9.41E−12 |
| H1-23L3K36 | H1-23 | 85 | L3-K36 | 89 | 1.30 | 9.16E−10 |
| H733 L3-K36 | H733 | 92 | L3-K36 | 89 | 1.13 | 3.34E−13 |
| H1-23K643 | H1-23 | 85 | K643 | 94 | 1.48 | 1.77E−10 |
| H733KPL | H733 | 92 | KPL | 87 | 1.34 | 4.58E−13 |

### Example 16

### Pharmacokinetics of Felinized Anti-NGF Antigen Binding Proteins

A group of three male and five female domestic short hair cats was dosed twice subcutaneously and once intravenously at 28 or 29 day intervals with ZTS-082 administered at 3.0 mg/kg. No mortality, adverse events, or hypersensitivity reactions occurred during the 84 day study period. Based on the concentration-time profiles, none of the animals appeared to develop anti-drug antibodies. ZTS-082 had a half-life of approximately 10.4±2.9 days. After subcutaneous administration, the bioavailability was approximately 76%±21% and peak serum concentrations were observed at 1-7 days after dosing.

‘Free’ ZTS-082 in feline serum was assayed using a sandwich ligand binding assay automated on a Gyrolab XPTM instrument. Key reagents included biotin-labeled canine NGF and Alexa Fluor®-labeled AffiniPure goat anti-cat IgG, Fc Fragment Specific. Quality control samples were prepared in feline serum. Standards covering the range 0.1-100 μg/mL were prepared daily in feline serum. The standards, QCs, and study samples were diluted 1:40 with 2% BSA in PBST and further diluted with an equal volume of Rexxip AN™ buffer (Gyros, Inc. The biotin-NGF capture agent was applied to the streptavidin-coated beads of a Gyrolab Bioaffy 200 nL CD. After washing, the samples were applied, followed by another wash, then the Alexa Fluor-anti-cat IgG detection antibody was applied, followed by another wash. The fluorescence signals were analyzed using the Gyros Evaluator by regression of the standards using a 5-parameter logistic curve. The range of quantitation was 0.391-100 μg/mL. The back-calculated concentrations of serum QCs containing 0.50, 5.0, and 50 μg/mL ZTS-082 averaged approximately 0.553, 4.65 and 50.5 μg/mL, respectively (n=4 each).

It did not appear that any of the animals dosed with ZTS-082 developed anti-drug antibodies since no unusual changes in the half-lives were observed over the course of the study (FIGS. 1)7A and B).

After intravenous administration, the clearance of ZTS-082 averaged approximately 4.0±1.2 mL/d/kg and the half-life averaged approximately 10.4±2.9 days. The subcutaneous bioavailability was approximately 76%±21% (average of both SC doses). Peak serum concentrations were observed at 1-7 days after subcutaneous administration.

What is claimed is:

1. An antigen binding protein that specifically binds Nerve Growth Factor (NGF) comprising:
a) a heavy chain variable region (VH) comprising:
i. a Complimentary Determining Region 1 (CDR1) comprising an amino acid sequence having at least about 90% sequence identity to the amino acid sequence comprising SEQ ID NO. 4;
ii. a Complimentary Determining Region 2 (CDR2) comprising an amino acid sequence having at least about 90% sequence identity to the amino acid sequence comprising SEQ ID NO. 5;
iii. a Complimentary Determining Region 3 (CDR3) comprising an amino acid sequence having at least about 90% sequence identity to the amino acid sequence comprising SEQ ID NO. 6; and
a. a light chain variable region (VL) comprising
i. a Complimentary Determining Region 1 (CDR1) comprising an amino acid sequence having at least about 90% sequence identity to the amino acid sequence comprising:
(X1)-A-S-Q-(X2)-I-(X3) -(X4) -(X5)-L-N (SEQ ID NO.177) wherein:
X1 comprises K or R,
X2 comprises S or D,
X3 comprises N or S,
X4 comprises H or N,
X5 comprises Y or N; and
ii. a Complimentary Determining Region 2 (CDR2) comprising an amino acid sequence having at least about 90% sequence identity to the amino acid sequence comprising:
Y-(X6)-S-(X7) -(X8)-H-S (SEQ ID NO. 178) wherein:
X6 comprises I or T
X7 comprises R or S
X8 comprises L or F; and
iii. a Complimentary Determining Region 3 (CDR3) comprising an amino acid sequence having at least about 90% sequence identity to the amino acid sequence comprising:
(X9)-(X10)-(X11)-(X12)-(X13)-(X14)-P-(X15)-(X16) (SEQ ID NO. 179) wherein
X9 comprises Q or H,
X10 comprises Q or R,
X11 comprises G or A,
X12 comprises D, S, T or N,
X13 comprises H, T or M,
X14 comprises F, L or S,
X15 comprises R, Y or G,
X16 comprises T or P; and
any variants thereof having one or more conservative amino acid substitutions in at least one of CDR1, CDR2 or CDR3 within any of the variable light or variable heavy chain regions of said antigen binding protein.

2. An antigen binding protein that specifically binds Nerve Growth Factor (NGF) comprising:
a. a heavy chain variable region (VH) comprising:
i. a Complimentary Determining Region 1 (CDR1) comprising an amino acid sequence having at least about 90% sequence identity to the amino acid sequence comprising SEQ ID NO. 4;
ii. a Complimentary Determining Region 2 (CDR2) comprising an amino acid sequence having at least about 90% sequence identity to the amino acid sequence comprising SEQ ID NO. 5;
iii. a Complimentary Determining Region 3 (CDR3) comprising an amino acid sequence having at least about 90% sequence identity to the amino acid sequence comprising SEQ ID NO. 6; and
b. a light chain variable region (VL) comprising
i. a Complimentary Determining Region 1 (CDR1) comprising an amino acid sequence having at least about 90% sequence identity to the amino acid sequence comprising:
(X1)-A-S-Q-(X2)-I-(X3)-(X4)-(X5)-L-N (SEQ ID NO. 180) wherein:
X1 comprises K or R,
X2 comprises S or D,
X3 comprises N or S,
X4 comprises H or N,
X5 comprises Y or N; and
ii. a Complimentary Determining Region 2 (CDR2) comprising an amino acid sequence having at least about 90% sequence identity to the amino acid sequence comprising:
T-(X6)-(X7)-L-(X8)-(X9) (SEQ ID NO. 181) wherein:
X6 comprises T, H, S or A,
X7 comprises R or S,
X8 comprises Q or H,
X9 comprises A, Q, G or V; and
iii. a Complimentary Determining Region 3 (CDR3) comprising an amino acid sequence having at least about 90% sequence identity to the amino acid sequence comprising:
(X10)-(X11)-(X12)-(X13)-(X14)-(X15)-P-(X16)-(X17) (SEQ ID NO. 182) wherein
X10 comprises Q or H,
X11 comprises Q or R,
X12 comprises G or A,
X13 comprises D, S, T or N,
X14 comprises H, T or M,
X15 comprises F, L or S,
X16 comprises R, Y or G,
X17 comprises T or P; and
any variants thereof having one or more conservative amino acid substitutions in at least one of CDR1, CDR2 or CDR3 within any of the variable light or variable heavy chain regions of said antigen binding protein.

3. An antigen binding protein that specifically binds Nerve Growth Factor (NGF) comprising:
a. a heavy chain variable region (VH) comprising:
i. a Complimentary Determining Region 1 (CDR1) comprising an amino acid sequence having at least about 90% sequence identity to the amino acid sequence comprising SEQ ID NO. 4;
ii. a Complimentary Determining Region 2 (CDR2) comprising an amino acid sequence having at least about 90% sequence identity to the amino acid sequence comprising SEQ ID NO. 5;
iii. a Complimentary Determining Region 3 (CDR3) comprising an amino acid sequence having at least about 90% sequence identity to the amino acid sequence comprising SEQ ID NO. 6; and
b. a light chain variable region (VL) selected from the group consisting of:
i. a light chain variable region comprising:
1. a CDR1 comprising an amino acid sequence having at least about 90% sequence identity to the amino acid sequence comprising SEQ ID NO. 7,
2. a CDR2 comprising an amino acid sequence having at least about 90% sequence identity to the amino acid sequence comprising SEQ ID. No. 8, and
3. a CDR3 comprising an amino acid sequence having at least about 90% sequence identity to the amino acid sequence comprising SEQ ID No.9;
ii. a light chain variable region comprising:
1. a CDR1 comprising an amino acid sequence having at least about 90% sequence identity to the amino acid sequence comprising SEQ ID NO. 10,
2. a CDR2 comprising an amino acid sequence having at least about 90% sequence identity to the amino acid sequence comprising SEQ ID. No. 11, and
3. a CDR3 comprising an amino acid sequence having at least about 90% sequence identity to the amino acid sequence comprising SEQ ID No.12;
iii. a light chain variable region comprising:
1. a CDR1 comprising an amino acid sequence having at least about 90% sequence identity to the amino acid sequence comprising SEQ ID NO. 13,
2. a CDR2 comprising an amino acid sequence having at least about 90% sequence identity to the amino acid sequence comprising SEQ ID. No. 14, and
3. a CDR3 comprising an amino acid sequence having at least about 90% sequence identity to the amino acid sequence comprising SEQ ID No.15;
iv. a light chain variable region comprising:
1. a CDR1 comprising an amino acid sequence having at least about 90% sequence identity to the amino acid sequence comprising SEQ ID NO. 16
2. a CDR2 comprising an amino acid sequence having at least about 90% sequence identity to the amino acid sequence comprising SEQ ID. No. 17
3. a CDR3 comprising an amino acid sequence having at least about 90% sequence identity to the amino acid sequence comprising SEQ ID No.18;
v. a light chain variable region comprising:
1. a CDR1 comprising an amino acid sequence having at least about 90% sequence identity to the amino acid sequence comprising SEQ ID NO. 19,
2. a CDR2 comprising an amino acid sequence having at least about 90% sequence identity to the amino acid sequence comprising f SEQ ID. No. 20, and
3. a CDR3 comprising an amino acid sequence having at least about 90% sequence identity to the amino acid sequence comprising SEQ ID No.21;
vi. a light chain variable region comprising:
1. a CDR1 comprising an amino acid sequence having at least about 90% sequence identity to the amino acid sequence comprising SEQ ID NO. 22,
2. a CDR2 comprising an amino acid sequence having at least about 90% sequence identity to the amino acid sequence comprising SEQ ID. No. 23, and
3. a CDR3 comprising an amino acid sequence having at least about 90% sequence identity to the amino acid sequence comprising SEQ ID No.24;
vii. a light chain variable region comprising:
1. a CDR1 comprising an amino acid sequence having at least about 90% sequence identity to the amino acid sequence comprising SEQ ID NO. 25,
2. a CDR2 comprising an amino acid sequence having at least about 90% sequence identity to the amino acid sequence comprising SEQ ID. No. 26, and
3. a CDR3 comprising an amino acid sequence having at least about 90% sequence identity to the amino acid sequence comprising SEQ ID No.27;
viii. a light chain variable region comprising:
1. a CDR1 comprising an amino acid sequence having at least about 90% sequence identity to the amino acid sequence comprising SEQ ID NO. 28,
2. aCDR2 comprising an amino acid sequence having at least about 90% sequence identity to the amino acid sequence comprising SEQ ID. No. 29, and
3. a CDR3 comprising an amino acid sequence having at least about 90% sequence identity to the amino acid sequence comprising SEQ ID No.30;
ix. a light chain variable region comprising:
1. a CDR1 comprising an amino acid sequence having at least about 90% sequence identity to the amino acid sequence comprising SEQ ID NO. 31,
2. a CDR2 comprising an amino acid sequence having at least about 90% sequence identity to the amino acid sequence comprising SEQ ID. No. 32, and
3. aCDR3 comprising an amino acid sequence having at least about 90% sequence identity to the amino acid sequence comprising SEQ ID No.33;
x. a light chain variable region comprising:
1. a CDR1 comprising an amino acid sequence having at least about 90% sequence identity to the amino acid sequence comprising SEQ ID NO. 34,
2. a CDR2 comprising an amino acid sequence having at least about 90% sequence identity to the amino acid sequence comprising SEQ ID. No. 35, and
3. a CDR3 comprising an amino acid sequence having at least about 90% sequence identity to the amino acid sequence comprising SEQ ID No.36;
xi. a light chain variable region comprising:
1. a CDR1 comprising an amino acid sequence having at least about 90% sequence identity to the amino acid sequence comprising SEQ ID NO. 37,
2. a CDR2 comprising an amino acid sequence having at least about 90% sequence identity to the amino acid sequence comprising SEQ ID. No. 38, and
3. a CDR3 comprising an amino acid sequence having at least about 90% sequence identity to the amino acid sequence comprising SEQ ID No.39;
xii. a light chain variable region comprising:
1. a CDR1 comprising an amino acid sequence having at least about 90% sequence identity to the amino acid sequence comprising SEQ ID NO. 40,
2. a CDR2 comprising S an amino acid sequence having at least about 90% sequence identity to the amino acid sequence comprising SEQ ID. No. 41, and
3. a CDR3 comprising an amino acid sequence having at least about 90% sequence identity to the amino acid sequence comprising SEQ ID No.42;
xiii. a light chain variable region comprising:
1. a CDR1 comprising an amino acid sequence having at least about 90% sequence identity to the amino acid sequence comprising SEQ ID NO. 43,
2. a CDR2 comprising an amino acid sequence having at least about 90% sequence identity to the amino acid sequence comprising SEQ ID. No. 44, and
3. a CDR3 comprising an amino acid sequence having at least about 90% sequence identity to the amino acid sequence comprising SEQ ID No.45; and
xiv. a light chain variable region comprising:
1. a CDR1 comprising an amino acid sequence having at least about 90% sequence identity to the amino acid sequence comprising SEQ ID NO. 46,
2. a CDR2 comprising an amino acid sequence having at least about 90% sequence identity to the amino acid sequence comprising SEQ ID. No. 47, and
3. a CDR3 comprising an amino acid sequence having at least about 90% sequence identity to the amino acid sequence comprising SEQ ID No.48; and
xv. a light chain variable region comprising:
1. a CDR1 comprising an amino acid sequence having at least about 90% sequence identity to the amino acid sequence comprising SEQ ID NO. 193,
2. a CDR2 comprising an amino acid sequence having at least about 90% sequence identity to the amino acid sequence comprising SEQ ID. No. 194, and
3. a CDR3 comprising an amino acid sequence having at least about 90% sequence identity to the amino acid sequence comprising SEQ ID No.195; and
any variants thereof having one or more conservative amino acid substitutions in at least one of CDR1, CDR2 or CDR3 within any of the variable light or variable heavy chain regions of said antigen binding protein.

4. The antigen binding protein of claim 3 wherein
a. the variable heavy chain comprises:
i. a CDR1 comprising an amino acid sequence having at least about 90% sequence identity to the amino acid sequence comprising SEQ ID NO. 4,
ii. a CDR2 comprising an amino acid sequence having at least about 90% sequence identity to the amino acid sequence comprising SEQ ID NO. 5, and
iii. a CDR3 comprising an amino acid sequence having at least about 90% sequence identity to the amino acid sequence comprising SEQ ID NO. 6; and
b. the variable light chain comprises:
i. a CDR1 comprising an amino acid sequence having at least about 90% sequence identity to the amino acid sequence comprising SEQ ID NO. 7,
ii. a CDR2 comprising an amino acid sequence having at least about 90% sequence identity to the amino acid sequence comprising SEQ ID NO. 8,
iii. a CDR3 comprising an amino acid sequence having at least about 90% sequence identity to the amino acid sequence comprising SEQ ID NO. 9; and
any variants thereof having one or more conservative amino acid substitutions in at least one of CDR1, CDR2 or CDR3 within the variable light or variable heavy chain regions of said antigen binding protein.

5. The antigen binding protein of claim 3 wherein
a. the variable heavy chain comprises:
i. a CDR1 comprising an amino acid sequence having at least about 90% sequence identity to the amino acid sequence comprising SEQ ID NO. 4
ii. a CDR2 comprising an amino acid sequence having at least about 90% sequence identity to the amino acid sequence comprising SEQ ID NO. 5, and
iii. a CDR3 comprising an amino acid sequence having at least about 90% sequence identity to the amino acid sequence comprising SEQ ID NO. 6; and
b. the variable light chain comprises:
i. a CDR1 comprising an amino acid sequence having at least about 90% sequence identity to the amino acid sequence comprising SEQ ID NO. 13,
ii. a CDR2 comprising an amino acid sequence having at least about 90% sequence identity to the amino acid sequence comprising SEQ ID NO. 14,
iii. a CDR3 comprising an amino acid sequence having at least about 90% sequence identity to the amino acid sequence comprising SEQ ID NO. 15; and
any variants thereof having one or more conservative amino acid substitutions in at least one of CDR1, CDR2 or CDR3 within the variable light or variable heavy chain regions of said antigen binding protein.

6. The antigen binding protein of claim 3 wherein
a. the variable heavy chain comprises:
i. a CDR1 comprising an amino acid sequence having at least about 90% sequence identity to the amino acid sequence comprising SEQ ID NO. 4
ii. a CDR2 comprising an amino acid sequence having at least about 90% sequence identity to the amino acid sequence comprising SEQ ID NO. 5, and
iii. a CDR3 comprising an amino acid sequence having at least about 90% sequence identity to the amino acid sequence comprising SEQ ID NO. 6; and
b. the variable light chain comprises:
i. a CDR1 comprising an amino acid sequence having at least about 90% sequence identity to the amino acid sequence comprising SEQ ID NO. 167,
ii. a CDR2 comprising an amino acid sequence having at least about 90% sequence identity to the amino acid sequence comprising SEQ ID NO. 168,
iii. a CDR3 comprising an amino acid sequence having at least about 90% sequence identity to the amino acid sequence comprising SEQ ID NO. 169; and
any variants thereof having one or more conservative amino acid substitutions in at least one of CDR1, CDR2 or CDR3 within the variable light or variable heavy chain regions of said antigen binding protein.

7. An antigen binding protein that specifically binds to Nerve Growth Factor (NGF) which comprises:
a. a heavy chain variable region (VH) having at least 90% sequence identity to the amino acid sequences comprising SEQ ID NO. 49; and
b. a light chain variable region (VL) having at least 90% sequence identity to the amino acid sequences selected from the group consisting of: SEQ ID NO.51; SEQ ID NO. 53; SEQ ID NO. 55; SEQ ID NO.57; SEQ ID NO. 59; SEQ ID NO.61; SEQ ID NO.63; SEQ ID NO. 65; SEQ ID NO.67; and SEQ ID NO. 175; and
any variants thereof having one or more conservative amino acid substitutions in at least one of the heavy chain variable regions or one of the light chain variable regions of said antigen binding protein.

8. The antigen binding protein of claim 3 wherein said antigen binding protein is speciated.

9. The antigen binding protein of claim 8 wherein the speciated antigen binding protein is a caninized, felinized or humanized antigen binding protein.

10. The antigen binding protein of claim 9 wherein the speciated antigen binding protein is caninized.

11. The antigen binding protein of claim 9 wherein the speciated antigen binding protein is felinized.

12. The antigen binding protein of claim 9 wherein the speciated antigen binding protein is humanized.

13. The antigen binding protein of any one of claims 3 wherein said antigen binding inhibits the binding between NGF and TrkA.

14. The antigen binding protein of claim 3 wherein said binding protein reduces or eliminates an NGF-related disorder.

15. The antigen binding protein of claim 14 wherein the NGF-related disorder is selected from the group consisting of: cardiovascular diseases, atherosclerosis, obesity, type 2 diabetes, metabolic syndrome, pain and inflammation.

16. The antigen binding protein of claim 15 wherein the NGF-related disorder is pain.

17. The antigen binding protein of claim 15, wherein said NGF-related disorder is a pain disorder and is selected from the group consisting of: osteoarthritis pain, rheumatoid arthritis pain, surgical and postsurgical pain, incisional pain, general inflammatory pain, cancer pain, pain from trauma, neuropathic pain, neuralgia, diabetic neuropathy pain, pain associated with rheumatic diseases, pain associated with musculoskeletal diseases, visceral pain, and gastrointestinal pain.

18. The antigen binding protein of claim 17 wherein the NGF-related disorder comprises osteoarthritis pain.

19. The antigen binding protein of claim 3 wherein said binding protein is selected from the group consisting of: a monoclonal antibody; a chimeric antibody, a single chain antibody, a tetrameric antibody, a tetravalent antibody, a multispecific antibody, a domain-specific antibody, a domain-deleted antibody, a fusion protein, an ScFc fusion protein, an Fab fragment, an Fab′ fragment, an F(ab′)₂ fragment, an Fv fragment, an ScFv fragment, an Fd fragment, a single domain antibody, a dAb fragment, a small modular immunopharmaceutical (SMIP) a nanobody, and IgNAR molecule.

20. The antigen binding protein of claim 19, wherein said antigen binding protein is a monoclonal antibody.

21. A pharmaceutical composition comprising a therapeutically effective amount of the antigen binding protein of claim 3, and a pharmaceutically acceptable carrier.

22. A host cell that produces the antigen binding protein of claim 3.

23. An isolated nucleic acid comprising a nucleic acid sequence encoding the antigen binding protein of claim 3 and any variants thereof having one or more nucleic acid substitutions resulting in the coding of conservative amino acid substitutions.

24. An isolated nucleic acid comprising a nucleic acid sequence having at least about 90% sequence identity to the nucleic acid sequence selected from the group consisting of: SEQ ID NO.50; SEQ ID NO.52; SEQ ID NO.54; SEQ ID NO.56; SEQ ID NO. 58; SEQ ID NO.60; SEQ ID NO.62; SEQ ID NO.64; SEQ ID NO. 66; SEQ ID NO.68; SEQ ID NO.86; SEQ ID NO.88; SEQ ID NO.90; SEQ ID NO.93; SEQ ID NO. 95; and SEQ ID NO. 176; and any variants thereof having one or more nucleic acid substitutions resulting in the coding of conservative amino acid substitutions.

25. A vector comprising the nucleic acid sequence of claim 24.

26. A host cell comprising the vector of claim 25.

27. A host cell comprising the nucleic acid of claim 24.

28. A method of producing the antigen binding protein of claim 3 comprising culturing the host cell of either one of claim 26 or 27 under conditions that result in production of the antigen binding protein, and isolating the antigen binding protein from the host cell or culture medium of the host cell.

29. A method of treating a subject for an NGF-related disorder comprising administering a therapeutically effective amount of the pharmaceutical composition of claim 21.

30. The method of claim 29 wherein the NGF-related disorder is selected from the group consisting of: cardiovascular diseases, atherosclerosis, obesity, type 2 diabetes, metabolic syndrome, pain and inflammation.

31. The method of claim 30 wherein the NGF-related disorder is pain.

32. The method of claim 31 wherein said NGF-related disorder is a pain disorder and is selected from the group consisting of: osteoarthritis pain, rheumatoid arthritis pain, surgical and postsurgical pain, incisional pain, general inflammatory pain, cancer pain, pain from trauma, neuropathic pain, neuralgia, diabetic neuropathy pain, pain associated with rheumatic diseases, pain associated with musculoskeletal diseases, visceral pain, and gastrointestinal pain.

33. The method of claim 32 wherein the NGF-related disorder comprises osteoarthritis pain.

34. A method of inhibiting NGF activity in a subject by administering the pharmaceutical composition of claim 21.

35. The method of claim 34 wherein the subject is selected from the group consisting of: canines, felines and humans.

36. The method of claim 35 wherein the subject comprises canines.

37. The method of claim 35 wherein the subject comprises felines.

38. The method of claim 35 wherein the subject comprises humans.


SEQUENCE LISTING (185 sequences)

SEQ ID NO: 1 - protein, 240 aa, human
MSMLFYTLITAFLIGIQAEPHSESNVPAGHTIPQAHWTKLQHSLDTALRRARSAPAAAIAARVAGQTRNITVDPRLFKKRRLRSPRVLFSTQPPREAADTQDLDFEVGGAAPFNRTHRSKRSSSHPIFHRGEFSVCDSVSVWVGDKTTATDIKGKEVMVLGEVNINNSVFKQYFFETKCRDPNPVDSGCRGIDSKHWNSYCTTTHTFVKALTMDGKQAAWRFIRIDTACVCVLSRKAVRR

SEQ ID NO: 2 - protein, 241 aa, CANINE
MSMLFYTLITALLIGIRAEPHPESHVPAGHAIPHAHWTKLQHSLDTALRRARSAPAGAIAARVTGQTRNITVDPKLFKKRRLRSPRVLFSTHPPPVAADAQDLDLEAGSTASVNRTHRSKRSSSHPVFHRGEFSVCDSVSVWVGDKTTATDIKGKEVMVLGEVNINNSVFKQYFFETKCRDPTPVDSGCRGIDSKHWNSYCTTTHTFVKALTMDGKQAAWRFIRIDTACVCVLSRKAGRRA

SEQ ID NO: 3 - protein, 241 aa, FELINE
MSMLSYTLITALLIGIQAEPHPESNVPAGHAIPQAHWTKLQHSLDTALRRARSTPAGAIAARVAGQTRNITVDPKLFKKRRLRSPRVLFSTHPPPVAADTQGLDLEAGGAASFNRTHRSKRSSSHPVFHRGEFSVCDSVSVWVGDKTTATDIKGKEVMVLGEVNINNSVFKQYFFETKCRDPTPVDSGCRGIDSKHWNSYCTTTHTFVKALTMDGKQAAWRFIRIDTACVCVLSRKAGRRA

SEQ ID NO: 4 - protein, 8 aa, MURINE
GFTLTQYG

SEQ ID NO: 5 - protein, 9 aa, MURINE
VIWATGATD

SEQ ID NO: 6 - protein, 13 aa, MURINE
DGWWYATSWYFDV

SEQ ID NO: 7 - protein, 13 aa, ARTIFICIAL
  RECOMBINANT
DGWWYATSWYFDV

SEQ ID NO: 8 - protein, 7 aa, ARTIFICIAL
  RECOMBINANT
YTSRLHS

SEQ ID NO: 9 - protein, 9 aa, ARTIFICIAL
  RECOMBINANT
QQGDHFPRT

SEQ ID NO: 10 - protein, 11 aa, ARTIFICIAL
  RECOMBINANT
RASQSISNNLN

SEQ ID NO: 11 - protein, 7 aa, ARTIFICIAL
  RECOMBINANT
YISSFHS

SEQ ID NO: 12 - protein, 9 aa, ARTIFICIAL
  RECOMBINANT
QQGDHFPYT

SEQ ID NO: 13 - protein, 11 aa, ARTIFICIAL
  RECOMBINANT
KASQDINHYLN

SEQ ID NO: 14 - protein, 7 aa, ARTIFICIAL
  RECOMBINANT
YTSSLHS

SEQ ID NO: 15 - protein, 9 aa, ARTIFICIAL
  RECOMBINANT
QQGDHFPRT

SEQ ID NO: 16 - protein, 11 aa, ARTIFICIAL
  RECOMBINANT
KASQSINHYLN

SEQ ID NO: 17 - protein, 7 aa, ARTIFICIAL
  RECOMBINANT
YTSRLHS

SEQ ID NO: 18 - protein, 9 aa, ARTIFICIAL
  RECOMBINANT
QQGSTLPRT

SEQ ID NO: 19 - protein, 11 aa, ARTIFICIAL
  RECOMBINANT
RASQDISNYLN

SEQ ID NO: 20 - protein, 7 aa, ARTIFICIAL
  RECOMBINANT
YTSRLHS

SEQ ID NO: 21 - protein, 9 aa, ARTIFICIAL
  RECOMBINANT
HRATTSPGP

SEQ ID NO: 22 - protein, 11 aa, ARTIFICIAL
  RECOMBINANT
KASQDINHYLN

SEQ ID NO: 23 - protein, 7 aa, ARTIFICIAL
  RECOMBINANT
YTSRLHS

SEQ ID NO: 24 - protein, 9 aa, ARTIFICIAL
  RECOMBINANT
QQGSTLPRT

SEQ ID NO: 25 - protein, 11 aa, ARTIFICIAL
  RECOMBINANT
RASQDISNYLN

SEQ ID NO: 26 - protein, 7 aa, ARTIFICIAL
  RECOMBINANT
YTSRLHS

SEQ ID NO: 27 - protein, 9 aa, ARTIFICIAL
  RECOMBINANT
QQGSTLPRT

SEQ ID NO: 28 - protein, 11 aa, ARTIFICIAL
  RECOMBINANT
RASQDISNYLN

SEQ ID NO: 29 - protein, 8 aa, ARTIFICIAL
  RECOMBINANT
YYTSSLHS

SEQ ID NO: 30 - protein, 9 aa, ARTIFICIAL
  RECOMBINANT
QQGSTLPRT

SEQ ID NO: 31 - protein, 11 aa, ARTIFICIAL
  RECOMBINANT
KASQSINHYLN

SEQ ID NO: 32 - protein, 7 aa, ARTIFICIAL
  RECOMBINANT
YISSFHS

SEQ ID NO: 33 - protein, 9 aa, ARTIFICIAL
  RECOMBINANT
QQSHTLPYT

SEQ ID NO: 34 - protein, 11 aa, ARTIFICIAL
  RECOMBINANT
KASQDINHYLN

SEQ ID NO: 35 - protein, 7 aa, ARTIFICIAL
  RECOMBINANT
YVTTLHA

SEQ ID NO: 36 - protein, 9 aa, ARTIFICIAL
  RECOMBINANT
QQGDHFPRT

SEQ ID NO: 37 - protein, 11 aa, ARTIFICIAL
  RECOMBINANT
RASQDISNYLN

SEQ ID NO: 38 - protein, 7 aa, ARTIFICIAL
  RECOMBINANT
KTNSLQT

SEQ ID NO: 39 - protein, 9 aa, ARTIFICIAL
  RECOMBINANT
QQGSTLPRT

SEQ ID NO: 40 - protein, 11 aa, ARTIFICIAL
  RECOMBINANT
RASQDISNYLN

SEQ ID NO: 41 - protein, 7 aa, ARTIFICIAL
  RECOMBINANT
YVTSLHA

SEQ ID NO: 42 - protein, 9 aa, ARTIFICIAL
  RECOMBINANT
QQGSTLPRT

SEQ ID NO: 43 - protein, 11 aa, ARTIFICIAL
  RECOMBINANT
RASQDISNYLN

SEQ ID NO: 44 - protein, 7 aa, ARTIFICIAL
  RECOMBINANT
YTSRLHS

SEQ ID NO: 45 - protein, 9 aa, ARTIFICIAL
  RECOMBINANT
QQGNMFPYT

SEQ ID NO: 46 - protein, 11 aa, ARTIFICIAL
  RECOMBINANT
KASQDINHYLN

SEQ ID NO: 47 - protein, 7 aa, ARTIFICIAL
  RECOMBINANT
YTSRLHS

SEQ ID NO: 48 - protein, 9 aa, ARTIFICIAL
  RECOMBINANT
QQGNMFPYT

SEQ ID NO: 49 - protein, 181 aa, ARTIFICIAL
  RECOMBINANT
EVQLVESGGDLARPGGSLKLSCVVSGFTLTQYGINWVRQAPGKGLQWVTVIWATGATDYNSALKSRFTVSRDNAMNTVYLQMNSLRVEDTAVYYCARDGWWYATSWYFDVWGQGTLVTVSSASTTAPSVFPLAPSCGSTSGSTVALACLVSGYFPEPVTVSWNSGSLTSGVHTFPSVLQSS

SEQ ID NO: 50 - DNA, 425 bp, ARTIFICIAL
  RECOMBINANT
ccaccatgaagcacctgtggttctttctgctgctggtggccgctcccagatgggtgctgagcgaggtgcagctggtggaatctggcggcgacctggccagacctggcggcagcctgaagctgagctgcgtggtgtccggcttcaccctgacccagtacggcatcaactgggtccgccaggcccctggcaagggcctgcagtgggtcacagtgatctgggccaccggcgccaccgactacaacagcgccctgaagtcccggttcaccgtgtctcgggacaacgccatgaacaccgtgtacctgcagatgaacagcctgcgggtggaagataccgccgtgtactactgcgccagagacggctggtggtacgccaccagctggtacttcgacgtgtggggccagggcacactggtcacagtctcgagc

SEQ ID NO: 51 - protein, 102 aa, ARTIFICIAL
  RECOMBINANT
DIVMTQTPLSLSVSPGEPASISCKASQDINHYLNWFRQKPDGTVKLLIYYTSRLHSGVPSRFSGSGSGTDFTLRISRVEADDAGVYYCQQGDHFPRTFGQGT

SEQ ID NO: 52 - DNA, 639 bp, ARTIFICIAL
  RECOMBINANT
gatattgtgatgacccagaccccgctgagcctgagcgtgagcccgggcgaaccggcgagcattagctgcaaagcgagccaggatattaaccattatctgaactggtttcgccagaaaccggatggcaccgtgaaactgctgatttattataccagccgcctgcatagcggcgtgccgagccgctttagcggcagcggcagcggcaccgattttaccctgcgcattagccgcgtggaagcggatgatgcgggcgtgtattattgccagcagggcgatcattttccgcgcacctttggccagggcaccaaactggaaattaaacgcaacgatgcgcagccggcggtgtatctgtttcagccgagcccggatcagctgcataccggcagcgcgagcgtggtgtgcctgctgaacagcttttatccgaaagatattaacgtgaaatggaaagtggatggcgtgattcaggataccggcattcaggaaagcgtgaccgaacaggataaagatagcacctatagcctgagcagcaccctgaccatgagcagcaccgaatatctgagccatgaactgtatagctgcgaaattacccataaaagcctgccgagcaccctgattaaaagctttcagcgcagcgaatgc

SEQ ID NO: 53 - protein, 102 aa, ARTIFICIAL
  RECOMBINANT
DIVMTQTPLSLSVSPGEPASISCRASQSISNNLNWFRQKPDGTVKLLIYYISSFHSGVPSRFSGSGSGTDFTLRISRVEADDAGVYYCQQGDHFPYTFGQGT

SEQ ID NO: 54 - DNA, 372 bp, ARTIFICIAL
  RECOMBINANT
aagcttgccaccatggtgctgcagacacaggtgttcatctctctgctgctgtggattagtggagcctacggcgacatcgtgatgacccagacacctctgtcactgagcgtgtccccaggggaacccgcctctatcagttgccgggccagccagagcatcagcaacaacctgaactggttcagacagaagccagatgggaccgtcaagctactgatctactacatcagctcgttccacagcggagtgccctctcgcttttcaggcagcgggtccggaacagactttactctgcggatctccagagtggaagccgacgatgctggcgtgtactattgccagcagggcgaccacttcccctacaccttcggccag

SEQ ID NO: 55 - protein, 102 aa, ARTIFICIAL
  RECOMBINANT
DIVMTQTPLSLSVSPGEPASISCKASQDINHYLNWFRQKPDGTVKLLIYYTSSLHSGVPSRFSGSGSGTDFTLRISRVEADDAGVYYCQQGDHFPRTFGQGT

SEQ ID NO: 56 - DNA, 425 bp, ARTIFICIAL; identical to SEQ ID NO: 50
  RECOMBINANT

SEQ ID NO: 57 - protein, 102 aa, ARTIFICIAL
  RECOMBINANT
DIVMTQTPLSLSVSPGEPASISCKASQSINHYLNWFRQKPDGTVKLLIYYTSRLHSGVPSRFSGSGSGTDFTLRISRVEADDAGVYYCQQGSTLPRTFGQGT

SEQ ID NO: 58 - DNA, 372 bp, ARTIFICIAL
  RECOMBINANT
gccaccatggtgctgcagacacaggtgttcatctctctgctgctgtggattagtggagcctacggcgacatcgtgatgacccagacacctctgtcactgagcgtgtccccaggggaacccgcctctatcagttgcaaggccagccagagcatcaaccactacctgaactggttcagacagaagccagatgggaccgtcaagctactgatctactacacatcaaggctgcattcaggagtgccctctcgcttttcaggcagcgggtccggaacagactttactctgcggatctccagagtggaagccgacgatgctggcgtgtactattgccaacaggggagtaccctgcccaggaccttcggccagggtacc

SEQ ID NO: 59 - protein, 101 aa, ARTIFICIAL
  RECOMBINANT
DIVMTQTPLSLSVSPGEPASISCRASQDISNYLNWFRQKPDGTVKLLIYYTSRLHSGVPSRFSGSGSGTDFTLRISRVEADDAGVYYCHRATTSPGPSARV

SEQ ID NO: 60 - DNA, 365 bp, ARTIFICIAL
  RECOMBINANT
gccaccatggtgctgcagacacaggtgttcatctctctgctgctgtggattagtggagcctacggcgacatcgtgatgacccagacacctctgtcactgagcgtgtccccaggggaacccgcctctatcagttgcagagcttctcaagatattagcaactatctgaattggttcagacagaagccagatgggaccgtcaagctactgatctactacacatcaaggctgcattcaggagtgccctctcgcttttcaggcagcgggtccggaacagactttactctgcggatctccagagtggaagccgacgatgctggcgtgtactattgccacagggcgaccacttcccccggaccttcggccag

SEQ ID NO: 61 - protein, 102 aa, ARTIFICIAL; identical to SEQ ID NO: 57
  RECOMBINANT

SEQ ID NO: 62 - DNA, 366 bp, ARTIFICIAL
  RECOMBINANT
gccaccatggtgctgcagacacaggtgttcatctctctgctgctgtggattagtggagcctacggcgacatcgtgatgacccagacacctctgtcactgagcgtgtccccaggggaacccgcctctatcagttgcagagcttctcaagatattagcaactatctgaattggttcagacagaagccagatgggaccgtcaagctactgatctactacacatcaaggctgcattcaggagtgccctctcgcttttcaggcagcgggtccggaacagactttactctgcggatctccagagtggaagccgacgatgctggcgtgtactattgccaacaggggagtaccctgcccaggaccttcggccag

SEQ ID NO: 63 - protein, 102 aa, ARTIFICIAL
  RECOMBINANT
DIVMTQTPLSLSVSPGEPASISCRASQDISNYLNWFRQKPDGTVKLLIYYTSRLHSGVPSRFSGSGSGTDFTLRISRVEADDAGVYYCQQGSTLPRTFGQGT

SEQ ID NO: 64 - DNA, 366 bp, ARTIFICIAL; identical to SEQ ID NO: 62
  RECOMBINANT

SEQ ID NO: 65 - protein, 102 aa, ARTIFICIAL
  RECOMBINANT
DIVMTQTPLSLSVSPGEPASISCRASQDISNYLNWFRQKPDGTVKLLIYYTSSLHSGVPSRFSGSGSGTDFTLRISRVEADDAGVYYCQQGSTLPRTFGQGT

SEQ ID NO: 66 - DNA, 639 bp, ARTIFICIAL
  RECOMBINANT
gatattgtgatgacccagaccccgctgagcctgagcgtgagcccgggcgaaccggcgagcattagctgccgcgcgagccaggatattagcaactatctgaactggtttcgccagaaaccggatggcaccgtgaaactgctgatttattataccagcagcctgcatagcggcgtgccgagccgctttagcggcagcggcagcggcaccgattttaccctgcgcattagccgcgtggaagcggatgatgcgggcgtgtattattgccagcagggcagcaccctgccgcgcacctttggccagggcaccaaactggaaattaaacgcaacgatgcgcagccggcggtgtatctgtttcagccgagcccggatcagctgcataccggcagcgcgagcgtggtgtgcctgctgaacagcttttatccgaaagatattaacgtgaaatggaaagtggatggcgtgattcaggataccggcattcaggaaagcgtgaccgaacaggataaagatagcacctatagcctgagcagcaccctgaccatgagcagcaccgaatatctgagccatgaactgtatagctgcgaaattacccataaaagcctgccgagcaccctgattaaaagctttcagcgcagcgaatgc

SEQ ID NO: 67 - protein, 102 aa, ARTIFICIAL
  RECOMBINANT
DIVMTQTPLSLSVSPGEPASISCKASQSINHYLNWFRQKPDGTVKLLIYYISSFHSGVPSRFSGSGSGTDFTLRISRVEADDAGVYYCQQSHTLPYTFGQGT

SEQ ID NO: 68 - DNA, 366 bp, ARTIFICIAL
  RECOMBINANT
gccaccatggtgctgcagacacaggtgttcatctctctgctgctgtggattagtggagcctacggcgacatcgtgatgacccagacacctctgtcactgagcgtgtccccaggggaacccgcctctatcagttgcaaggccagccagagcatcaaccactacctgaactggttcagacagaagccagatgggaccgtcaagctactgatctactacatcagctcgttccacagcggagtgccctctcgcttttcaggcagcgggtccggaacagactttactctgcggatctccagagtggaagccgacgatgctggcgtgtactattgccagcagagccacaccctgccctacaccttcggccag

SEQ ID NO: 69 - protein, 102 aa, ARTIFICIAL
  RECOMBINANT
DIVMTQTPLSLSVSPGEPASISCKASQDINHYLNWFRQKPDGTVKLLIYYVTSLHAGVPSRFSGSGSGTDFTLRISRVEADDAGVYYCQQGDHFPRTFGQGT

SEQ ID NO: 70 - DNA, 639 bp, ARTIFICIAL
  RECOMBINANT
gatattgtgatgacccagaccccgctgagcctgagcgtgagcccgggcgaaccggcgagcattagctgcaaagcgagccaggatattaaccattatctgaactggtttcgccagaaaccggatggcaccgtgaaactgctgatttattatgtgaccagcctgcatgcgggcgtgccgagccgctttagcggcagcggcagcggcaccgattttaccctgcgcattagccgcgtggaagcggatgatgcgggcgtgtattattgccagcagggcgatcattttccgcgcacctttggccagggcaccaaactggaaattaaacgcaacgatgcgcagccggcggtgtatctgtttcagccgagcccggatcagctgcataccggcagcgcgagcgtggtgtgcctgctgaacagcttttatccgaaagatattaacgtgaaatggaaagtggatggcgtgattcaggataccggcattcaggaaagcgtgaccgaacaggataaagatagcacctatagcctgagcagcaccctgaccatgagcagcaccgaatatctgagccatgaactgtatagctgcgaaattacccataaaagcctgccgagcaccctgattaaaagctttcagcgcagcgaatgc

SEQ ID NO: 71 - protein, 102 aa, ARTIFICIAL
  RECOMBINANT
DIVMTQTPLSLSVSPGEPASISCRASQDISNYLNWFRQKPDGTVKLLIYKTNSLQTGVPSRFSGSGSGTDFTLRISRVEADDAGVYYCQQGSTLPRTFGQGT

SEQ ID NO: 72 - DNA, 639 bp, ARTIFICIAL
  RECOMBINANT
gatattgtgatgacccagaccccgctgagcctgagcgtgagcccgggcgaaccggcgagcattagctgccgcgcgagccaggatattagcaactatctgaactggtttcgccagaaaccggatggcaccgtgaaactgctgatttataaaaccaacagcctgcagaccggcgtgccgagccgctttagcggcagcggcagcggcaccgattttaccctgcgcattagccgcgtggaagcggatgatgcgggcgtgtattattgccagcagggcagcaccctgccgcgcacctttggccagggcaccaaactggaaattaaacgcaacgatgcgcagccggcggtgtatctgtttcagccgagcccggatcagctgcataccggcagcgcgagcgtggtgtgcctgctgaacagcttttatccgaaagatattaacgtgaaatggaaagtggatggcgtgattcaggataccggcattcaggaaagcgtgaccgaacaggataaagatagcacctatagcctgagcagcaccctgaccatgagcagcaccgaatatctgagccatgaactgtatagctgcgaaattacccataaaagcctgccgagcaccctgattaaaagctttcagcgcagcgaatgc

SEQ ID NO: 73 - protein, 102 aa, ARTIFICIAL
  RECOMBINANT
DIVMTQTPLSLSVSPGEPASISCRASQDISNYLNWFRQKPDGTVKLLIYYVTSLHAGVPSRFSGSGSGTDFTLRISRVEADDAGVYYCQQGSTLPRTFGQGT

SEQ ID NO: 74 - DNA, 639 bp, ARTIFICIAL
  RECOMBINANT
gatattgtgatgacccagaccccgctgagcctgagcgtgagcccgggcgaaccggcgagcattagctgccgcgcgagccaggatattagcaactatctgaactggtttcgccagaaaccggatggcaccgtgaaactgctgatttattatgtgaccagcctgcatgcgggcgtgccgagccgctttagcggcagcggcagcggcaccgattttaccctgcgcattagccgcgtggaagcggatgatgcgggcgtgtattattgccagcagggcagcaccctgccgcgcacctttggccagggcaccaaactggaaattaaacgcaacgatgcgcagccggcggtgtatctgtttcagccgagcccggatcagctgcataccggcagcgcgagcgtggtgtgcctgctgaacagcttttatccgaaagatattaacgtgaaatggaaagtggatggcgtgattcaggataccggcattcaggaaagcgtgaccgaacaggataaagatagcacctatagcctgagcagcaccctgaccatgagcagcaccgaatatctgagccatgaactgtatagctgcgaaattacccataaaagcctgccgagcaccctgattaaaagctttcagcgcagcgaatgc

SEQ ID NO: 75 - protein, 102 aa, ARTIFICIAL
  RECOMBINANT
EIVMTQSPASLSLSQEEKVTITCRASQDISNYLNWYQQKPGQAPKLLIYYTSRLHSGVPSRFSGSGSGTDFSFTISSLEPEDVAVYYCQQGNMFPYTFGGGT

SEQ ID NO: 76 - DNA, 367 bp, ARTIFICIAL
  RECOMBINANT
ggccaccatgagtgttcctacccaagtgctgggactgctgctgctgtggctgacagatgctcggtgcgagatagtcatgacccagtcaccggcatctctgagcctgagccaggaagagaaggtaactatcacgtgtcgggcctctcaggacattagcaactacctgaattggtatcagcagaaaccaggacaggcccccaagttgttgatatactacacttcccgcctgcacagtggggtcccctcccgattcagcggatccgggtccggcacggacttcagctttactatctccagtttggagcccgaagatgttgctgtgtattactgtcagcagggtaatatgtttccgtatacattcggcgga

SEQ ID NO: 77 - protein, 102 aa, ARTIFICIAL
  RECOMBINANT
EIVMTQSPASLSLSQEEKVTITCKASQDINHYLNWYQQKPGQAPKLLIYYTSRLHSGVPSRFSGSGSGTDFSFTISSLEPEDVAVYYCQQGNMFPYTFGGGT

SEQ ID NO: 78 - DNA, 378 bp, ARTIFICIAL
  RECOMBINANT
agcttggccaccatgagtgttcctacccaagtgctgggactgctgctgctgtggctgacagatgctcggtgcgagatagtcatgacccagtcaccggcatctctgagcctgagccaggaagagaaggtaactatcacgtgtaaggccagccaggacatcaaccactacctgaactggtatcagcagaaaccaggacaggcccccaagttgttgatatactacacttcccgcctgcacagtggggtcccctcccgattcagcggatccgggtccggcacggacttcagctttactatctccagtttggagcccgaagatgttgctgtgtattactgtcagcagggtaatatgtttccgtatacattcggcggaggtacc

SEQ ID NO: 79 - protein, 8 aa, ARTIFICIAL
  RECOMBINANT
GFTLTQYG

SEQ ID NO: 80 - protein, 9 aa, ARTIFICIAL
  RECOMBINANT
VIWATGATD

SEQ ID NO: 81 - protein, 13 aa, ARTIFICIAL
  RECOMBINANT
DGWWYATSWYFDV

SEQ ID NO: 82 - protein, 11 aa, ARTIFICIAL
  RECOMBINANT
KASQDINHYLN

SEQ ID NO: 83 - protein, 7 aa, ARTIFICIAL
  RECOMBINANT
YTSRLHS

SEQ ID NO: 84 - protein, 9 aa, ARTIFICIAL
  RECOMBINANT
QQGDHFPRT

SEQ ID NO: 85 - protein, 121 aa, ARTIFICIAL
  RECOMBINANT
DVQLVESGGDLVQPGGSLRLTCVASGFTLTQYGINWVRQAPGKGLQWVAVIWATGATDYNSALKSRFTISRDNAKNTLYLQMNSLKTEDTATYYCARDGWWYATSWYFDVWGQGALVTVSS

SEQ ID NO: 86 - DNA, 420 bp, ARTIFICIAL
  RECOMBINANT
atggagtggtcttgggtctttctgttctttctgagtgttaccaccggcgtgcactcagacgtgcagctggtggaatctggcggcgacctggtgcagcctggcggctctctgagactgacctgcgtggcctccggcttcaccctgacccagtacggcatcaactgggtgcgacaggcccctggcaagggcctgcagtgggtggccgtgatctgggccaccggcgccaccgactacaactccgccctgaagtcccggttcaccatcagccgggacaacgccaagaacaccctgtacctgcagatgaactccctgaaaaccgaggacaccgccacctactactgcgccagggacggctggtggtacgccacctcctggtacttcgacgtgtggggccagggcgctctggtgacagtctcgagc

SEQ ID NO: 87 - protein, 102 aa, ARTIFICIAL
  RECOMBINANT
DIVMTQTPLSLSVTPGEPASISCKASQDINHYLNWYLQKPDGTVKLLIYYTSRLHSGVPSRFSGSGSGTDFTLRISRVEADDVGVYYCQQGDHFPRTFGPGT

SEQ ID NO: 88 - DNA, 366 bp, ARTIFICIAL
  RECOMBINANT
atgagtgttcctacccaagtgctgggactgctgctgctgtggctgacagatgctcggtgcgacatcgtgatgacccagaccccactgtccctgtccgtgacacctggcgagcctgcctccatctcctgcaaggcctcccaggacatcaaccactacctgaactggtatctgcagaagcccgacggcaccgtgaagctgctgatctactacacctcccggctgcactccggcgtgccctccagattctccggctctggctccggcaccgacttcaccctgcggatctcccgggtggaagccgacgacgtgggcgtgtactactgccagcagggcgaccacttcccccggacctttggccctggtacc

SEQ ID NO: 89 - protein, 102 aa, ARTIFICIAL
  RECOMBINANT
DIVMTQTPLSLPVTPGEPASISCKASQDINHYLNWYLQKPGQSPRLLIYYTSRLHSGVPSRFSGSGSGTDFTLRISSVEADDVGVYYCQQGDHFPRTFGQGT

SEQ ID NO: 90 - DNA, 366 bp, ARTIFICIAL
  RECOMBINANT
atgagtgttcctacccaagtgctgggactgctgctgctgtggctgacagatgctcggtgcgacatcgtgatgacccagacaccactgtctctgcctgtaactccgggagaaccagccagcattagttgtaaggctagccaggacatcaaccactatctgaactggtatctgcagaaacctggccaatcaccgcgcctgctgatctattacacctctcgactgcattctggagtcccatccaggttctcagggtccgggtccggcactgacttcaccttgcgcatatcttcagtggaagccgatgacgtcggagtttactattgtcaacagggcgaccactttccacggacattcggacagggtacc

SEQ ID NO: 91 - DNA, 366 bp, ARTIFICIAL; identical to SEQ ID NO: 90
  RECOMBINANT

SEQ ID NO: 92 - protein, 121 aa, ARTIFICIAL
  RECOMBINANT
DVQLVESGGDLVKPGGSLRLTCVASGFTLTQYGINWVRQAPGKGLQWVAVIWATGATDYNSALKSRFTMSRDNARNTLYLQMNSLKTEDTATYYCARDGWWYATSWYFDVWGQGTLVTVSS

SEQ ID NO: 93 - DNA, 420 bp, ARTIFICIAL
  RECOMBINANT
atggagtggtcttgggtctttctgttctttctgagtgttaccaccggcgtgcactcagacgtgcagctggtggaatctggcggcgacctggtgaaacctggcggctctctgagactgacctgcgtggcctccggcttcaccctgacccagtacggcatcaactgggtgcgacaggcccctggcaagggcctgcagtgggtggccgtgatctgggccaccggcgccaccgactacaactccgccctgaagtcccggttcaccatgagccgggacaacgcccggaacaccctgtacctgcagatgaactccctgaaaaccgaggacaccgccacctactactgcgccagggacggctggtggtacgccacctcctggtacttcgacgtgtggggccagggcaccctggtgacagtctcgagc

SEQ ID NO: 94 - protein, 102 aa, ARTIFICIAL
  RECOMBINANT
EIQMTQSPSSLSASPGDRVTITCKASQDINHYLNWYQQKPGKVPKLLIYYTSRLHSGVPSRFSGSGSGTDFTLTISSLEPEDAATYYCQQGDHFPRTFGGGT

SEQ ID NO: 95 - DNA, 366 bp, ARTIFICIAL
  RECOMBINANT
atgagtgttcctacccaagtgctgggactgctgctgctgtggctgacagatgctcggtgcgagatccagatgacccagtccccatcctccctgtccgcctctcccggcgacagagtgacaatcacatgcaaggcctcccaggacatcaaccactacctgaactggtatcagcagaagcccggcaaagtgcctaagctgctgatctactacacctcccggctgcactccggcgtgccctccagattctccggctctggctccggcaccgacttcaccctgaccatctccagcctggaacccgaggacgccgccacctactactgccagcagggcgaccacttcccccggacctttggcggaggtacc

SEQ ID NO: 96 - protein, 121 aa, ARTIFICIAL
  RECOMBINANT
EVKLQESGPGLVAPSQSLSITCTVSGFSLTGYGVNWVRQPPGKGLEWLGMIWGDGSTDYNSALKSRLSISKDNSKSQVFLKMNSLQTDDTARYYCARDGYYYGTTWYFDVWGAGTTVTVSS

SEQ ID NO: 97 - DNA, 363 bp, ARTIFICIAL
  RECOMBINANT
gaagtgaaactgcaggaaagcggcccgggcctggtggcgccgagccagagcctgagcattacctgcaccgtgagcggctttagcctgaccggctatggcgtgaactgggtgcgccagccgccgggcaaaggcctggaatggctgggcatgatttggggcgatggcagcaccgattataacagcgcgctgaaaagccgcctgagcattagcaaagataacagcaaaagccaggtgtttctgaaaatgaacagcctgcagaccgatgataccgcgcgctattattgcgcgcgcgatggctattattatggcaccacctggtattttgatgtgtggggcgcgggcaccaccgtgaccgtgagcagc

SEQ ID NO: 98 - protein, 119 aa, ARTIFICIAL
  RECOMBINANT
QVQLKESGPGLVAPSQSLSITCTVSGFSLTGYGVNWVRQPPGKGLEWLGMIWGDGSTDYNSALKSRLSISKDNSKSQVFLKMNSLQTDDTARYYCARDGYYYGTTWYFDVWGAGTTVTV

SEQ ID NO: 99 - DNA, 357 bp, ARTIFICIAL
  RECOMBINANT
caggtgcagctgaaagaaagcggcccgggcctggtggcgccgagccagagcctgagcattacctgcaccgtgagcggctttagcctgaccggctatggcgtgaactgggtgcgccagccgccgggcaaaggcctggaatggctgggcatgatttggggcgatggcagcaccgattataacagcgcgctgaaaagccgcctgagcattagcaaagataacagcaaaagccaggtgtttctgaaaatgaacagcctgcagaccgatgataccgcgcgctattattgcgcgcgcgatggctattattatggcaccacctggtattttgatgtgtggggcgcgggcaccaccgtgaccgtg

SEQ ID NO: 100 - protein, 102 aa, ARTIFICIAL
  RECOMBINANT
DIVMTQSTSSLSASLGDRVTISCRASQDISNYLNWYQQKPDGTIKLLIYYTSRLHSGVPSRFSGSGSGTDYSLTISNLEQEDIATYFCQQGSTLPRTFGGGT

SEQ ID NO: 101 - DNA, 306 bp, ARTIFICIAL
  RECOMBINANT
gatattgtgatgacccagagcaccagcagcctgagcgcgagcctgggcgatcgcgtgaccattagctgccgcgcgagccaggatattagcaactatctgaactggtatcagcagaaaccggatggcaccattaaactgctgatttattataccagccgcctgcatagcggcgtgccgagccgctttagcggcagcggcagcggcaccgattatagcctgaccattagcaacctggaacaggaagatattgcgacctatttttgccagcagggcagcaccctgccgcgcacctttggcggcggcacc

SEQ ID NO: 102 - protein, 102 aa, ARTIFICIAL; identical to SEQ ID NO: 100
  RECOMBINANT

SEQ ID NO: 103 - DNA, 306 bp, ARTIFICIAL; identical to SEQ ID NO: 101
  RECOMBINANT

SEQ ID NO: 104 - protein, 120 aa, ARTIFICIAL
  RECOMBINANT
EVKLEESGPGLVAPSQSLSITCTVSGFSLTGYGVNWVRQPPGKGLEWLGMIWGDGSTDYNSALKSRLNISKDNSKSQVFLKMDSLQTDDTARYYCARGGYDYDVPFFDYWGQGTTLTVSS

SEQ ID NO: 105 - DNA, 360 bp, ARTIFICIAL
  RECOMBINANT
gaagtgaaactggaagaaagcggcccgggcctggtggcgccgagccagagcctgagcattacctgcaccgtgagcggctttagcctgaccggctatggcgtgaactgggtgcgccagccgccgggcaaaggcctggaatggctgggcatgatttggggcgatggcagcaccgattataacagcgcgctgaaaagccgcctgaacattagcaaagataacagcaaaagccaggtgtttctgaaaatggatagcctgcagaccgatgataccgcgcgctattattgcgcgcgcggcggctatgattatgatgtgccgttttttgattattggggccagggcaccaccctgaccgtgagcagc

SEQ ID NO: 106 - protein, 120 aa, ARTIFICIAL; identical to SEQ ID NO: 104
  RECOMBINANT

SEQ ID NO: 107 - DNA, 360 bp, ARTIFICIAL; identical to SEQ ID NO: 105
  RECOMBINANT

SEQ ID NO: 108 - protein, 102 aa, ARTIFICIAL
  RECOMBINANT
DIVMTQTTSSLSASLGDRVTISCRASQDISNYLNWYQQKPDGTVKLLIYYTSRLHSGVPSRFSGSGSGTDYSLTISNLEQEDIATYFCQQGNMFPYTLGGGT

SEQ ID NO: 109 - DNA, 306 bp, ARTIFICIAL
  RECOMBINANT
gatattgtgatgacccagaccaccagcagcctgagcgcgagcctgggcgatcgcgtgaccattagctgccgcgcgagccaggatattagcaactatctgaactggtatcagcagaaaccggatggcaccgtgaaactgctgatttattataccagccgcctgcatagcggcgtgccgagccgctttagcggcagcggcagcggcaccgattatagcctgaccattagcaacctggaacaggaagatattgcgacctatttttgccagcagggcaacatgtttccgtataccctgggcggcggcacc

SEQ ID NO: 110 - protein, 102 aa, ARTIFICIAL; identical to SEQ ID NO: 108
  RECOMBINANT

SEQ ID NO: 111 - DNA, 306 bp, ARTIFICIAL; identical to SEQ ID NO: 109
  RECOMBINANT

SEQ ID NO: 112 - protein, 121 aa, ARTIFICIAL
  RECOMBINANT
EVQLEQSGPGLVAPSQSLSITCTVSGFSLTGYGVNWVRQPPGKGLEWLGMIWGDGSTDYNSALKSRLSISKDNSKSQVFLKMNSLQTDDTARYYCARDGYYYGTTWYFDVWGAGTTVTVSS

SEQ ID NO: 113 - DNA, 363 bp, ARTIFICIAL
  RECOMBINANT
gaagtgcagctggaacagagcggcccgggcctggtggcgccgagccagagcctgagcattacctgcaccgtgagcggctttagcctgaccggctatggcgtgaactgggtgcgccagccgccgggcaaaggcctggaatggctgggcatgatttggggcgatggcagcaccgattataacagcgcgctgaaaagccgcctgagcattagcaaagataacagcaaaagccaggtgtttctgaaaatgaacagcctgcagaccgatgataccgcgcgctattattgcgcgcgcgatggctattattatggcaccacctggtattttgatgtgtggggcgcgggcaccaccgtgaccgtgagcagc

SEQ ID NO: 114 - protein, 102 aa, ARTIFICIAL
  RECOMBINANT
DIVLTQSTSSLSASLGDRVTISCRASQDISNYLNWYQQKPDGTIKLLIYYTSRLHSGVPSRFSGSGSGTDYSLTISNLEQEDIATYFCQQGSTLPRTFGGGT

SEQ ID NO: 115 - DNA, 306 bp, ARTIFICIAL
  RECOMBINANT
gatattgtgctgacccagagcaccagcagcctgagcgcgagcctgggcgatcgcgtgaccattagctgccgcgcgagccaggatattagcaactatctgaactggtatcagcagaaaccggatggcaccattaaactgctgatttattataccagccgcctgcatagcggcgtgccgagccgctttagcggcagcggcagcggcaccgattatagcctgaccattagcaacctggaacaggaagatattgcgacctatttttgccagcagggcagcaccctgccgcgcacctttggcggcggcacc

SEQ ID NO: 116 - protein, 114 aa, ARTIFICIAL
  RECOMBINANT
EVQLQESGAELVKPGASVKLSCKASGYTFTNYWMHWVKQRPGQGLEWIGHIDPSDGETHYNQKFKDKATLTVDKSSSTAYMQLTGLTSEDSAVYYCARFLPDYWGQGTSVTVSS

SEQ ID NO: 117 - DNA, 342 bp, ARTIFICIAL
  RECOMBINANT
gaagtgcagctgcaggaaagcggcgcggaactggtgaaaccgggcgcgagcgtgaaactgagctgcaaagcgagcggctatacctttaccaactattggatgcattgggtgaaacagcgcccgggccagggcctggaatggattggccatattgatccgagcgatggcgaaacccattataaccagaaatttaaagataaagcgaccctgaccgtggataaaagcagcagcaccgcgtatatgcagctgaccggcctgaccagcgaagatagcgcggtgtattattgcgcgcgctttctgccggattattggggccagggcaccagcgtgaccgtgagcagc

SEQ ID NO: 118 - protein, 114 aa, ARTIFICIAL; identical to SEQ ID NO: 116
  RECOMBINANT

SEQ ID NO: 119 - DNA, 342 bp, ARTIFICIAL; identical to SEQ ID NO: 117
  RECOMBINANT

SEQ ID NO: 120 - protein, 103 aa, ARTIFICIAL
  RECOMBINANT
DIVLTQTPAIMSASPGEKVTMTCRASSSVSSIYLHWYQQKPGSSPKLWIYSTSNLASGVPARFSGSGSGTSYSLTVSSVEAEDAATYYCQLYDNSPLTFGAGT

SEQ ID NO: 121 - DNA, 309 bp, ARTIFICIAL
  RECOMBINANT
gatattgtgctgacccagaccccggcgattatgagcgcgagcccgggcgaaaaagtgaccatgacctgccgcgcgagcagcagcgtgagcagcatttatctgcattggtatcagcagaaaccgggcagcagcccgaaactgtggatttatagcaccagcaacctggcgagcggcgtgccggcgcgctttagcggcagcggcagcggcaccagctatagcctgaccgtgagcagcgtggaagcggaagatgcggcgacctattattgccagctgtatgataacagcccgctgacctttggcgcgggcacc

SEQ ID NO: 122 - protein, 103 aa, ARTIFICIAL; identical to SEQ ID NO: 120
  RECOMBINANT

SEQ ID NO: 123 - DNA, 309 bp, ARTIFICIAL; identical to SEQ ID NO: 121
  RECOMBINANT

SEQ ID NO: 124 - protein, 121 aa, ARTIFICIAL
  RECOMBINANT
EVQLEESGPGLVAPSQSLSITCTVSGFSLTGYGVNWVRQPPGKGLEWLGMIWGDGSTDYNSALKSRLSISKDNSKSQVFLKMNSLQTDDTARYYCARDGYYYGTTWYFDVWGAGTTVTVSS

SEQ ID NO: 125 - DNA, 363 bp, ARTIFICIAL
  RECOMBINANT
gaagtgcagctggaagaaagcggcccgggcctggtggcgccgagccagagcctgagcattacctgcaccgtgagcggctttagcctgaccggctatggcgtgaactgggtgcgccagccgccgggcaaaggcctggaatggctgggcatgatttggggcgatggcagcaccgattataacagcgcgctgaaaagccgcctgagcattagcaaagataacagcaaaagccaggtgtttctgaaaatgaacagcctgcagaccgatgataccgcgcgctattattgcgcgcgcgatggctattattatggcaccacctggtattttgatgtgtggggcgcgggcaccaccgtgaccgtgagcagc

SEQ ID NO: 126 - protein, 107 aa, ARTIFICIAL
  RECOMBINANT
DIVITQTPLSLPVSLGDQASISCRSSQSIVHINRHTYLGWYLQKPGQSLKLLIYGVSNRFSGVPDRFSGSGSGTDFTLKISRVEAEDMGVYYCFQGTHVPFTFGSGT

SEQ ID NO: 127 - DNA, 321 bp, ARTIFICIAL
  RECOMBINANT
gatattgtgattacccagaccccgctgagcctgccggtgagcctgggcgatcaggcgagcattagctgccgcagcagccagagcattgtgcatattaaccgccatacctatctgggctggtatctgcagaaaccgggccagagcctgaaactgctgatttatggcgtgagcaaccgctttagcggcgtgccggatcgctttagcggcagcggcagcggcaccgattttaccctgaaaattagccgcgtggaagcggaagatatgggcgtgtattattgctttcagggcacccatgtgccgtttacctttggcagcggcacc

SEQ ID NO: 128 - protein, 107 aa, ARTIFICIAL; identical to SEQ ID NO: 126
  RECOMBINANT

SEQ ID NO: 129 - DNA, 321 bp, ARTIFICIAL; identical to SEQ ID NO: 127
  RECOMBINANT

SEQ ID NO: 130 - protein, 120 aa, ARTIFICIAL
  RECOMBINANT
EVKLEESGPGLVAPSQSLSITCTVSGFSLTGYGVNWVRQPPGKGLEWLGMIWGDGSTDYNSALKSRLSISKDNSKSQVFLKMNSLQTDDTARYYCARGGYDYDVSFFDYWGQGTTLTVSS

SEQ ID NO: 131 - DNA, 360 bp, ARTIFICIAL
  RECOMBINANT
gaagtgaaactggaagaaagcggcccgggcctggtggcgccgagccagagcctgagcattacctgcaccgtgagcggctttagcctgaccggctatggcgtgaactgggtgcgccagccgccgggcaaaggcctggaatggctgggcatgatttggggcgatggcagcaccgattataacagcgcgctgaaaagccgcctgagcattagcaaagataacagcaaaagccaggtgtttctgaaaatgaacagcctgcagaccgatgataccgcgcgctattattgcgcgcgcggcggctatgattatgatgtgagcttttttgattattggggccagggcaccaccctgaccgtgagcagc

SEQ ID NO: 132 - protein, 120 aa, ARTIFICIAL; identical to SEQ ID NO: 130
  RECOMBINANT

SEQ ID NO: 133 - DNA, 360 bp, ARTIFICIAL; identical to SEQ ID NO: 131
  RECOMBINANT

SEQ ID NO: 134 - protein, 102 aa, ARTIFICIAL
  RECOMBINANT
DIVLTQTTSSLSASLGDRVTISCRASQDISNYLNWYQQKPDGTVKLLIYYTSRFHSGVPSRFSGSGSGTDYSLTISNLEHEDIATYFCQQGNTLPYTFGGGT

SEQ ID NO: 135 - DNA, 306 bp, ARTIFICIAL
  RECOMBINANT
gatattgtgctgacccagaccaccagcagcctgagcgcgagcctgggcgatcgcgtgaccattagctgccgcgcgagccaggatattagcaactatctgaactggtatcagcagaaaccggatggcaccgtgaaactgctgatttattataccagccgctttcatagcggcgtgccgagccgctttagcggcagcggcagcggcaccgattatagcctgaccattagcaacctggaacatgaagatattgcgacctatttttgccagcagggcaacaccctgccgtatacctttggcggcggcacc

SEQ ID NO: 136 - protein, 102 aa, ARTIFICIAL; identical to SEQ ID NO: 134
  RECOMBINANT

SEQ ID NO: 137 - DNA, 306 bp, ARTIFICIAL; identical to SEQ ID NO: 135
  RECOMBINANT

SEQ ID NO: 138 - protein, 121 aa, ARTIFICIAL
  RECOMBINANT
QVKLEESGPGLVAPSQSLSITCTVSGFSLTGYGVNWVRQPPGKGLEWLGMIWGDGSTDYNSALKSRLSISKDNSKSQVFLKMNSLQTDDTARYYCARDGYYYGTTWYFDVWGAGTTVTVSS

SEQ ID NO: 139 - DNA, 363 bp, ARTIFICIAL
  RECOMBINANT
caggtgaaactggaagaaagcggcccgggcctggtggcgccgagccagagcctgagcattacctgcaccgtgagcggctttagcctgaccggctatggcgtgaactgggtgcgccagccgccgggcaaaggcctggaatggctgggcatgatttggggcgatggcagcaccgattataacagcgcgctgaaaagccgcctgagcattagcaaagataacagcaaaagccaggtgtttctgaaaatgaacagcctgcagaccgatgataccgcgcgctattattgcgcgcgcgatggctattattatggcaccacctggtattttgatgtgtggggcgcgggcaccaccgtgaccgtgagcagc

SEQ ID NO: 140 - protein, 102 aa, ARTIFICIAL
  RECOMBINANT
DIVLTQTTSSLSASLGDRVTISCRASQDISNYLNWYQQKPDGTIKLLIYYTSRLHSGVPSRFSGSGSGTDYSLTISNLEQEDIATYFCQQGSTLPRTFGGGT

SEQ ID NO: 141 - DNA, 306 bp, ARTIFICIAL
  RECOMBINANT
gatattgtgctgacccagaccaccagcagcctgagcgcgagcctgggcgatcgcgtgaccattagctgccgcgcgagccaggatattagcaactatctgaactggtatcagcagaaaccggatggcaccattaaactgctgatttattataccagccgcctgcatagcggcgtgccgagccgctttagcggcagcggcagcggcaccgattatagcctgaccattagcaacctggaacaggaagatattgcgacctatttttgccagcagggcagcaccctgccgcgcacctttggcggcggcacc

SEQ ID NO: 142 - protein, 121 aa, ARTIFICIAL
  RECOMBINANT
EVQLQQSGPGLVAPSQSLSITCTVSGFSLTGYGVNWVRQPPGKGLEWLGMIWGDGSTDYNSALKSRLSISKDNSKSQVFLKMNSLQTDDTARYYCARDGYYYGTTWYFDVWGAGTTVTVSS

SEQ ID NO: 143 - DNA, 363 bp, ARTIFICIAL
  RECOMBINANT
gaagtgcagctgcagcagagcggcccgggcctggtggcgccgagccagagcctgagcattacctgcaccgtgagcggctttagcctgaccggctatggcgtgaactgggtgcgccagccgccgggcaaaggcctggaatggctgggcatgatttggggcgatggcagcaccgattataacagcgcgctgaaaagccgcctgagcattagcaaagataacagcaaaagccaggtgtttctgaaaatgaacagcctgcagaccgatgataccgcgcgctattattgcgcgcgcgatggctattattatggcaccacctggtattttgatgtgtggggcgcgggcaccaccgtgaccgtgagcagc

SEQ ID NO: 144 - protein, 102 aa, ARTIFICIAL
  RECOMBINANT
DIQMTQTTSSLSASLGDRVTISCRASQDISNYLNWYQQKPDGTIKLLIYYTSRLHSGVPSRFSGSGSGTDYSLTISNLEQEDIATYFCQQGSTLPRTFGGGT

SEQ ID NO: 145 - protein, 10 aa, ARTIFICIAL
  RECOMBINANT
GFSLTGYGVN

SEQ ID NO: 146 - protein, 16 aa, ARTIFICIAL
  RECOMBINANT
MIWGDGSTDYNSALKS

SEQ ID NO: 147 - protein, 13 aa, ARTIFICIAL
  RECOMBINANT
DGYYYGTTWYFDV

SEQ ID NO: 148 - protein, 11 aa, ARTIFICIAL
  RECOMBINANT
RASQDISNYLN

SEQ ID NO: 149 - protein, 7 aa, ARTIFICIAL
  RECOMBINANT
YTSRLHS

SEQ ID NO: 150 - protein, 9 aa, ARTIFICIAL
  RECOMBINANT
QQGSTLPRT

SEQ ID NO: 151 - protein, 12 aa, ARTIFICIAL
  RECOMBINANT
GGYDYDVPFFDY

SEQ ID NO: 152 - protein, 9 aa, ARTIFICIAL
  RECOMBINANT
QQGNMFPYT

SEQ ID NO: 153 - protein, 12 aa, ARTIFICIAL
  RECOMBINANT
GGYDYDVSFFDY

SEQ ID NO: 154 - protein, 16 aa, ARTIFICIAL
  RECOMBINANT
RSSQSIVHINRHTYLG

SEQ ID NO: 155 - protein, 7 aa, ARTIFICIAL
  RECOMBINANT
GVSNRFS

SEQ ID NO: 156 - protein, 9 aa, ARTIFICIAL
  RECOMBINANT
FQGTHVPFT

SEQ ID NO: 157 - protein, 9 aa, ARTIFICIAL
  RECOMBINANT
QQGNTLPYT

SEQ ID NO: 158 - protein, 335 aa, CANINE
ASTTAPSVFPLAPSCGSTSGSTVALACLVSGYFPEPVTVSWNSGSLTSGVHTFPSVLQSSGLYSLSSMVTVPSSRWPSETFTCNVAHPASKTKVDKPVPKRENGRVPRPPDCPKCPAPEAAGAPSVFIFPPKPKDTLLIARTPEVTCVVVDLDPEDPEVQISWFVDGKQMQTAKTQPREEQFNGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQESLSHSPGK

SEQ ID NO: 159 - DNA, 1005 bp, CANINE
gcctcaacaactgctcctagcgtgtttcccctggcccctagctgcggaagtacctcaggcagcacagtggccctggcttgtctggtgtctggatatttccctgagccagtgaccgtgagttggaacagcggctctctgacctccggggtgcacacatttccatctgtgctgcagtctagtggcctgtactccctgtcaagcatggtgactgtgccttcctctaggtggccatcagaaactttcacctgcaacgtggcccatcccgccagcaagaccaaagtggacaagcccgtgcctaaaagggagaatggaagggtgccaagaccacctgattgccctaagtgtccagctccagaagcggcgggagcaccaagcgtgttcatctttccacccaagcccaaagacacactgctgattgctagaactcccgaggtgacctgcgtggtggtggacctggatccagaggaccccgaagtgcagatctcctggttcgtggatgggaagcagatgcagacagccaaaactcagcctcgggaggaacagtttaacggaacctatagagtggtgtctgtgctgccaattggacaccaggactggctgaagggcaaacagtttacatgcaaggtgaacaacaaggccctgcctagtccaatcgagaggactatttcaaaagctaggggacaggctcatcagccttccgtgtatgtgctgcctccatcccgggaggaactgtctaagaacacagtgagtctgacttgtctgatcaaagatttctttccccctgacattgatgtggagtggcagagcaatgggcagcaggagccagaatccaagtacagaaccacaccaccccagctggacgaagatggctcctatttcctgtacagtaagctgtcagtggacaaatctaggtggcagcgcggggatacctttatctgcgccgtgatgcacgaggctctgcacaatcattacacacaagaaagtctgtcacatagccccggcaag

SEQ ID NO: 160 - protein, 106 aa, CANINE
GQPKASPSVTLFPPSSEELGANKATLVCLISDFYPSGVTVAWKADGSPVTQGVETTKPSKQSNNKYAASSYLSLTPDKWKSHSSFSCLVTHEGSTVEKKVAPAECS

SEQ ID NO: 161 - DNA, 318 bp, CANINE
ggccagccgaaagcgagcccgagcgtgaccctgtttccgccgagcagcgaagaactgggcgcgaacaaagcgaccctggtgtgcctgattagcgatttttatccgagcggcgtgaccgtggcgtggaaagcggatggcagcccggtgacccagggcgtggaaaccaccaaaccgagcaaacagagcaacaacaaatatgcggcgagcagctatctgagcctgaccccggataaatggaaaagccatagcagctttagctgcctggtgacccatgaaggcagcaccgtggaaaaaaaagtggcgccggcggaatgcagc

SEQ ID NO: 162 - protein, 335 aa, FELINE
ASTTAPSVFPLAPSCGTTSGATVALACLVLGYFPEPVTVSWNSGALTSGVHTFPAVLQASGLYSLSSMVTVPSSRWLSDTFTCNVAHPPSNTKVDKTVRKTDHPPGPKPCDCPKCPPPEMLGGPSIFIFPPKPKDTLSISRTPEVTCLVVDLGPDDSDVQITWFVDNTQVYTAKTSPREEQFNSTYRVVSVLPILHQDWLKGKEFKCKVNSKSLPSPIERTISKAKGQPHEPQVYVLPPAQEELSRNKVSVTCLIKSFHPPDIAVEWEITGQPEPENNYRTTPPQLDSDGTYFVYSKLSVDRSHWQRGNTYTCSVSHEALHSHHTQKSLTQSPGK

SEQ ID NO: 163 - DNA, 1005 bp, FELINE
gcctccaccacggccccatcggtgttcccactggcccccagctgcgggaccacatctggcgccaccgtggccctggcctgcctggtgttaggctacttccctgagccggtgaccgtgtcctggaactccggcgccctgaccagcggtgtgcacaccttcccggccgtcctgcaggcctcggggctgtactctctcagcagcatggtgacagtgccctccagcaggtggctcagtgacaccttcacctgcaacgtggcccacccgcccagcaacaccaaggtggacaagaccgtgcgcaaaacagaccacccaccgggacccaaaccctgcgactgtcccaaatgcccaccccctgagatgcttggaggaccgtccatcttcatcttccccccaaaacccaaggacaccctctcgatttcccggacgcccgaggtcacatgcttggtggtggacttgggcccagatgactccgatgtccagatcacatggtttgtggataacacccaggtgtacacagccaagacgagtccgcgtgaggagcagttcaacagcacctaccgtgtggtcagtgtcctccccatcctacaccaggactggctcaaggggaaggagttcaagtgcaaggtcaacagcaaatccctcccctcccccatcgagaggaccatctccaaggccaaaggacagccccacgagccccaggtgtacgtcctgcctccagcccaggaggagctcagcaggaacaaagtcagtgtgacctgcctgatcaaatccttccacccgcctgacattgccgtcgagtgggagatcaccggacagccggagccagagaacaactaccggacgaccccgccccagctggacagcgacgggacctacttcgtgtacagcaagctctcggtggacaggtcccactggcagaggggaaacacctacacctgctcggtgtcacacgaagctctgcacagccaccacacacagaaatccctcacccagtctccgggtaaa

SEQ ID NO: 164 - protein, 110 aa, FELINE
RSDAQPSVFLFQPSLDELHTGSASIVCILNDFYPKEVNVKWKVDGVVQNKGIQESTTEQNSKDSTYSLSSTLTMSSTEYQSHEKFSCEVTHKSLASTLVKSFQRSECQRE

SEQ ID NO: 165 - protein, 110 aa, FELINE; identical to SEQ ID NO: 164

SEQ ID NO: 166 - DNA, 336 bp, FELINE
cggagtgatgctcagccatctgtctttctcttccaaccatctctggacgagttacatacaggaagtgcctctatcgtgtgcatattgaatgacttctaccccaaagaggtcaatgtcaagtggaaagtggatggcgtagtccaaaacaaaggcatccaggagagcaccacagagcagaacagcaaggacagcacctacagcctcagcagcaccctgacgatgtccagtacggagtaccaaagtcatgaaaagttctcctgcgaggtcactcacaagagcctggcctccaccctcgtcaagagcttccagaggagcgagtgtcagagagagtgatga

SEQ ID NO: 167 - protein, 11 aa, ARTIFICIAL
  RECOMBINANT
KASQDINHYLN

SEQ ID NO: 168 - protein, 6 aa, ARTIFICIAL
  RECOMBINANT
TTRLQA

SEQ ID NO: 169 - protein, 9 aa, ARTIFICIAL
  RECOMBINANT
QQGDHFPRT

SEQ ID NO: 170 - protein, 6 aa, ARTIFICIAL
  RECOMBINANT
THSLHN

SEQ ID NO: 171 - protein, 6 aa, ARTIFICIAL
  RECOMBINANT
TSSLHG

SEQ ID NO: 172 - protein, 6 aa, ARTIFICIAL
  RECOMBINANT
TSSLHQ

SEQ ID NO: 173 - protein, 6 aa, ARTIFICIAL
  RECOMBINANT
TSSLHV

SEQ ID NO: 174 - protein, 6 aa, ARTIFICIAL
  RECOMBINANT
TASLHQ

SEQ ID NO: 175 - protein, 102 aa, ARTIFICIAL
  RECOMBINANT
DIVMTQTPLSLSVSPGEPASISCKASQDINHYLNWFRQKPDGTVKLLIYTTRLQASGVPSRFSGSGSGTDFTLRISRVEADDAGVYYCQQGDHFPRTFGQGT

SEQ ID NO: 176 - DNA, 306 bp, ARTIFICIAL
  RECOMBINANT
gatattgtgatgacccagaccccgctgagcctgagcgtgagcccgggcgaaccggcgagcattagctgcaaagcgagccaggatattaaccattatctgaactggtttcgccagaaaccggatggcaccgtgaaactgctgatttataccacccgcctgcaggcgagcggcgtgccgagccgctttagcggcagcggcagcggcaccgattttaccctgcgcattagccgcgtggaagcggatgatgcgggcgtgtattattgccagcagggcgatcattttccgcgcacctttggccagggcacc

SEQ ID NO: 177 - skipped

SEQ ID NO: 178 - protein, 7 aa, ARTIFICIAL
  Y-(X6)-S-(X7) -(X8)-H-S
  VARIANT (2)..(2): REPLACE= Ior T
  VARIANT (4)..(4): REPLACE= R or S
  VARIANT (5)..(5): REPLACE= L or F
YXSXXHS

SEQ ID NO: 179 - protein, 9 aa, ARTIFICIAL
  (X9) -(X10) -(X11) -(X12) -(X13) -(X14)-P-(X15) -(X16)
  VARIANT (1)..(1): REPLACE= Q or H
  VARIANT (2)..(2): REPLACE= Q or R
  VARIANT (3)..(3): REPLACE= G or A
  VARIANT (4)..(4): REPLACE= D,S,T or N
  VARIANT (5)..(5): REPLACE= H,T or M
  VARIANT (6)..(6): REPLACE= F, L or S
  VARIANT (8)..(8): REPLACE= R,Y, or G
  VARIANT (9)..(9): REPLACE= T or P
XXXXXXPXX

SEQ ID NO: 180 - protein, 11 aa, ARTIFICIAL
  (X1)-A-S-Q-(X2)-I-(X3) -(X4) -(X5)-L-N
  VARIANT (1)..(1): REPLACE= K or R
  VARIANT (5)..(5): REPLACE=S or D
  VARIANT (7)..(7): REPLACE=N or S
  VARIANT (8)..(8): REPLACE=H or N
  VARIANT (9)..(9): REPLACE=Y or N
XASQXIXXXLN

SEQ ID NO: 181 - protein, 6 aa, ARTIFICIAL
  T-(X6) -(X7)-L-(X8) -(X9)
  VARIANT (2)..(2): REPLACE= T,H,S, or A
  VARIANT (3)..(3): REPLACE= R or S
  VARIANT (5)..(5): REPLACE= Q or H
  VARIANT (6)..(6): REPLACE= A,Q,G or V
TXXLXX

SEQ ID NO: 182 - protein, 9 aa, ARTIFICIAL
  (X10) -(X11) -(X12) -(X13) -(X14) -(X15)-P-(X16) -(X17)
  VARIANT (1)..(1): REPLACE= Q or H
  VARIANT (2)..(2): REPLACE= Q or R
  VARIANT (3)..(3): REPLACE= G or A
  VARIANT (4)..(4): REPLACE= D,S,T or N
  VARIANT (5)..(5): REPLACE= H,T or M
  VARIANT (6)..(6): REPLACE= F,L or S
  VARIANT (8)..(8): REPLACE=R,Y or G
  VARIANT (9)..(9): REPLACE= T or P
XXXXXXPXX

SEQ ID NO: 183 - protein, 102 aa, ARTIFICIAL; identical to SEQ ID NO: 175
  RECOMBINANT

SEQ ID NO: 184 - protein, 335 aa, CANINE; identical to SEQ ID NO: 158

SEQ ID NO: 185 - DNA, 1005 bp, CANINE; identical to SEQ ID NO: 159